(19) United States
(12) Patent Application Publication
(10) Pub. No.: US 2009/0142294 A1
(43) Pub. Date: Jun. 4, 2009

(54) PARAMETER SELECTED GM-CSF, IL-3, IL-4, IL-5 AND CHIMERAS THEREOF FOR THERAPEUTIC AND DIAGNOSTIC PURPOSES

(75) Inventors: John D. Priest, Balmain (AU); Alan D. Watts, Dee Why (AU); Jason S. Whittaker, Redfern East (AU); Glenn R. Pilkington, Rye (AU); Catherine A. Liddell, Kingsgrove (AU); Ingrid Boehm, Bondi Junction (AU)

Correspondence Address: SEED INTELLECTUAL PROPERTY LAW GROUP PLLC, 701 FIFTH AVE, SUITE 5400, SEATTLE, WA, 98104, US

(73) Assignee: APOLLO LIFE SCIENCES LIMITED, Beaconsfield (AU)

(21) Appl. No.: 11/814,476

(22) PCT Filed: Jan. 25, 2006
(86) PCT No.: PCT/AU06/00092

§ 371 Date: Nov. 10, 2008

Related U.S. Application Data
(60) Provisional application No. 60/646,907, filed on Jan. 25, 2005.
(60) Provisional application No. 60/654,054, filed on Feb. 16, 2005.
(60) Provisional application No. 60/656,120, filed on Feb. 23, 2005.
(60) Provisional application No. 60/661,138, filed on Mar. 9, 2005.

(30) Foreign Application Priority Data
Nov. 10, 2005 (AU) 2005906320
Nov. 10, 2005 (AU) 2005906321
Nov. 15, 2005 (AU) 2005906338

(51) Int. Cl. A61K 38/20; C07K 14/54; A61K 39/395; G01N 33/53; C12N 15/11; C07K 16/18

(52) U.S. Cl. 424/85.2; 530/351; 530/391.1; 424/178.1; 536/23.5; 435/7.21

(57) ABSTRACT

The present invention relates generally to the fields of proteins, diagnostics, therapeutics and nutrition. More particularly, the present invention provides an isolated protein molecule in or related to the short chain 4 helix bundle superfamily such as GM-CSF, IL-3, IL-4 and IL-5 or chimeric molecules thereof comprising at least a portion of the protein molecule, such as GM-CSF-Fc, IL-3-Fc, IL-4-Fc and IL-5-Fc; wherein the protein or chimeric molecule thereof has a profile of measurable physiochemical parameters, wherein the profile is indicative of, associated with or forms the basis of one or more pharmacological traits. The present invention further contemplates the use of the isolated protein or chimeric molecule thereof in a range of diagnostic, prophylactic, therapeutic, nutritional and/or research applications.

### BACKGROUND OF THE INVENTION

1. Field of the Invention

The present invention relates generally to the fields of proteins, diagnostics, therapeutics and nutrition. More particularly, the present invention provides an isolated protein molecule in or related to the short chain 4 helix bundle superfamily such as GM-CSF, IL-3, IL-4 and IL-5 or chimeric molecules thereof comprising at least a portion of the protein molecule, such as GM-CSF-Fc, IL-3-Fc, IL-4-Fc and IL-5-Fc; wherein the protein or chimeric molecule thereof has a profile of measurable physiochemical parameters, wherein the profile is indicative of, associated with or forms the basis of one or more pharmacological traits. The present invention further contemplates the use of the isolated protein or chimeric molecule thereof in a range of diagnostic, prophylactic, therapeutic, nutritional and/or research applications.

2. Description of the Prior Art

Reference to any prior art in this specification is not, and should not be taken as an acknowledgment or any form of suggestion that this prior art forms a part of the common general knowledge.

Cytokines and growth factors involved in hematopoiesis are pleotropic. They contribute to blood cell proliferation, differentiation and development. Additionally, they play a major role in immunity by performing tasks such as stimulating phagocytosis and cytotoxicity of white blood cells, and promoting the production of other cytokines. Examples of cytokines and growth factors involved in hematopoiesis include GM-CSF, Interleukin-3 (IL-3), Interleukin-4 (IL-4) and Interleukin-5 (IL-5). These cytokines belong to the short chain 4 helix bundle superfamily.

GM-CSF is a glycoprotein that is involved in hematopoiesis, cell migration and immunity. GM-CSF is crucial for the growth and development of granulocyte and macrophage progenitor cells. GM-CSF acts synergistically with EPO in the proliferation of erythroid and megakaryocytic progenitor cells. It stimulates the proliferation, differentiation and phagocytic activity of neutrophilic, eosinophilic, and monocytic lineages and the cytotoxicity of eosinophils. GM-CSF also facilitates antigen presenting cell function by up-regulating the expression of MHCII and stimulates production of pro-inflammatory cytokines. Many of the clinical uses of human recombinant GM-CSF are based upon its myeloproliferative effects. GM-CSF is used in the treatment of several types of leukaemia, including acute myeloid leukaemia and acute lymphoblastic leukaemia, and various pathologies associated with anti-cancer chemotherapy, such as leukopenia and neutropenia.

Human IL-3 is a glycoprotein containing two N-linked glycosylation sites and exists as a monomer. IL-3 is predominantly produced by activated T cells however expression has also been detected in monocytes and macrophages, NK-cells, mast cells, endothelial cells, and keratinocytes. IL-3 was originally cloned as a mast cell growth factor. However, it has subsequently been shown to exhibit pleiotropic characteristics promoting the proliferation, maturation, and survival of progenitor cells of the myeloid, erythroid, and megakaryocyte lineage. IL-3 is a priming factor for hematopoietic stem cells in vitro and in vivo, and up-regulates the expression of receptors for other colony stimulating factors. It promotes phagocytosis in stimulated macrophages and up-regulates secretion of cytokines IL-1, IL-6, and TNF. IL-3 stimulation of mast cells induces the synthesis of histamines and the expression of complement factor C3a receptors on basophils. IL-3 has been shown to recruit eosinophils and promotes increased platelet levels and neutrophil numbers. The major potential for IL-3 in clinical applications is dependent upon its capability to promote the survival, proliferation and maintenance of hematopoietic progenitor cells. It is therefore effective either alone or in combination with other growth factors such as GM-CSF and G-CSF in treating bone marrow disorders such as myelodysplastic syndromes (MDS), and associated peripheral blood cytopenias, anemia, leukopenia and thrombocytopenia.

Human IL-4 is a glycoprotein that is structurally related to GM-CSF, M-CSF, and growth hormone. IL-4 enhances the proliferation and maturation of human thymocytes, up regulating T cell antigens (CD3, CD5 and the TCR). Additionally IL-4 may mediate B cell differentiation and isotype switching following activation stimuli. In non-leukocyte somatic cells, IL-4 acts as chemo-attractant for fibroblasts and stimulates dermal fibroblasts to secrete extra cellular matrix proteins such as collagen and fibronectin. IL-4 also stimulates the up-regulation of vascular cell adhesion molecule-1 (VCAM-1) in endothelial cells and consequently increases the adhesiveness of endothelial cells for T cells, basophils and eosinophils. IL-4 also has a direct inhibitory effect on the in vitro growth of human malignant cells including, colon cancer, human renal cancer, malignant melanoma and breast cancer cells.

Human IL-5 is a homodimer glycoprotein, which is predominantly produced by T-lymphocytes and mast cells, and to a lesser extent by eosinophils, natural killer cells and endothelial cells. The major in vivo function of IL-5 is the production, survival, differentiation and activation of eosinophils as well as promotion of eosinophil degranulation. These characteristics are exhibited by the immune system in response to parasitic infection and during allergic states. The specificity of IL-5 for promoting eosinophilia has been demonstrated by studies that show IL-5 neutralizing antibodies inhibit eosinophil production following parasitic infection. Additionally, IL-5 has also been shown to activate eosinophils, which is displayed as membrane ruffling, elongation, granule localization and increased oxidative metabolism. Furthermore, IL-5 has been reported to display chemotactic activity for eosinophils and the ability to promote histamine production in basophils.

The biological effector functions exerted by proteins via interaction with their respective binding proteins means that the short chain 4 helix bundle superfamily and its related proteins and their respective ligands or receptors may have significant potential as therapeutic agents to modulate physiological processes. However, minor changes to the molecule such as primary, secondary, tertiary or quaternary structure and co- or post-translational modification patterns can have a significant impact on the activity, secretion, antigenicty and clearance of the protein. It is possible, therefore, that the proteins can be generated with specific primary, secondary, tertiary or quaternary structure, or co- or post-translational structure or make-up that confer unique or particularly useful properties. There is consequently a need to evaluate the physiochemical properties of proteins under different conditions of production to determine whether they have useful physiochemical characteristics or other pharmacological traits.

The problem to date is that production of commercially available proteins are carried out in cells derived from species that are evolutionary distant to humans, cells such as bacteria, yeast, fungi, and insect. These cells express proteins that either lack glycosylation or exhibit glycosylation repertoires that are distinct to human cells and this impacts substantially on their clinical utility. For example, proteins expressed in yeast or fungi systems such as Aspergillus possess a high density of mannose which makes the protein therapeutically useless (Herscovics et al. FASEB J 7:540-550, 1993).

Even in non-human mammalian expression systems such as Chinese hamster ovary (CHO) cells, significant differences in the glycosylation patterns are documented compared with that of human cells. For example, most mammals, including rodents, express the enzyme (α 1,3) galactotransferase, which generates Gal (α 1,3)-Gal (β 1,4)-GlcNAc oligosaccharides on glycoproteins. However in humans, apes and Old World monkeys, the expression of this enzyme has become inactivated through a frameshift mutation in the gene. (Larsen et al. J Biol Chem 265:7055-7061, 1990) Although most of the CHO cell lines used for recombinant protein synthesis, such as Dux-B11, have inactivated the gene expressing (α 1,3) Galactotransferase, they still lack a functional (α 2,6) sialyltransferase enzyme for synthesis of (α 2,6)-linked terminal sialic acids which are present in human cells. Furthermore, the sialic acid motifs present on CHO cell expressed glycoproteins proteins are prone to degradation by a CHO cell endogenous sialidase (Gramer et al. Biotechnology 13 (7):692-8, 1995). As a result, proteins produced from these non-human expression systems will exhibit physiochemical and pharmacological characteristics such as half-life, antigenicity, stability and functional potency that are distinct from human cell-derived proteins.

The recent advancement of stem cell technology has substantially increased the potential for utilizing stem cells in applications such as transplantation therapy, drug screening, toxicology studies and functional genomics. However, stem cells are routinely maintained in culture medium that contains non-human proteins and are therefore not suitable for clinical applications due to the possibility of contamination with non-human infectious material. Furthermore, culturing of stem cells in non-human derived media may result in the incorporation of non-human carbohydrate moieties thus compromising transplant application (Martin et al. Nature Medicine 11 (2):228-232, 2005). Hence, the use of specific human-derived proteins in the maintenance and/or differentiation of stem cells will ameliorate the incorporation of xenogeneic proteins and enhance stem cell clinical utility.

Accordingly, there is a need to develop proteins and their receptors which have particularly desired physiochemical and pharmacological properties for use in diagnostic, prophylactic, therapeutic and/or nutritional research applications and the present invention provides proteins belonging to the short chain 4 helix bundle superfamily and its related proteins for clinical, commercial and research applications.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention relates generally to an isolated protein or chimeric molecule thereof in or related to the short chain 4 helix bundle superfamily comprising a profile of physiochemical parameters, wherein the profile is indicative of, associated with, or forms the basis of one or more distinctive pharmacological traits. More particularly, the present invention provides an isolated protein or chimeric molecule thereof selected from the list of GM-CSF, GM-CSF-Fc, IL-3, IL-3-Fc, IL-4, IL-4-Fc, IL-5 and IL-5-Fc comprising a physiochemical profile comprising a number of measurable physiochemical parameters, {[Pₓ]₁, [Pₓ]₂, . . . [Pₓ]ₙ,}, wherein Pₓ represents a measurable physiochemical parameter and “n” is an integer ≧1, wherein each parameter between and including [Pₓ]₁ to [Pₓ]ₙ is a different measurable physiochemical parameter, wherein the value of any one or more of the measurable physiochemical characteristics is indicative of, associated with, or forms the basis of, a distinctive pharmacological trait, T_{y}, or series of distinctive pharmacological traits {[T_{y}]₁, [T_{y}]₂, . . . [T_{y}]ₘ} wherein T_{y} represents a distinctive pharmacological trait and m is an integer ≧1 and each of [T_{y}]₁ to [T_{y}]ₘ is a different pharmacological trait.

As used herein the term “distinctive” with regard to a pharmacological trait of a protein or chimeric molecule thereof of the present invention refers to one or more pharmacological traits of a protein or chimeric molecule thereof which are distinctive for the particular physiochemical profile. In a particular embodiment, one or more of the pharmacological traits of an isolated protein or chimeric molecule thereof is different from, or distinctive relative to a form of the same protein or chimeric molecule thereof produced in a prokaryotic or lower eukaryotic cell or even a higher eukaryotic cell of a non-human species. In another embodiment, the pharmacological traits of a subject isolated protein or chimeric molecule thereof contribute to a desired functional outcome. As used herein, the term “measurable physiochemical parameters” or Px refers to one or more measurable characteristics of the isolated protein or chimeric molecule thereof. In a particular embodiment of the present invention, the measurable physiochemical parameters of a subject isolated protein or chimeric molecule thereof contribute to or are otherwise responsible for the derived pharmacological trait, Ty.

An isolated protein or chimeric molecule of the present invention comprises physiochemical parameters (Pₓ) which taken as a whole define protein molecule or chimeric molecule. The physiochemical parameters may be selected from the group consisting of apparent molecular weight (P₁), isoelectric point (pI) (P₂), number of isoforms (P₃), relative intensities of the different number of isoforms (P₄), percentage by weight carbohydrate (P₅), observed molecular weight following N-linked oligosaccharide deglycosylation (P₆), observed molecular weight following N-linked and O-linked oligosaccharide deglycosylation (P₇), percentage acidic monosaccharide content (P₈), monosaccharide content (P₉), sialic acid content (P₁₀), sulfate and phosphate content (P₁₁), Ser/Thr:GalNAc ratio (P₁₂), neutral percentage of N-linked oligosaccharide content (P₁₃), acidic percentage of N-linked oligosaccharide content (P₁₄), neutral percentage of O-linked oligosaccharide content (P₁₅), acidic percentage of O-linked oligosaccharide content (P₁₆), ratio of N-linked oligosaccharides (P₁₇), ratio of O-linked oligosaccharides (P₁₈), structure of N-linked oligosaccharide fraction (P₁₉), structure of O-linked oligosaccharide fraction (P₂₀), position and make up of N-linked oligosaccharides (P₂₁), position and make up of O-linked oligosaccharides (P₂₂), co-translational modification (P₂₃), post-translational modification (P₂₄), acylation (P₂₅), acetylation (P₂₆), amidation (P₂₇), deamidation (P₂₈), biotinylation (P₂₉), carbamylation or carbamoylation (P₃₀), carboxylation (P₃₁), decarboxylation (P₃₂), disulfide bond formation (P₃₃), fatty acid acylation (P₃₄), myristoylation (P₃₅), palmitoylation (P₃₆), stearoylation (P₃₇), formylation (P₃₈), glycation (P₃₉), glycosylation (P₄₀), glycophosphatidylinositol anchor (P₄₁), hydroxylation (P₄₂), incorporation of selenocysteine (P₄₃), lipidation (P₄₄), lipoic acid addition (P₄₅), methylation (P₄₆), N- or C-terminal blocking (P₄₇), N- or C-terminal removal (P₄₈), nitration (P₄₉), oxidation of methionine (P₅₀), phosphorylation (P₅₁), proteolytic cleavage (P₅₂), prenylation (P₅₃), farnesylation (P₅₄), geranyl geranylation (P₅₅), pyridoxal phosphate addition (P₅₆), sialylation (P₅₇), desialylation (P₅₈), sulfation (P₅₉), ubiquitinylation or ubiquitination (P₆₀), addition of ubiquitin-like molecules (P₆₁), primary structure (P₆₂), secondary structure (P₆₃), tertiary structure (P₆₄), quaternary structure (P₆₅), chemical stability (P₆₆), thermal stability (P₆₇). A list of these parameters is summarized in Table 2.

In a particular embodiment, a GM-CSF of the present invention is characterized by a profile of one or more physiochemical parameters (Pₓ) and pharmacological traits (T_{y}) comprising an apparent molecular weight (P₁) of 5 to 60 such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 56, 57, 58, 59, 60 and in a particular embodiment 16-40 kDa. The pI (P₂) of GM-CSF of the present invention is about 1 to 10, such as 1, 2, 3, 4, 5, 6, 7, 8, 9, 10, and in a particular embodiment 2-7 with at least 1 to 36 isoforms such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and in a particular embodiment 10-30 isoforms (P₃). The percentage by weight carbohydrate (P₅) of the GM-CSF of the present invention is about 1 to 99, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and in a particular embodiment 0-76% and in a further embodiment 0-65%. The observed molecular weight of the molecule after the N-linked oligosaccharides are removed (P₆) is 15-30 kD and in a particular embodiment is between 15-25 kD and the observed molecular weight of the molecule after the N-linked and O-linked oligosaccharides are removed (P₇) is 14-25 kD and in a particular embodiment is between 14 and 20 kD. The percentage acidic monosaccharide content (P₈) of the GM-CSF of the present invention is about 2 to 20% such as 2, 3, 4, 5, 6, 7, 8, 9, 10, 11, 12, 13, 14, 15, 16, 17, 18, 19, 20% and in a particular embodiment 6-11%. Monosaccharide (P₉) and sialic acid (P₁₀) content of the GM-CSF of the present invention, when normalized to GalNAc, is 1 to 0-3 fucose, 1 to 1-16 GlcNAc, 0.1 to 0.1-9 galactose, 1 to 0.1-9 mannose and 1 to 0-5 NeuNAc and in a particular embodiment is 1 to 0.1-1.5 fucose, 1 to 2-12 GlcNAc, 1 to 1.0-6.0 galactose, 1 to 1.0-6.0 mannose and 1 to 0-3.0 NeuNAc; when normalized to 3 times of mannose, is 3 to 0-5 fucose, 3 to 0.1-3 GalNAc, 3 to 2-15 GlcNAc, 3 to 1-6 galactose and 3 to 0-4 NeuNAc and in a particular embodiment is 3 to 0.1-2.5 fucose, 3 to 0.5-2.5 GalNAc, 3 to 5.0-10.0 GlcNAc, 3 to 2.0-5.0 galactose and 3 to 0-3.0 NeuNAc. Neutral percentage of N-linked oligosaccharides (P₁₃) is about 40 to 90%, such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 49 to 83%, in an additional embodiment 54 to 78%, and in a further embodiment 59 to 73%. Acidic percentage of N-linked oligosaccharides (P₁₄) is about 10% to 70%, in a particular embodiment 17% to 51%, in a an additional embodiment 22% to 46%, and in a further embodiment 27 to 41%. Neutral percentage of O-linked oligosaccharides (P₁₅) is about 5 to 90% such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 9 to 82%, in an additional embodiment 29 to 62% in a further embodiment 34 to 57%. Acidic percentage of O-linked oligosaccharides (P₁₆) is about 10 to 100%, in a particular embodiment 18 to 91%, in an additional embodiment 38 to 71% and in a further embodiment 43 to 66%. The sites of N-glycosylation (P₂₁) of the GM-CSF of the present invention include N-44 and N-54 (numbering from the start of the signal sequence) identified by PMF after PNGase treatment. The serum/plasma stability (T₁₀) of GM-CSF of the present invention is distinct from that of human GM-CSF expressed in non human cells, in particular the GM-CSF of the present invention exhibited greater proliferative activity on TF-1 cells following a 24 hour incubation in fetal calf serum than human GM-CSF produced from E. coli

The proliferation ability (T₃₂) of the GM-CSF of the present invention is distinct from that of a human GM-CSF expressed in a non-human cell system, in particular, the proliferation ability (T₃₂) of the GM-CSF of the present invention is greater than that of a human GM-CSF expressed in a non-human cell system. The differentiation ability (T₃₃) of the GM-CSF of the present invention is distinct from that of a human GM-CSF expressed in a non-human cell system, in particular the GM-CSF of the present invention had a greater capacity to induce colony formation in TF-1 cells than human GM-CSF expressed in E. coli.

In a particular embodiment, an IL-3 molecule of the present invention is characterized by a profile of one or more physiochemical parameters (Pₓ) and pharmacological traits (T_{y}) comprising an apparent molecular weight (P₁) of 1 to 250, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250 kDa and in a particular embodiment 15 to 35 kDa. The pI (P₂) of IL-3 molecule is 2 to 14 such as 2, 3, 4, 5, 6, 7, 8, 9, 10, 11, 12, 13, 14 and in a particular embodiment 3.5-7.5 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 5-15 isoforms (P₃). The percentage by weight carbohydrate (P₅) of the IL-3 molecule of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 0 to 60%. The observed molecular weight of the IL-3 of the present invention when the N-linked oligosaccharides are removed (P₆) is between 10 and 25 kDa. Monosaccharide content (P₉) of the IL-3 molecule of the present invention, when normalized to GalNAc, are 1 to 0.1-8 fucose, 1 to 0.1-7 GlcNAc, 1 to 0.1-3 galactose, 1 to 0.1-3 mannose and 1 to 0-5 NeuAc; and in a particular embodiment 1 to 2-6 fucose, 1 to 3-5 GlcNAc, 1 to 0.5-2 galactose, 1 to 0.5-2 mannose and 1 to 0-2 NeuNAc; when normalized to 3 times of mannose, are 3 to 2-25 fucose, 3 to 0.1-6 GalNAc, 3 to 4-21 GlcNAc, 3 to 0.1-9 galactose and 3 to 0-5 NeuAc; in a particular embodiment 3 to 5-16 fucose, 3 to 2-4 GalNAc, 3 to 9-14 GlcNAc, 3 to 3-6 galactose and 3 to 0.1-2 NeuAc. The sialic acid content (P₁₀) expressed as a percentage of the monosaccharide content of the IL-3 molecule of the present invention is 0 to 50%, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% and in a particular embodiment 0 to 20%. Neutral percentage of N-linked oligosaccharides (P₁₃) of the IL-3 molecule of the present invention is 70 to 100%, in a particular embodiment 75 to 95% and in an additional embodiment 80 to 90%. Acidic percentage of N-linked oligosaccharides (P₁₄) of the IL-3 molecule of the present invention is 0 to 30%, in a particular embodiment 5 to 25% and in an additional embodiment 10 to 20%. The immunoreactivity profile (T₁₃) of the IL-3 of the present invention is distinct from that of a human IL-3 expressed in a non-human cell system, in particular, the protein concentration of the IL-3 of the present invention is underestimated when assayed using an ELISA kit which contains a human IL-3 expressed in a non-human cell system. In addition, the immunoreactivity profile (T₁₃) of the IL-3 of the present invention is distinct from that of a human IL-3 expressed in insect cells. The proliferation ability (T₃₂) of the IL-3 of the present invention is distinct from that of a human IL-3 expressed in a non-human cell system, in particular, the proliferation ability (T₃₂) of the IL-3 of the present invention is greater than that of a human IL-3 expressed in a non-human cell system.

In a particular embodiment, a GM-CSF of the present invention is characterized by a profile of one or more physiochemical parameters (Pₓ) and pharmacological traits (T_{y}) comprising an apparent molecular weight (P₁) of 5 to 60 such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 56, 57, 58, 59, 60 and in a particular embodiment 16-40 kDa. The pI (P₂) of GM-CSF of the present invention is about 1 to 10, such as 1, 2, 3, 4, 5, 6, 7, 8, 9, 10, and in a particular embodiment 2-7 with at least 1 to 36 isoforms such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and in a particular embodiment 10-30 isoforms (P₃). The percentage by weight carbohydrate (P₅) of the GM-CSF of the present invention is about 1 to 99, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and in a particular embodiment 0-76% and in a further embodiment 0-65%. The observed molecular weight of the molecule after the N-linked oligosaccharides are removed (P₆) is 10-35 kDa and in a particular embodiment is between 12-30 kDa and the observed molecular weight of the molecule after the N-linked and O-linked oligosaccharides are removed (P₇) is 9-30 kDa and in a particular embodiment is between 11 and 25 kDa. The percentage acidic monosaccharide content (P₈) of the GM-CSF of the present invention is about 2 to 20% such as 2, 3, 4, 5, 6, 7, 8, 9, 10, 11, 12, 13, 14, 15, 16, 17, 18, 19, 20% and in a particular embodiment 6-11%. Monosaccharide (P₉) and sialic acid (P₁₀) content of the GM-CSF of the present invention, when normalized to GalNAc, is 1 to 0-3 fucose, 1 to 1-16 GlcNAc, 0.1 to 0.1-9 galactose, 1 to 0.1-9 mannose and 1 to 0-5 NeuNAc and in a particular embodiment is 1 to 0.1-1.5 fucose, 1 to 2-12 GlcNAc, 1 to 1.0-6.0 galactose, 1 to 1.0-6.0 mannose and 1 to 0-3.0 NeuNAc; when normalized to 3 times of mannose, is 3 to 0-5 fucose, 3 to 0.1-3 GalNAc, 3 to 2-15 GlcNAc, 3 to 1-6 galactose and 3 to 0-4 NeuNAc and in a particular embodiment is 3 to 0.1-2.5 fucose, 3 to 0.5-2.5 GalNAc, 3 to 5.0-10.0 GlcNAc, 3 to 2.0-5.0 galactose and 3 to 0-3.0 NeuNAc. Neutral percentage of N-linked oligosaccharides (P₁₃) is about 40 to 90%, such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 49 to 83%, in an additional embodiment 54 to 78%, and in a further embodiment 59 to 73%. Acidic percentage of N-linked oligosaccharides (P₁₄) is about 10% to 70%, in a particular embodiment 17% to 51%, in a an additional embodiment 22% to 46%, and in a further embodiment 27 to 41%. Neutral percentage of O-linked oligosaccharides (P₁₅) is about 5 to 90% such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 9 to 82%, in an additional embodiment 29 to 62% in a further embodiment 34 to 57%. Acidic percentage of O-linked oligosaccharides (P₁₆) is about 10 to 100%, in a particular embodiment 18 to 91%, in an additional embodiment 38 to 71% and in a further embodiment 43 to 66%. The sites of N-glycosylation (P₂₁) of the GM-CSF of the present invention include N-44 and N-54 (numbering from the start of the signal sequence) identified by PMF after PNGase treatment. The serum/plasma stability (T₁₀) of GM-CSF of the present invention is distinct from that of human GM-CSF expressed in non human cells, in particular the GM-CSF of the present invention exhibited greater proliferative activity on TF-1 cells following a 24 hour incubation in fetal calf serum than human GM-CSF produced from E. coli cells. The proliferation ability (T₃₂) of the GM-CSF of the present invention is distinct from that of a human GM-CSF expressed in a non-human cell system, in particular, the proliferation ability (T₃₂) of the GM-CSF of the present invention is 5-12 times greater than that of a human GM-CSF expressed in E. coli cells. The differentiation ability (T₃₃) of the GM-CSF of the present invention is distinct from that of a human GM-CSF expressed in a non-human cell system, in particular the GM-CSF of the present invention has a 1.5-2 fold greater capacity to induce colony formation in TF-1 cells than human GM-CSF expressed in E. coli cells.

In a particular embodiment, an IL-3 molecule of the present invention is characterized by a profile of one or more physiochemical parameters (Pₓ) and pharmacological traits (T_{y}) comprising an apparent molecular weight (P₁) of 1 to 250, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250 kDa and in a particular embodiment 15 to 35 kDa. The pI (P₂) of IL-3 molecule is 2 to 14 such as 2, 3, 4, 5, 6, 7, 8, 9, 10, 11, 12, 13, 14 and in a particular embodiment 3.5-7.5 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 5-15 isoforms (P₃). The percentage by weight carbohydrate (P₅) of the IL-3 molecule of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 0 to 60%. The observed molecular weight of the IL-3 of the present invention when the N-linked oligosaccharides are removed (P₆) is between 8 and 30 kDa and in a particular embodiment, between 10 and 25 kDa. Monosaccharide content (P₉) of the IL-3 molecule of the present invention, when normalized to GalNAc, are 1 to 0.1-8 fucose, 1 to 0.1-7 GlcNAc, 1 to 0.1-3 galactose, 1 to 0.1-3 mannose and 1 to 0-5 NeuAc; and in a particular embodiment 1 to 2-6 fucose, 1 to 3-5 GlcNAc, 1 to 0.5-2 galactose, 1 to 0.5-2 mannose and 1 to 0-2 NeuNAc; when normalized to 3 times of mannose, are 3 to 2-25 fucose, 3 to 0.1-6 GalNAc, 3 to 4-21 GlcNAc, 3 to 0.1-9 galactose and 3 to 0-5 NeuAc; in a particular embodiment 3 to 5-16 fucose, 3 to 2-4 GalNAc, 3 to 9-14 GlcNAc, 3 to 3-6 galactose and 3 to 0.1-2 NeuAc. The sialic acid content (P₁₀) expressed as a percentage of the monosaccharide content of the IL-3 molecule of the present invention is 0 to 50%, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% and in a particular embodiment 0 to 20%. Neutral percentage of N-linked oligosaccharides (P₁₃) of the IL-3 molecule of the present invention is 70 to 100%, in a particular embodiment 75 to 95% and in an additional embodiment 80 to 90%. Acidic percentage of N-linked oligosaccharides (P₁₄) of the IL-3 molecule of the present invention is 0 to 30%, in a particular embodiment 5 to 25% and in an additional embodiment 10 to 20%. The immunoreactivity profile (T₁₃) of the IL-3 of the present invention is distinct from that of a human IL-3 expressed in a non-human cell system, in particular, the protein concentration of the IL-3 of the present invention is underestimated when assayed using an ELISA kit which contains a human IL-3 expressed in a non-human cell system. In addition, the immunoreactivity profile (T₁₃) of the IL-3 of the present invention is distinct from that of a human IL-3 expressed in insect cells. The proliferation ability (T₃₂) of the IL-3 of the present invention is distinct from that of a human IL-3 expressed in a non-human cell system, in particular, the proliferation ability (T₃₂) of the IL-3 of the present invention is 1.1-2.5 times greater than that of a human IL-3 expressed in E. coli cells.

In a particular embodiment, an IL-4 molecule of the present invention is characterized by a profile of one or more physiochemical parameters (Pₓ) and pharmacological traits (T_{y}) comprising an apparent molecular weight (P₁) of 1 to 120, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, kDa and in a particular embodiment 12 to 24 kDa. The pI (P₂) of IL-4 is 2 to 14 such as 2, 3, 4, 5, 6, 7, 8, 9, 10, 11, 12, 13, 14 and in a particular embodiment 8 to 11 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 1-3 isoforms (P₃). The percentage by weight carbohydrate (P₅) of the IL-4 of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 0 to 25%. The observed molecular weight of the IL-4 of the present invention when the N-linked oligosaccharides are removed (P₆) is between 8 and 24 kDa and in a particular embodiment, between 10 and 20 kDa. The observed molecular weight of the IL-4 of the present invention when the N-linked oligosaccharides and O-linked oligosaccharides re removed (P₇) is between 8 and 22 kDa and in a particular embodiment, between 10 and 18 kDa. Neutral percentage of N-linked oligosaccharides (P₁₃) of the IL-4 of the present invention is 50 to 100%, in a particular embodiment 65 to 100% and in an additional embodiment 70 to 100%. Acidic percentage of N-linked oligosaccharides (P₁₄) of the IL-4 of the present invention is 0 to 50% in a particular embodiment 0 to 45% and in an additional embodiment 0 to 30%. The sites of N-glycosylation (P₂₁) of the IL-4 of the present invention include N-62 (numbering from the start of the signal sequence) identified by PMF after PNGase treatment. The sites of disulfide bond formation (P₃₃) include Cys27-Cys151, Cys48-Cys89 and Cys70-Cys123 (cysteine residues numbered from the start of the signal sequence).

In one embodiment, the immunoreactivity profile (T₁₃) of the IL-4 of the present invention is distinct from that of a human IL-4 expressed in a non-human cell system, in particular, the protein concentration of the IL-4 of the present invention is underestimated when assayed using an ELISA kit which contains a human IL-4 expressed in a non-human cell system. The proliferation ability (T₃₂) of the IL-4 of the present invention is distinct from that of a human IL-4 expressed in non-human cell systems, in particular, the proliferation ability (T₃₂) of the IL-4 of the present invention is 25-54 times greater than that of a human IL-4 expressed in E. coli cells; and the proliferation ability (T₃₂) of the IL-4 of the present invention is up to 1.75 fold greater proliferative activity than human IL-4 expressed in CHO cells. Further, the proliferation ability (T₃₂) of the IL-4 of the present invention is distinct from that of a human IL-4 expressed in a non-human cell system after extended pre-incubation at elevated temperatures, in particular, the proliferation ability (T₃₂) of the IL-4 of the present invention is 13-30 fold greater on TF-1 cells following a 4 day pre-incubation at 37° C. in cell culture medium than human GM-CSF expressed in E. coli cells.

In a particular embodiment, an IL-5 of the present invention is characterized by a profile of one or more physiochemical parameters (Pₓ) comprising an apparent molecular weight (P₁) of 1 to 250, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250 kDa and in a particular embodiment 15 to 25 kDa. The pI (P₂) of IL-5 of the present invention is 2 to 14 such as 2, 3, 4, 5, 6, 7, 8, 9, 10, 11, 12, 13, 14 and in a particular embodiment 4 to 9 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 5-12 isoforms (P₃). The percentage by weight carbohydrate (P₅) of the IL-5 of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 10 to 50%. The observed molecular weight of the IL-5 of the present invention when the N-linked oligosaccharides are removed (P₆) is between 9 to 30 kDa and in a particular embodiment, between 11 and 25 kDa. The observed molecular weight of the IL-5 of the present invention when the N-linked oligosaccharides and O-linked oligosaccharides re removed (P₇) is between 8 to 27 kDa and in a particular embodiment, between 10 and 22 kDa. Monosaccharide content (P₉) of the IL-5 of the present invention, when normalized to GalNAc, are 1 to 0.1-3 fucose, 1 to 0.5-7 GlcNAc, 1 to 0.05-3 galactose, 1 to 0.1-3 mannose and 1 to 0-5 NeuNAc; and in a particular embodiment 1 to 0-0.5 fucose, 1 to 2-4.5 GlcNAc, 1 to 1-2 galactose, 1 to 1-2 mannose and 1 to 0.1-1 NeuNAc; when normalized to 3 times of mannose, are 3 to 0.1-3 fucose, 3 to 0.1-4 GalNAc, 3 to 1-17 GlcNAc, 3 to 1-8 galactose and 3 to 0-5 NeuNAc; in a particular embodiment 3 to 0-1 fucose, 3 to 2-3 GalNAc, 3 to 3-12 GlcNAc, 3 to 2-5 galactose and 3 to 0.2-1 NeuNAc. The sialic acid content (P₁₀) expressed as a percentage of the monosaccharide content of the IL-5 of the present invention is 0 to 50%, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% and in a particular embodiment 2 to 10%. The sulfate content (P₁₁) of the IL-5 of the present invention, when normalized to GalNAc, are 1 to 2-14 sulfate; and in a particular embodiment 1 to 5-10 sulfate; when normalized to 3 times of mannose, are 3 to 7-36 sulfate and in a particular embodiment 3 to 12-24 sulfate. The sulfation (P₅₉) expressed as a percentage of the monosaccharide content of IL-5 of the present invention is 5-35% and in a particular embodiment 10-25%. Neutral percentage of N-linked oligosaccharides (P₁₃) of the IL-5 of the present invention is 30 to 90%, in a particular embodiment 40 to 80% and in an additional embodiment 50 to 75%. Acidic percentage of N-linked oligosaccharides (P₁₄) of the IL-5 of the present invention is 10 to 70%, in a particular embodiment 20 to 60% and in an additional embodiment 25 to 50%. Neutral percentage of O-linked oligosaccharides (P₁₅) of the IL-5 of the present invention is 40 to 100%, in a particular embodiment 50 to 100% and in an additional embodiment 60 to 100%. Acidic percentage of O-linked oligosaccharides (P₁₆) of the IL-5 of the present invention is 0 to 60%, in a particular embodiment 0 to 50% and in an additional embodiment 0 to 40%.

In a particular embodiment, the present invention contemplates an isolated form of protein or chimeric molecule thereof in or related to the short chain 4 helix bundle superfamily selected from the group comprising GM-CSF, GM-CSF-Fc, IL-3, IL-3-Fc, IL-4, IL-4-Fc, IL-5 and IL-5-Fc. An isolated protein or chimeric molecule of the present invention comprises distinctive pharmacological traits selected from the group comprising or consisting of therapeutic efficiency (T₁), effective therapeutic dose (TCID₅₀) (T₂), bioavailability (T₃), time between dosages to maintain therapeutic levels (T₄), rate of absorption (T₅), rate of excretion (T₆), specific activity (T₇), thermal stability (T₈), lyophilization stability (T₉), serum/plasma stability (T₁₀), serum half-life (T₁₁), solubility in blood stream (T₁₂), immunoreactivity profile (T₁₃), immunogenicity (T₁₄), inhibition by neutralizing antibodies (T_{14A}), side effects (T₁₅), receptor/ligand binding affinity (T₁₆), receptor/ligand activation (T₁₇), tissue or cell type specificity (T₁₈), ability to cross biological membranes or barriers (i.e. gut, lung, blood brain barriers, skin etc) (T₁₉), angiogenic ability (T_{19A}), tissue uptake (T₂₀), stability to degradation (T₂₁), stability to freeze-thaw (T₂₂), stability to proteases (T₂₃), stability to ubiquitination (T₂₄), ease of administration (T₂₅), mode of administration (T₂₆), compatibility with other pharmaceutical excipients or carriers (T₂₇), persistence in organism or environment (T₂₈), stability in storage (T₂₉), toxicity in an organism or environment and the like (T₃₀).

In addition, the protein or chimeric molecule of the present invention may have altered biological effects on different cells types (T₃₁), including without being limited to human primary cells, such as lymphocytes, erythrocytes, retinal cells, hepatocytes, neurons, keratinocytes, endothelial cells, endodermal cells, ectodermal cells, mesodermal cells, epithelial cells, kidney cells, liver cells, bone cells, bone marrow cells, lymph node cells, dermal cells, fibroblasts, T-cells, B-cells, plasma cells, natural killer cells, macrophages, granulocytes, neutrophils, Langerhans cells, dendritic cells, eosinophils, basophils, mammary cells, lobule cells, prostate cells, lung cells, oesophageal cells, pancreatic cells, Beta cells (insulin secreting cells), hemangioblasts, muscle cells, oval cells (hepatocytes), mesenchymal cells, brain microvessel endothelial cells, astrocytes, glial cells, various stem cells including adult and embryonic stem cells, various progenitor cells; and other human immortal, transformed or cancer cell lines.

The biological effects on the cells include effects on proliferation (T₃₂), differentiation (T₃₃), apoptosis (T₃₄), growth in cell size (T₃₅), cytokine adhesion (T₃₆), cell adhesion (T₃₇), cell spreading (T₃₈), cell motility (T₃₉), migration and invasion (T₄₀), chemotaxis (T₄₁), cell engulfment (T₄₂), signal transduction (T₄₃), recruitment of proteins to receptors/ligands (T₄₄), activation of the JAK/STAT pathway (T₄₅), activation of the Ras-erk pathway (T₄₆), activation of the AKT pathway (T₄₇), activation of the PKC pathway (T₄₈), activation of the PKA pathway (T₄₉), activation of src (T₅₀), activation of fas (T₅₁), activation of TNFR (T₅₂), activation of NFkB (T₅₃), activation of p38MAPK (T₅₄), activation of c-fos (T₅₅), secretion (T₅₆), receptor internalization (T₅₇), receptor cross-talk (T₅₈), up or down regulation of surface markers (T₅₉), alteration of FACS front/side scatter profiles (T₆₀), alteration of subgroup ratios (T₆₁), differential gene expression (T₆₂), cell necrosis (T₆₃), cell clumping (T₆₄), cell repulsion (T₆₅), binding to heparin sulfates (T₆₆), binding to glycosylated structures (T₆₇), binding to chondroitin sulfates (T₆₈), binding to extracellular matrix (such as collagen, fibronectin) (T₆₉), binding to artificial materials (such as scaffolds) (T₇₀), binding to carriers (T₇₁), binding to co-factors (T₇₂) the effect alone or in combination with other proteins on stem cell proliferation, differentiation and/or self-renewal (T₇₃) and the like. These are summarized in Table 3.

The present invention further provides a chimeric molecule comprising an isolated protein or a fragment thereof, such as an extra-cellular domain of a membrane bound protein, linked to the constant (Fc) or framework region of a human immunoglobulin via one or more protein linker. Such a chimeric molecule is also referred to herein as protein-Fc. Examples of such protein-Fc contemplated by the present invention include GM-CSF-Fc, IL-3-Fc, IL-4-Fc and IL-5-Fc.

Such protein-Fc has a profile of measurable physiochemical parameters indicative of or associated with one or more distinctive pharmacological traits of the isolated protein-Fc. Other chimeric molecules contemplated by the present invention include the protein or protein-Fc or a fragment thereof, linked to a lipid moiety such as a polyunsaturated fatty acid molecule. Such lipid moieties may be linked to an amino acid residue in the backbone of the molecule or to a side chain of such an amino acid residue.

The present invention further provides a chimeric molecule comprising an isolated protein or a fragment thereof, such as an extra-cellular domain of a membrane bound protein, linked to the constant (Fc) or framework region of a mammalian immunoglobulin via one or more protein linker. In another aspect, the mammal Fc or framework region of the immunoglobulin is derived from a mammal selected from the group consisting of primates, including humans, marmosets, orangutans and gorillas, livestock animals (e.g. cows, sheep, pigs, horses, donkeys), laboratory test animals (e.g. mice, rats, guinea pigs, hamsters, rabbits, companion animals (e.g. cats, dogs) and captured wild animals (e.g. rodents, foxes, deer, kangaroos). In another embodiment the Fc or framework region is a human immunoglobulin. In a particular embodiment the mammal is a human. Such a chimeric molecule is also referred to herein as protein-Fc. Other chimeric molecules contemplated by the present invention include the protein or protein-Fc or a fragment thereof linked to a lipid moiety such as a polyunsaturated fatty acid molecule. Such lipid moieties may be linked to an amino acid residue in the background of the molecule or to a side chain of such an amino acid residue. The chimeric molecules of the present invention, including GM-CSF-Fc, IL-3-Fc, IL-4-Fc and IL-5-Fc have a profile of measurable physiochemical parameters indicative of or associated with one or more distinctive pharmacological traits of the isolated protein-Fc.

Accordingly, the present invention provides an isolated polypeptide encoded by a nucleotide sequence selected from the list consisting of SEQ ID NOs: 25, 27, 29, 31, 35, 37, 39, 41, 43, 45, 47, 51, 53, 55, 57, 61, 63, 65, 67, or a nucleotide sequence having at least about 65% identity to any one of the above-listed sequence or a nucleotide sequence capable of hybridizing to any one of the above sequences or their complementary forms under low stringency conditions.

Another aspect of the present invention provides an isolated polypeptide encoded by a nucleotide sequence selected from the list consisting of SEQ ID NOs: 69, 70, 71, 72 following splicing of their respective mRNA species by cellular processes.

Yet another aspect of the present invention provides an isolated polypeptide comprising an amino acid sequence selected from the list consisting of SEQ ID NOs: 26, 28, 30, 32, 36, 38, 40, 42, 44, 46, 48, 52, 54, 56, 58, 62, 64, 66, 68, or an amino acid sequence having at least about 65% similarity to one or more of the above sequences.

The present invention further contemplates a pharmaceutical composition comprising at least part of the protein or chimeric molecule thereof, together with a pharmaceutically acceptable carrier, co-factor and/or diluent.

With respect to the primary structure, the present invention provides an isolated protein or chimeric molecule thereof, or a fragment thereof, encoded by a nucleotide sequence selected from the list consisting of SEQ ID NOs: 25, 27, 29, 31, 35, 37, 39, 41, 43, 45, 47, 51, 53, 55, 57, 61, 63, 65, 67, or a nucleotide sequence having at least about 60% identity to any one of the above-listed sequence or a nucleotide sequence capable of hybridizing to any one of the above sequences or their complementary forms under low stringency conditions.

Still, another aspect of the present invention provides an isolated nucleic acid molecule encoding protein or chimeric molecule thereof or a functional part thereof comprising a sequence of nucleotides having at least 60% similarity selected from the list consisting of SEQ ID NOs: 25, 27, 29, 31, 35, 37, 39, 41, 43, 45, 47, 51, 53, 55, 57, 61, 63, 65, 67 or after optimal alignment and/or being capable of hybridizing to one or more of SEQ ID NOs: 25, 27, 29, 31, 35, 37, 39, 41, 43, 45, 47, 51, 53, 55, 57, 61, 63, 65, 67 or their complementary forms under low stringency conditions.

In a particular embodiment, the present invention is directed to an isolated nucleic acid molecule comprising a sequence of nucleotides encoding a protein or chimeric molecule in or related to the short chain 4 helix bundle superfamily, selected from the group comprising GM-CSF, GM-CSF-Fc, IL-3, IL-3-Fc, IL-4, IL-4-Fc, IL-5 and IL-5-Fc, or a fragment thereof, an amino acid sequence substantially as set forth in one or more of SEQ ID NOs: 26, 28, 30, 32, 36, 38, 40, 42, 44, 46, 48, 52, 54, 56, 58, 62, 64, 66, 68 or an amino acid sequence having at least about 60% similarity to one or more of SEQ ID NOs: 26, 28, 30, 32, 36, 38, 40, 42, 44, 46, 48, 52, 54, 56, 58, 62, 64, 66, 68 after alignment.

In another aspect, the present invention provides an isolated nucleic acid molecule encoding a protein or chimeric molecule in or related to the short chain 4 helix bundle superfamily, selected from the group comprising GM-CSF, IL-3, IL-4 and IL-5, or a fragment thereof, comprising a sequence of nucleotides selected from the group consisting of SEQ ID NOs: 27, 29, 37, 39, 41, 43, 53, 55, 63, or 65, linked directly or via one or more nucleotide sequences encoding protein linkers known in the art to nucleotide sequences encoding the constant (Fc) or framework region of a human immunoglobulin, substantially as set forth in one or more of SEQ ID NOs:1, 3, 5, 7, 9, 11, 13, 15, 17 or 19. In a particular embodiment, the protein linker comprises IP, GSSNT, TRA or VDGIQWIP.

In another aspect, the present invention provides an isolated protein in or related to the short chain 4 helix bundle superfamily, selected from the group comprising GM-CSF, IL-3, IL-4 and IL-5, or a fragment thereof, comprising an amino acid sequence selected from the group consisting of SEQ ID NOs: 28, 30, 38, 40, 42, 44, 54, 56, 64, or 66 linked directly or via one or more protein linkers known in the art, to the constant (Fc) or framework region of a human immunoglobulin, substantially as set forth in one or more of SEQ ID NOs:2, 4, 6, 8, 10, 12, 14, 16, 18 or 20.

The present invention further extends to uses of an isolated protein or chimeric molecule thereof or nucleic acid molecules encoding same in diagnostic, prophylactic, therapeutic, nutritional and/or research applications. More particularly, the present invention extends to a method of treating or preventing a condition or ameliorating the symptoms of a condition in an animal subject, said method comprising administering to said animal subject an effective amount of an isolated protein or chimeric molecule thereof.

In addition, the present invention extends to uses of a protein or chimeric molecule thereof for screening small molecules, which may have a variety of diagnostic, prophylactic, therapeutic, nutritional and/or research applications.

The present invention further contemplates using an isolated protein or chimeric molecule thereof as immunogens to generate antibodies for therapeutic or diagnostic applications.

The present invention further contemplates using an isolated protein or chimeric molecule thereof in culture mediums for stem cells used in stem cell or related therapy.

The subject invention also provides the use of a protein or chimeric molecule thereof in the manufacture of a formulation for diagnostic, prophylactic, therapeutic, nutritional and/or research applications.

The subject invention also provides a human derived protein or chimeric molecule thereof for use as a standard protein in an immunoassay and kits thereof. The subject invention also extends to a method for determining the level of human cell-expressed human protein or chimeric molecule thereof in a biological preparation.

TABLE 1
Sequence Identifier

| Sequence Identifier | Sequence |
|---|---|
| SEQ ID NO: 1 | Human IgG1 Fc nucleotide sequence |
| SEQ ID NO: 2 | Human IgG1 Fc amino acid sequence |
| SEQ ID NO: 3 | Human IgG1 Fc nucleotide sequence (variant) |
| SEQ ID NO: 4 | Human IgG1 Fc amino acid sequence (variant) |
| SEQ ID NO: 5 | Human IgG2 Fc nucleotide sequence |
| SEQ ID NO: 6 | Human IgG2 Fc amino acid sequence |
| SEQ ID NO: 7 | Human IgG3 Fc nucleotide sequence |
| SEQ ID NO: 8 | Human IgG3 Fc amino acid sequence |
| SEQ ID NO: 9 | Human IgG4 Fc nucleotide sequence |
| SEQ ID NO: 10 | Human IgG4 Fc amino acid sequence |
| SEQ ID NO: 11 | Human IgA1 Fc nucleotide sequence |
| SEQ ID NO: 12 | Human IgA1 Fc amino acid sequence |
| SEQ ID NO: 13 | Human IgA2 Fc nucleotide sequence |
| SEQ ID NO: 14 | Human IgA2 Fc amino acid sequence |
| SEQ ID NO: 15 | Human IgM Fc nucleotide sequence |
| SEQ ID NO: 16 | Human IgM Fc amino acid sequence |
| SEQ ID NO: 17 | Human IgE Fc nucleotide sequence |
| SEQ ID NO: 18 | Human IgE Fc amino acid sequence |
| SEQ ID NO: 19 | Human IgD Fc nucleotide sequence |
| SEQ ID NO: 20 | Human IgD Fc amino acid sequence |
| SEQ ID NO: 21 | Human IgG1 Fc Apollo forward primer (pIRESbleo GSSNT |
| | cloning site) (nucleotide sequence) |
| SEQ ID NO: 22 | Human IgG1 Fc Apollo reverse primer (pIRESbleo GSSNT |
| | cloning site) (nucleotide sequence) |
| SEQ ID NO: 23 | GM-CSF forward primer (nucleotide sequence) |
| SEQ ID NO: 24 | GM-CSF reverse primer (nucleotide sequence) |
| SEQ ID NO: 25 | GM-CSF nucleotide sequence (signal peptide) |
| SEQ ID NO: 26 | GM-CSF amino acid sequence (signal peptide) |
| SEQ ID NO: 27 | GM-CSF nucleotide sequence (mature peptide) |
| SEQ ID NO: 28 | GM-CSF amino acid sequence (mature peptide) |
| SEQ ID NO: 29 | GM-CSF nucleotide sequence (signal peptide + mature peptide) |
| SEQ ID NO: 30 | GM-CSF amino acid sequence (signal peptide + mature peptide) |
| SEQ ID NO: 31 | GM-CSF-Fc nucleotide sequence for whole construct (signal |
| | peptide + coding peptide + linker + IgG1 Fc) |
| SEQ ID NO: 32 | GM-CSF-Fc amino acid sequence for whole construct (signal |
| | peptide + coding peptide + linker + IgG1 Fc) |
| SEQ ID NO: 33 | IL-3 forward primer (nucleotide sequence) |
| SEQ ID NO: 34 | IL-3 reverse primer (nucleotide sequence) |
| SEQ ID NO: 35 | IL-3 nucleotide sequence (signal peptide) |
| SEQ ID NO: 36 | IL-3 amino acid sequence (signal peptide) |
| SEQ ID NO: 37 | IL-3 nucleotide sequence (mature peptide) |
| SEQ ID NO: 38 | IL-3 amino acid sequence (mature peptide) |
| SEQ ID NO: 39 | IL-3 nucleotide sequence (mature peptide) (variant) |
| SEQ ID NO: 40 | IL-3 amino acid sequence (mature peptide) (variant) |
| SEQ ID NO: 41 | IL-3 nucleotide sequence (signal peptide + mature peptide) |
| SEQ ID NO: 42 | IL-3-amino acid sequence (signal peptide + mature peptide) |
| SEQ ID NO: 43 | IL-3-Fc nucleotide sequence (signal peptide + mature peptide |
| | (variant)) |
| SEQ ID NO: 44 | IL-3-Fc amino acid sequence (signal peptide + mature peptide |
| | (variant)) |
| SEQ ID NO: 45 | IL-3-Fc nucleotide sequence for whole construct (signal peptide + |
| | mature peptide + GSSNT linker IgG1 Fc) |
| SEQ ID NO: 46 | IL-3-Fc amino acid sequence for whole construct (signal peptide + |
| | mature peptide + GSSNT linker IgG1 Fc) |
| SEQ ID NO: 47 | IL-3-Fc nucleotide sequence for whole construct (signal peptide + |
| | mature peptide (variant) + GSSNT linker IgG1 Fc) |
| SEQ ID NO: 48 | IL-3-Fc amino acid sequence for whole construct (signal peptide + |
| | mature peptide (variant) + GSSNT linker IgG1 Fc) |
| SEQ ID NO: 49 | IL-4 forward primer (nucleotide sequence) |
| SEQ ID NO: 50 | IL-4 reverse primer (nucleotide sequence) |
| SEQ ID NO: 51 | IL-4 nucleotide sequence (signal peptide) |
| SEQ ID NO: 52 | IL-4 amino acid sequence (signal peptide) |
| SEQ ID NO: 53 | IL-4 nucleotide sequence (mature peptide) |
| SEQ ID NO: 54 | IL-4 amino acid sequence (mature peptide) |
| SEQ ID NO: 55 | IL-4 nucleotide sequence (signal peptide + mature peptide) |
| SEQ ID NO: 56 | IL-4 amino acid sequence (signal peptide + mature peptide) |
| SEQ ID NO: 57 | IL-4-Fc nucleotide sequence for whole construct (signal peptide + |
| | mature peptide + GSSNT linker + IgG1 Fc) |
| SEQ ID NO: 58 | IL-4-Fc amino acid sequence for whole construct (signal peptide + |
| | mature peptide + GSSNT linker + IgG1 Fc) |
| SEQ ID NO: 59 | IL-5 forward primer (nucleotide sequence) |
| SEQ ID NO: 60 | IL-5 reverse primer (nucleotide sequence) |
| SEQ ID NO: 61 | IL-5 nucleotide sequence (signal peptide) |
| SEQ ID NO: 62 | IL-5 amino acid sequence (signal peptide) |
| SEQ ID NO: 63 | IL-5 nucleotide sequence (mature peptide) |
| SEQ ID NO: 64 | IL-5 amino acid sequence (mature peptide) |
| SEQ ID NO: 65 | IL-5 nucleotide sequence (signal peptide + mature peptide) |
| SEQ ID NO: 66 | IL-5 amino acid sequence (signal peptide + mature peptide) |
| SEQ ID NO: 67 | IL-5-Fc nucleotide sequence for whole construct (signal peptide + |
| | mature peptide + GSSNT linker IgG1 Fc) |
| SEQ ID NO: 68 | IL-5-Fc amino acid sequence for whole construct (signal peptide + |
| | mature peptide + GSSNT linker IgG1 Fc) |
| SEQ ID NO: 69 | GM-CSF genomic nucleotide sequence |
| SEQ ID NO: 70 | IL-3 genomic nucleotide sequence |
| SEQ ID NO: 71 | IL-4 genomic nucleotide sequence |
| SEQ ID NO: 72 | IL-5 genomic nucleotide sequence |

TABLE 2
List of physiochemical parameters

| | Physiochemical | | | | |
|---|---|---|---|---|---|
| Pₓ | Parameter | GM-CSF | IL-3 | IL-4 | IL-5 |
| P₁ | Apparent molecular | 16-40 kDa | 15-35 kDa | 12-24 kDa | 15-25 kDa |
| | weight | | | | |
| P₂ | Isoelectric point (pI) | 2-7 | 3.5-7.5 | 8-11 | 4-9 |
| P₃ | Number of isoforms | 10-30 | 5-15 | 1-3 | 5-12 |
| P₄ | Relative intensities of | | | | |
| | the different number of | | | | |
| | isoforms | | | | |
| P₅ | Percentage by weight | 0-65% | 0-60% | 0-25% | 10-50% |
| | carbohydrate | | | | |
| P₆ | Observed molecular | 12-30 kDa | 10-25 kDa | 10-20 kDa | 11-25 kDa |
| | weight following N- | | | | |
| | linked oligosaccharide | | | | |
| | deglycosylation | | | | |
| P₇ | Observed molecular | 11-25 kDa | | 10-18 kDa | 10-22 kDa |
| | weight following N- | | | | |
| | linked oligosaccharide | | | | |
| | deglycosylation and O- | | | | |
| | linked oligosaccharide | | | | |
| | deglycosylation | | | | |
| P₈ | Percentage acidic | 6-11% | | | |
| | monosaccharide | | | | |
| | content | | | | |
| P₉ | Monosaccharide | When | When | | When |
| | content | normalised to | normalised | | normalised |
| | | GalNAc: | to GalNAc: | | to GalNAc: |
| | | 1 to 0.1-1.5 | 1 to 2-6 | | 1 to 0-0.5 |
| | | fucose, 1 to | fucose, 1 to | | fucose, 1 to |
| | | 2-12 | 3-5 | | 2-4.5 |
| | | GlcNAc, 1 to | GlcNAc, 1 | | GlcNAc, 1 to |
| | | 1.0-6.0 | to 0.5-2 | | 1-2 |
| | | galactose, 1 | galactose, 1 | | galactose, 1 |
| | | to 1.0-6.0 | to 0.5-2 | | to 1-2 |
| | | mannose, 1 | mannose, 1 | | mannose, 1 |
| | | to 0-3.0 | to 0-2 | | to 0.1-1 |
| | | NeuNAc; | NeuNAc; | | NeuNAc; |
| | | When | When | | When |
| | | normalised to | normalised | | normalised |
| | | 3 x mannose: | to 3 x | | to 3 x |
| | | 3 to 0.1-2.5 | mannose: | | mannose: |
| | | fucose, 3 to | 3 to 5-16 | | 3 to 0-1 |
| | | 0.5-2.5 | fucose, 3 to | | fucose, 3 to |
| | | GalNAc, 3 to | 2-4 | | 2-3 GalNAc, |
| | | 5.0-10.0 | GalNAc, 3 | | 3 to 3-12 |
| | | GlcNAc, 3 to | to 9-14 | | GlcNAc, 3 to |
| | | 2.0-5.0 | GlcNAc, 3 | | 2-5 |
| | | galactose, 3 | to 3-6 | | galactose, 3 |
| | | to 0-3.0 | galactose, 3 | | to 0.2-1 |
| | | NeuNAc. | to 0.1-2 | | NeuNAc. |
| | | | NeuAc. | | |
| P₁₀ | Sialic acid content | | When | | When |
| | | | expressed as | | expressed as |
| | | | a percentage | | a percentage |
| | | | of the | | of the |
| | | | monosaccharide | | monosaccharide |
| | | | content, | | content, |
| | | | 0-20%. | | 2-10%. |
| P₁₁ | Sulfate and phosphate | | | | When |
| | content | | | | normalised |
| | | | | | to GalNAc: |
| | | | | | 1 to 5-10 |
| | | | | | sulfate; |
| | | | | | When |
| | | | | | normalised |
| | | | | | to 3 x |
| | | | | | mannose: |
| | | | | | 3 to 12-24 |
| | | | | | sulfate. |
| P₁₂ | Ser/Thr:GalNAc ratio | | | | |
| P₁₃ | Neutral percentage of | 59-73% | 80-90% | 70-100% | 50-75% |
| | N-linked | | | | |
| | oligosaccharide | | | | |
| | content | | | | |
| P₁₄ | Acidic percentage of | 27-41% | 10-20% | 0-30% | 25-50% |
| | N-linked | | | | |
| | oligosaccharide | | | | |
| | content | | | | |
| P₁₅ | Neutral percentage of | 34-57% | | | 60-100% |
| | O-linked | | | | |
| | oligosaccharide | | | | |
| | content | | | | |
| P₁₆ | Acidic percentage of | 43-66% | | | 0-40% |
| | O-linked | | | | |
| | oligosaccharide | | | | |
| | content | | | | |
| P₁₇ | Ratio of N-linked | | | | |
| | oligosaccharides | | | | |
| P₁₈ | Ratio of O-linked | | | | |
| | oligosaccharides | | | | |
| P₁₉ | Structure of N-linked | | | | |
| | fraction | | | | |
| P₂₀ | Structure of O-linked | | | | |
| | fraction | | | | |
| P₂₁ | Position and make up | Includes N- | | Includes N- | |
| | of N-linked | 44 and N-54 | | 62 | |
| | oligosaccharides | (numbering | | (numbering | |
| | | from the start | | from the | |
| | | of the signal | | start of the | |
| | | sequence) | | signal | |
| | | identified by | | sequence) | |
| | | PMF after | | identified | |
| | | PNGase | | by PMF | |
| | | treatment. | | after | |
| | | | | PNGase | |
| | | | | treatment. | |
| P₂₂ | Position and make up | | | | |
| | of O-linked | | | | |
| | oligosaccharides | | | | |
| P₂₃ | Co-translational | | | | |
| | modification | | | | |
| P₂₄ | Post-translational | | | | |
| | modification | | | | |
| P₂₅ | Acylation | | | | |
| P₂₆ | Acetylation | | | | |
| P₂₇ | Amidation | | | | |
| P₂₈ | Deamidation | | | | |
| P₂₉ | Biotinylation | | | | |
| P₃₀ | Carbamylation or | | | | |
| | carbamoylation | | | | |
| P₃₁ | Carboxylation | | | | |
| P₃₂ | Decarboxylation | | | | |
| P₃₃ | Disulfide bond | | | The sites of | |
| | formation | | | disulfide | |
| | | | | bond | |
| | | | | formation | |
| | | | | (P₃₃) | |
| | | | | include | |
| | | | | Cys27-Cys151, | |
| | | | | Cys48-Cys89 | |
| | | | | and | |
| | | | | Cys70-Cys123 | |
| | | | | (cysteine | |
| | | | | residues | |
| | | | | numbered | |
| | | | | from the | |
| | | | | start of the | |
| | | | | signal | |
| | | | | sequence) | |
| P₃₄ | Fatty acid acylation | | | | |
| P₃₅ | Myristoylation | | | | |
| P₃₆ | Palmitoylation | | | | |
| P₃₇ | Stearoylation | | | | |
| P₃₈ | Formylation | | | | |
| P₃₉ | Glycation | | | | |
| P₄₀ | Glycosylation | | | | |
| P₄₁ | Glycophosphatidylinositol | | | | |
| | anchor | | | | |
| P₄₂ | Hydroxylation | | | | |
| P₄₃ | Incorporation of | | | | |
| | selenocysteine | | | | |
| P₄₄ | Lipidation | | | | |
| P₄₅ | Lipoic acid addition | | | | |
| P₄₆ | Methylation | | | | |
| P₄₇ | N or C terminal | | | | |
| | blocking | | | | |
| P₄₈ | N or C terminal | | | | |
| | removal | | | | |
| P₄₉ | Nitration | | | | |
| P₅₀ | Oxidation of | | | | |
| | methionine | | | | |
| P₅₁ | Phosphorylation | | | | |
| P₅₂ | Proteolytic cleavage | | | | |
| P₅₃ | Prenylation | | | | |
| P₅₄ | Farnesylation | | | | |
| P₅₅ | Geranyl geranylation | | | | |
| P₅₆ | Pyridoxal phosphate | | | | |
| | addition | | | | |
| P₅₇ | Sialylation | | | | |
| P₅₈ | Desialylation | | | | |
| P₅₉ | Sulfation | | | | When |
| | | | | | expressed as |
| | | | | | a percentage |
| | | | | | of the |
| | | | | | monosaccharide |
| | | | | | content, |
| | | | | | 10-25%. |
| P₆₀ | Ubiquitinylation or | | | | |
| | ubiquitination | | | | |
| P₆₁ | Addition of ubiquitin- | | | | |
| | like molecules | | | | |
| P₆₂ | Primary structure | | | | |
| P₆₃ | Secondary structure | | | | |
| P₆₄ | Tertiary structure | | | | |
| P₆₅ | Quaternary structure | | | | |
| P₆₆ | Chemical stability | | | | |
| P₆₇ | Thermal stability | | | | |

TABLE 3
List of Pharmacological traits

| | Pharmacological | | | | |
|---|---|---|---|---|---|
| T_{y} | trait | GM-CSF | IL-3 | IL-4 | IL-5 |
| T₁ | Therapeutic | | | | |
| | efficiency | | | | |
| T₂ | Effective | | | | |
| | therapeutic | | | | |
| | dose (TCID₅₀) | | | | |
| T₃ | Bioavailability | | | | |
| T₄ | Time between | | | | |
| | dosages to | | | | |
| | maintain | | | | |
| | therapeutic | | | | |
| | levels | | | | |
| T₅ | Rate of | | | | |
| | absorption | | | | |
| T₆ | Rate of | | | | |
| | excretion | | | | |
| T₇ | Specific | | | | |
| | activity | | | | |
| T₈ | Thermal | | | | |
| | stability | | | | |
| T₉ | Lyophilization | | | | |
| | stability | | | | |
| T₁₀ | Serum/plasma | Greater | | | |
| | stability | proliferative | | | |
| | | activity on | | | |
| | | TF-1 cells | | | |
| | | following a | | | |
| | | 24 hour | | | |
| | | incubation in | | | |
| | | fetal calf | | | |
| | | serum than | | | |
| | | human GM- | | | |
| | | CSF | | | |
| | | produced | | | |
| | | from E. coli | | | |
| | | cells | | | |
| T₁₁ | Serum half-life | | | | |
| T₁₂ | Solubility in | | | | |
| | blood stream | | | | |
| T₁₃ | Immunoreactivity | | Underestimation | Underestimation | |
| | Profile | | of protein | of protein | |
| | | | concentration | concentration | |
| | | | when assayed | when assayed | |
| | | | using an | using an | |
| | | | ELISA with a | ELISA with a | |
| | | | standard | standard | |
| | | | expressed in | expressed in | |
| | | | non-human | non-human | |
| | | | cells; Distinct | cells. | |
| | | | from that of a | | |
| | | | human IL-3 | | |
| | | | expressed in | | |
| | | | insect cells. | | |
| T₁₄ | Immunogenicity | | | | |
| T_{14A} | Inhibitable by | | | | |
| | neutralizing | | | | |
| | antibodies | | | | |
| T₁₅ | Side effects | | | | |
| T₁₆ | Receptor/ligand | | | | |
| | binding | | | | |
| | affinity | | | | |
| T₁₇ | Receptor/ligand | | | | |
| | activation | | | | |
| T₁₈ | Tissue or cell | | | | |
| | type specificity | | | | |
| T₁₉ | Ability to cross | | | | |
| | biological | | | | |
| | membranes or | | | | |
| | barriers (i.e. | | | | |
| | gut, lung, | | | | |
| | blood brain | | | | |
| | barriers, skin | | | | |
| | etc) | | | | |
| T_{19A} | Angiogenic | | | | |
| | ability | | | | |
| T₂₀ | Tissue uptake | | | | |
| T₂₁ | Stability to | | | | |
| | degradation | | | | |
| T₂₂ | Stability to | | | | |
| | freeze-thaw | | | | |
| T₂₃ | Stability to | | | | |
| | proteases | | | | |
| T₂₄ | Stability to | | | | |
| | ubiquitination | | | | |
| T₂₅ | Ease of | | | | |
| | administration | | | | |
| T₂₆ | Mode of | | | | |
| | administration | | | | |
| T₂₇ | Compatibility | | | | |
| | with other | | | | |
| | pharmaceutical | | | | |
| | excipients or | | | | |
| | carriers | | | | |
| T₂₈ | Persistence in | | | | |
| | organism or | | | | |
| | environment | | | | |
| T₂₉ | Stability in | | | | |
| | storage | | | | |
| T₃₀ | Toxicity in an | | | | |
| | organism or | | | | |
| | environment | | | | |
| | and the like | | | | |
| T₃₁ | Altered | | | | |
| | biological | | | | |
| | effects on | | | | |
| | different cells | | | | |
| | types | | | | |
| T₃₂ | Proliferation | 5-12 times | 1.1-2.5 times | 25-54 times | |
| | | greater | greater | greater | |
| | | proliferative | proliferative | proliferative | |
| | | activity on | activity on | activity on TF- | |
| | | TF-1 cells | M-NFS-60 | 1 cells than | |
| | | than human | cells than | human IL-4 | |
| | | GM-CSF | human IL-3 | expressed in E. coli | |
| | | expressed in | expressed in | cells; up to | |
| | | E. coli cells. | E. coli cells. | 1.75 fold | |
| | | | | greater | |
| | | | | proliferative | |
| | | | | activity than | |
| | | | | human IL-4 | |
| | | | | expressed in | |
| | | | | CHO cells; 13-30 | |
| | | | | fold greater | |
| | | | | proliferative | |
| | | | | activity on TF- | |
| | | | | 1 cells | |
| | | | | following a 4 | |
| | | | | day pre- | |
| | | | | incubation at | |
| | | | | 37° C. in cell | |
| | | | | culture | |
| | | | | medium than | |
| | | | | human GM- | |
| | | | | CSF produced | |
| | | | | from E. coli | |
| | | | | cells. | |
| T₃₃ | Differentiation | 1.5-2 fold | | | |
| | | greater | | | |
| | | capacity to | | | |
| | | induce | | | |
| | | colony | | | |
| | | formation in | | | |
| | | TF-1 cells | | | |
| | | over a range | | | |
| | | of | | | |
| | | concentrations | | | |
| | | than human | | | |
| | | GM-CSF | | | |
| | | expressed in | | | |
| | | E. coli cells. | | | |
| T₃₄ | Apoptosis | | | | |
| T₃₅ | Growth in cell | | | | |
| | size | | | | |
| T₃₆ | Cytokine | | | | |
| | adhesion | | | | |
| T₃₇ | Cell adhesion | | | | |
| T₃₈ | Cell spreading | | | | |
| T₃₉ | Cell motility | | | | |
| T₄₀ | Migration and | | | | |
| | invasion | | | | |
| T₄₁ | Chemotaxis | | | | |
| T₄₂ | Cell | | | | |
| | engulfment | | | | |
| T₄₃ | Signal | | | | |
| | transduction | | | | |
| T₄₄ | Recruitment of | | | | |
| | proteins to | | | | |
| | receptors/ligands | | | | |
| T₄₅ | Activation of | | | | |
| | the JAK/STAT | | | | |
| | pathway | | | | |
| T₄₆ | Activation of | | | | |
| | the Ras-erk | | | | |
| | pathway | | | | |
| T₄₇ | Activation of | | | | |
| | the AKT | | | | |
| | pathway | | | | |
| T₄₈ | Activation of | | | | |
| | the PKC | | | | |
| | pathway and | | | | |
| | PKA pathway | | | | |
| T₄₉ | Activation of | | | | |
| | the PKA | | | | |
| | pathway | | | | |
| T₅₀ | Activation of | | | | |
| | src | | | | |
| T₅₁ | Activation of | | | | |
| | fas | | | | |
| T₅₂ | Activation of | | | | |
| | TNFR | | | | |
| T₅₃ | Activation of | | | | |
| | NFkB | | | | |
| T₅₄ | Activation of | | | | |
| | p38MAPK | | | | |
| T₅₅ | Activation of | | | | |
| | c-fos | | | | |
| T₅₆ | Secretion | | | | |
| T₅₇ | Receptor | | | | |
| | internalization | | | | |
| T₅₈ | Receptor | | | | |
| | cross-talk | | | | |
| T₅₉ | Up or down | | | | |
| | regulation of | | | | |
| | surface | | | | |
| | markers | | | | |
| T₆₀ | Alteration of | | | | |
| | FACS | | | | |
| | front/side | | | | |
| | scatter profiles | | | | |
| T₆₁ | Alteration of | | | | |
| | subgroup | | | | |
| | ratios | | | | |
| T₆₂ | Differential | | | | |
| | gene | | | | |
| | expression | | | | |
| T₆₃ | Cell necrosis | | | | |
| T₆₄ | Cell clumping | | | | |
| T₆₅ | Cell repulsion | | | | |
| T₆₆ | Binding to | | | | |
| | heparin | | | | |
| | sulfates | | | | |
| T₆₇ | Binding to | | | | |
| | glycosylated | | | | |
| | structures | | | | |
| T₆₈ | Binding to | | | | |
| | chondroitin | | | | |
| | sulfates | | | | |
| T₆₉ | Binding to | | | | |
| | extracellular | | | | |
| | matrix (such as | | | | |
| | collagen, | | | | |
| | fibronectin) | | | | |
| T₇₀ | Binding to | | | | |
| | artificial | | | | |
| | materials (such | | | | |
| | as scaffolds) | | | | |
| T₇₁ | Binding to | | | | |
| | carriers | | | | |
| T₇₂ | Binding to co- | | | | |
| | factors | | | | |
| T₇₃ | The effect | | | | |
| | alone or in | | | | |
| | combination | | | | |
| | with other | | | | |
| | proteins on | | | | |
| | stem cell | | | | |
| | proliferation, | | | | |
| | differentiation | | | | |
| | and/or self- | | | | |
| | renewal. | | | | |

A list of abbreviations commonly used herein is provided in Tables 4 and 5.

TABLE 4
Abbreviations and alternate names

| Abbreviation | Description |
|---|---|
| AAA | Amino Acid Analysis |
| AFC | Affinity Chromatography |
| bFGF | Basic Fibroblast Growth Factor, FGF2 |
| BSA | Bovine Serum Albumin |
| cDLC | Combinatorial Dye Ligand Chromatography |
| CRD | Carbohydrate Recognition Domain |
| CSF | Colony Stimulating Factor |
| DCS | Donor Calf Serum |
| DeoxGlc | 2-deoxyglucose |
| DLC | Dye Ligand pseudoaffinity Chromatography |
| DSC | Differential Scanning Calorimetry |
| ECD | Extracellular domain |
| EGF | Epidermal Growth Factor |
| ELISA | Enzyme-Linked Immunosorbent Assays |
| EPO | Erythropoietin |
| EST | Expressed Sequence Tags |
| Fc | Fragment Crystallizable or Immunoglobulin constant region |
| FCS | Fetal Calf Serum |
| FGF2 | Basic Fibroblast Growth Factor, bFGF |
| FTIS | Fourier Transform Infrared Spectroscopy |
| Fuc | Fucose |
| G-CSF | Granulocyte Colony Stimulating Factor |
| GM-CSF | Granulocyte-macrophage colony stimulating factor (GMCSF); |
| | colony stimulating factor 2 (granulocyte-macrophage) (CSF2) |
| | (approved gene symbol); burst promoting activity (BPA); colony |
| | stimulating factor-alpha or beta (CSF-alpha or CSF-beta); |
| | colony stimulating factor 2 (CSF-2); eosinophil colony |
| | stimulating factor (Eo-CSF); eosinophil viability enhancing |
| | factor; eosinophil stimulation promoter (ESP); granulocyte |
| | macrophage colony stimulating activity (GM-CSA); |
| | hematopoietic cell growth factor (HCGF); histamine-producing |
| | cell stimulating factor (HCSF); KM102 burst promoting activity |
| | (KM102-BPA); keratinocyte-derived T-cell growth factor |
| | (KTGF); leukemic blast growth factor (LBGF); macrophage |
| | fusion factor (MFF); macrophage granulocyte inducer (MGI- |
| | 1GM); neutrophil migration inhibition factor from T- |
| | lymphocytes or T-cell derived neutrophil migration inhibition |
| | factor (NIF-T); pluripoietin alpha; WEHI-3B differentiation |
| | inducing factor. |
| Gal | Galactose |
| GalNAc, galactosamine | 2-deoxy, 2 amino galactose |
| GFC | Gel Filtration Chromatography |
| GlcA | Glucuronic acid |
| GlcNAc, glucosamine | 2-deoxy, 2 amino glucose |
| Glc | Glucose |
| GM-CSF | Granulocyte-Macrophage Colony Stimulating Factor |
| HBS | Hepes Buffered Saline |
| hES | Human Embryonic Stem Cells |
| HIC | Hydrophobic Interaction Chromatography |
| HPAEC-PAD | High-pH anion-exchange chromatography with pulsed |
| | amperometric detection |
| HPLC | High Pressure Liquid Chromatography or High Performance |
| | Liquid Chromatography |
| HSA | Human Serum Albumin |
| HTS | High Throughput Screening |
| IdoA | Iduronic acid |
| IEC | Ion Exchange Chromatography |
| IEF | Isoelectric focussing |
| IFN | Interferon |
| Ig | Immunoglobulin |
| IL | Interleukin |
| IL-3 | Interleukin 3 (IL3); burst promoting activity (BP); blood |
| | progenitor activator (BPA); burst promoting activity (BPA); |
| | colony forming unit spleen (CFU-S); colony forming unity |
| | stimulating activity (CFU-SA); colony stimulating factor-2- |
| | alpha (CSF-2-alpha); colony stimulating factor-2-beta (CSF-2- |
| | beta); epidermal cell IL-3 (EC IL-3); erythroid colony |
| | stimulating factor (ECSF); epidermal keratinocyte (EK) derived |
| | basophil promoting activity; eosinophil colony stimulating factor |
| | (Eo-CSF); hematopoietic cell growth factor (HCGF); histamine- |
| | producing cell stimulating factor (HCSF); hematopoietin-2 |
| | (HP2); hematopoietic cell growth factor (HPGF); maturation |
| | inducing activity; mast cell growth factor (MCGF); multi-colony |
| | stimulating activity (MCSA); megakayocyte colony stimulating |
| | activity (MEG-CSA); megakayocyte colony stimulating factor |
| | (MEG-CSF); mast cell growth factor (MFG); mixed colony |
| | stimulating factor; multi-colony stimulating factor (multi-CSF); |
| | multi-lineage hemopoietic growth factor (multi-HGF); |
| | multipoietin; natural cytotoxic (NC) cell growth factor; |
| | neutrophil-granulocyte colony stimulating factor; P cell |
| | stimulating activity (PCSA); P cell stimulating factor (PCSF); P- |
| | Factor; pluripotential stem cell supporting factor; persisting cell |
| | stimulating factor (PSF); progenitor stimulating factor (PSF); |
| | panspecfic hemopoietin (PSH); synergistic activity (SA); stem |
| | cell activating factor (SAF); thyl inducing factor; WEHI 3 |
| | growth factor; WEHI 3 hematopoeitic growth factor; WEHI 3 |
| | growth factor (WGF). |
| IL-4 | Interleukin 4 (IL4); MHC class II Ia inducing factor (IaIF); B- |
| | cell differentiation factor-epsilon (BCGF-epsilon); B-cell |
| | differentiation factor-gamma (BCDF-gamma); B-cell growth |
| | factor-gamma (BCGF-gamma); B-cell growth factor-1 (BCGF- |
| | 1); B-cell stimulating factor-1 (BSF-1); B-cell stimulatory factor |
| | p1 (BSF-p1); EL4-BCGF (EL4 B-cell growth factor); Hodgkin's |
| | cell growth factor (HCGF); IgE enhancing factor (IgE-EF); |
| | IgG1-enhancing factor; IgG1-induction factor; low molecular |
| | weight B-cell growth factor (LMW-BCGF); mast cell growth |
| | factor-2 (MCGF-2); macrophage fusion factor (MFF); T-cell |
| | growth factor-2 (TCGF-2); Binetrakin (Sch39400). |
| IL-5 | Interleukin 5 (IL5); B151 T-cell replacing factor (B151-TRF); B |
| | cell differentiation factors (BCDF); B-cell differentiation factor- |
| | mu (BCDF-mu); B-cell differentiation factor-alpha (BCDF- |
| | alpha); B-cell growth factor-2 (BCGF-2); B-cell growth and |
| | differentiation factor (BGDF); eosinophil differentiation factor |
| | (EDF); Bosinophil colony stimulating factor (Eo-CSF); IgA |
| | enhancing factor (IgA-EF); killer helper factor (KHF); T-cell |
| | replacing factor-1 (TRF-1); Eosinophil stimulation promoter |
| | (ESP). |
| lacNAc | N-acetyl lactosamine |
| lacdiNAc | N,N′-diacetyllactosediamine |
| LC | Liquid Chromatography |
| MALDI-TOF | Matrix-Assisted Laser Desorption Ionization - Time of Flight |
| Man | Mannose |
| MCC | Metal Chelating Chromatography |
| MS | Mass Spectroscopy |
| NacSial, NeuAc or | N-acetyl neuraminic acid |
| NeuNAc | |
| NGlySial, NeuGc or | N-glycolyl neuraminic acid |
| NeuGly | |
| PBS | Phosphate Buffered Saline |
| PCS | Photon Correlation Spectroscopy |
| PDGF-AA | Platelet Derived Growth Factor A homodimer |
| PNGase | Peptide-N4-(N-acetyl-β-D-glucosaminyl) Asparagine Amidase |
| RMLP | Receptor Mediated Ligand Chromatography |
| RPC | Reversed Phase Chromatography |
| SDS PAGE | Sodium Dodecyl Sulfate Polyacrylamide Gel Electrophoresis |
| SEC | Size Exclusion Chromatography |
| Sia | Sialic acid |
| TCA | Trichloroacetic acid |
| TFF | Tangential flow filtration |
| TGF | Transforming Growth Factor |
| TNF | Tumor Necrosis Factor |
| TNFR | Tumor Necrosis Factor Receptor |
| Xyl | Xylose |

TABLE 5
Abbreviations for amino acids

| | 3 Letter | 1 Letter |
|---|---|---|
| Amino Acid | Code | Code |
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 6
Stem cell list
Cell type

General Stem Cell Types
Embryonic stem cells
Somatic stem cells
Germ stem cells
Human embryonic stem cells
Human epidermal stem cells
Adipose derived stem cells
Brain
Adult neural stem cells
Human neurons
Human astrocytes
Epidermis
Human keratinocyte stem cells
Human keratinocyte transient amplifying cells
Human melanocyte stem cells
Human melanocytes
Skin
Human foreskin fibroblasts
Pancreas
Human duct cells
Human pancreatic islets
Human pancreatic β-cells
Kidney
Human adult renal stem cells
Human embryonic renal epithelial stem cells
Human kidney epithelial cells
Liver
Human hepatic oval cells
Human hepatocytes
Human bile duct epithelial cells
Human embryonic endodermal stem cells
Human adult hepatocyte stem cells (existence controversial)
Breast
Human mammary epithelial stem cells
Lung
Bone marrow-derived stem cells
Human lung fibroblasts
Human bronchial epithelial cells
Human alveolar type II pneumocytes
Muscle
Human skeletal muscle stem cells (satellite cells)
Heart
Human cardiomyocytes
Bone marrow mesenchymal stem cells
Simple Squamous Epithelial cells
Descending Aortic Endothelial cells
Aortic Arch Endothelial cells
Aortic Smooth Muscle cells
Eye
Limbal stem cells
Corneal epithelial cells
CD34+ hematopoietic stem cells
Mesenchymal stem cells
Osteoblasts (precursor is mesenchymal stem cell)
Peripheral blood mononuclear progenitor cells (hematopoietic stem cells)
Osteoclasts (precursor is above cell type)
Stromal cells
Spleen
Human splenic precursor stem cells
Human splenocytes
Immune cells
Human CD4+ T-cells
Human CD8+ T-cells
Human NK cells
Human monocytes
Human macrophages
Human dendritic cells
Human B-cells
Nose
Goblet cells (mucus secreting cells of the nose)
Pseudostriated ciliated columnar cells (located below olfactory region in
the nose)
Pseudostratified ciliated epithelium (cells that line the nasopharangeal
tubes)
Trachea
Stratified Epithelial cells (cells that line and structure the trachea)
Ciliated Columnar cells (cells that line and structure the trachea)
Goblet cells (cells that line and structure the trachea)
Basal cells (cells that line and structure the trachea)
Oesophagus
Cricopharyngeus muscle cells
Reproduction
Female primary follicles
Male spermatogonium

### BRIEF DESCRIPTION OF THE FIGURES

FIG. 1 is a diagrammatic representation of the cloning process for inserting cDNA encoding a protein of the present invention into the pIRESbleo3 or pIRESbleo3-Fc vector.

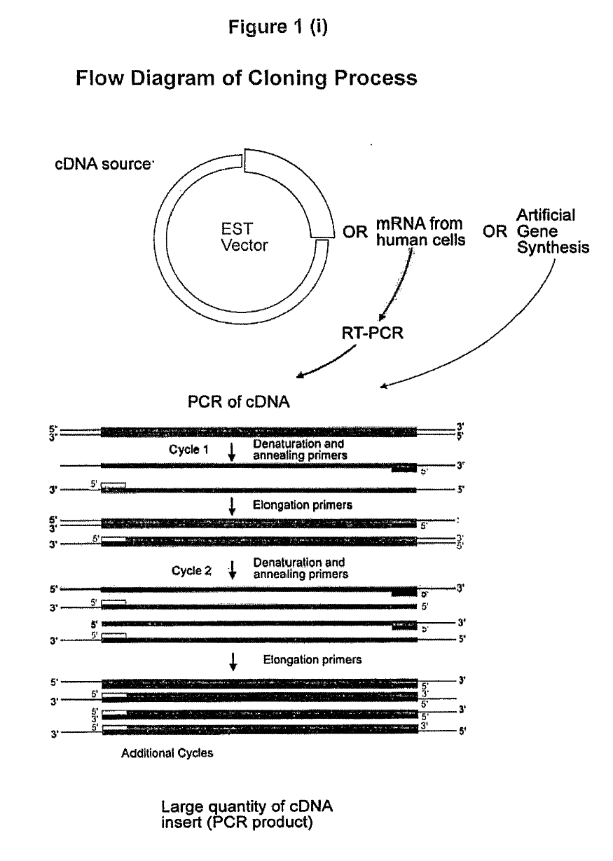

FIG. 2 is a graphical representation comparing the proliferation of TF1 cells by GM-CSF of the present invention and human GM-CSF expressed using non-human systems.

FIG. 3 is a graphical representation comparing the serum stability of GM-CSF of the present invention and human GM-CSF expressed using non-human systems, as determined by induction of TF-1 cell proliferation.

FIG. 4 is a graphical representation showing the differentiation and proliferation of granulocyte and macrophage colonies by GM-CSF of the present invention and human GM-CSF expressed using non-human systems.

FIG. 5 is a graphical representation comparing the proliferation of M-NFS-60 cells by IL-3 of the present invention and human IL-3 expressed using non-human systems.

FIG. 6(a) is a graphical representation comparing the proliferation of TF-1 cells by IL-4 of the present invention (filled circles) and human IL-4 expressed in E. coli (triangles) and Chinese Hamster Ovary (CHO) cells (open circles).

FIG. 6(b) is a graphical representation comparing the proliferation of TF-1 cells by IL-4 of the present invention (circles) and human IL-4 expressed in E. coli (squares) following IL-4 pre-incubation in cell culture medium for 4 days at 37° C.

FIG. 7 is a graphical representation showing the in vitro comparison of immunoreactivity profiles between IL-3 of the present invention (squares) and human IL-3 expressed using E. Coli cells (diamonds) and insect cells (triangles).

FIG. 8 is a graphical representation showing the in vitro comparison of immunoreactivity profiles between IL-4 of the present invention (triangles) and human IL-4 expressed using non-human systems (squares).

### DETAILED DESCRIPTION OF THE PREFERRED EMBODIMENTS

It is to be understood that unless otherwise indicated, the subject invention is not limited to specific formulations, manufacturing methods, diagnostic methods, assay protocols, nutritional protocols, or research protocols or the like as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context already dictates otherwise. Thus, for example, reference to “a protein”, “a cytokine” or “a chimeric molecule” or “a receptor” includes a single protein, cytokine or receptor or chimeric molecule as well as two or more proteins, cytokines or receptors or chimeric molecules; a “physiochemical parameter” includes a single parameter as well as two or more parameters and so forth.

The terms “compound”, “active agent”, “chemical agent”, “pharmacologically active agent”, “medicament”, “active” and “drug” are used interchangeably herein to refer to a chemical compound and in particular a protein or chimeric molecule thereof that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

Reference to a “compound”, “active agent”, “chemical agent” “pharmacologically active agent”, “medicament”, “active” and “drug” includes combinations of two or more actives such as two or more cytokines. A “combination” also includes multi-part such as a two-part composition where the agents are provided separately and given or dispensed separately or admixed together prior to dispensation.

For example, a multi-part pharmaceutical pack may have two or more proteins or chimeric molecules in or related to the short chain 4 helix bundle superfamily, selected from the group comprising GM-CSF, GM-CSF-Fc, IL-3, IL-3-Fc, IL-4, IL-4-Fc, IL-5 and IL-5-Fc separately maintained.

The terms “effective amount” and “therapeutically effective amount” of an agent as used herein mean a sufficient amount of the protein or chimeric molecule thereof, alone or in combination with other agents to provide the desired therapeutic or physiological effect or outcome. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or underlying cause, prevention of the occurrence of symptoms of the condition and/or their underlying cause and improvement or remediation or amelioration of damage following a condition.

“Treating” a subject may involve prevention of a condition or other adverse physiological event in a susceptible individual as well as treatment of a clinically symptomatic individual by ameliorating the symptoms of the condition.

A “subject” as used herein refers to an animal, in a particular embodiment, a mammal and in a further embodiment human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods of the present invention have applications in human medicine, veterinary medicine as well as in general, domestic or wild animal husbandry.

As indicated above, in a particular embodiment, the animals are humans or other primates such as orangutans, gorillas, marmosets, livestock animals, laboratory test animals, companion animals or captive wild animals, as well as avian species.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, fish, and amphibians including Xenopus spp prokaryotes and non-mammalian eukaryotes.

The term “cytokine” is used in its most general sense and includes any of various proteins secreted by cells to regulate the immune system, modulate the functional activities of individual cells and/or tissues, and/or induce a range of physiological responses. As used herein the term “cytokine” should be understood to refer to a “complete” cytokine as well as fragments, derivatives or homologs or chimeras thereof comprising one or more amino acid additions, deletions or substitutions, but which substantially retain the biological activity of the complete cytokine.

A “cytokine receptor” is a cell membrane associated or soluble portion of the cytokine receptor involved in cytokine signalling or regulation. As used herein the term “cytokine receptor” should be understood to refer to a “complete” cytokine receptor as well as fragments, derivatives or homologs or chimeras thereof comprising one or more amino acid additions, deletions or substitutions, but which substantially retain the biological activity of the complete cytokine receptor.

The term “protein” is used in its most general sense and includes cytokines and cytokine receptors. As used herein, the term “protein” should be understood to refer to a “complete” protein as well as fragments, derivatives or homologs or chimeras thereof comprising one or more amino acid additions, deletions or substitutions, but which substantially retain the biological activity of the complete protein.

The present invention contemplates an isolated protein or chimeric molecule thereof having a profile of measurable physiochemical parameters (Pₓ), wherein the profile is indicative of, associated with or forms the basis of one or more distinctive pharmacological traits (T_{y}). The isolated protein or chimeric molecule is a protein in or related to the short chain 4 helix bundle superfamily, selected from the group comprising GM-CSF, GM-CSF-Fc, IL-3, IL-3-Fc, IL-4, IL-4-Fc, IL-5 and IL-5-Fc. As used herein, the terms GM-CSF, GM-CSF-Fc, IL-3, IL-3-Fc, IL-4, IL-4-Fc, IL-5 and IL-5-Fc includes reference to the whole polypeptide as well as fragments thereof.

More particularly, the present invention provides an isolated protein or chimeric molecule thereof having a physiochemical profile comprising an array of measurable physiochemical parameters, {[Pₓ]₁, [Pₓ]₂, . . . [Pₓ]ₙ,}, wherein Pₓ represents a measurable physiochemical parameter and “n” is an integer ≧1, wherein each of [Pₓ]₁ to [Pₓ]ₙ is a different measurable physiochemical parameter, wherein the value of any one or more of the measurable physiochemical characteristics is indicative of, associated with, or forms the basis of, a distinctive pharmacological trait, T_{y}, or a number of distinctive pharmacological traits {[T_{y}]₁, [T_{y}]₂, . . . [T_{y}]ₘ} wherein T_{y} represents a distinctive pharmacological trait and m is an integer ≧1 and each of [T_{y}]₁ to [T_{y}]ₘ is a different pharmacological trait.

As used herein, the term “measurable physiochemical parameters” (Pₓ) refers to one or more measurable characteristics of an isolated protein or chimeric molecule thereof. Exemplary “distinctive measurable physiochemical parameters” include, but are not limited to apparent molecular weight (P₁), isoelectric point (pI) (P₂), number of isoforms (P₃), relative intensities of the different number of isoforms (P₄), percentage by weight carbohydrate (P₅), observed molecular weight following N-linked oligosaccharide deglycosylation (P₆), observed molecular weight following N-linked and O-linked oligosaccharide deglycosylation (P₇), percentage acidic monosaccharide content (P₈), monosaccharide content (P₉), sialic acid content (P₁₀), sulfate and phosphate content (P₁₁), Ser/Thr:GalNAc ratio (P₁₂), neutral percentage of N-linked oligosaccharide content (P₁₃), acidic percentage of N-linked oligosaccharide content (P₁₄), neutral percentage of O-linked oligosaccharide content (P₁₅), acidic percentage of O-linked oligosaccharide content (P₁₆), ratio of N-linked oligosaccharides (P₁₇), ratio of O-linked oligosaccharides (P₁₈), structure of N-linked oligosaccharide fraction (P₁₉), structure of O-linked oligosaccharide fraction (P₂₀), position and make up of N-linked oligosaccharides (P₂₁), position and makeup of O-linked oligosaccharides (P₂₂), co-translational modification (P₂₃), post-translational modification (P₂₄), acylation (P₂₅), acetylation (P₂₆), amidation (P₂₇), deamidation (P₂₈), biotinylation (P₂₉), carbamylation or carbamoylation (P₃₀), carboxylation (P₃₁), decarboxylation (P₃₂), disulfide bond formation (P₃₃), fatty acid acylation (P₃₄), myristoylation (P₃₅), palmitoylation (P₃₆), stearoylation (P₃₇), formylation (P₃₈), glycation (P₃₉), glycosylation (P₄₀), glycophosphatidylinositol anchor (P₄₁), hydroxylation (P₄₂), incorporation of selenocysteine (P₄₃), lipidation (P₄₄), lipoic acid addition (P₄₅), methylation (P₄₆), N or C terminal blocking (P₄₇), N or C terminal removal (P₄₈), nitration (P₄₉), oxidation of methionine (P₅₀), phosphorylation (P₅₁), proteolytic cleavage (P₅₂), prenylation (P₅₃), farnesylation (P₅₄), geranyl geranylation (P₅₅), pyridoxal phosphate addition (P₅₆), sialylation (P₅₇), desialylation (P₅₈), sulfation (P₅₉), ubiquitinylation or ubiquitination (P₆₀), addition of ubiquitin-like molecules (P₆₁), primary structure (P₆₂), secondary structure (P₆₃), tertiary structure (P₆₄), quaternary structure (P₆₅), chemical stability (P₆₆), thermal stability (P₆₇). A summary of these parameters is provided is Table 2.

The term “distinctive pharmacological traits” would be readily understood by one of skill in the art to include any pharmacological or clinically relevant property of the protein or chimeric molecule of the present invention. Exemplary “pharmacological traits” which in no way limit the invention include: therapeutic efficiency (T₁), effective therapeutic dose (TCID₅₀) (T₂), bioavailability (T₃), time between dosages to maintain therapeutic levels (T₄), rate of absorption (T₅), rate of excretion (T₆), specific activity (T₇), thermal stability (T₈), lyophilization stability (T₉), serum/plasma stability (T₁₀), serum half-life (T₁₁), solubility in blood stream (T₁₂), immunoreactivity profile (T₁₃), immunogenicity (T₁₄), inhibition by neutralizing antibodies (T_{14A}), side effects (T₁₅), receptor/ligand binding affinity (T₁₆), receptor/ligand activation (T₁₇), tissue or cell type specificity (T₁₈), ability to cross biological membranes or barriers (i.e. gut, lung, blood brain barriers, skin etc) (T₁₉), angiogenic ability (T_{19A}), tissue uptake (T₂₀), stability to degradation (T₂₁), stability to freeze-thaw (T₂₂), stability to proteases (T₂₃), stability to ubiquitination (T₂₄), ease of administration (T₂₅), mode of administration (T₂₆), compatibility with other pharmaceutical excipients or carriers (T₂₇), persistence in organism or environment (T₂₈), stability in storage (T₂₉), toxicity in an organism or environment and the like (T₃₀).

In addition, the protein or chimeric molecule of the present invention may have altered biological effects on different cells types (T₃₁), including but not limited to human primary cells, such as lymphocytes, erythrocytes, retinal cells, hepatocytes, neurons, keratinocytes, endothelial cells, endodermal cells, ectodermal cells, mesodermal cells, epithelial cells, kidney cells, liver cells, bone cells, bone marrow cells, lymph node cells, dermal cells, fibroblasts, T-cells, B-cells, plasma cells, natural killer cells, macrophages, neutrophils, granulocytes Langerhans cells, dendritic cells, eosinophils, basophils, mammary cells, lobule cells, prostate cells, lung cells, oesophageal cells, pancreatic cells, Beta cells (insulin secreting cells), hemangioblasts, muscle cells, oval cells (hepatocytes), mesenchymal cells, brain microvessel endothelial cells, astrocytes, glial cells, various stem cells including adult and embryonic stem cells, various progenitor cells; and other human immortal, transformed or cancer cell lines. The biological effects on the cells include effects on proliferation (T₃₂), differentiation (T₃₃), apoptosis (T₃₄), growth in cell size (T₃₅), cytokine adhesion (T₃₆), cell adhesion (T₃₇), cell spreading (T₃₈), cell motility (T₃₉), migration and invasion (T₄₀), chemotaxis (T₄₁), cell engulfment (T₄₂), signal transduction (T₄₃), recruitment of proteins to receptors/ligands (T₄₄), activation of the JAK/STAT pathway (T₄₅), activation of the Ras-erk pathway (T₄₆), activation of the AKT pathway (T₄₇), activation of the PKC pathway (T₄₈), activation of the PKA pathway (T₄₉), activation of src (T₅₀), activation of fas (T₅₁), activation of TNFR (T₅₂), activation of NFkB (T₅₃), activation of p38MAPK (T₅₄), activation of c-fos (T₅₅), secretion (T₅₆), receptor internalization (T₅₇), receptor cross-talk (T₅₈), up or down regulation of surface markers (T₅₉), alteration of FACS front/side scatter profiles (T₆₀), alteration of subgroup ratios (T₆₁), differential gene expression (T₆₂), cell necrosis (T₆₃), cell clumping (T₆₄), cell repulsion (T₆₅), binding to heparin sulfates (T₆₆), binding to glycosylated structures (T₆₇), binding to chondroitin sulfates (T₆₈), binding to extracellular matrix (such as collagen, fibronectin) (T₆₉), binding to artificial materials (such as scaffolds) (T₇₀), binding to carriers (T₇₁), binding to co-factors (T₇₂), the effect alone or in combination with other proteins on stem cell proliferation, differentiation and/or self-renewal (T₇₃) and the like. A summary of these traits is provided in Table 3.

As used herein the term “distinctive” with regard to a pharmacological trait of a protein or a chimeric molecule of the present invention refers to one or more pharmacological traits of the protein or chimeric molecule thereof, which are distinctive for the particular physiochemical profile. In a particular embodiment, one or more of the pharmacological traits of the isolated protein or chimeric molecule thereof is different from, or distinctive relative to a form of the same protein or chimeric molecule produced in a prokaryotic or lower eukaryotic cell or even a higher non-human eukaryotic cell. In a particular embodiment, the pharmacological traits of the subject isolated protein or chimeric molecule thereof are substantially similar to or functionally equivalent to a naturally occurring protein.

As used herein the term “prokaryote” refers to any prokaryotic cell, which includes any bacterial cell (including actinobacterial cells) or archaeal cell. The meaning of the term “non-mammalian eukaryote”, as used herein is self-evident. However, for clarity, this term specifically includes any non-mammalian eukaryote including: yeasts such as Sacchamomyces spp. or Pichea spp.; other fungi; insects, including Drosophila spp. and insect cell cultures; fish, including Danio spp.; amphibians, including Xenopus spp.; plants and plant cell cultures.

Reference to a “stem cell” includes embryonic or adult stem cells and includes those stem cells listed in Table 6. A protein or chimeric molecule of the present invention may be used alone or in a cocktail of proteins to induce one or more of stem cell proliferation, differentiation or self-renewal.

Primary structure of a protein or chimeric molecule thereof may be measured as an amino acid sequence. Secondary structure may be measured as the number and/or relative position of one or more protein secondary structures such as α-helices, parallel β-sheets, antiparallel β-sheets or turns. Tertiary structure describes the folding of the polypeptide chain to assemble the different secondary structure elements in a particular arrangement. As helices and sheets are units of secondary structure, so the domain is the unit of tertiary structure. In multi-domain proteins, tertiary structure includes the arrangement of domains relative to each other. Accordingly, tertiary structure may be measured as the presence, absence, number and/or relative position of one or more protein “domains”. Exemplary domains which in no way limit the present invention include: lone helices, helix-turn-helix domains, four helix bundles, DNA binding domains, three helix bundles, Greek key helix bundles, helix-helix packing domains, β-sandwiches, aligned β-sandwiches, orthogonal β-sandwiches, β-barrels, up and down antiparallel β-sheets, Greek key topology domains, jellyroll topology domains, β-propellers, β-trefoils, β-Helices, Rossman folds, α/β horseshoes, α/β barrels, α+β topologies, disulphide rich folds, serine proteinase inhibitor domains, sea anemone toxin domains, EGF-like domains, complement C-module domain, wheat plant toxin domains, Naja (Cobra) neurotoxin domains, green mamba anticholinesterase domains, Kringle domains, mucin like region, globular domains, spacer regions. Quaternary structure is described as the arrangement of different polypeptide chains within the protein structure, with each chain possessing individual primary, secondary and tertiary structure elements. Examples include either homo- or hetero-oligomeric multimerization (e.g. dimerization or trimerization).

With respect to the primary structure, the present invention provides an isolated protein or chimeric molecule thereof, or a fragment thereof, encoded by a nucleotide sequence selected from the list consisting of SEQ ID NOs: 25, 27, 29, 31, 35, 37, 39, 41, 43, 45, 47, 51, 53, 55, 57, 61, 63, 65, 67, or a nucleotide sequence having at least about 60% identity to any one of the above-listed sequence or a nucleotide sequence capable of hybridizing to any one of the above sequences or their complementary forms under low stringency conditions.

Another aspect of the present invention provides an isolated polypeptide encoded by a nucleotide sequence selected from the list consisting of SEQ ID NOs: 69, 70, 71, 72 following splicing of their respective mRNA species by cellular processes.

Still, another aspect of the present invention provides an isolated nucleic acid molecule encoding protein or chimeric molecule thereof or a functional part thereof comprising a sequence of nucleotides having at least 60% similarity selected from the list consisting of SEQ ID NOs: 25, 27, 29, 31, 35, 37, 39, 41, 43, 45, 47, 51, 53, 55, 57, 61, 63, 65, 67 or after optimal alignment and/or being capable of hybridizing to one or more of SEQ ID NOs: 25, 27, 29, 31, 35, 37, 39, 41, 43, 45, 47, 51, 53, 55, 57, 61, 63, 65, 67 or their complementary forms under low stringency conditions.

In a particular embodiment, the present invention is directed to an isolated nucleic acid molecule comprising a sequence of nucleotides encoding a protein or chimeric molecule thereof, or a fragment thereof, an amino acid sequence substantially as set forth in one or more of SEQ ID NOs: 26, 28, 30, 32, 36, 38, 40, 42, 44, 46, 48, 52, 54, 56, 58, 62, 64, 66, 68 or an amino acid sequence having at least about 60% similarity to one or more of SEQ ID NOs: 26, 28, 30, 32, 36, 38, 40, 42, 44, 46, 48, 52, 54, 56, 58, 62, 64, 66, 68 after optimal alignment.

In another aspect, the present invention provides an isolated nucleic acid molecule encoding a protein molecule, or a fragment thereof, comprising a sequence of nucleotides selected from the group consisting of SEQ ID NOs: 27, 29, 37, 39, 41, 43, 53, 55, 63 or 65, linked directly or via one or more nucleotide sequences encoding protein linkers known in the art to nucleotide sequences encoding the constant (Fc) or framework region of a human immunoglobulin, substantially as set forth in one or more of SEQ ID NOs: 1, 3, 5, 7, 9, 11, 13, 15, 17 or 19.

In another aspect, the present invention provides an isolated protein molecule, or a fragment thereof, comprising an amino acid sequence selected from the group consisting of SEQ ID NOs: 28, 30, 38, 40, 42, 44, 54, 56, 64 or 66 linked directly or via one or more protein linkers known in the art, to the constant (Fc) or framework region of a human immunoglobulin, substantially as set forth in one or more of SEQ ID NOs: 2, 4, 6, 8, 10, 12, 14, 16, 18 or 20.

Another aspect of the present invention provides an isolated protein or chimeric molecule thereof, or a fragment thereof, comprising an amino acid sequence selected from the list consisting of SEQ ID NOs: 26, 28, 30, 32, 36, 38, 40, 42, 44, 46, 48, 52, 54, 56, 58, 62, 64, 66, 68, or an amino acid sequence having at least about 65% similarity to one or more of the above sequences.

In a particular embodiment, percentage amino acid similarity or nucleotide identity levels include at least about 61% or at least about 62% or at least about 63% or at least about 64% or at least about 65% or at least about 66% or at least about 67% or at least about 68% or at least about 69% or at least about 70% or at least about 71% or at least about 72% or at least about 73% or at least about 74% or at least about 75% or at least about 76% or at least about 77% or at least about 78% or at least about 79% or at least about 80% or at least about 81% or at least about 82% or at least about 83% or at least about 84% or at least about 85% or at least about 86% or at least about 87% or at least about 88% or at least about 89% or at least about 90% or at least about 91% or at least about 92% or at least about 93% or at least about 94% or at least about 95% or at least about 96% or at least about 97% or at least about 98% or at least about 99% similarity or identity.

A “derivative” of a polypeptide of the present invention also encompasses a portion or a part of a full-length parent polypeptide, which retains partial transcriptional activity of the parent polypeptide and includes a variant. Such “biologically-active fragments” include deletion mutants and small peptides, for example, for at least 10, in a particular embodiment, at least 20 and in a further embodiment at least 30 contiguous amino acids, which exhibit the requisite activity. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

The term “variant” refers, therefore, to nucleotide sequences displaying substantial sequence identity with reference nucleotide sequences or polynucleotides that hybridize with a reference sequence under stringency conditions that are defined hereinafter. The terms “nucleotide sequence”, “polynucleotide” and “nucleic acid molecule” may be used herein interchangeably and encompass polynucleotides in which one or more nucleotides have been added or deleted, or replaced with different nucleotides. In this regard, it is well understood in the art that certain alterations inclusive of mutations, additions, deletions and substitutions can be made to a reference nucleotide sequence whereby the altered polynucleotide retains the biological function or activity of the reference polynucleotide or the encoded polypeptide. The term “variant” also includes naturally occurring allelic variants.

The nucleic acid molecules of the present invention may be in the form of a vector or other nucleic acid construct.

In one embodiment, the vector is DNA and may optionally comprise a selectable marker.

Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistance genes such as the neomycin resistance gene (neo) and the hygromycin resistance gene (hyg). Selectable markers also include genes conferring the ability to grown on certain media substrates such as the tk gene (thymidine kinase) or the hprt gene (hypoxanthine phosphoribosyltransferase) which confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). Other selectable markers for use in mammalian cells and plasmids carrying a variety of selectable markers are described in Sambrook et al. Molecular Cloning—A Laboratory Manual, Cold Spring Harbour, New York, USA, 1990.

The selectable marker may depend on its own promoter for expression and the marker gene may be derived from a very different organism than the organism being targeted (e.g. prokaryotic marker genes used in targeting mammalian cells). However, it is favorable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin). A number of other variations may be employed to enhance expression of the selectable markers in animal cells, such as the addition of a poly(A) sequence and the addition of synthetic translation initiation sequences. Both constitutive and inducible promoters may be used.

The genetic construct of the present invention may also comprise a 3′ non-translated sequence. A 3′ non-translated sequence refers to that portion of a gene comprising a DNA segment that contains a polyadenylation signal and any other regulatory signals capable of affecting mRNA processing or gene expression. The polyadenylation signal is characterized by affecting the addition of polyadenylic acid tracts to the 3′ end of the mRNA precursor. Polyadenylation signals are commonly recognized by the presence of homology to the canonical form 5′ AATAAA-3′ although variations are not uncommon.

Accordingly, a genetic construct comprising a nucleic acid molecule of the present invention, operably linked to a promoter, may be cloned into a suitable vector for delivery to a cell or tissue in which regulation is faulty, malfunctioning or non-existent, in order to rectify and/or provide the appropriate regulation. Vectors comprising appropriate genetic constructs may be delivered into target eukaryotic cells by a number of different means well known to those skilled in the art of molecular biology.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particular embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl Acids Res 25:389, 1997). A detailed discussion of sequence analysis can be found in Unit 19.3 of Ausubel et al. (In: Current Protocols in Molecular Biology, John Wiley & Sons Inc. 1994-1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software Engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 or 0.9 M for hybridization, and at least about 0.5 M to at least about 0.9 M salt, such as 0.5, 0.6, 0.7, 0.8 or 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C) % (Marmur and Doty, J Mol Biol 5:109, 1962). However, the Tₘ of a duplex DNA decreases by 1° C. with every increase of 1% in the number of mismatch base pairs (Bonner and Laskey, Eur J Biochem 46:83, 1974. Formamide is optional in these hybridization conditions. Accordingly, in a particular embodiment levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

As used herein, the terms “co- or post-translational modifications” refer to covalent modifications occurred during or after translation of the peptide chain. Exemplary co- or post-translational modifications include but are not limited to acylation (including acetylation), amidation or deamidation, biotinylation, carbamylation (or carbamoylation), carboxylation or decarboxylation, disulfide bond formation, fatty acid acylation (including myristoylation, palmitoylation and stearoylation), formylation, glycation, glycosylation, hydroxylation, incorporation of selenocysteine, lipidation, lipoic acid addition, methylation, N- or C-terminal blocking, N- or C-terminal removal, nitration, oxidation of methionine, phosphorylation, proteolytic cleavage, prenylation (including farnesylation, geranyl geranylation), pyridoxal phosphate addition, sialylation or desialylation, sulfation, ubiquitinylation (or ubiquitination) or addition of ubiquitin-like proteins.

Acylation involves the hydrolysis of the N-terminus initiator methionine and the addition of an acetyl group to the new N-termino amino acid. Acetyl Co-A is the acetyl donor for acylation.

Amidation is the covalent linkage of an amide group to the carboxy terminus of a peptide and is frequently required for biological activity and stability of a protein. Deamidation is the hydrolytic removal of an amide group. Deamidation of amide containing amino acid residues is a rare modification that is performed by the organism to re-arrange the 3D structure and alter the charge ratio/pI.

Biotinylation is a technique whereby biotinyl groups are incorporated into molecules, either that catalyzed by holocarboxylase synthetase during enzyme biosynthesis or that undertaken in vitro to visualise specific substrates by incubating them with biotin-labeled probes and avidin or streptavidin that has been linked to any of a variety of substances amenable to biochemical assay.

Carbamylation (or carbamoylation) is the transfer of the carbamoyl from a carbamoyl-containing molecule (e.g., carbamoyl phosphate) to an acceptor moiety such as an amino group.

Carboxylation of glutamic acid residues is a vitamin K dependent reaction that results in the formation of a gamma carboxyglutamic acid (Gla residue). Gla residues within several proteins of the blood-clotting cascade are necessary for biological function of the proteins. Carboxylation can also occur to aspartic acid residues.

Disulfide bonds are covalent linkages that form when the thiol groups of two cysteine residues are oxidized to a disulfide. Many mammalian proteins contain disulfide bonds, and these are crucial for the creation and maintenance of tertiary structure of the protein, and thus biological activity.

Protein synthesis in bacteria involves formylation and deformylation of N-terminal methionines. This formylation/deformylation cycle does not occur in cytoplasm of eukaryotic cells and is a unique feature of bacterial cells. In addition to the hydroxylation that occurs on glycine residues as part of the amidation process, hydroxylation can also occur in proline and lysine residues catalysed by prolyl and lysyl hydroxylase (Kivirikko et al. FASEB Journal 3:1609-1617, 1989).

Glycation is the uncontrolled, non-enzymatic addition of glucose or other sugars to the amino acid backbone of protein.

Glycosylation is the addition of sugar units to the polypeptide backbone and is further described hereinafter.

Hydroxylation is a reaction which is dependent on vitamin C as a co-factor. Adding to the importance of hydroxylation as a post-translation modification is that hydroxy-lysine serves as an attachment site for glycosylation.

Selenoproteins are proteins which contain selenium as a trace element by the incorporation of a unique amino acid, selenocysteine, during translation. The tRNA for selenocysteine is charged with serine and then enzymatically selenylated to produce the selenocysteinyl-tRNA. The anticodon of selenocysteinyl-tRNA interacts with a stop codon in mRNA (UGA) instead of a serine codon. An element in the 3′ non-translated region (UTR) of selenoprotein mRNAs determines whether UGA is read as a stop codon or as a selenocysteine codon.

Lipidation is a generic term that encompasses the covalent attachment of lipids to proteins, this includes fatty acid acylation and prenylation.

Fatty acid acylation involves the covalent attachment of fatty acids such as the 14 carbon Myristic acid (Myristoylation), the 16 carbon Palmitic acid (Palmitoylation) and the 18 carbon Stearic acid (Stearoylation). Fatty acids are linked to proteins in the pre-Golgi compartment and may regulate the targeting of proteins to membranes (Blenis and Resh Curr Opin Cell Biol 5(6):984-9, 1993). Fatty acid acylation is, therefore, important in the functional activity of a protein (Bernstein Methods Mol Biol 237:195-204, 2004).

Prenylation involves the addition of prenyl groups, namely the 15 carbon farnesyl or the 20 carbon geranyl-geranyl group to acceptor proteins. The isoprenoid compounds, including farnesyl diphosphate or geranylgeranyl diphosphate, are derived from the cholesterol biosynthetic pathway. The isoprenoid groups are attached by a thioether link to cysteine residues within the consensus sequence CAAX, (where A is any aliphatic amino acid, except alanine) located at the carboxy terminus of proteins. Prenylation enhances proteins ability to associate with lipid membranes and all known GTP-binding and hydrolyzing proteins (G proteins) are modified in this way, making prenylation crucial for signal transduction. (Rando Biochim Biophys Acta 1300(1):5-16, 1996; Gelb et al. Curr Opin Chem Biol 2(1):40-8, 1998).

Lipoic acid is a vitamin-like antioxidant that acts as a free radical scavenger. Lipoyl-lysine is formed by attaching lipoic acid through an amide bond to lysine by lipoate protein ligase.

Protein methylation is a common modification that can regulate the activity of proteins or create new types of amino acids. Protein methyltransferases transfer a methyl group from S-adenosyl-L-methionine to nucleophilic oxygen, nitrogen, or sulfur atoms on the protein. The effects of methylation fall into two general categories. In the first, the relative levels of methyltransferases and methylesterases can control the extent of methylation at a particular carboxyl group, which in turn regulates the activity of the protein. This type of methylation is reversible. The second group of protein methylation reactions involves the irreversible modification of sulfur or nitrogen atoms in the protein. These reactions generate new amino acids with altered biochemical properties that alter the activity of the protein (Clarke Curr Opin Cell Biol 5:977 983, 1993).

Protein nitration is a significant post-translational modification, which operates as a transducer of nitric oxide signalling. Nitration of proteins modulates catalytic activity, cell signalling and cytoskeletal organization.

Phosphorylation refers to the addition of a phosphate group by protein kinases. Serine, threonine and tyrosine residues are the amino acids subject to phosphorylation. Phosphorylation is a critical mechanism, which regulates biological activity of a protein.

A majority of proteins are also modified by proteolytic cleavage. This may simply involve the removal of the initiation methionine. Other proteins are synthesized as inactive precursors (proproteins) that are activated by limited or specific proteolysis. Proteins destined for secretion or association with membranes (preproteins) are synthesized with a signal sequence of 12-36 predominantly hydrophobic amino acids, which is cleaved following passage through the ER membrane.

Pyridoxal phosphate is a co-enzyme derivative of vitamin B6 and participates in transaminations, decarboxylations, racemizations, and numerous modifications of amino acid side chains. All pyridoxal phosphate-requiring enzymes act via the formation of a Schiff base between the amino acid and coenzyme. Most enzymes responsible for attaching the pyridoxal-phosphate group to the lysine residue are self activating.

Sialylation refers to the attachment of sialic acid to the terminating positions of a glycoprotein via various sialyltransferase enzymes; and desialylation refers the removal of sialic acids. Sialic acids include but are not limited to, N-acetyl neuraminic acid (NeuAc) and N-glycolyl neuraminic acid (NeuGc). Sialyl structures that result from the sialylation of glycoproteins include sialyl Lewis structures, for example, sialyl Lewis a and sialyl Lewis x, and sialyl T structures, for example, Sialyl-TF and Sialyl Tn.

Sulfation occurs at tyrosine residues and is catalyzed by the enzyme tyrosylprotein sulfotransferase which occurs in the trans-Golgi network. It has been determined that 1 in 20 of the proteins secreted by HepG2 cells and 1 in 3 of those secreted by fibroblasts contain at least one tyrosine sulfate residue. Sulfation has been shown to influence biological activity of proteins. Of particular interest is that the CCR5, a major HIV co-receptor, was shown to be tyrosine-sulfated and that sulfation of one or more tyrosine residues in the N-terminal extracellular domain of CCR5 are required for optimal binding of MIP-1 alpha/CCL3, MIP-1 beta/CCL4, and RANTES/CCL5 and for optimal HIV co-receptor function (Moore J Biol Chem 278(27):24243-24246, 2003). Sulfation can also occur on sugars. In addition, sulfation of a carbohydrate moiety of a glycoprotein can occur by the action of glycosulfotransferases such as GalNAc(β1-4)GlcNAc(β1-2)Manα4 sulfotransferase.

Post-translational modifications can encompass protein-protein linkages. Ubiquitin is a 76 amino acid protein which both self associates and covalently attaches to other proteins in mammalian cells. The attachment is via a peptide bond between the C-terminus of ubiquitin and the amino group of lysine residues in other proteins. Attachment of a chain of ubiquitin molecules to a protein targets it for proteolysis by the proteasome and is an important mechanism for regulating the steady state levels of regulatory proteins e.g. with respect to the cell cycle (Wilkinson Annu Rev Nutr 15:161-89, 1995). In contrast, mono-ubiquitination can play a role in direct regulation of protein function. Ubiquitin-like proteins that can also be attached covalently to proteins to influence their function and turnover include NEDD-8, SUMO-1 and Apg12.

Glycosylation is the addition of sugar residues in the polypeptide backbone. Sugar residues, such as monosaccharides, disaccharides and oligosaccharides include but are not limited to: fucose (Fuc), galactose (Gal), glucose (Glc), galactosamine (GalNAc), glucosamine (GlcNAc), mannose (Man), N-acetyl-lactosamine (lacNAc) N,N′-diacetyllactosediamine (lacdiNAc). These sugar units can attach to the polypeptide back bones in at least seven ways, namely,
(1) via an N-glycosidic bond to the R-group of an asparagine residue in the consensus sequence Asn-X-Ser; Asn-X-Thr; or Asn-X-Cys (N-glycosylation).
(2) via an O-glycosidic bond to the R-group of serine, threonine, hydroxyproline, tyrosine or hydroxylysine (O-glycosylation).
(3) via the R-group of tyrosine in C-linked mannose;
(4) as a glycophosphatidylinositol anchor used to secure some proteins to cell membranes;
(5) as a single monosaccharide attachment of GlcNAc to the R-group of serine or threonine. This linkage is often reversibly associated with attachment of inorganic phosphate (Yin-o-Yang);
(6) attachment of a linear polysaccharide to serine, threonine or asparagine (proteoglycans);
(7) via a S-glycosidic bond to the R-group of cysteine.

The glycosylation structure can comprise one or more of the following carbohydrate antigenic determinants in Table 7.

TABLE 7
List of carbohydrate antigenic determinants

| Antigenic Name | Antigenic Glycan Structure |
|---|---|
| Blood group H(O), type | Fuc(α1-2)Gal(β1-3)GlcNAc-R |
| 1 | |
| Blood group H(O), type | Fuc(α1-2)Gal(β1-4)GlcNAc-R |
| 2 | |
| Blood group A, type 1 | GalNAc(α1-3)[Fuc(α1-2)]Gal(β1-3)GlcNAc-R |
| Blood group A, type 2 | GalNAc(α1-3)[Fuc(α1-2)]Gal(β1-4)GlcNAc-R |
| Blood group B, type 1 | Gal(α1-3)[Fuc(α1-2)]Gal(β1-3)GlcNAc-R |
| Blood group B, type 2 | Gal(α1-3)[Fuc(α1-2)]Gal(β1-4)GlcNAc-R |
| Blood group i | [Gal(β1-4)GlcNAc(β1-3)]ₙGal(β1-R |
| Blood group I | Gal(β1-4)GlcNAc(β1-3)[Gal(β1-4)GlcNAc(β1- |
| | 6)]Gal(β1-4)GlcNAc(β1-3)Gal(β1-R |
| Lewis a (Le^{a}) | Gal(β1-3)[Fuc(α1-4)]GlcNAc-R |
| Sialyl Lewis a (sLe^{a}) | NeuAc(α2-3)Gal(β1-3)[Fuc(α1-4)]GlcNAc-R |
| Lewis b (Le^{b}) | Fuc(α1-2)Gal(β1-3)[Fuc(α1-4)]GlcNAc-R |
| Lewis x (Le^{x}) | Gal(β1-4)[Fuc(α1-3)]GlcNAc-R |
| Sialyl Lewis x (sLe^{x}) | NeuAc(α2-3)Gal(β1-4)[Fuc(α1-3)]GlcNAc-R |
| Lewis y (Le^{y}) | Fuc(α1-2)Gal(β1-4)[Fuc(α1-3)]GlcNAc-R |
| Forssman | GalNAc(α1-3)GalNAc(β1-3)Gal-R |
| Thomsen-Friedenreich | Gal(β1-3)GalNAc(α1-O)-Ser/Thr |
| (TF or T) | |
| Sialyl-TF (sTF) or Sialyl- | Gal(β1-3)[NeuAc(α2-6)]GalNAc(α1-O)- |
| T (sT) | Ser/Thr |
| Tn | GalNAc(α1-O)-Ser/Thr |
| Sialyl Tn (sTn) | NeuAc(α2-6)GalNAc(α1-O)-Ser/Thr |

The carbohydrates will also contain several antennary structures, including mono, bi, tri and tetra outer structures.

Glycosylation may be measured by the presence, absence or pattern of N-linked glycosylation, O-linked glycosylation, C-linked mannose structure, and glycophosphatidylinositol anchor; the percentage of carbohydrate by mass; Ser/Thr-GalNAc ratio; the proportion of mono, bi, tri and tetra sugar structures or by lectin or antibody binding.

Sialylation of a protein may be measured by the immunoreactivity of the protein with an antibody directed against a particular sialyl structure. For example, Lewis x specific antibodies react with CEACAM1 expressed from granulocytes but not with recombinant human CEACAM1 expressed in 293 cells (Lucka et al. Glycobiology 15(1):87-100, 2005). Alternatively, the presence of sialylated structures on a protein may be detected by a combination of glycosidase treatment followed by a suitable measurement procedure such as mass spectroscopy (MS), high performance liquid chromatography (HPLC) or glyco mass fingerprinting (GMF).

The apparent molecular weight of a protein includes all elements of a protein complex (cofactors and non-covalently bonded domains) and all co- or post-translational modifications (addition or removal of covalently bonded groups to and from peptide). Apparent molecular weight is often affected by co- or post-translational modifications. A protein's apparent molecular weight may be determined by SDS-PAGE (sodium dodecyl sulfate polyacrylamide gel electrophoresis), which is also the second dimension on its two-dimensional counterpart, 2D-PAGE (two-dimensional polyacrylamide gel electrophoresis). It may be determined more accurately, however, by mass spectrometry (MS)— either by Matrix-Assisted Laser Desorption Ionization-Time of Flight (MALDI-TOF) MS, which produces charged molecular ions or the more sensitive Electrospray Ionization (ESI) MS, which produces multiple-charged peaks. The apparent molecular weights of the protein or chimeric molecule thereof may be within the range of 1 to 1000 kDa. Accordingly, the isolated protein or chimeric molecule of the present invention has a apparent molecular weight of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 kDa. The molecular weight or molecular mass of a protein may be determined by any convenient means such as electrophoresis, mass spectrometry, gradient ultracentrifugation.

The isoelectric point (or pI) of a protein is the pH at which the protein carries no net charge. This attribute may be determined by isoelectric focusing (IEF), which is also the first dimension of 2D-PAGE. Experimentally determined pI values are affected by a range of co- or post-translational modifications and therefore the difference between an experimental pI and theoretical pI may be as high as 5 units. Accordingly, an isolated protein or chimeric molecule of the present invention may have a pI of 0, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0.

As used herein, the term “isoform” means multiple molecular forms of a given protein, and includes proteins differing at the level of (1) primary structure (such as due to alternate RNA splicing, or polymorphisms); (2) secondary structure (such as due to different co- or post translational modifications); and/or (3) tertiary or quaternary structure (such as due to different sub-unit interactions, homo- or hetero-oligomeric multimerization). In particular, the term “isoform” includes glycoform, which encompasses a protein or chimeric molecule thereof having a constant primary structure but differing at the level of secondary or tertiary structure or co- or post-translational modification such as different glycosylation forms.

Chemical stability of a protein may be measured as the “half-life” of the protein in a particular solvent or environment. Typically, proteins with a molecular weight of less than 50 kDa have a half-life of approximately 5 to 20 minutes. The proteins or chimeric molecules of the present invention are contemplated to have a half-life of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 hours. Another particularly convenient measure of chemical stability is the resistance of a protein or chimeric molecule thereof to protease digestion, such as trypsin or chymotrypsin digestion.

The binding affinity of a protein or chimeric molecule thereof to its ligand or receptor may be measured as the equilibrium dissociation constant (Kd) or functionally equivalent measure.

The solubility of a protein may be measured as the amount of protein that is soluble in a given solvent and/or the rate at which the protein dissolves. Furthermore, the rate and or level of solubility of a protein or chimeric molecule thereof in solvents of differing properties such as polarity, pH, temperature and the like may also provide measurable physiochemical characteristics of the protein or chimeric molecule thereof.

Any “measurable physiochemical parameters” may be determined, measured, quantified or qualified using any methods known to one of skill in the art. Described below is a range of methodologies which may be useful in determining, measuring, quantifying or qualifying one or more measurable physiochemical parameters of an isolated protein or chimeric molecule thereof. However, it should be understood that the present invention is in no way limited to the particular methods described, or to the measurable physiochemical parameters that are measurable using these methods.

Glycoproteins can be said to have two basic components that interact with each other to create the molecule as a whole—the amino acid sequence and the carbohydrate or sugar side chains. The carbohydrate component of the molecule exists in the form of monosaccharide or oligosaccharide side chains attached to the amine side chain of Asn or the hydroxyl side chain of Ser/Thr residues of the amino acid backbone by N- or O-linkages, respectively. A monosaccharide is the term given to the smallest unit of a carbohydrate that is regarded as a sugar, having the basic formula of (CH₂O)ₙ and most often forming a ring structure of 5 or 6 atoms (pentoses and hexoses respectively). Oligosaccharides are combinations of monosaccharides forming structures of varying complexities that may be either linear or branched but which generally do not have long chains of tandem repeating units (such as is the case for polysaccharides). The level of branching that the oligosaccharide contains as well as the terminal and branching substitutions dramatically affect the properties of the glycoprotein as a whole, and play an important role in the biological function of the molecule. Oligosaccharides are manufactured and attached to the amino acid backbone in the endoplasmic reticulum (ER) and Golgi apparatus of the cell. Different organisms and cell types have different ratios of glycotransferases and endoglycosidases and exoglycosidases and therefore produce different oligosaccharide structures. One of the fundamental defence mechanisms of the body is the detection and destruction of aberrant isoforms and as such it is important to have correct glycosylation of a biological therapeutic not only to increase effectiveness but also to decrease detection by neutralizing antibodies.

Glycan chains are often expressed in a branched fashion, and even when they are linear, such chains are often subject to various modifications. Thus, the complete sequencing of oligosaccharides is difficult to accomplish by a single method and therefore requires iterative combinations of physical and chemical approaches that eventually yield the details of the structure under study.

Determination of the glycosylation pattern of a protein can be performed using a number of different systems, for example using SDS-PAGE. This technique relies on the fact that glycosylated proteins often migrate as diffuse bands by SDS-PAGE. Differentiation between different isoforms are performed by treating a protein with a series of agents. For example, a marked decrease in band width and change in migration position after digestion with peptide-N4-(N-acetyl-β-D-glucosaminyl) asparagine amidase (PNGase) is considered diagnostic of N-linked glycosylation.

To determine the composition of N-linked glycosylation, N-linked oligosaccharides are removed from the protein with PNGase cloned from Flavobacterium meningosepticum and expressed in E. coli. The removed N-linked oligosaccharides may be recovered from Alltech Carbograph SPE Carbon columns (Deerfield, Ill., USA) as described by Packer et al. Glycoconj J 5(8):737-47, 1998. The sample can then be taken for monosaccharide analysis, sialic acid analysis or sulfate analysis on a Dionex system with a GP50 pump ED50 pulsed Amperometric or conductivity detector and a variety of pH anion exchange columns.

The extent of O-linked glycosylation may be determined by first removing O-linked oligosaccharides from the target protein by β-elimination. The removed O-linked oligosaccharides may be recovered from Alltech Carbograph SPE Carbon columns (Deerfield, Ill., USA) as described by Packer et al. (1998, supra). The sample can then be taken for monosaccharide analysis, sialic acid analysis or sulfate analysis on a Dionex system with a GP50 pump ED50 pulsed Amperometric or conductivity detector and a variety of pH anion exchange columns.

Monosaccharide subunits of an oligosaccharide have variable sensitivities to acid and thus can be released from the target protein by mild trifluoro-acetic acid (TFA) conditions, moderate TFA conditions, and strong hydrochloric acid (HCl) conditions. The monosaccharide mixtures are then separated by high pH anion exchange chromatography (HPAEC) using a variety of column media, and detected using pulsed amperometric electrochemical detection (PAD).

High-pH anion-exchange chromatography with pulsed amperometric detection (HPAEC-PAD) has been extensively used to determine monosaccharide composition. Fluorophore-based labeling methods have been introduced and many are available in kit form. A distinct advantage of fluorescent methods is an increase in sensitivity (about 50-fold). One potential disadvantage is that different monosaccharides may demonstrate different selectivity for the fluorophore during the coupling reaction, either in the hydrolyzate or in the external standard mixture. However, the increase in sensitivity and the ability to identify which monosaccharides are present from a small portion of the total amount of available glycoprotein, as well as the potential for greater sensitivity using laser-induced fluorescence, makes this approach attractive. In addition a conductivity detector may be used to determine the sulfate and phosphate composition. By using standards, the peak areas can be calculated to total amounts of each monosaccharide present. These data can indicate the level of N- and O-linked glycosylation, the extent of sialylation, and in combination with amino acid composition, percent by weight glycosylation, percent by weight acidic glycoproteins.

Monosaccharide composition analysis of small amounts of protein is best performed with PVDF (PSQ) membranes, after electroblotting, or, if smaller aliquots are to be analyzed, on dot blots. PVDF is an ideal matrix for carbohydrate analysis because neither monosaccharides nor oligosaccharides bind to the membrane, once released by acid or enzymatic hydrolysis.

Determination of the oligosaccharide content of the target molecule is performed by a number of techniques. The sugars are first removed from the amino acid backbone by enzymatic (such as digestion with PNGase)) or chemical (such as beta elimination with hydroxide) means. The sugars may be stabilised by reduction or labeled with a fluorophore for ease of detection. The resultant free oligosaccharides are then separated either by high pH anion exchange chromatography with pulsed amperometric electrochemical detection (HPAEC-PAD), which can be used with known standards to determine the ratios of the various structures and levels of sialylation, or by fluorophore assisted carbohydrate electrophoresis (FACE) a process similar to SDS-PAGE separation of proteins. In this process the oligosaccharides are labeled with a fluorophore that imparts electrophoretic mobility. They are separated on high percentage polyacrylamide gels and the resultant band pattern provides a profile of the oligosaccharide content of the target molecule. By using standards it is possible to gain some information on the actual structures present or the bands can be cut and analysed using mass spectrometry to determine each of their structures.

Fluorophore assisted carbohydrate electrophoresis (FACE) is a polyacrylamide gel electrophoresis system designed to separate individual oligosaccharides that have been released from a glycoconjugate. Oligosaccharides are removed from the sample protein by either chemical or enzymatic means in such a way as to retain the reducing terminus. Oligosaccharides are then either digested into monosaccharides or left intact and labeled with a fluorophore (either charged or non charged). High percentage polyacrylamide gels and various buffer systems are used to migrate the oligosaccharides/monosaccharides which migrate relative to their size/composition in much the same way as proteins. Sugars are visualised by densitometry and relative amounts of sugars can be determined by fluorophore detection. This process is compatible with MALDI-TOF MS, hence the method can be used to elucidate actual structures.

Quartz crystal microbalance and surface plasmon resonance (QCM and SPR, respectively) are two methods of obtaining biological information through the physiochemical properties of a molecule. Both measure protein-protein interactions indirectly through the change that the interaction causes in the physical characteristics of a prefabricated chip. In QCM a single crystal quartz wafer is treated with a receptor/antibody etc which interacts with the ligand of interest. This chip is oscillated by the microbalance and the frequency of the chip recorded. The protein of interest is allowed to pass over the chip and the interaction with the bound molecule causes the frequency of the wafer to change. By changing the conditions by which the ligand interacts with the chip, it is possible to determine the binding characteristics of the target molecule.

Apparent molecular weight is also a physiochemical property which can be used to determine the similarities between the protein or chimeric molecule of the present invention and those produced using alternative means.

As used herein, the term “molecular weight” is defined as the sum of atomic weights of the constituent atoms in a molecule, sometimes also referred to as “molecular mass” (Mr). Molecular weight can be determined theoretically by summing the atomic masses of the constituent atoms in a molecule. The term “apparent molecular weight” is defined as the molecular weight determined by one or more analytical techniques such as SDS page or ultra centrifugation and depends on the relationship between the molecule and the detection system. The apparent molecular weight of a protein or chimeric molecule thereof can be determined using any one of a range of experimental methods. Analytical methods for determining the molecular weight of a protein include, without being limited to, size-exclusion chromatography (SEC), gel electrophoresis, Rayleigh light scattering, analytical ultracentrifugation, and, to some extent, time-of-flight mass spectrometry.

Gel electrophoresis is a process of determining some of the physiochemical properties (specifically apparent molecular weight and pI) of a protein and in the case of 2 dimensional electrophoresis to separate the molecule into isoforms, thereby providing information on the post-translational modifications of the protein product. Specifically, electrophoresis is the process of forcing a charged molecule (such as protein or DNA) to migrate through a gel matrix (most commonly polyacrylamide or agarose) by applying an electric potential through its body. The most common forms of electrophoresis used on proteins are isoelectric focussing, native, and SDS polyacrylamide gel electrophoresis. In isoelectric focussing a protein is placed into a polyacrylamide gel that has a pH gradient across its length. The protein will migrate to the point in the gel where it has a net charge of zero thereby giving its isoelectric point.

Glyco mass fingerprinting (GMF) is the process by which the oligosaccharide profile of a protein or one of its isoforms is identified by electrophoresis followed by specific mass spectrometric techniques. Sample protein is purified either by 1D SDS-PAGE for total profile determination or 2D gel electrophoresis for specific isoform characterization. The protein band/spot is excised from the gel and de-stained to remove contaminants. The sugars are released by chemical or enzymatic means and desalted/separated using a nanoflow LC system and a graphitised carbon column. The LC flow can be directly injected into an electrospray mass spectrometer that is used to determine the mass and subsequently the identity of the oligosaccharides present on the sample. This provides a profile or fingerprint of each isoform which can be combined with quantitative techniques such as Dionex analysis to determine the total composition of the molecule being tested.

Primary structure can be evaluated in determining the physiochemical properties of the protein or chimeric molecule of the present invention.

The primary structure of a protein or chimeric molecule thereof can be assayed using one or more of the following systems.

Information on the primary structure of a protein or chimeric molecule thereof can be determined using a combination of mass spectrometry (MS), DNA sequencing, amino acid composition, protein sequencing and peptide mass fingerprinting.

To determine the sequence of the amino acid backbone either N-terminal chemical sequencing, tandem mass spectrometry sequencing, or a combination of both is used. N-terminal chemical sequencing utilises Edman chemistry (Edman P. “Sequence determination” Mol Biol Biochem Biophys 8:211-55, 1970), which states that the peptide bond between the N-terminal amino acid and the amino acid in position 2 of the protein is weaker than all other peptide bonds in the sequence. By using moderate acidic conditions the N-terminal amino acid is removed derivatised with a fluorophore (FTIC) and the retention time on a reversed-phase HPLC column determined, and compared to a standard to identify what the amino acid is. This method will determine the actual primary structure of the molecule but is not quantitative. Alternatively tandem mass spectrometry in conjunction with nanoflow liquid chromatography may be used (LC-MS/MS). In this process the protein is digested into peptides using specific endoproteases and the molecular weight of the peptides determined. High energy collision gases such as nitrogen or argon are then used to break the peptide bonds and the masses of the resultant peptides measured. By calculating the change in mass of the peptides it is possible to determine the sequence of each of the peptides (each amino acid has a unique mass). By using different proteases the peptides may then be overlapped to determine their order and thus the entire sequence of the protein.

Clearly, the combination of enzymatic digestion, chemical derivatization, liquid chromatography (LC)/MS and tandem MS provides an extremely powerful tool for AA sequence analysis. For example, the detailed structure of recombinant soluble CD4 receptor was characterized by a combination of methods, which confirmed over 95% of the primary sequence of this 369 AA glycoprotein and showed the whole nature of both N- and C-termini, the positions of attachment of the glycans, the structures of the glycans and the correct assignment of the disulfide bridges (Carr et al. J Biol Chem 264(35):21286-21295, 1989).

Mass spectrometry (MS) is the process of measuring the mass of a molecule through extrapolation of its behavior in a charged environment under a vacuum. MS is very useful in stability studies and quality control. The method first requires digestion of samples by proteolytic enzymes (trypsin, V8 protease, chymotrypsin, subtilisin, and clostripain) (Franks et al. Characterization of proteins, Humana Press, Clifton, N.J., 1988; Hearn et al. Methods in Enzymol 104:190-212, 1984) and then separation of digested samples by reverse phase chromatography (RPC). With tryptic digestion in conjunction with LC-MS, the peptide map can be used to monitor the genetic stability, the homogeneity of production lots, and protein stability during fermentation, purification, dosage form manufacture and storage.

Before a mass analysis, several ways are used to interface a HPLC to a mass spectrometer: 1) direct liquid injection; 2) supercritical fluid; 3) moving belt system; 4) thermospray. The HPLC/MS interface used in Caprioli's work used a fused silica capillary column to transport the eluate from the column to the tip of the sample probe in the ionization chamber of the mass spectrometer. The probe tip is continuously bombarded with energetic Xe atoms, causing sputtering of the sample solution as it emerges from the tip of the capillary. The mass is then analyzed by the instrument (Caprioli et al. Biochem Biophys Res Commun 146:291-299, 1987).

MS/MS and LC/MS interfaces expand the potential applications of MS. MS/MS allows direct identification of partial to full sequence for peptides up to 25 AAs, sites of deamidation and isomerization (Carr et al. Anal Chem 63:2802-2824, 1991). Coupled with RPC or capillary electrophoresis (CE), MS can perform highly sensitive analysis of proteins (Figeys and Aebersold, Electrophoresis 19:885-892, 1998; Nguyen et al. J Chromatogr A 705:21-45, 1995). LC/MS allows LC methodology to separate peptides before entering the MS, such as the continuous flow FAB interfaced with microbore HPLC (Caprioli et al. 1987, supra). The latter “interface” allows the sequencing of individual peptides from complex mixtures: Fragmentation of the peptides selected by the first MS is followed by passing through a cloud of ions in a collision cell: CID (collision induced dissociation). The collision generates characteristic set of fragments, from which the sequence may be deduced, without knowing other information, such as the cDNA sequence. In a single MS experiment, an unfractionated mixture of peptides (e.g. from an enzyme digest) is injected and the masses of the major ions are compared with those predicted from the cDNA sequence. The sequence of the recombinant human interleukin-2 was verified by fast atom bombardment (FAB)-MS analysis of CNBr and proteolytic digests (Fukuhara et al. J Biol Chem 260:10487-10494, 1985).

Electrospray ionization MS (ESI-MS) uses an aerosol of solution protein to introduce into a needle under a high voltage, generating a series of charged peaks of the same molecules with various charges. Because each peak generated from the differently charged species produces an estimation of the molecular weights, these estimations can be combined to increase the overall precision of the molecular weight estimation. Matrix Assisted Laser Desorption Ionization MS (MALDI-MS) uses a high concentration of a chromophore. A higher intensity laser pulse will be absorbed by the matrix and the energy absorbed evaporates part of the matrix and carries the protein sample with it into the vapor phase almost entirely. The resulting ions are then analyzed in a time of flight MS. The mild ionization may enhance the capacity of the method to provide quaternary structure information. MALDI-MS can be run rapidly in less than 15 minutes. It does not need to fragment the molecules and the result is easy to interpret as a densitometric scan of an SDS-PAGE gel, with a mass range up to over 100 kDa.

Amino acid sequence can be predicted by sequencing DNA that encodes a protein or chimeric molecule thereof. However, occasionally the actual protein sequence may be different. Traditionally, DNA sequencing reactions are just like the PCR reactions for replicating DNA (DNA denaturation, replication). By DNA cloning technology, the gene is cloned, and the nucleotide sequence determined.

The amino acid sequence of a protein or chimeric molecule thereof can be assayed using one or more of the following systems.

Full sequence description of the protein or chimeric molecule thereof is usually required to describe the product. Amino acid sequencing includes: in gel tryptic digestion, fractionation of the digested peptides by RPC-HPLC, screening the peptide peaks that have the most symmetrical absorbance profile by MALDI-TOF MS, and the first peptide (N-terminal) by Edman degradation. Edman chemically derived primary sequence data is the classical method to identify proteins at the molecular level. MALDI-TOF MS can be used for N-terminal sequence analysis. However, all enzymatic digests for HPLC and peptide sequencing are recommended to first be subjected to MS or MS/MS protein identification to decrease the time and cost. The internal amino acid sequences from SDS-PAGE-separated proteins are obtained by elution of the peptides with HPLC separation after an in situ tryptic or lysyl endopeptidase digestion in the gel matrix.

Internal sequencing of the standard peptide is recommended to be run with the analyzed samples to maintain the instruments at the peak performance. More than 80% of higher eukaryotic proteins are reported to have blocked amino-termini that prevent direct amino acid sequencing. When a blocked eukaryotic protein is encountered, the presence of the sequence of the internal standard assures that the instrument is operating properly.

Edman degradation can be used for direct N-terminal sequencing with a chemical procedure, which derivatizes the N-terminal amino acids to release the amino acids and expose the amino terminal of the next AAs. The Edman sequencing includes: 1) By microbore HPLC, N-terminal sequence analysis is repeated by Edman chemistry cycles. Every cycle of the Edman chemistry can identify one amino acid. 2) After in-gel or PVDF bound protein digestions followed by HPLC separation of the resulting peptides, internal protein sequence analysis is conducted by Edman degradation chemistry.

Microbore HPLC and capillary HPLC are used for analysis and purification of peptide mixtures using RPC-HPLC. In-gel samples and PVDF samples are purified using different columns. MALDI-TOF MS analysis can be used for N-terminal analysis after HPLC fractionation. The selection criteria are: 1) The apparent purity of the HPLC fraction. 2) The mass and thus the estimated length of the peptide. The peptide mass information is useful for confirming the Edman sequencing amino acid assignments, and also in the possible detection of co- or post-translational modifications.

In-gel digests are suitable for purification on the higher sensitivity HPLC system. The internal protein sequence analysis is first enzymatically digested by SDS-PAGE. Proteins in an SDS-PAGE mini-gel can be reliably digested in-gel only with trypsin. The peptide fragments are purified by RPC-HPLC and then analyzed by MALDI-TOF MS, screening for peptides suitable for Edman sequence analysis. Proteins in a gel can only be analyzed by internal sequencing analysis, but very accurate peptides masses can be obtained, which provides additional information useful in both amino acid assignment and database searching.

PVDF-bound proteins are suitable for both N-terminal and internal Edman sequencing analysis. PVDF-bound proteins are digested with the proper enzyme (trypsin, endoproteinase Lys-C, endoproteinase Glu-C, clostripain, endoproteinase Asp-N, thermolysin) and a non-ionic detergent such as hydrogenated Triton X-100. In PVDF bound proteins, the detergents used for releasing digested peptides from the membrane can interfere with MALDI-TOF MS analysis. Before the enzyme is added, Cys is reduced with DTT and alkylated with iodoacetamide to generate carboxyamidomethyl Cys, which can be identified during N-terminal sequence analysis.

To determine the amino acid composition of a protein or chimeric molecule thereof, the sample is hydrolyzed using phenol catalyzed strong hydrochloric acid (HCl) acidic conditions in the gaseous phase. Once the hydrolysis is performed the liberated amino acids are derivatised with a fluorophore compound that imparts a specific reversed phase characteristic on the combined molecule. The derivatized amino acids are separated using reversed phase high performance liquid chromatography (RP-HPLC) and detected with a fluorescence detector. By using external and internal standards it is possible to calculate the amount of each amino acid present in the sample from the observed peak area. This information may be used to determine sample identity and to quantify the amount of protein present in the sample. For instance, discrepancies between theoretical and actual results can be used to initially identify the possibility of a de-amidation site. In combination with monosaccharide analysis it may determine the composition % by weight glycosylation and percent by weight acidic glycoproteins. This method is limited in the information that it can provide on the actual sequence of the backbone however as there is inherent variability due to environmental contaminants and occasional destruction of amino acids. For example, it is not possible for this method to detect point mutations in the sequence.

Peptide mass fingerprinting (PMF) is another method by which the identity of a protein or chimeric molecule thereof may be determined. The procedure involves an initial separation of the sample by electophoretic means (either 1 or 2 dimensional), excision of the spot/band from the gel and digestion with a specific endoprotease (typically porcine trypsin). Peptides are eluted from the gel fragment and analysed by mass spectrometry to determine the peptide masses present. The resultant peptide masses are then compared to a database of theoretical mass fragments for all reported proteins (or in the case of constructs for the theoretical peptide masses of the designed sequence). The technique relies on the fact that the “fingerprint” of a protein (i.e. its combination of peptide masses) is unique. Identity can be confidently determined (greater than 90% accuracy) with as little as 4 peptides and 30% sequence coverage. Modifications such as lipid moieties and de-amidation can be identified during the PMF stage of analysis. Peaks that do not correspond to those of the identified protein are further analysed by tandem mass spectrometry (MS-MS), a technique that uses the energy created by the impact of a collision gas to break the weaker bond of the PTM. The newly freed molecule and the original peptide are then re-analysed for mass to identify the post-translational modification and the peptide fragment to which it was attached.

HPLC is classified into different modes depending on the size, charge, hydrophobicity, function or specific content of the target biomolecules. Generally, two or more chromatographic methods are used to purify a protein. It is of paramount importance to consider both the characteristics of the protein and the sample solvent when the chromatographic modes are selected.

Secondary structures of a protein or chimeric molecule of the present invention can also be evaluated in characterising their properties.

The secondary structure of a protein or chimeric molecule thereof can be assayed using one or more of the following systems.

To study the secondary structures of proteins, most commonly several spectroscopic methods should be applied and compared. Electromagnetic energy can be defined as a continuous waveform of radiation, depending on the size and shape of the wave. Different spectroscopic methods use different electromagnetic energy.

The wavelength, is the extent of a single wave of radiation (the distance between two successive maxima of the waves). When the radiant energy increases, the wavelength becomes shorter. The relationship between frequency and wavenumber is:

Wavenumber (cm⁻¹)=Frequency (s⁻¹)/The speed of light (cm/s).

The absorption of electromagnetic radiation by molecules includes vibrational and rotational transitions, and electronic transitions. Infrared (IR) and Raman spectroscopy are most commonly used to measure the vibrational energies of molecules in order to determine secondary structure. However, they are different in their approach to determine molecular absorbance.

The energy of the scattered radiation is less than the incident radiation for the Stokes line. The energy of the scattered radiation is more than the incident radiation for the anti-Stokes line. The energy increase or decrease from the excitation is related to the vibrational energy spacing in the ground electronic state of the molecule. Therefore, the wavenumber of the Stokes and anti-Stokes lines are a direct measure of the vibrational energies of the molecule.

Only the Stokes shift is observed in a Raman spectrum. The Stokes lines are at smaller wavenumbers (or higher wavelengths) than the exciting light. A high power excitation source, such as a laser, should be used to enhance the efficiency of Raman scattering. The excitation source should be monochromatic because we are interested in the energy (wavenumber) difference between the excitation and the Stokes lines.

For a vibrational motion to be IR active, the dipole moment of the molecule must change. Therefore, the symmetric stretch in carbon dioxide is not IR active because there is no change in the dipole moment. The asymmetric stretch is IR active due to a change in dipole moment. For a vibration to be Raman active, the polarizability of the molecule must change with the vibrational motion. The symmetric stretch in carbon dioxide is Raman active because the polarizability of the molecule change. Thus, Raman spectroscopy complements IR spectroscopy (Herzberg et al. Infrared and Raman Spectra of Polyatomic Molecules, Van Nostrand Reinhold, New York, N.Y., 1945). For example, IR is not able to detect a homonuclear diatomic molecule due to the lack of dipole moments, but Raman spectroscopy can detect it because the molecular polarizability is changed by stretching and contraction of the bond, further, the interactions between electrons and nuclei are changed.

For highly symmetric polyatomic molecules with a center of inversion (such as benzene), it is more likely that bands active in the IR spectrum are not active in the Raman spectrum or vice-versa. In molecules with little or no symmetry, modes are likely to be active in both infrared and Raman spectroscopy.

IR spectroscopy measures the wavelength and intensity of the absorption of infrared light by a sample. Infrared light is so energetic that it can excite the molecular vibrations to higher energy levels. Both infrared and RAMAN spectroscopy measure the vibrations of bond lengths and angles.

IR characterizes vibrations in molecules by measuring the absorption of light of certain energies corresponding to the vibrational excitation of the molecule from v=0 to v=1 (or higher) states. There are selection rules that govern the ability of a molecule to be detected by infrared spectroscopy—Not all of the normal modes of vibration can be excited by infrared radiation (Herzberg et al. 1945, supra).

IR spectra can provide qualitative and quantitative information of the secondary structures of proteins, such as α helix, β sheet, β turn and disordered structure. The most informative IR bands for protein analysis are amide I (1620-1700 cm⁻¹), amide II (1520-1580 cm⁻¹) and amide III (1220-1350 cm⁻¹). Amide I is the most intense absorption band in proteins. It consists of stretching vibration of the C═O (70-85% and C—N groups (10-20%). The exact band position is dictated by the backbone conformation and the hydrogen bonding pattern. Amide II is more complex than Amide I. Amide II is governed by in-plane N—H bending (40-60%), C—N (18-40%) and C—C (10%) stretching vibrations. Amide III bands are not very useful (Krimm and Bandekar, Adv Protein Chem 38:181-364, 1986). Most of the β-sheet structures of FTIR amide I band usually are located at about 1629 cm⁻¹ with a minimum of 1615 cm⁻¹ and a maximum of 1637 cm⁻¹; the minor component may show peaks around 1696 cm⁻¹ (lowest value 1685 cm⁻¹). α-helix is mainly found at 1652 cm⁻¹. An absorption near 1680 cm⁻¹ is now assigned to β turns.

The principle of Raman scattering is different from that of infrared absorption. Raman spectroscopy measures the wavelength and intensity of inelastically scattered light from molecules. The Raman scattered light occurs at wavelengths that are shifted from the incident light by the energies of molecular vibrations.

To be Raman active, for the vibration to be inelastically scattered, a change in polarizability during the vibration is essential. In the symmetric stretch, the strength of electron binding is different between the minimum and maximum internuclear distances. Therefore the polarizability changes during the vibration, and this vibrational mode scatters Raman light, the vibration is Raman active. In the asymmetric stretch the electrons are more easily polarized in the bond that expands but are less easily polarized in the bond that compresses. There is no overall change in polarizability and the asymmetric stretch is Raman inactive (Herzberg et al. 1945, supra).

Circular dichroism can be used to detect any asymmetrical structures, such as proteins. Optically active chromophores absorb different amount of right and left polarized light, this absorbance difference results in either a positive or negative absorption spectrum (Usually, the right polarized spectrum is subtracted from the left polarized spectrum). Commonly, the far UV or amide region (190-250 nm) is mainly contributed from peptide bonds, providing information on the environment of the carbonyl group of the amide bond and consequently the secondary structure of the protein. α helix usually displays two negative peaks at 208, 222 nm (Holzwarth et al. J Am Chem Soc 178:350, 1965), β sheet displays one negative peak at 218 nm, random coils has a negative peak at 196 nm. Near UV region peaks are (250-350 nm) contributed from the environment of the aromatic chromophores (Phe, Tyr, Trp). Disulfide bonds give rise to minor CD bands around 250 nm.

Intense dichroism is commonly associated with the side-chain structures being held tightly in a highly folded, three-dimensional structure. Denaturation of the protein mostly releases the steric hindrance, a weaker CD spectrum is obtained along with an increasing degree of denaturation. For example, the side chain CD spectrum of hGH is quite sensitive to the partial denaturation by adding denaturants. Some reversible chemical alterations of the molecules, such as reduction of the disulfide bonds, or alkaline titrations will change the side-chain CD spectrum. For hGH, these spectral difference can be caused by entirely the removal of a chromophores, or by affecting changes in the particular chromophore's CD response, but not by the gross denaturation or conformational changes (Aloj et al. J Biol Chem 247:1146-1151, 1971).

UV absorption spectroscopy is one of the most significant methods to determine protein properties. It can provide information about protein concentrations and the immediate environments of chromophoric groups. Proteins functional groups, such as amino, alcoholic (or phenolic) hydroxyl, carbonyl, carboxyl, or thiol can be transformed into strong chromophores. Visible and near UV spectroscopy are used to monitor two types of chromophores: metalloproteins (more than 400 nm) and proteins that contains Phe, Trp, Tyr residues (260-280 nm). The change in UV or fluorescence signal can be negative or positive, depending on the protein sequence and solution properties.

Fluorescence measures the emission energy after the molecule has been irradiated into an excited state. Many proteins emitted fluorescence in the range of 300 to 400 nm when excited at 250 to 300 nm from their aromatic amino acids. Only proteins with Phe, Trp, Tyr residues can be measured with the order of intensity Trp>>Tyr>>Phe. Fluorescence spectra can reflect the microenvironments information that are affected by the folding of the proteins. For example, a buried Trp is usually in a hydrophobic environment and will fluoresce at maximum 325 to 330 nm range, but an exposed residue or free amino acids fluoresces at around 350 to 355 nm. An often used agent to probe protein unfolding is Bis-ANS. The fluorescence of Bis-ANS is pH-independent. Even though its signal is weak in water, it can be increased significantly by binding to unfolding-exposed hydrophobic sites in proteins (James and Bottomley Arch Biochem Biophy 356:296-300, 1998).

Effective quenching of Tyr and Trp in the folded proteins causes significant signal increase upon unfolding. A simple solute may cause the change also. To maximize detection sensitivity, a signal ratio can be used. For example, In the study of rFXIII unfolding, a ratio of fluorescence intensity at 350 nm to that at 330 nm was used (Kurochkin et al. J Mol Biol 248:414-430, 1995). Conformational changes may be studied by means of excitation-energy transfer between a fluorescent donor and an absorbing acceptor, because the efficiency of transfer depends on the distance between the two chromophores (Honroe et al. Biochem J 258:199-204, 1989). Fluorescence was used to probe a-Antitrypsin conformation (Kwon and Yu, Biophim Biophys Acta 1335:265-272, 1997), to determine Tm of HSA (Farruggia et al. Int J Biol Macromol 20:43-51, 1997), and to detect MerP unfolding interactions (Aronsson et al. FEBS Lett 411:359-364, 1997).

At neutral pH, the intensity of the fluorescence emission spectrum is in the order of Trp>Tyr. At acidic pH, due to the conformational changes which disrupts the energy transfer, the fluorescence from Tyr dominates over Trp. Fluorescence studies also confirm the presence of intermediates in the guanidine-induced unfolding transition of the proteins. Tertiary and quaternary structures of the physiochemical forms of a protein or chimeric molecule of the present invention are also important in ascertaining their function.

The tertiary and quaternary structures of a protein or chimeric molecule thereof can be assayed using one or more of the following systems.

NMR and X-ray crystallography are the most often used techniques to study the 3D structure of proteins. Other less detailed methods to probe protein tertiary structure include CD in near UV region, second-derivative of UV spectroscopy (Ackland et al. J Chromatogr 540:187-198, 1991) and fluorescence.

NMR is one of the main experimental methods for molecular structure and intermolecular interactions in structural biology. In addition to studying protein structures, NMR can also be utilised to study the carbohydrate structures of a protein or chimeric molecule of the present invention. NMR spectroscopy is routinely used by chemists to study chemical structure using simple one-dimensional techniques. The structure of more complicated molecules can also be determined by two-dimensional techniques. Time domain NMR are used to probe molecular dynamics in solutions. Solid state NMR is used to determine the molecular structure of solids. NMR can be used to study structural and dynamic properties of proteins, nucleic acids, a variety of low molecular weight compounds of biological, pharmacological and medical interests. However, not all nuclei possess the correct property in order to be read by NMR, i.e., not all nuclei posses spin, which is required for NMR. The spin causes the nucleus to produce an NMR signal, functioning as a small magnetic field.

The crystal structure of a protein or chimeric molecule thereof can be assayed using one or more of the following systems.

X-ray crystallography is an experimental technique that applies the fact that X-rays are diffracted by crystals. X-rays have the appropriate wavelength (in the Ångström range, ˜10-8 cm) to be scattered by the electron cloud of an atom of comparable size. The electron density can be reconstructed based on the diffraction pattern obtained from X-ray scattering off the periodic assembly of molecules or atoms in the crystal. Additional phase information either from the diffraction data or from supplementing diffraction experiments should be obtained to complete the reconstruction. A model is then progressively built into the experimental electron density, refined against the data and the result is a very accurate molecular structure.

X ray diffraction has been developed to study the structure of all states of matter with any beam, e.g., ions, electrons, neutrons, and protons, with a wavelength similar to the distance between the atomic or molecular structures of interest.

Light scattering spectroscopy is based on the simple principle that larger particles scatter light more than the smaller particles. A slope base line in the 310-400 nm region originates from light scattering when large particles, such as aggregates, present in the solution (Schmid et al. Protein structure, a practical approach, Creighton Ed., IRI Press, Oxford, England, 1989)

Light scattering spectroscopy can be used to estimate the molecular weight of a protein and is a simple tool to monitor protein quaternary structure or protein aggregation. The degree of protein aggregation can be indicated by simple turbidity measurement. Final product pharmaceutical solutions are subjected to inspection of clarity because most aggregated proteins are present as haze and opalescence. Quasielastic light scattering spectroscopy (QELSS), sometimes called photon correlation spectroscopy (PCS), or dynamic light scattering (DLS), is a noninvasive probe of diffusion in complex fluids for macromolecules (proteins, polysaccharides, synthetic polymers, micelles, colloidal particles and aggregations). In most cases, light scattering spectroscopy yields directly the mutual diffusion coefficient of the scattering species. When applied to dilute monodisperse solutions, the diffusion coefficient obtained by QELSS can estimate the size. With polydisperse system, it estimates the width of molecular weight distribution. For accurate measurement, 200-500 mW laser power is mandatory, conventional Ar+/Kr+ gas lasers are widely used (Phillies Anal Chem 62:1049A-1057A, 1990). Protein aggregation was detected by human relaxin (Li et al. Biochemistry 34:5762-5772, 1995).

Stability of a protein or chimeric molecule thereof is also an important determinant of function. Methods for analysing such characteristics include DSC, TGA and freeze-dry cryostage microscopy, analysis of freeze-thaw resistance, and protease resistance.

A protein or chimeric molecule of the present invention may be more stable for lyophilization (freeze drying). Lyophilization is used to enhance the stability and/or shelf life of the product as it is stored in powder rather than liquid form. The process involves an initial freezing of the sample, then removal of the liquid by evaporation under vacuum. The end result is a dessicated “cake” of protein and excipients (other substances used in the formulation). The consistency of the resulting cake is critical for successful reconstitution. The lyophilization process can result in changes to the protein, especially aggregate formation though crosslinking, but also deamidation and other modifications. These can reduce efficacy by either losses, reduced activity or by inducing immune reactions against aggregates. In order to test lyophilization stability, the protein can be formulated for lyophilization using standard stabilizers (e.g. mannitol, trehalose, Tween 80, human serum albumin and the like). After lyophilization, the amount of protein recovered can be assayed by ELISA, while its activity can be assayed by a suitable bioassay. Aggregates of the protein can be detected by HPLC or Western blot analysis.

Prior to lyophilization, the Tg or Te (define Tg or Te) of the formulation should be determined to set the maximum allowable temperature of the product during primary drying. Also, information about the crystallinity or amorphousity of the formulation helps to design the lyophilization cycle in a more rationale manner. Product information on these thermal parameters can be obtained by using differential scanning calorimetry (DSC), thermogravimetric analysis (TGA) or freeze-dry cryostage microscope.

Differential Scanning Calorimetry (DSC) is a physical thermo-analytical method to measure, characterize and analyze thermal properties of materials and determine the heat capacities, melting enthalpies and transition points accordingly. DSC scans through a temperature range at a linear rate. Individual heaters within the instrument provide heat to sample and reference pans separately, based on the “power compensated null balance” principle. During a physical transition, the absorption or evolution of the energy causes an imbalance in the amount of energy supplied to that of the sample holder. Depending on the varying thermal behavior of the sample, the energy will be taken or diffused from the sample, and the temperature difference will be sensed as an electrical signal to the computer. As a result, an automatic adjustment of the heaters makes the temperature of the sample holder identical to the reference holder. The electrical power needed for the compensation is equivalent to the calorimetric effect.

The purity of an organic substance can be estimated by DSC based on the shape and temperature of the DSC melting endotherm. The power-compensated DSC provides very high resolution compared to a heat flux DSC under the identical conditions. More well-defined and more accurate partial areas of melting can be generated from power-compensated DSC because the partial areas of melting are not “smeared” over a narrow temperature interval, as for the lesser-resolved heat flux DSC. The power-compensated DSC produces inherently better partial melting areas and therefore better purity analysis. By the help of StepScan DSC, the power-compensated DSC can provide a direct heat capacity measurement using the traditional and time-proven means without the need for deconvolution or the extraction of sine wave amplitudes.

Thermogravimetric Analysis (TGA) measures sample mass loss and the rate of weight loss as a function of temperature or time.

As DSC, freeze-dry cryostage can reach a wide temperature range rapidly. Currently, as an preformulation and formulation study tool, simulating the lyophilization cycle in a freeze dry cryostage provides the best platform to study thermal parameters of the protein formulations on a miniature scale. Freeze dry microscope can predict the influence of formulations and process factors on freezing and drying. Only a 2-3 mL sample is required for a cryostage study, which makes this technique a valuable tool to study scarce, difficult-to-obtain drugs. It is a good tool to study the effect of freezing, rate, drying rate, thawing rate on the lyophilization cycle. Annealing research may be advanced by the studies from freeze-dry cryostage microscope. Because of extensive applications of lyophilization technology, and larger demand to stabilize the extremely expensive drugs (such as proteins and gene therapy drugs), it is expected that an in-process microscopic monitor should be realized in the pharmaceutical industries soon.

The freeze-thaw resistance of a protein or chimeric molecule thereof can be assayed using one or more of the following systems.

Co- or post translational modification such as glycosylation may protect proteins from repeated freeze/thaw cycles. To determine this, a protein or chimeric molecule of the present invention can be compared to carrier-free E. coli-produced counterparts. A protein or chimeric molecule thereof are diluted into suitable medium (e.g. cell growth medium, PBS or the like) then frozen by various methods, for instance, snap frozen in liquid nitrogen, slowly frozen by being placed at −70 degrees or rapidly frozen on dry ice. The samples are then thawed either rapidly at room temperature or slowly at 4 degrees. Some samples are then refrozen and the process are repeated for a number of cycles. The amount of protein present can be measured by ELISA, and the activity measured in a suitable bioassay chosen by a skilled artisan. The amount of activity/protein remaining is compared to the starting material to determine the resistance over many the freeze/thaw cycles.

A protein or chimeric molecule of the present invention may have altered thermal stability in solution. The thermal stability of the present invention may be determined in vitro as follows.

A protein or chimeric molecule of the present invention can be mixed into buffer e.g. phosphate buffered saline containing carrier protein e.g. human serum albumin and incubated at a particular temperature for a particular time (e.g. 37 degrees for 7 days). The amount of protein or chimeric molecule thereof remaining after this treatment can be determined by ELISA and compared to material stored at −70 degrees. The biological activity of the remaining protein or chimeric molecule thereof is determined by performing a suitable bioassay chosen by a person skilled in the relevant art.

The protease resistance of a protein or chimeric molecule thereof can be assayed using one or more of the following systems.

To compare protease resistance, solution containing a protein or chimeric molecule of the present invention and solution containing E. coli expressed counterparts can be incubated with a protease of choice (e.g. unpurified serum proteases, purified proteases, recombinant proteases) for different time periods. The amount of protein remaining is measured by an appropriate ELISA (e.g. one in which the epitopes recognized by the capture and detection antibodies are separated by the protease cleavage site), and the activity of the remaining protein or chimeric molecule thereof is determined by a suitable bioassay chosen by a skilled artisan.

The bioavailability of a protein or chimeric molecule thereof can be assayed using one or more of the following systems.

Bioavailability is the degree to which a drug or other substance becomes available to the target tissue after administration. Bioavailability may depend on half life of the drug or its ability to reach the target tissue.

Compositions comprising a protein or chimeric molecule of the present invention is injected subcutaneously or intramuscularly. The levels of the protein or its chimeric molecule can then be measured in the blood by ELISA or radioactive counts. Alternatively, the blood samples can be assayed for activity of the protein by a suitable bioassay chosen by a skilled artisan, for instance, stimulation of proliferation of a particular target cell population. As the sample will be from plasma or serum, there may be a number of other molecules that could be responsible for the output activity. This can be controlled by using a neutralizing antibody to the protein being tested. Hence, any remaining bioactivity is due to the other serum components.

The stability or half-life of a protein or chimeric molecule thereof can be assayed using one or more of the following systems.

A protein or chimeric molecule of the present invention may have altered half-life in serum or plasma. The half-life of the present invention may be determined in vitro as follows. Composition containing the protein or chimeric molecule of the present invention can be mixed into human serum/plasma and incubated at a particular temperature for a particular time (e.g. 37 degrees for 4 hours, 12 hours etc). The amount of protein or chimeric molecule thereof remaining after this treatment can be determined by ELISA. The biological activity of the remaining protein or chimeric molecule thereof is determined by performing a suitable bioassay chosen by a person skilled in the relevant art. The serum chosen may be from a variety of human blood groups (e.g. A, B, AB, O etc.)

The half-life of a protein or chimeric molecule thereof can also be determined in vivo. Composition containing a protein or chimeric molecule thereof, which may be labeled by a radioactive tracer or other means, can be injected intravenously, subcutaneously, retro-orbitally, tail vein, intramuscularly or intraperitoneally) into the species of choice for the study, for instance, mouse, rat, pig, primate, human. Blood samples are taken at time points after injection and assayed for the presence of the protein or chimeric molecule thereof (either by ELISA or by TCA-precipitable radioactive counts). A comparison composition consisting of E. Coli or CHO-produced protein or chimeric molecule thereof can be run as a control.

To determine the half-life of protein or chimeric molecule of the present invention, in vivo, male Wag/Rij rats, or other suitable animals can be injected intravenously with a protein or chimeric molecule thereof.

Just before the administration of the substrate, 200 μl of EDTA blood is sampled as negative control. At various time points after the injection, 200 μl EDTA blood can be taken from the animals using the same technique. After the last blood sampling, the animals are sacrificed. The specimen is centrifuged for 15 min at RT within 30 min of collection. The plasma samples are tested in a specific ELISA to determine the concentration of protein or chimeric molecule of the present invention in each sample.

A protein or chimeric molecule of the present invention may cross the blood brain barrier.

An in vitro assay to determine if protein or chimeric molecule of the present invention binds human brain endothelial cells can be tested using the following assays.

Radiolabeled protein or chimeric molecule of the present invention can be tested for its ability to bind to human brain capillary endothelial cells. An isolated protein or chimeric molecule of the present invention can be custom conjugated with radiolabel to a specific activity using a method known in the art, for instance, with ¹²⁵I by the chloramine T method, or with ³H.

Primary cultures of human brain endothelial cells can be grown in flat-bottom 96-well plates until five days post-confluency then lightly fixed using acetone. Cells are lysed, transferred to glass fibre membranes. Radiolabeled protein or chimeric molecule of the present invention can be detected using a liquid scintillation counter.

In vivo assays for the determination of protein or chimeric molecule of the present invention binding to human brain endothelial cells can be tested using the following assays.

A human-specific protein or chimeric molecule of the present invention are tested for binding to human brain capillaries using sections of human brain tissue that are fresh frozen (without fixation), sectioned on a cryostat, placed on glass slides and fixed in acetone. Binding of ³H-protein or chimeric molecule of the present invention is examined on brain sections using quantitative autoradiography.

In vivo assay can be used to measure tissue distribution and blood clearance of human-specific protein or chimeric molecule of the present invention in a primate system.

A protein or chimeric molecule of the present invention is used to determine the tissue distribution and blood clearance of ¹⁴C-labeled protein or chimeric molecule of the present invention in 2 male cynomolgus monkeys or other suitable primates. protein or chimeric molecule of the present invention is administered concurrently with a ³H-labeled control protein to the animals with an intravenous catheter. During the course of the study, blood samples are collected to determine the clearance of the proteins from the circulation. At 24 hours post-injection, the animals are euthanized and selected organs and representative tissues collected for the determination of isotope distribution and clearance by combustion. In addition, capillary depletion experiments are performed to samples from different regions of the brain in accordance with Triguero, et al. J of Neurochemistry 54:1882-1888, 1990. This method removes greater than 90% of the vasculature from the brain homogenate (Triguero et al. cited supra).

The time-dependent redistribution of the radiolabeled protein or chimeric molecule of the present invention from the capillary fraction to the parenchyma fraction is consistent with the time dependent migration of a protein or chimeric molecule of the present invention across the blood-brain barrier.

A protein or chimeric molecule of the present invention may promote or inhibit angiogenesis.

The angiogenic potential of the protein or chimeric molecule of the present invention may be assessed methods known in the art. For example, the extent of angiogenesis may be measured by microvessel sprouting in a model of angiogenesis. In this assay, rat fat microvessel fragments (RFMFs) are isolated as described in Shepherd et al. Arterioscler Thromb Vasc Biol 24:898-904, 2004. Epididymal fat pads are harvested from euthanized animals, minced and digested in collagenase. RFMFs and single cells are separated from lipids and adipocytes by centrifugation and suspended in 0.1% BSA in PBS. The RFMF suspension is sequentially filtered to remove tissue debris, single cells, and red blood cells from the fragments. RFMFs are suspended in cold, pH-neutralized rat-tail type 1 collagen at 15,000 RFMF/ml and plated into wells (for example, 0.25 ml/well) of 48-well plate for culture. After polymerization of the collagen, an equal volume of DMEM containing 10% FBS is added to each gel. After formation of the gels, vascular extensions characteristic of angiogenic sprouts appear by day 4 of culture. These sprouts are readily distinguished from the parent vessel fragment by the absence of the rough, smooth-muscle associated appearance. The RFMF 3-D cultures can be treated with the protein or chimeric molecule of the present invention and vessel sprout lengths can be measured at day 5 and 6 of culture.

The angiogenic potential of the protein or chimeric molecule of the present invention may also be assessed by an in vivo angiogenesis assay described in Guedez et al. Am J Pathol 162:1431-1439, 2003. This assay consists of subcutaneous implantation of semiclosed silicone cylinders (angioreactors) into nude mice. Angioreactors are filled with extracellular matrix premixed with or without the protein or chimeric molecule of the present invention. Vascularization within angioreactors is quantified by the intravenous injection of fluorescein isothiocyanate (FITC)-dextran before their recovery, followed by spectrofluorimetry. Angioreactors examined by immunofluorescence is able to show cells and invading angiogenic vessels at different developmental stages.

A protein or chimeric molecule of the present invention may have a distinct immunoreactivity profile determined by immunoassay techniques, which involve the interaction of the molecule with one or more antibodies directed against the molecule. Examples of immunoassay techniques include enzyme-linked immunoabsorbant assays (ELISA), dot blots and immunochromatographic assays such as lateral flow tests or strip tests.

The level of the protein or chimeric molecule thereof may be measured using an immunoassay procedure, for example, a commerically purchased ELISA kit. The protein or chimeric molecule of the present invention may have a different immunoreactivity profile to non-human cell expressed protein or chimeric molecule thereof due to the specificity of the antibodies provided in an immunoassay kit. For instance, the capture and/or detection antibodies of the immunoassay may be antibodies specifically directed against non-human cell expressed human protein or chimeric molecule thereof.

In addition, incorrect folding of the non-human cell expressed human protein or chimeric molecule thereof may result in the exposure of antigenic epitopes which are not exposed on the correctly folded human cell expressed human protein or chimeric molecule thereof. Incorrect folding may arise through, for instance, overproduction of heterologous proteins in the cytoplasm of non-human cells, for example, E. coli (Baneyx Current Opinion in Biotechnology, 10:411-421, 1999). Further, non-human cell expressed human protein or chimeric molecule thereof may have a different pattern of post-translational modifications to that of the protein or chimeric molecule of the present invention. For example, the non-human cell expressed human protein or chimeric molecule thereof may exhibit abnormal quantities and/or types of carbohydrate structures, phosphate, sulfate, lipid or other residues. This may result in the exposure of antigenic epitopes which are not exposed on the protein or chimeric molecule of the present invention. Conversely, an altered pattern of post-translational modifications may result in an absence of antigenic epitopes on the protein or chimeric molecule of the present invention which are exposed on the non-human cell expressed human protein or chimeric molecule thereof.

Any one of, or combination of, the above-mentioned factors may lead to inaccurate measurements of:
(a) naturally occurring human protein in laboratory samples or human tissues, or
(b) human cell expressed recombinant human protein or chimeric molecule thereof in laboratory samples, human tissues or in human embryonic stem cell (hES) culture media.

The immunoreactivity profile of a human cell expressed human protein or chimeric molecule thereof, as determined by the use of a suitable immunoassay, may provide an indication of the protein's immunogenicity in the human, as described hereinafter.

Most biologic products elicit a certain level of antibody response against them. The antibody response can, in some cases, lead to potentially serious side effects and/or loss of efficacy. For instance, some patients treated with recombinant protein or chimeric molecule thereof expressed from non-human cells may generate neutralizing antibodies particularly during long-term therapeutic use and thereby reducing the protein's efficacy and or contribute to side effects. The protein or chimeric protein molecule expressed from human cells is unlikely to generate neutralizing antibodies therefore increasing its therapeutic efficacy compared with non-human cell expressed protein or chimeric molecule thereof.

The immunogenicity of protein or chimeric molecule thereof can be assayed using one or more of the following systems.

Most biologic products elicit a certain level of antibody response against them. The antibody response can, in some cases, lead to potentially serious side effects and/or loss of efficacy. For instance, some patients treated with recombinant EPO will generate neutralizing antibodies that also cross-react with the patient's own EPO. In this case, they can develop pure red cell aplasia and be resistant to EPO treatment, resulting in a need for constant dialysis.

Immunogenicity is the property of being able to evoke an immune response within an organism. Immunogenicity depends partly upon the size of the substance in question and partly upon how unlike host molecules it is. A protein or chimeric molecule thereof may have altered immunogenicity due to its novel physiochemical characteristics. For instance, the glycosylation structure of a protein or chimeric molecule thereof may shield or obscure the epitope(s) recognized by the antibody and therefore preventing or reducing antibody binding to the protein or chimeric molecule thereof. Alternatively, some antibodies may recognize a glycopeptide epitope not present in the non-glycosylated version of the protein.

The ability of patient samples to recognize a protein or chimeric molecule thereof with a distinctive physiochemical form can be determined by various immunoassays, as described herein. A properly designed immunoassay involves considerations directing to appropriate detection, quantitation and characterization of antibody responses. A number of recommendations for the design and optimization of immunoassays are outlined in Mire-Sluis et al. J Immunol Methods 289(1-2):1-16, 2004, which is incorporated by reference.

The use of protein or chimeric molecule thereof on therapeutic implants can be assayed using one or more of the following systems.

The present invention extends to the use of a protein or chimeric molecule thereof to manipulate stem cells. A major therapeutic use of stem cells is in regeneration of tissue, cartilage or bone. In one embodiment, the cells are likely to be introduced to the body in a biocompatible three-dimensional matrix. The implant will consist of a mixture of cells, the scaffold, growth factors and accessory components such as biodegradable polymers, proteoglycans and the like. Incorporation of a protein or chimeric molecule thereof into these matrices during their construction is proposed to regulate the behavior of the cells. Such implants may be used for the formation of bone, the growth of neurons from progenitor cells, chondrocyte implantation for cartilage replacement and other applications. Human cell-derived proteins may reduce the quantity and/or variety of xenogeneic proteins from stem cell culture conditions and thereby reduce the risks of infection by non-human pathogens.

A protein or chimeric molecule of the present invention may interact differently with the matrix used for the formation of the implant, as well as regulating the cells incorporated within the implant. It is anticipated that the combination of a protein or chimeric molecule of the present invention with the implant components will result in one or more of the following pharmacological traits, such as higher proliferation, enhanced differentiation, maintenance in a desired state of differentiation, greater lineage specificity of differentiation, enhanced secretion of matrix components, better 3-dimensional structure formation, enhanced signaling, better structural performance, reduced toxicity, reduced side effects, reduced inflammation, reduced immune cell infiltrate, reduced rejection, longer duration of the implant, longer function of the implant, better stimulation of the cells surrounding the implant, better tissue regeneration, better organ function, or better tissue remodeling.

The effects of protein or chimeric molecule thereof on differential gene expression can be assayed using one or more of the following systems.

The differences in gene expression can be analyzed in cells exposed to a protein or chimeric molecule thereof.

Microarray technology enables the simultaneous determination of the mRNA expression of almost all genes in an organism's genome. This method uses gene “chips” in which oligonucleotides corresponding to the sequences of different genes are attached to a solid support. Labeled cDNA derived from mRNA isolated from the cell or tissue of interest is incubated with the chips to allow hybridisation between cDNA and the attached complementary sequence. A control is also used, and following hybridisation and washing the signal from both is compared. Specialised software is used to determine which genes are up or down regulated or which have unchanged expression. Many thousands of genes can be analysed on each chip. For example using Affymetrix technology, the Human Genome U133 (HG-U133) Set, consisting of two GeneChip (registered trade mark) arrays, contains almost 45,000 probe sets representing more than 39,000 transcripts derived from approximately 33,000 well-substantiated human genes. The GeneChip (registered trade mark) Mouse Genome 430 2.0 contains over 39,000 transcripts on a single array.

This type of analysis reveals changes in the global mRNA expression pattern and therefore differences in the expression of genes not known to be controlled by a particular stimulus may be uncovered. This technology is hence suitable to analyze the induced gene expression associated with protein or chimeric molecule of the present invention.

The definition of known and novel genes regulated by the particular stimulus will assist in the identification of the biochemical pathways that are important in the biological activity of the particular protein or chimeric molecule of the present invention. This information will be useful in the identification of novel therapeutic targets.

The system could also be used to look at differences in gene expression induced by a protein or chimeric molecule of the present invention as compared to commercially available products.

The effects of protein or chimeric molecule thereof on binding ability can be assayed using one or more of the following systems.

The binding ability of a protein or chimeric molecule of the present invention to various substances, including extracellular matrix, artificial materials, heparin sulfates, carriers or co-factors can be investigated.

The effects of a protein or chimeric molecule thereof on the ability of a particular protein to bind an extracellular matrix can be determined using the following assays.

A surface is coated with extracellular matrix proteins, including but not limited to collagen, vitronectin, fibronectin, laminin, in an appropriate buffer. The unbound sites can be blocked by methods known in the art, for instance, by incubation with BSA solution. The surface is washed, for instance, with PBS solutions, then a solution containing the protein to be tested, for instance a protein or chimeric molecule of the present invention, is added to the surface. After coating, the surface is washed and incubated with an antibody that recognizes a protein or chimeric molecule thereof. Bound antibody is then detected, for instance, by an enzyme-linked secondary antibody that recognizes the primary antibody. The bound antibodies are visualized by incubating with the appropriate substrate and observing a colour change reaction. Glycosylated proteins may adhere more strongly to the extracellular matrix proteins than unglycosylated proteins.

Alternatively, an equivalent amount (specified by ELISA concentration or bioassay activity units) of a protein or chimeric molecule of the present invention, or a counterpart protein or chimeric molecule thereof expressed by non-human cells, are incubated with matrix coated wells, then following washing of the wells the amount bound is determined by ELISA. The amount bound can be indirectly measured by a drop in ELISA reactivity following incubation of the sample with the coated surface.

The ability of protein or chimeric molecule thereof to bind artificial materials can be assayed using one or more of the following systems.

In order to determine the binding ability of a protein or chimeric molecule thereof to artificial materials, a surface is coated with artificial material, including but not limited to metals, scaffolds, in an appropriate buffer. The surface is washed, for instance, with PBS solutions, then a solution containing the protein to be tested, for instance a protein or chimeric molecule of the present invention, is added to the surface. After coating, the surface is washed and incubated with an antibody that recognizes a protein or chimeric molecule thereof. Bound antibody is then detected, for instance, by a enzyme-linked secondary antibody that recognizes the primary antibody. The bound antibodies are visualized by incubating with the appropriate substrate and observing a color change reaction.

Alternatively, an equivalent amount (specified by ELISA concentration or bioassay activity units) of a protein or chimeric molecule of the present invention, and a counterpart protein or chimeric molecule thereof expressed by non-human cells, are incubated with wells coated by artificial materials, the wells are then washed and the amount bound is determined by ELISA. The amount bound can be indirectly measured by a drop in ELISA reactivity following incubation of the sample with the coated surface.

Ability to bind to artificial surfaces may have biological consequences, for instance, in stent coating. Alternatively, a scaffold coated with a protein or chimeric molecule of the present invention is used to seed cells on. The cell growth and differentiation is then monitored and compared to uncoated or differentially coated scaffolds.

The ability of protein or chimeric molecule thereof to bind to heparin sulfates can be assayed using one or more of the following systems.

A protein or chimeric molecule of the present invention is expected to interact differentially with heparin sulfates due to their physiochemical form. These differences are expected to be evident in experimental models of cell proliferation, differentiation, migration and the like. The combination of a protein or chimeric molecule thereof with heparin sulfates is expected to have distinctive pharmacological traits for a given treatment. This may be an increase in serum half-life, bioavailability, reduced immune-related clearance, greater efficacy, reduced dosage fewer side effects and related advantages.

The ability of protein or chimeric molecule thereof to bind to carriers or co-factors can be assayed using one or more of the following systems.

Proteins or chimeric molecules thereof will be bound to other molecules when they are present in plasma. These molecules may be termed “carriers” or “co-factors” and will influence such factors as bioavailability or serum half life.

Incubating purified versions of the proteins in plasma and analyzing the resulting solution by size exclusion chromatography can determine the interaction of a protein or chimeric molecule of the present invention with their binding partners. If the protein or chimeric molecule thereof binds a co-factor, the resulting complex will have a larger molecular weight, resulting in an altered elution time. The complex can be compared for biological activity, in vitro or in vivo half-life and bioavailability.

The effects of protein or chimeric molecule thereof on bioassays can be assayed using one or more of the following systems.

Various bioassays can be performed to test the activity of a protein or chimeric molecule of the present invention, including assays on cell proliferation, cell differentiation, cell apoptosis, cell size, cytokine/cytokine receptor adhesion, cell adhesion, cell spreading, cell motility, migration and invasion, chemotaxis, ligand-receptor binding, receptor activation, signal transduction, and alteration of subgroup ratios.

The effects of protein or chimeric molecule thereof on cell proliferation can be assayed using one or more of the following systems.

Cells, in a particular embodiment, exponentially growing cells, are incubated in a growth medium in the presence of a protein or chimeric molecule of the present invention. This can be performed in flasks or 96 well plates. The cells are grown for a period of time and then the number of cells is determined by either a direct (e.g. cell counting) or an indirect (MTT, MTS, tritiated thymidine) method. The increase or decrease in proliferation is determined by comparison with a medium only control assay. Different concentrations of protein or chimeric molecule thereof can be used in parallel series of experiments to get a dose response profile. This can be used to determine the ED50 and ED100 (the dose required to generate the half maximal and maximal response effectively).

The effects of protein or chimeric molecule thereof on cell differentiation or maintenance of cells in an undifferentiated state can be assayed using one or more of the following systems.

Cells are incubated in a growth medium in the presence of a protein or chimeric molecule of the present invention. After a suitable period of time, the cells are assayed for indicators of differentiation. This may be the expression of particular markers on the cell surface, cytoplasmic markers, an alteration in the cell dimensions, shape or cytoplasmic characteristics. The markers may include proteins, sugar structures (e.g. glycosaminocglycans such as heparin sulfates, chondroitin sulfates etc.) lipids (glycosphingolipids or lipid bilayer components). These changes can be assayed by a number of techniques including microscopy, western blot, FACS staining or forward/side scatter profiles.

The effects of protein or chimeric molecule thereof on cell apoptosis can be assayed using one or more of the following systems.

Apoptosis is defined as programmed cell death, and is distinct from other methods of cell death such as necrosis. It is characterized by defined changes in the cells, such as activation of signaling pathways (e.g. Fas, TNFR) resulting in the activation of a subset of proteases know as caspases. Initiator caspase activation leads to the activation of the executioner caspases which cleave a variety of cellular proteins resulting in nuclear fragmentation, cleavage of nuclear lamins, blebbing of the cytoplasm and destruction of the cell. Apoptosis can be induced by protein ligands such as FasL, TNFa and lymphotoxin or by signals such as UV light and substances causing DNA damage.

Cells are incubated in a growth medium in the presence of protein or chimeric molecule thereof and or other agents as suitable for the assay. For instance, the presence of agents able to block transcription (actinomycin D) or translation (cycloheximide) may be required. Following incubation for an appropriate period, the number of cells is determined by a suitable method. A decrease in cell number may indicate apoptosis. Other indications of apoptosis may be obtained by staining of the cells, for instance, for annexins or observing characteristic laddering patterns of DNA. Further evidence for the confirmation of apoptosis may be achieved by preventing the expression of apoptotic markers by incubating with cell permeable caspases inhibitors (e.g. z-VAD FMK), then assaying for apoptotic markers.

A protein or chimeric molecule of the present invention may prevent apoptosis by providing a survival signal through cellular survival pathways such as the Bcl2 or Akt pathways. Activation of these pathways can be confirmed by western blotting for an increase in cellular Bcl2 expression, or for an increase in the activated (phosphorylated) form of Akt using a phospho-specific antibody directed against Akt.

For this assay, cells are incubated in the presence or absence of the survival factor (e.g. IL-3 and certain immune cells). A proportion of cells incubated in the absence of the survival factor will die by apoptosis upon extended culture, whereas cells incubated in sufficient quantities of survival factor will survive or proliferate. Activation of the cellular pathways responsible for these effects can be determined by western blotting, immunocytochemistry and FACS analysis.

The effects of a protein or chimeric molecule thereof on the inhibition of apoptosis can be assayed using one or more of the following systems.

A protein or chimeric molecule of the present invention is tested for in vitro activity to protect rat-, mouse- and human cortical neural cells from cell death under hypoxic conditions and with glucose deprivation. For this, neural cell cultures are prepared from rat embryos. To evaluate the effects of the protein or chimeric molecule of the present invention, the cells are maintained in modular incubator chambers in a water-jacketed incubator for up to 48 hours at 37° C., in serum-free medium with 30 mM glucose and humidified 95% air/5% CO₂ (normoxia) or in serum-free medium without glucose and humidified 95% N₂/5% CO₂ (hypoxia and glucose deprivation), in the absence or presence of the protein or chimeric molecule of the present invention. The cell cultures are exposed to hypoxia and glucose deprivation for less than 24 hour and thereafter returned to normoxic conditions for the remainder of 24 hour. The cytotoxicity is analyzed by the fluorescence of Alamar blue, which reports cell viability as a function of metabolic activity.

In another method, the neural cell cultures are exposed for 24 hours to 1 mM L-glutamate or a-amino-3-hydroxy-5-methylisoxazole-4-propionic acid (AMPA) under normoxic conditions, in the absence or presence of various concentrations of the protein or chimeric molecule of the present invention. The cytotoxicity is analyzed by the fluorescence of Alamar blue, which reports cell-viability as a function of metabolic activity.

A protein or its chimeric molecule may affect the growth, apoptosis, development, or differentiation of a variety of cells. These changes can be reflected by, among other measurable parameters, changes in the cell size and changes in cytoplasmic complexity, which are due to intracellular organelle development. For instance, keratinocytes induced to differentiate by suspension culture exhibit downregulation of surface markers such as β1 integrins, an increase in cell size and cytoplasmic complexity. The effects of a protein or chimeric molecule thereof on cell size, or cytoplasmic complexity can be assayed using one or more of the following systems.

FACS measures the amount of light scattered off by a cell when a beam of laser is incident on it. An argon laser providing light with a wavelength of 488 nm is frequently used. The larger the size of the cell, the greater the disruption of the beam of light in the forward direction, hence the level of forward scatter corresponds to the size of the cell. In order to measure changes in cell size, cells treated with a protein or chimeric molecule of the present invention are diluted in sheath fluid and injected into the flow cytometer (FACSVantage SE, Becton Dickinson). Untreated cells act as a control. The cells pass through a beam of light and the amount of forward scattering of the light corresponds to the size of the cells.

Changes in intracellular organelle growth and development (cytoplasmic complexity) can also be measured by FACS. The intracellular organelles of the cell scatter light sideways. Hence, change in cytoplasmic complexity can be measured by the amount of side scattering of light by the cells by the above method, and the level of complexity of intracellular organelles and the level of granularity of the cell can be estimated by measuring the level of side scatter of light given off by the cells.

The effect of a protein or chimeric molecule thereof on cell size or cytoplasmic complexity can be assessed by using FACS to compare the profiles given off by, for instance, 20,000 treated cells with the signals emitted by identical number of untreated cells. By comparing the signals from the different treated populations of cells, the relative changes in cell size and cytoplasmic complexity can be determined.

The effects of a protein or chimeric molecule thereof on cell growth, apoptosis, development, or differentiation can be assayed using one or more of the following systems.

Protein-induced apoptosis and changes in cell growth or cycles can be assessed by labeling the DNA of treated cells with dyes such as propidium iodine which has an excitation wavelength in the range of 488 nm and emission at 620 nm. Cells undergoing apoptosis has condensed DNA as well as different size and granularity. These factors give specific forward and size scatter profiles as well as fluorescence signal, and hence the population of cells undergoing apoptosis can be differentiated from normal cells. The amount of DNA in a cell also reflects which state of the cell cycle the cell is in. For instance, a cell in G₂ stage will have twice the amount of DNA as a cell in G₀ state. This will be reflected by a doubling of the fluorescence signal given off by a cell in G₂ phase. The effect of a protein or chimeric molecule thereof can be assessed by using FACS to compare the fluorescence signals given off by for instance, 20,000 treated cells with the signals emitted by identical number of untreated cells.

The protein or its chimeric molecule of the present invention may also alter the expression of various proteins. The effects of the protein or chimeric molecule thereof on protein expression by cells can be assayed using one or more of the following systems.

To assess the increase and decrease in expression of a protein in an entire cell, the cells can be fixed and permeabilised, then incubated with fluorescence conjugated antibody targeting the epitope of the protein of interest. A large variety of fluorescent labels can be used with an Argon laser system. Fluorescent molecules such as FITC, Alexa Fluor 488, Cyanine 2, Cyanine 3 are commonly used for this experiment. This method can also be used to estimate the changes in expression of surface markers and proteins by labeling non-permeabilised cells where only the epitope exposed on the cell surface can be labeled with antibodies. The effect of a protein or chimeric molecule thereof can be assessed by using FACS to compare the fluorescence signals given off by, for instance, 20,000 treated cells with the signals emitted by identical number of untreated cells.

The effects of a protein or its chimeric molecule on ligand/receptor adhesion can be assayed using one or more of the following systems.

A protein or chimeric molecule of the present may be more or less adhesive to substrates compared to those of a previously known physiochemical form. The interaction may be with protein receptors for sugar structures (e.g. selecting, such as L-selectin and P-selectin), with extracellular matrix components such as fibronectin, collagens, vitronectins, and laminins, or with non-protein components such as sugar molecules (heparin sulfates, other glycosaminoglycans).

A protein or chimeric molecule thereof may also interact differently with non-biological origin materials such as tissue culture plastics, medical device components (e.g. stents or other implants) or dental materials. In the case of medical devices this may alter the engraftment rates, the interaction of the implant with particular classes of cell type or the type of linkage formed with the body.

Any suitable assays for protein adhesion can be employed. For instance, a solution containing a protein or chimeric molecule of the present invention is incubated with a binding partner, in a particular embodiment, on an immobilised surface. Following incubation, the amount of the protein or the chimeric molecule present in the solution is assayed by ELISA and the difference between the amount remaining and the starting material is what has bound to the binding partner. For instance, the interaction between the protein or the chimeric molecule and an extracellular matrix protein could be determined by first coating wells of a 96 well plate with the ECM protein (e.g. fibronectin). Non-specific binding is then blocked by incubation with a BSA solution. Following washing, a known concentration of a protein or its chimeric molecule solution is added for a defined period. The solution is then removed and assayed for the amount of protein or its chimeric molecule remaining in solution. The amount bound to the ECM protein can be determined by incubating the wells with an antibody to a protein or its chimeric molecule, then detecting with an appropriate system (either a labeled secondary antibody or by biotin-avidin enzyme complexes such as those used for ELISA).

Methods for determining the amount bound to other surfaces may involve hydrolyzing a protein or its chimeric molecule from the inert implant surface, then measuring the amino acids present in the solution.

The effects of a protein or a chimeric molecule thereof on cell adhesion can be assayed using one or more of the following systems.

Cell adhesion to matrix (e.g. extracellular matrix components such as fibronectin, vitronectin, collagen, laminin etc.) is mediated at least in part by the integrin molecules. Integrin molecules consist of alpha and beta subunits, and the particular combinations of alpha and beta subunit give rise to the binding specificity to a particular ligand (e.g. a2b1 integrin binds collagen, a5b1 binds fibronectin etc). The integrins subunits have large extracellular domains responsible for binding ligand, and shorter cytoplasmic domains responsible for interaction with the cytoskeleton. In the presence of ligand, the cytoplasmic domains are responsible for the induction of signal transduction events (“outside in signaling”). The affinity of integrins for their ligands can be modulated by extracellular signaling events that in turn lead to changes in the cytoplasmic tails of the integrins (“inside out signaling”).

Incubation with a protein or chimeric molecule of the present invention can potentially alter cell adhesion in a number of ways. First, it can alter qualitatively the expression of particular integrin subsets, leading to changes in binding ability. Secondly, the amount of a particular integrin expressed may alter, leading to altered cell binding to its target matrix. Thirdly, the affinity of a particular integrin may be altered without changing its surface expression (inside-out signaling). All these changes may alter the binding of cells to either a spectrum of ligands, or alter the binding to a particular ligand.

A protein or chimeric molecule of the present invention can be tested in Cell-ECM adhesion assays which are generally performed in 96 well plate. Wells are coated with matrix, then unbound sites within the wells are blocked with BSA. A defined number of cells are incubated with the coated wells, then unbound cells are washed away and the bound cells incubated in the presence or absence of the protein or the chimeric molecule thereof. The number of cells is determined by an indirect method such as MTT/MTS. Alternatively, the cells are labeled with a radioactive label (e.g. ⁵¹Cr) and a known amount of radioactivity (i.e. cells) is added to each well. The amount of bound radioactivity is determined and calculated as a percentage of the amount loaded.

Cells also adhere to other cells, for instance, adhesion of one population of cells to a monolayer of another type of cells. To assay for this, the suspension cells added to the monolayer cells would be labeled with radioactivity. The cells are then incubated in the presence or absence of a protein or chimeric molecule thereof. The unbound cells would be washed away and the remaining mixed population of cells can be lysed and assayed for the amount of radioactivity present.

The effects of a protein or chimeric molecule thereof on cell spreading can be assayed using one or more of the following systems.

A protein or chimeric molecule of the present invention may have altered effects on cell spreading. Initiation of cell spreading is a key step in cell motility and invasive behavior. Cells spreading can be initiated in vitro in a number of ways. Plating a suspension of cells onto ECM components will result in attachment and ligand binding by integrin receptors. This initiates signal transduction events resulting in the activation of a family of the Cdc42, Rac and Rho small GTPases. Activation of these proteins results in actin polymerization and an extension of a lamellipodium, resulting in gradual flattening of the cells and contact of more integrins with their receptors. Eventually the cells have flattened totally and formed focal adhesions (large structures containing integrins and signaling proteins). Cell spreading can also be initiated by stimulation of adherent cells with growth factors, again resulting in activation of the Cdc42/Rac/Rho proteins and lamellipodium formation.

Cell spreading can be quantitated by examining a large number of cells at different time points following stimulation with a protein or chimeric molecule thereof. The area of each cell can be determined using image analysis programs and the percentage of cells spread as well as the degree of cell spreading can be compared with time. More rapid spreading may be initiated by a higher activation of the Cdc42/Rac/Rho pathways, alternatively, temporal, qualitative and quantitative differences in their activation may be observed with a protein or chimeric molecule of the present invention. This in turn may reflect differences in the signaling events induced by the protein or chimeric molecule of the present invention.

The effects of a protein or a chimeric molecule thereof on cell motility, migration and invasion can be assayed using one or more of the following systems.

Cells adherent to a tissue culture dish do not remain statically anchored to one spot, but rather constantly extend and retract portions of their cell body. When viewed under time-lapse photography, the cells can be observed to move around the dish, either as isolated single cells or as a cell colony. This motion may be either “random walk” (i.e. not directed in a particular direction), or directional. Both types of motion can be increased by the addition of growth factors. Time-lapse photography can be used to quantitate the overall distance covered by the cells in a given time period, as well as the overall directionality.

In the case of directional migration, cells will move towards a source of chemoattractant by sensing the chemical gradient and orienting their migration machinery towards it. In many instances, the chemoattractant is a growth factor. Directional migration can be quantitated by providing a source of chemoattractant (e.g. via a thin pipette) then imaging the cells migrating towards it with time-lapse photography.

An alternative system for determining directed migration is the Boyden chamber assay. In this assay, cells are placed in an upper chamber that is connected to a lower chamber via small holes in the partitioning membrane. Growth medium is put in both chambers, but chemoattractant is added only to the lower chamber, resulting in a diffusion gradient between the two chambers. The cells are attracted to the growth factor source and migrate through the holes in the separation membrane and on to the lower side of the membrane. After a number of hours, the membrane is removed and the number of cells that has migrated onto the bottom of the membrane is determined.

The process of cellular invasion utilises many of the same components as migration. Cell invasion can be modeled using layers of extracellular matrix through which the cells invade. For instance, Matrigel is a mixture of basement membrane components (ECM components, growth factors etc.) that is liquid at 4 degrees but rapidly sets at 37 degrees to form a gel. This can be used to coat the upper surface of a Boyden chamber, and the chemoattractant added to the lower layer. For cells to pass onto the lower surface of the membrane, they must degrade the matrigel using enzymes such as collagenases and matrix metalloproteinases (MMPs) as well as migrating directionally towards the chemoattractant. This assay mimics the various processes required for cellular invasion.

The effects of a protein or a chimeric molecule thereof on chemotaxis can be assayed using one or more of the following systems.

The migration of cells toward the chemoattractant can be measured in vitro in a Boyden chamber. A protein or chimeric molecule of the present in invention is placed in the lower chamber and an appropriate target cell population is placed in the upper chamber. To mimic the in vitro process of immune cells migrating from the blood to sites of inflammation, migration through a layer of cells may be measured. Coating the upper surface of the well of the Boyden chamber with a confluent sheet of cells, for instance, epithelial, endothelial or fibroblastic cells, will prevent direct migration of immune cells through the holes in the well. Instead, the cells will need to adhere to the monolayer and migrate through it towards the protein to be tested. The presence of cells on the under surface of the Boyden chamber or in the medium in the lower well in only those wells treated with the protein or chimeric molecule thereof is indicative of the chemotactic ability of the protein or the chimeric molecule. To show that the effect is specific to a protein or chimeric molecule thereof, a neutralising antibody can be incubated with the protein in the lower chamber.

Alternatively, to test the ability of a substance (chemical, protein, sugar) to prevent chemotaxis, the substance is included in the lower chamber of the Boyden chamber along with a solution containing known chemotactic ability (this may be a specific chemokine, conditioned medium from a cell source or cells secreting a range of chemokines). A susceptible target cell population is then added to the upper chamber and the assay performed as described above.

The effects of a protein or chimeric molecule thereof on ligand-receptor binding can be assayed using one or more of the following systems.

A protein or chimeric molecule of the present invention may have different ligand-receptor binding abilities. Ligand-receptor binding can be measured by various parameters, for instance, the dissociation constant (Kd), dissociation rate constant (off rate) (k⁻), association rate constant (on rate) (k⁺). Differences in ligand-receptor binding may correlate with different timing and activation of signaling, leading to different biological outcomes.

Ligand-receptor binding can be measured and analysed by either Scatchard plot or by other means such as Biacore.

For Scatchard analysis, a protein or its chimeric molecule, labeled with, for instance, radioactively labeled (eg, ¹²⁵I), is incubated in the presence of differing amounts of cold competitor of a protein or its chimeric molecule, with cells, or extracts thereof, expressing the corresponding ligand or receptor. The amount of specifically bound labeled protein or its chimeric molecule is determined and the binding parameters calculated.

For the Biacore, the corresponding recombinant ligand or receptor of the protein or its chimeric molecule is coupled to the detection unit. Solutions containing a protein or chimeric molecule thereof of choice are then passed over the detection cell and binding is determined by a change in the properties of the detection unit. On rates can be determined by passing solutions containing the protein or the chimeric molecule over the detection cell until a fixed reading is recorded (when the available sites are all occupied). A solution not containing the protein or the chimeric molecule is then passed over the cell and the protein dissociates from the corresponding ligand or receptor, giving the off rate.

The effects of a protein or chimeric molecule thereof on receptor activation can be assayed using one or more of the following systems.

Interaction with a protein or a chimeric molecule thereof and its corresponding ligand or receptor may be paralleled by differences in the signaling events induced from the cell's endogenous protein. The timing of interaction may be characteristic of a protein or chimeric molecule thereof as definitely on/off rates or dissociation constants.

Activated receptors are often internalized by the cells. The receptor/ligand complex can then be dissociated (e.g., be lowering the pH within cellular vesicles, resulting in detachment of the ligand) and the receptor recycled to the cell surface. Alternatively, the complex may be targeted for destruction. In this case the receptors are effectively down-regulated and unable to generate more signal, whereas when they are recycled they are able to repeat the signaling process. Differential receptor binding or activation may result in the receptor being switched from a destruction to a recycling pathway, resulting in a stronger biological response.

The effects of a protein or a chimeric molecule thereof on signal transduction can be assayed using one or more of the following systems.

Binding of ligands or receptors to the protein or its chimeric molecule thereof may initiate signaling, which may include reverse signaling, through a variety of cytoplasmic proteins. Reverse signaling occurs when a membrane-bound form of a ligand transduces a signal following binding by a soluble or membrane bound version of its receptor. Reverse signaling can also occur after binding of the membrane bound ligand by an antibody. These signaling events (including reverse signaling events) lead to changes in gene and protein expression. Hence, a protein or chimeric molecule of the present invention can induce or inhibit different signal transductions in various pathways or other signal transduction events, such as the activation of JAK/STAT pathway, Ras-erk pathway, AKT pathway, the activation of PKC, PKA, Src, Fas, TNFR, NFkB, p38MAPK, c-Fos, recruitment of proteins to receptors, receptor phosphorylation, receptor internalization, receptor cross-talk or secretion.

The ligands or receptors recruited to the protein or chimeric molecule thereof may be unique to the protein or chimeric molecule of the present invention, due to different conformations of the ligand or receptors being induced. One way of assaying for these differences is to immunoprecipitate the ligand or receptor using an antibody crosslinked to sepahrose beads. Following immunoprecipitation and washing, the proteins are loaded on a 2D gel and the comparative spot patterns are analysed. Different spots can be cut out and identified by mass spectrometry.

The effects of a protein or chimeric molecule thereof on up regulation and down regulation of surface markers can be assayed using one or more of the following systems.

Cells may have a variety of responses to the protein or chimeric molecule of the present invention. There are a range of proteins on cell surfaces responsible for communication between the cells and the extracellular environment. Through regulated processes of endocytosis and exocytosis, various proteins are transported to and from the cell surface. Typical proteins found on the cells surface includes receptors, binding proteins, regulatory proteins and signaling molecules. Changes in expression and degradation rate of the proteins also changes the level of the proteins on the cell surface. Some proteins are also stored in intracellular reservoirs where specific signals can induce trafficking of proteins between this storage and the cellular membrane.

Cells are incubated for an appropriate amount of time in medium containing a protein or chimeric molecule of the present invention and their responses can be compared with cells exposed to the same medium without the protein or chimeric molecule of the present invention. The proteins on the cell membrane can be solubilised and separated from the cells by centrifugation. The level of expression of a specific protein can be measured by Western blotting. Cells can also be labeled with fluorescence conjugated antibodies, and visualized under confocal microscopy system or counted by fluorescence activated cell sorting (FACS). This will detect any changes in expression and distribution of proteins on the cells. By using multiple antibodies, changes in protein interaction can also be studied by confocal microscopy and immuno-precipitation. Similarly, these experiments can be extended to in vivo animal models. Cells from specific part of animals treated with the protein or chimeric molecule of the present invention may be extracted and examined with identical methodologies.

Cells induced to differentiate in vitro or in vivo by the addition of the protein or chimeric molecule of the present invention will express differentiation markers that distinguish them from the untreated cells. Some cells, for instance, progenitor or stem cells, can differentiate into many subpopulations, distinguishable by their surface markers. A protein or chimeric molecule of the present invention may stimulate the progenitor cells to differentiate into subgroups in a particular ratio.

The protein of the present invention and its chimeric molecule may have effects upon cell repulsion.

The effects of the protein or its chimeric molecule on the modulation of the growth and guidance of cells and neurons is a convenient assay for cell repulsion.

Disrupting the interactions between subunits and other components of a protein leads to a way to inhibit the biological effects of the protein or its chimeric molecule. Compounds inhibiting such biological effects are identified by a number of ways.

High throughput screening programs use a library of small chemical entities (chemicals or peptides) to generate lead compounds for clinical development. A number of assays can be used to screen a library compounds for their ability to affect a biologically relevant endpoint. Each potential compound in a library is tested with a particular assay in a single well, and the ability of the compound to affect the assay determined. Some examples of the assays are provided below:

For this assay, cells are plated into a microtitre plate (96 plate, 384 plate or the like). The cells will have a readout mechanism for activation of a protein or chimeric molecule thereof. This may involve assaying for cell growth, assaying for stimulation of a particular pathway (e.g., FRET based techniques), assaying for induction of a reporter gene (e.g., CAT, beta-galactosidase, fluorescent proteins), assaying for apoptosis and assaying for differentiation. Cells are then exposed to the protein or chimeric molecule of the present invention in the presence or absence of a particular small molecule. The drug can be added before, after or during the addition of the protein or chimeric molecule thereof. After an appropriate period of time, the individual wells are read using an appropriate method (eg, Fluorescence for FRET or induction of fluorescent proteins, cell number by MTT, beta-galactosidase activity etc). Control wells without addition of any drug or cytokine serve as comparisons. Any molecule able to inhibit the receptor/cytokine complex will give a different readout to the control wells. Further experiments will be required to show specificity of the inhibition. Alternatively, the drug could affect the detection method by a non-cytokine, non-receptor mechanism (a false positive).

A receptor of the protein or chimeric molecule thereof is immobilised on a solid surface. A protein or its chimeric molecule and the compound to be tested are then added. This can be performed by adding a protein or its chimeric molecule first, then the compound; the compound first, then a protein or its chimeric molecule; or the compound and the protein or its chimeric molecule can be added together. Bound protein or the chimeric molecule is then detected by an appropriate detection antibody. The detection antibody can be labeled with an enzyme (e.g., alkaline phosphatase or Horse-radish peroxidase for colorimetric detection) or a fluorescent tag for fluorescence detection. Alternatively, a protein or its chimeric molecule can be labeled (e.g., Biotin, radioactive labeling) and be detected with an appropriate technique (e.g., for Biotin labeling, streptavidin linked to a colorimetric detection system, for radiolabeling the complex is solubilised and counted). Inhibition of protein binding is measured by a drop in the reading compared to the control wells.

Soluble receptors of the protein or chimeric molecules thereof are bound to beads. This binding reaction can be either an adsorption process or involve chemically linking them to the plate. The beads are incubated with the protein or the chimeric molecules and a candidate compound in an appropriate well. This can be performed as the protein or the chimeric molecules first, then compound; compound first then the protein or the chimeric molecules; or compound and the protein or the chimeric molecules together. A fluorescently labeled detection antibody that recognizes a protein or chimeric molecule thereof is then added. The unbound antibody is removed and the beads are passed through a FACS. The amount of fluorescence detected will decrease if a compound inhibits the interaction of a protein or chimeric molecule thereof with its receptor.

To enable screening of multiple interactions between protein and its corresponding ligand/receptor against one inhibitory compound, the ability of the FACS machine to analyse scatter profiles is used. A bead with a larger diameter will have a different scatter profile to that of a smaller bead, and this can be separated out for analysis (“gating”).

A number of different proteins, one of which is the protein or chimeric molecule of the present invention, are each linked to beads of a particular diameter. A mixture of ligands/receptors to the above-mentioned proteins are then added to the bead mixture in the presence of one candidate compound. The bound ligands/receptors are then detected using a specific secondary antibodies that is fluorescently labeled. The antibodies can be all labeled with the same detection fluorophore. The ability of the compound to prevent binding of a protein to its ligand/receptor is then determined by running the sample though a FACS machine and gating for each known bead size. The individual binding results are then analysed separately. The major benefit of this method of analysis is that the screening each compound can be tested in parallel with a number of proteins to decrease the time taken for screening proportionally.

A protein or chimeric molecule thereof may also be characterised by its crystal structure. The physiochemical form of a protein or its chimeric molecule may provide a unique 3D crystal structure. In addition, the crystal structure of the protein-ligand/receptor complex may also be generated using a protein or chimeric molecule of the present invention. Since the present invention provides a protein or a chimeric molecule thereof which is substantially similar to a human naturally occurring form, the complex is likely to be a more reflective representation of the in vivo structure of the naturally occurring protein-ligand/receptor complex. Once a crystal structure has been obtained, interactions between a protein or its chimeric molecule and potential compounds inhibiting such interactions can be identified.

Once potential compounds are identified by high throughput screening or from the crystal structure of the protein-ligand/receptor complex, a process of rational drug design can begin.

There are several steps commonly taken in the design of a mimetic from a compound having a given desired property. First, the particular parts of the compound that are critical and/or important in determining the desired property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic. Modeling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9:19-21, 1991). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al. Science 249:527-533, 1990). In addition, target molecules may be analyzed by an alanine scan (Wells, Methods Enzymol 202:2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

In one aspect, the protein or chimeric molecule of the present invention is used as an immunogen to generate antibodies. The physiochemical form of a protein or chimeric molecule of the present invention may raise antibodies to the protein or the chimeric molecule; glycopeptides specific to the protein or chimeric molecule of the present invention; or antibodies directed to another co- or post-translationally modified peptide within the protein or chimeric molecule thereof.

The protein of the present invention or its chimeric molecule may present epitopes not normally accessible (but possibly present) in vivo. For instance, there may be regions within a receptor domain that are normally in contact with another component of a heteromeric receptor. These epitopes may be used to generate monoclonal antibodies that cross react with the endogenous receptor. Such antibodies may block interaction of one receptor component with another and therefore prevent signal transduction. This may be therapeutically useful in the case of overexpression of a cytokine or receptor. The antibodies may also be therapeutically useful in diseases where the receptor is overexpressed and signals without needing the ligand.

The antibodies are also useful to detect the levels of the protein or chimeric molecule thereof during the treatment of the disease (e.g., serum levels for half-life determination).

In addition, the antibodies are useful as diagnostic for determining the presence of a protein or chimeric molecule of the present invention in a particular sample.

Reference to an “antibody” or “antibodies” includes reference to all the various forms of antibodies, including but not limited to: full antibodies (e.g. having an intact Fc region), including, for example, monoclonal antibodies; antigen-binding antibody fragments, including, for example, Fv, Fab, Fab′ and F(ab′)₂ fragments; humanized antibodies; human antibodies (e.g., produced in transgenic animals or through phage display); and immunoglobulin-derived polypeptides produced through genetic engineering techniques. Unless otherwise specified, the terms “antibody” or “antibodies” and as used herein encompasses both full antibodies and antigen-binding fragments thereof.

Unless stated otherwise, specificity in respect of an antibody of the present invention is intended to mean that the antibody binds substantially only to its target antigen with no appreciable binding to unrelated proteins. However, it is possible that an antibody will be designed or selected to bind to two or more related proteins. A related protein includes different splice variants or fragments of the same protein or homologous proteins from different species. Such antibodies are still considered to have specificity for those proteins and are encompassed by the present invention. The term “substantially” means in this context that there is no detectable binding to a non-target antigen above basal, i.e. non-specific, levels.

The antibodies of the present invention may be prepared by well-known procedures. See, for example, Monoclonal Antibodies, Hybridomas: A New Dimension in Biological Analyses, Kennet et al. (eds.), Plenum Press, New York (1980); and Antibodies: A Laboratory Manual, Harlow and Lane (eds.), Cold Spring Harbor Laboratory Press, Cold Spring Harbor, N.Y., (1988).

One method for producing an antibody of the present invention comprises immunizing a non-human animal, such as a mouse or a transgenic mouse, with a protein or chimeric molecule of the present invention, or immunogenic parts thereof, such as, for example, a peptide containing the receptor binding domain, whereby antibodies directed against the polypeptide of a protein or its chimeric molecule, or immunogenic parts thereof, are generated in the animal. Various means of increasing the antigenicity of a particular protein or its chimeric molecule, such as administering adjuvants or conjugated antigens, comprising the antigen against which an antibody response is desired and another component, are well known to those in the art and may be utilized. Immunizations typically involve an initial immunization followed by a series of booster immunizations. Animals may be bled and the serum assayed for antibody titer. Animals may be boosted until the titer plateaus. Conjugates may be made in recombinant cell culture as protein fusions. Also, aggregating agents such as alum are suitably used to enhance the immune response.

Both polyclonal and monoclonal antibodies can be produced by this method. The methods for obtaining both types of antibodies are well known in the art. Polyclonal antibodies are less favored but are relatively easily prepared by injection of a suitable animal with an effective amount of a protein or chimeric molecule of the present invention, or immunogenic parts thereof, collecting serum from the animal and isolating specific antibodies to a protein or chimeric molecule thereof by any of the known immunoadsorbent techniques. Antibodies produced by this technique are generally less favoured, because of the potential for heterogeneity of the product.

The use of monoclonal antibodies is particularly favored because of the ability to produce them in large quantities and the homogeneity of the product. Monoclonal antibodies may be produced by conventional procedures.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. Nature 256:495 (1975), or may be made by recombinant DNA methods (see, e.g., U.S. Pat. No. 4,816,567). The “monoclonal antibodies” may also be isolated from phage antibody libraries using for example, the techniques described in Clackson et al. Nature 352:624-628, 1991 and Marks et al. J Mol Biol 222:581-597, 1991.

The present invention contemplates a method for producing a hybridoma cell line which comprises immunizing a non-human animal, such as a mouse or a transgenic mouse, with a protein or chimeric molecule of the present invention; harvesting spleen cells from the immunized animal; fusing the harvested spleen cells to a myeloma cell line to generate hybridoma cells; and identifying a hybridoma cell line that produces a monoclonal antibody that binds a protein or chimeric molecule thereof.

Such hybridoma cell lines and the monoclonal antibodies produced by them are encompassed by the present invention. Monoclonal antibodies secreted by the hybridoma cell lines are purified by conventional techniques. Hybridomas or the monoclonal antibodies produced by them may be screened further to identify monoclonal antibodies with particularly desirable properties, such as the ability to inhibit cytokine-signaling through its receptor.

A protein or chimeric molecule thereof or immunogenic part thereof that may be used to immunize animals in the initial stages of the production of the antibodies of the present invention should be from a human-expressed source.

Antigen-binding fragments of antibodies of the present invention may be produced by conventional techniques. Examples of such fragments include, but are not limited to, Fab, Fab′, F(ab′)₂ and Fv fragments, including single chain Fv fragments (termed sFv or scFv). Antibody fragments and derivatives produced by genetic engineering techniques, such as disulfide stabilized Fv fragments (dsFv), single chain variable region domain (Abs) molecules, minibodies and diabodies are also contemplated for use in accordance with the present invention.

Such fragments and derivatives of monoclonal antibodies directed against a protein or chimeric molecule thereof may be prepared and screened for desired properties, by known techniques, including the assays herein described. The assays provide the means to identify fragments and derivatives of the antibodies of the present invention that bind to a protein or chimeric molecule thereof, as well as identify those fragments and derivatives that also retain the activity of inhibiting signaling by a protein or chimeric molecule thereof. Certain of the techniques involve isolating DNA encoding a polypeptide chain (or a portion thereof) of a mAb of interest, and manipulating the DNA through recombinant DNA technology. The DNA may be fused to another DNA of interest, or altered (e.g. by mutagenesis or other conventional techniques) to add, delete, or substitute one or more amino acid residues.

DNA encoding antibody polypeptides (e.g. heavy or light chain, variable region only or full length) may be isolated from B-cells of mice that have been immunized with a protein or chimeric molecule of the present invention. The DNA may be isolated using conventional procedures. Phage display is another example of a known technique whereby derivatives of antibodies may be prepared. In one approach, polypeptides that are components of an antibody of interest are expressed in any suitable recombinant expression system, and the expressed polypeptides are allowed to assemble to form antibody molecules.

Single chain antibodies may be formed by linking heavy and light chain variable region (Fv region) fragments via an amino acid bridge (short peptide linker), resulting in a single polypeptide chain. Such single-chain Fvs (scFvs) have been prepared by fusing DNA encoding a peptide linker between DNAs encoding the two variable region polypeptides (VL and VH). The resulting antibody fragments can form dimers or trimers, depending on the length of a flexible linker between the two variable domains (Kortt et al. Protein Engineering 10:423, 1997). Techniques developed for the production of single chain antibodies include those described in U.S. Pat. No. 4,946,778; Bird (Science 242:423, 1988), Huston et al. (Proc Natl Acad Sci USA 85:5879, 1988) and Ward et al. (Nature 334:544, 1989). Single chain antibodies derived from antibodies provided herein are encompassed by the present invention.

In one embodiment, the present invention provides antibody fragments or chimeric, recombinant or synthetic forms of the antibodies that bind to the protein or chimeric molecule of the present invention and inhibit signaling by the protein or its chimeric molecule.

Techniques are known for deriving an antibody of a different subclass or isotype from an antibody of interest, i.e., subclass switching. Thus, IgG1 or IgG4 monoclonal antibodies may be derived from an IgM monoclonal antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody.

Recombinant DNA techniques may be employed. Cloned DNA encoding particular antibody polypeptides may be employed in such procedures, e.g. DNA encoding the constant region of an antibody of the desired isotype.

The monoclonal production process described above may be used in animals, for example mice, to produce monoclonal antibodies. Conventional antibodies derived from such animals, for example murine antibodies, are known to be generally unsuitable for administration to humans as they may cause an immune response. Therefore, such antibodies may need to be modified in order to provide antibodies suitable for administration to humans. Processes for preparing chimeric and/or humanized antibodies are well known in the art and are described in further detail below.

The monoclonal antibodies herein specifically include “chimeric” antibodies in which the variable domain of the heavy and/or light chain is identical with or homologous to corresponding sequences in antibodies derived from a non-human species (e.g., murine), while the remainder of the chain(s) is identical with or homologous to corresponding sequences in antibodies derived from humans, as well as fragments of such antibodies, so long as they exhibit the desired biological activity (U.S. Pat. No. 4,816,567; and Morrison et al. Proc Natl Acad Sci USA 81:6851-6855, 1984).

“Humanized” forms of non-human (e.g., murine) antibodies are chimeric antibodies which contain minimal sequence derived from the non-human immunoglobulin. For the most part, humanized antibodies are human immunoglobulins (recipient antibody) in which the complementarity determining regions (CDRs) of the recipient are replaced by the corresponding CDRs from a non-human species (donor antibody) such as mouse, rat, rabbit or nonhuman primate having the desired properties, for example specificity, and affinity. In some instances, framework region residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the complementarity determining regions correspond to those of a non-human immunoglobulin and all or substantially all of the framework region residues are those of a human immunoglobulin sequence. The humanized antibody optionally also will comprise at least a portion of an immunoglobulin constant region (Fc), typically that of a human immunoglobulin. For further details, see Jones et al. Nature 321:522-525, 1986; Reichmann et al. Nature 332:323-329, 1988; Presta, Curr Op Struct Biol 2:593-596, 1992; Liu et al. Proc Natl Acad Sci USA 84:3439, 1987; Larrick et al. Bio/Technology 7:934, 1989; and Winter and Harris, TIPS 14:139, 1993.

In a further embodiment, the present invention provides an immunoassay kit with the ability to assay the level of human protein expressed from human cells present in a biological preparation, including a biological preparation comprising the naturally occurring human protein.

A biological preparation which can be assayed using the immunoassay kit of the present invention includes but is not limited to laboratory samples, cells, tissues, blood, serum, plasma, urine, stool, saliva and sputum.

The immunoassay kit of the present invention comprises a solid phase support matrix, not limited to but including a membrane, dipstick, bead, gel, tube or a multi-well, flat-bottomed, round-bottomed or v-bottomed microplate, for example, a 96-well microplate; a preparation of antibody directed against the human protein of interest (the capture antibody); a preparation of blocking solution (for example, BSA or casein); a preparation of secondary antibody (the detection antibody), also directed against the human protein of interest and conjugated to a suitable detection molecule (for example, alkaline phosphatase); a solution of chromagenic substrate (for example, nitro blue tetrazolium); a solution of additional substrate (for example, 5-bromo-4-chloro-3-indolyl phosphate); a stock solution of substrate buffer (for example, 0.1M Tris-HCL (pH 7.5) and 0.1M NaCl, 50 mM MgCl₂); a preparation of the protein or chimeric molecule of the present invention with known concentration (the standard); and instructions for use.

A suitable detection molecule may be chosen from the list consisting an enzyme, a dye, a fluorescent molecule, a chemiluminescent, an isotope or such agents as colloidal gold conjugated to molecules including, but not limited to, such molecules as staphylococcal protein A or streptococcal protein G.

In a particular embodiment, the capture and detection antibodies are monoclonal antibodies, the production of which comprises immunizing a non-human animal, such as a mouse or a transgenic mouse, with a protein or chimeric molecule of the present invention, followed by standard methods, as hereinbefore described. Monoclonal antibodies may alternatively be produced by recombinant methods, as hereinbefore described and may comprise human or chimeric antibody portions or domains.

In another embodiment, the capture and detection antibodies are polyclonal antibodies, the production of which comprises immunizing a non-human animal, such as a mouse, rabbit, goat or horse, with a protein or chimeric molecule of the present invention, followed by standard methods, as hereinbefore described.

The components of the immunoassay kit are provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution provide for each reagent solution having the appropriate concentration for combining with the biological preparation to be tested.

The instructions for use may detail the method for using the immunoassay kit of the present invention. For example, the instructions for use may describe the method for coating the solid phase support matrix with a prepared solution of capture antibody under suitable conditions, for example, overnight at 4° C. The instructions for use may further detail blocking non-specific protein binding sites with the prepared blocking solution; adding and incubating serially diluted sample containing the protein or chimeric protein of the present invention under suitable conditions, for example, 1 hour at 37° C. or 2 hours at room temperature, followed by a series of washes using a suitable buffer known in the art, for example, a solution of 0.05% Tween 20 in 0.1M PBS (pH 7.2). In addition, the instructions may provide that a preparation of detection antibody is applied followed by incubation under suitable conditions, for example, 1 hour at 37° C. or 2 hours at room temperature, followed by a further series of washes. A working solution of detection buffer is prepared from the supplied detection substrate(s) and substrate buffer, then added to each well under a suitable conditions ranging from 5 minutes at room temperature to 1 hour at 37° C. The chromatogenic reaction may be halted with the addition of 1N NaOH or 2NH₂SO₄.

In an alternative embodiment, the instructions for use may provide the simultaneous addition of any combination of any or all of the above components to be added in predetermined ratios, with the relative amounts of the various reagents suitably varied to provide for concentrations in solution of the reagents that substantially maximize the formation of a measurable signal from formation of a complex.

The level of colored product, or fluorescent or chemiluminescent or radioactive or other signal generated by the bound, conjugated detection reagents can be measured using an ELISA-plate reader or spectrophotometer, at an appropriate optical density (OD), or as emitted light, using a spectrophotometer, fluorometer or flow cytometer, at an appropriate wavelength, or using a radioactivity counter, at an appropriate energy spectrum, or by a densitometer, or visually by comparison to a chart or guide. A serially diluted solution of the standard preparation is assayed in parallel with the above sample. A standard curve or chart is generated and the level of the protein or chimeric molecule thereof present within the sample can be interpolated from the standard curve or chart.

The subject invention also provides a human derived protein or chimeric molecule thereof for use as a standard protein in an immunoassay. The present invention further extends to a method for determining the level of human cell-expressed human protein or chimeric molecule thereof in a biological preparation comprising a suitable assay for measuring the human protein or the chimeric molecule wherein the assay comprises (a) combining the biological preparation with one or more antibodies directed against the human protein or chimeric molecule thereof; (b) determining the level of binding of the or each antibody to the human protein or the chimeric molecule in the biological preparation; (c) combining a standard human protein or a chimeric molecule sample with one or more antibodies directed against the human protein or the chimeric molecule; (d) determining the level of binding of the or each antibody to the standard human protein or the chimeric molecule sample; (e) comparing the level of the or each antibody bound to the human protein or the chimeric molecule in the biological preparation to the level of the or each antibody bound to the standard human protein or chimeric molecule sample.

In particular, the standard human protein or chimeric molecule sample is a preparation comprising the protein or chimeric molecule of the present invention.

The biological preparation includes but is not limited to laboratory samples, cells, tissues, blood, serum, plasma, urine, stool, saliva and sputum. The biological preparation is bound to one or more capture antibody as described hereinbefore or by methods known in the art. For instance, the solid phase support matrix is first coated with a prepared solution of capture antibody under suitable conditions (for example, overnight at 4° C.); followed by blocking non-specific protein binding sites with the prepared blocking solution; then adding and incubating serially diluted sample containing a protein or chimeric molecule of the present invention under suitable conditions (for example, 1 hour at 37° C. or 2 hours at room temperature), followed by a series of washes using a suitable buffer known in the art (for example, a solution of 0.05% Tween 20 in 0.1M PBS (pH 7.2)).

The biological preparation is then combined with one or more detection antibodies conjugated to a suitable detection molecule as described herein. For instance, applying a preparation of detection antibody followed by incubation under suitable conditions (for example, 1 hour at 37° C. or 2 hours at room temperature), followed by a further series of washes.

Determination of the level of binding may be carried out as described hereinbefore or by methods known in the art. For instance, a working solution of detection buffer is prepared from the detection substrate(s) and substrate buffer, then adding to each well under a suitable conditions ranging from 5 minutes at room temperature to 1 hour at 37° C. The chromatogenic reaction may be halted with the addition of 1N NaOH or 2NH₂SO₄.

In a particular embodiment, the present invention contemplates an isolated protein or chimeric molecule as hereinbefore described.

In a particular embodiment, a GM-CSF of the present invention is characterized by a profile of one or more physiochemical parameters (Pₓ) and pharmacological traits (T_{y}) comprising an apparent molecular weight (P₁) of 5 to 60 such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 56, 57, 58, 59, 60 and in a particular embodiment 16-40 kDa. The pI (P₂) of GM-CSF of the present invention is about 1 to 10, such as 1, 2, 3, 4, 5, 6, 7, 8, 9, 10, and in a particular embodiment 2-7 with at least 1 to 36 isoforms such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 and in a particular embodiment 10-30 isoforms (P₃). The percentage by weight carbohydrate (P₅) of the GM-CSF of the present invention is about 1 to 99, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and in a particular embodiment 0-76% and in a further embodiment 0-65%. The observed molecular weight of the molecule after the N-linked oligosaccharides are removed (P₆) is 10-35 kDa and in a particular embodiment is between 12-30 kDa and the observed molecular weight of the molecule after the N-linked and O-linked oligosaccharides are removed (P₇) is 9-30 kDa and in a particular embodiment is between 11 and 25 kDa. The percentage acidic monosaccharide content (P₈) of the GM-CSF of the present invention is about 2 to 20% such as 2, 3, 4, 5, 6, 7, 8, 9, 10, 11, 12, 13, 14, 15, 16, 17, 18, 19, 20% and in a particular embodiment 6-11%. Monosaccharide (P₉) and sialic acid (P₁₀) content of the GM-CSF of the present invention, when normalized to GalNAc, is 1 to 0-3 fucose, 1 to 1-16 GlcNAc, 0.1 to 0.1-9 galactose, 1 to 0.1-9 mannose and 1 to 0-5 NeuNAc and in a particular embodiment is 1 to 0.1-1.5 fucose, 1 to 2-12 GlcNAc, 1 to 1.0-6.0 galactose, 1 to 1.0-6.0 mannose and 1 to 0-3.0 NeuNAc; when normalized to 3 times of mannose, is 3 to 0-5 fucose, 3 to 0.1-3 GalNAc, 3 to 2-15 GlcNAc, 3 to 1-6 galactose and 3 to 0-4 NeuNAc and in a particular embodiment is 3 to 0.1-2.5 fucose, 3 to 0.5-2.5 GalNAc, 3 to 5.0-10.0 GlcNAc, 3 to 2.0-5.0 galactose and 3 to 0-3.0 NeuNAc. Neutral percentage of N-linked oligosaccharides (P₁₃) is about 40 to 90%, such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 49 to 83%, in an additional embodiment 54 to 78%, and in a further embodiment 59 to 73%. Acidic percentage of N-linked oligosaccharides (P₁₄) is about 10% to 70%, in a particular embodiment 17% to 51%, in a an additional embodiment 22% to 46%, and in a further embodiment 27 to 41%. Neutral percentage of O-linked oligosaccharides (P₁₅) is about 5 to 90% such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%, in a particular embodiment 9 to 82%, in an additional embodiment 29 to 62% in a further embodiment 34 to 57%. Acidic percentage of O-linked oligosaccharides (P₁₆) is about 10 to 100%, in a particular embodiment 18 to 91%, in an additional embodiment 38 to 71% and in a further embodiment 43 to 66%. The sites of N-glycosylation (P₂₁) of the GM-CSF of the present invention include N-44 and N-54 (numbering from the start of the signal sequence) identified by PMF after PNGase treatment. The serum/plasma stability (T₁₀) of GM-CSF of the present invention is distinct from that of human GM-CSF expressed in non human cells, in particular the GM-CSF of the present invention exhibited greater proliferative activity on TF-1 cells following a 24 hour incubation in fetal calf serum than human GM-CSF produced from E. coli cells. The proliferation ability (T₃₂) of the GM-CSF of the present invention is distinct from that of a human GM-CSF expressed in a non-human cell system, in particular, the proliferation ability (T₃₂) of the GM-CSF of the present invention is 5-12 times greater than that of a human GM-CSF expressed in E. coli cells. The differentiation ability (T₃₃) of the GM-CSF of the present invention is distinct from that of a human GM-CSF expressed in a non-human cell system, in particular the GM-CSF of the present invention has a 1.5-2 fold greater capacity to induce colony formation in TF-1 cells than human GM-CSF expressed in E. coli cells.

In a particular embodiment, an IL-3 molecule of the present invention is characterized by a profile of one or more physiochemical parameters (Pₓ) and pharmacological traits (T_{y}) comprising an apparent molecular weight (P₁) of 1 to 250, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250 kDa and in a particular embodiment 15 to 35 kDa. The pI (P₂) of IL-3 molecule is 2 to 14 such as 2, 3, 4, 5, 6, 7, 8, 9, 10, 11, 12, 13, 14 and in a particular embodiment 3.5-7.5 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 5-15 isoforms (P₃). The percentage by weight carbohydrate (P₅) of the IL-3 molecule of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 0 to 60%. The observed molecular weight of the IL-3 of the present invention when the N-linked oligosaccharides are removed (P₆) is between 8 and 30 kDa and in a particular embodiment, between 10 and 25 kDa. Monosaccharide content (P₉) of the IL-3 molecule of the present invention, when normalized to GalNAc, are 1 to 0.1-8 fucose, 1 to 0.1-7 GlcNAc, 1 to 0.1-3 galactose, 1 to 0.1-3 mannose and 1 to 0-5 NeuAc; and in a particular embodiment 1 to 2-6 fucose, 1 to 3-5 GlcNAc, 1 to 0.5-2 galactose, 1 to 0.5-2 mannose and 1 to 0-2 NeuNAc; when normalized to 3 times of mannose, are 3 to 2-25 fucose, 3 to 0.1-6 GalNAc, 3 to 4-21 GlcNAc, 3 to 0.1-9 galactose and 3 to 0-5 NeuAc; in a particular embodiment 3 to 5-16 fucose, 3 to 2-4 GalNAc, 3 to 9-14 GlcNAc, 3 to 3-6 galactose and 3 to 0.1-2 NeuAc. The sialic acid content (P₁₀) expressed as a percentage of the monosaccharide content of the IL-3 molecule of the present invention is 0 to 50%, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% and in a particular embodiment 0 to 20%. Neutral percentage of N-linked oligosaccharides (P₁₃) of the IL-3 molecule of the present invention is 70 to 100%, in a particular embodiment 75 to 95% and in an additional embodiment 80 to 90%. Acidic percentage of N-linked oligosaccharides (P₁₄) of the IL-3 molecule of the present invention is 0 to 30%, in a particular embodiment 5 to 25% and in an additional embodiment 10 to 20%. The immunoreactivity profile (T₁₃) of the IL-3 of the present invention is distinct from that of a human IL-3 expressed in a non-human cell system, in particular, the protein concentration of the IL-3 of the present invention is underestimated when assayed using an ELISA kit which contains a human IL-3 expressed in a non-human cell system. In addition, the immunoreactivity profile (T₁₃) of the IL-3 of the present invention is distinct from that of a human IL-3 expressed in insect cells. The proliferation ability (T₃₂) of the IL-3 of the present invention is distinct from that of a human IL-3 expressed in a non-human cell system, in particular, the proliferation ability (T₃₂) of the IL-3 of the present invention is 1.1-2.5 times greater than that of a human IL-3 expressed in E. coli cells.

In a particular embodiment, an IL-4 molecule of the present invention is characterized by a profile of one or more physiochemical parameters (Pₓ) and pharmacological traits (T_{y}) comprising an apparent molecular weight (P₁) of 1 to 120, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, kDa and in a particular embodiment 12 to 24 kDa. The pI (P₂) of IL-4 is 2 to 14 such as 2, 3, 4, 5, 6, 7, 8, 9, 10, 11, 12, 13, 14 and in a particular embodiment 8 to 11 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 1-3 isoforms (P₃). The percentage by weight carbohydrate (P₅) of the IL-4 of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 0 to 25%. The observed molecular weight of the IL-4 of the present invention when the N-linked oligosaccharides are removed (P₆) is between 8 and 24 kDa and in a particular embodiment, between 10 and 20 kDa. The observed molecular weight of the IL-4 of the present invention when the N-linked oligosaccharides and O-linked oligosaccharides re removed (P₇) is between 8 and 22 kDa and in a particular embodiment, between 10 and 18 kDa. Neutral percentage of N-linked oligosaccharides (P₁₃) of the IL-4 of the present invention is 50 to 100%, in a particular embodiment 65 to 100% and in an additional embodiment 70 to 100%. Acidic percentage of N-linked oligosaccharides (P₁₄) of the IL-4 of the present invention is 0 to 50% in a particular embodiment 0 to 45% and in an additional embodiment 0 to 30%. The sites of N-glycosylation (P₂₁) of the IL-4 of the present invention include N-62 (numbering from the start of the signal sequence) identified by PMF after PNGase treatment. The sites of disulfide bond formation (P₃₃) include Cys27-Cys151, Cys48-Cys89 and Cys70-Cys123 (cysteine residues numbered from the start of the signal sequence).

In one embodiment, the immunoreactivity profile (T₁₃) of the IL-4 of the present invention is distinct from that of a human IL-4 expressed in a non-human cell system, in particular, the protein concentration of the IL-4 of the present invention is underestimated when assayed using an ELISA kit which contains a human IL-4 expressed in a non-human cell system. The proliferation ability (T₃₂) of the IL-4 of the present invention is distinct from that of a human IL-4 expressed in non-human cell systems, in particular, the proliferation ability (T₃₂) of the IL-4 of the present invention is 25-54 times greater than that of a human IL-4 expressed in E. coli cells; and the proliferation ability (T₃₂) of the IL-4 of the present invention is up to 1.75 fold greater proliferative activity than human IL-4 expressed in CHO cells. Further, the proliferation ability (T₃₂) of the IL-4 of the present invention is distinct from that of a human IL-4 expressed in a non-human cell system after extended pre-incubation at elevated temperatures, in particular, the proliferation ability (T₃₂) of the IL-4 of the present invention is 13-30 fold greater on TF-1 cells following a 4 day pre-incubation at 37° C. in cell culture medium than human GM-CSF expressed in E. coli cells.

In a particular embodiment, an IL-5 of the present invention is characterized by a profile of one or more physiochemical parameters (Pₓ) comprising an apparent molecular weight (P₁) of 1 to 250, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250 kDa and in a particular embodiment 15 to 25 kDa. The pI (P₂) of IL-5 of the present invention is 2 to 14 such as 2, 3, 4, 5, 6, 7, 8, 9, 10, 11, 12, 13, 14 and in a particular embodiment 4 to 9 with about 2 to 50, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 isoforms and in a particular embodiment 5-12 isoforms (P₃). The percentage by weight carbohydrate (P₅) of the IL-5 of the present invention is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and in a particular embodiment 10 to 50%. The observed molecular weight of the IL-5 of the present invention when the N-linked oligosaccharides are removed (P₆) is between 9 to 30 kDa and in a particular embodiment, between 11 and 25 kDa. The observed molecular weight of the IL-5 of the present invention when the N-linked oligosaccharides and O-linked oligosaccharides re removed (P₇) is between 8 to 27 kDa and in a particular embodiment, between 10 and 22 kDa. Monosaccharide content (P₉) of the IL-5 of the present invention, when normalized to GalNAc, are 1 to 0.1-3 fucose, 1 to 0.5-7 GlcNAc, 1 to 0.05-3 galactose, 1 to 0.1-3 mannose and 1 to 0-5 NeuNAc; and in a particular embodiment 1 to 0-0.5 fucose, 1 to 2-4.5 GlcNAc, 1 to 1-2 galactose, 1 to 1-2 mannose and 1 to 0.1-1 NeuNAc; when normalized to 3 times of mannose, are 3 to 0.1-3 fucose, 3 to 0.1-4 GalNAc, 3 to 1-17 GlcNAc, 3 to 1-8 galactose and 3 to 0-5 NeuNAc; in a particular embodiment 3 to 0-1 fucose, 3 to 2-3 GalNAc, 3 to 3-12 GlcNAc, 3 to 2-5 galactose and 3 to 0.2-1 NeuNAc. The sialic acid content (P₁₀) expressed as a percentage of the monosaccharide content of the IL-5 of the present invention is 0 to 50%, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% and in a particular embodiment 2 to 10%. The sulfate content (P₁₁) of the IL-5 of the present invention, when normalized to GalNAc, are 1 to 2-14 sulfate; and in a particular embodiment 1 to 5-10 sulfate; when normalized to 3 times of mannose, are 3 to 7-36 sulfate and in a particular embodiment 3 to 12-24 sulfate. The sulfation (P₅₉) expressed as a percentage of the monosaccharide content of IL-5 of the present invention is 5-35% and in a particular embodiment 10-25%. Neutral percentage of N-linked oligosaccharides (P₁₃) of the IL-5 of the present invention is 30 to 90%, in a particular embodiment 40 to 80% and in an additional embodiment 50 to 75%. Acidic percentage of N-linked oligosaccharides (P₁₄) of the IL-5 of the present invention is 10 to 70%, in a particular embodiment 20 to 60% and in an additional embodiment 25 to 50%. Neutral percentage of O-linked oligosaccharides (P₁₅) of the IL-5 of the present invention is 40 to 100%, in a particular embodiment 50 to 100% and in an additional embodiment 60 to 100%. Acidic percentage of O-linked oligosaccharides (P₁₆) of the IL-5 of the present invention is 0 to 60%, in a particular embodiment 0 to 50% and in an additional embodiment 0 to 40%.

In one embodiment, the protein or chimeric molecule of the present invention contains at least one of the following structures in the N-linked fraction (P₁₉). In these representations, “u” or “?” represents that the anomeric configuration is either a or b, and/or the linkage position is 2, 3, 4, and/or 6.

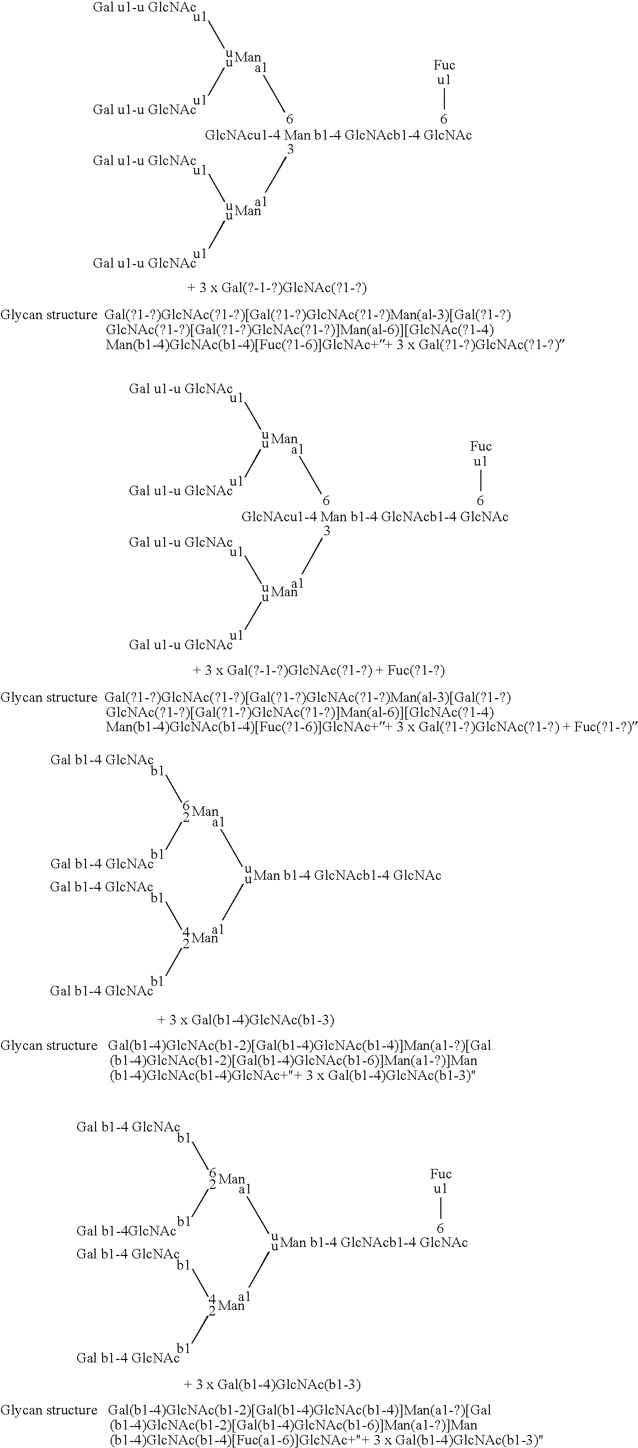

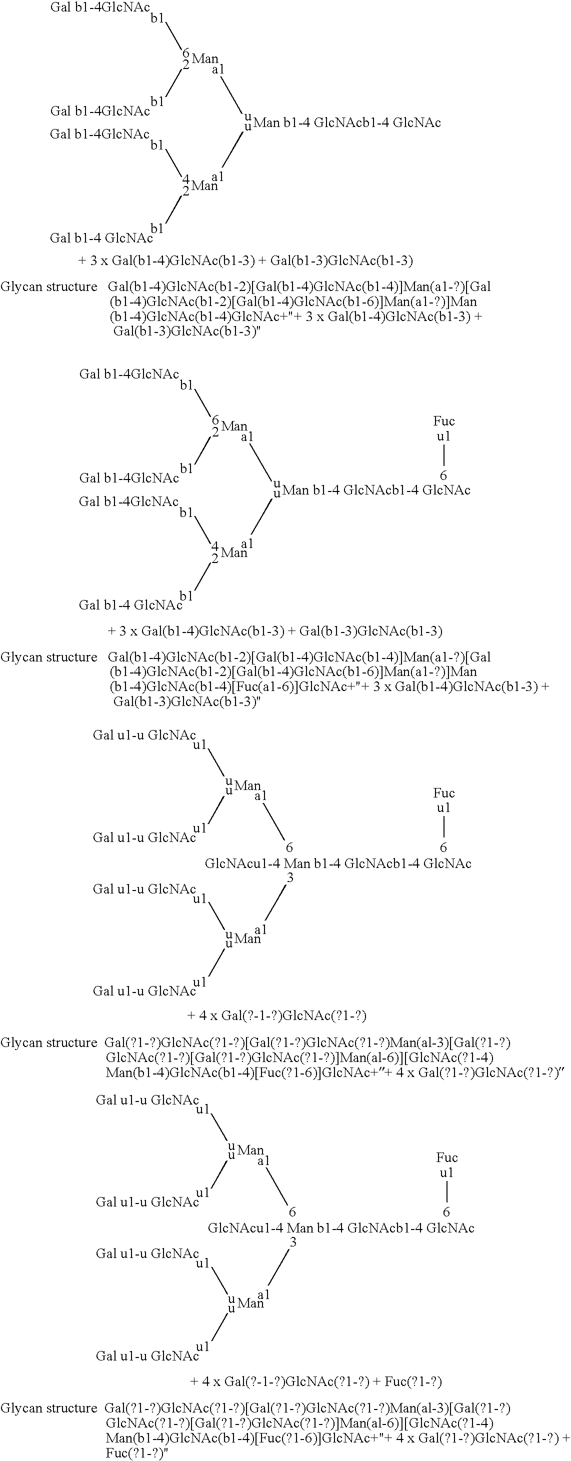

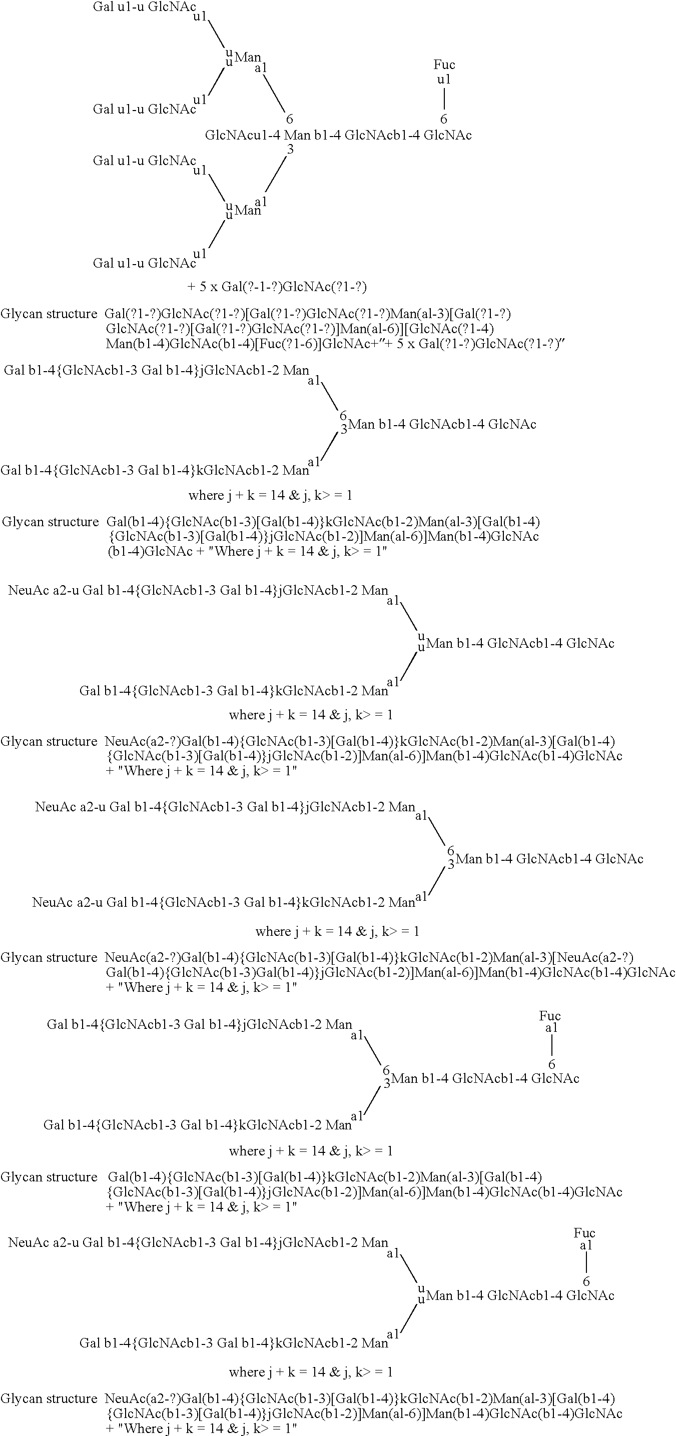

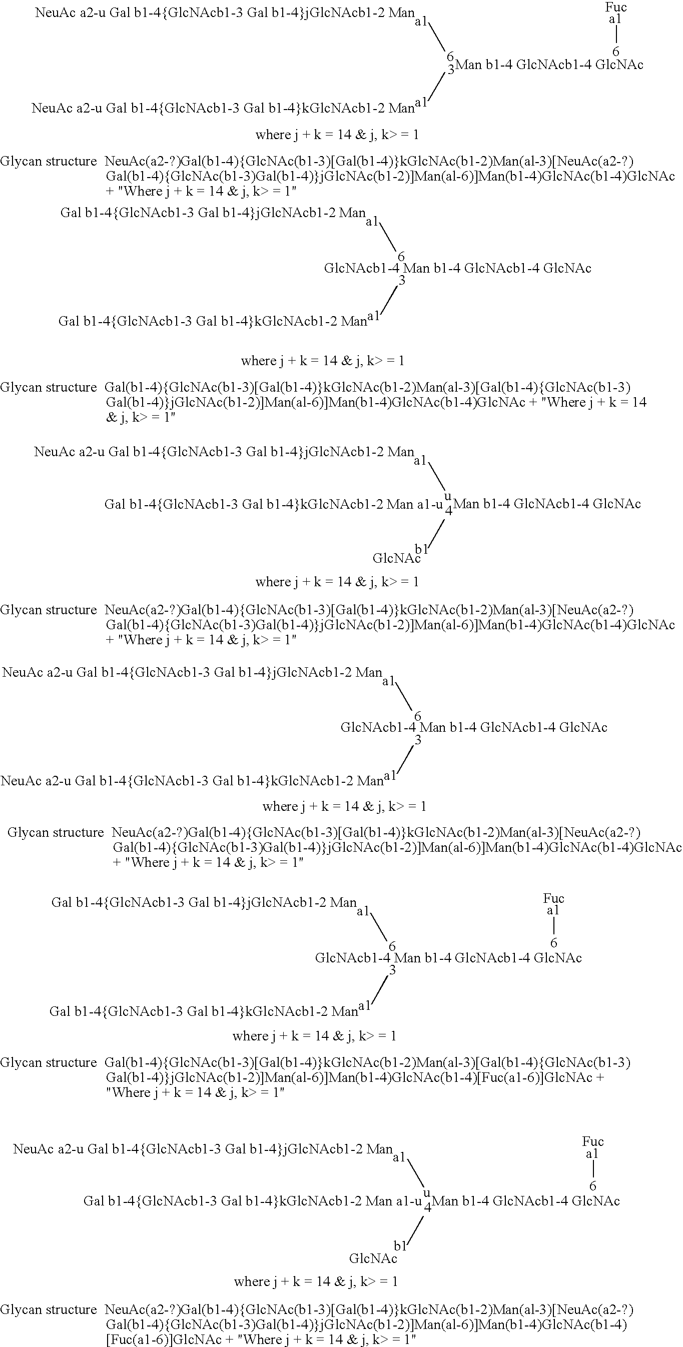

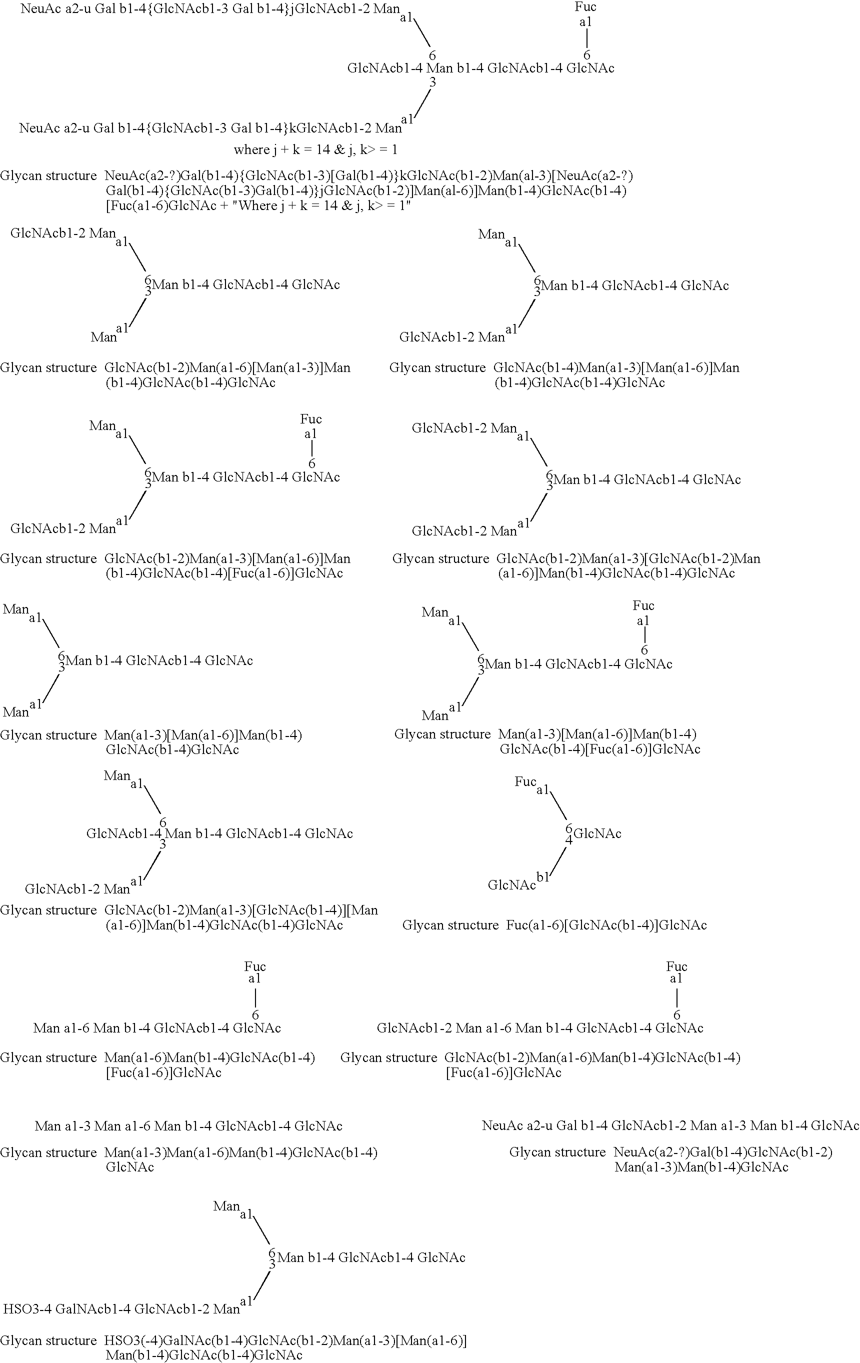

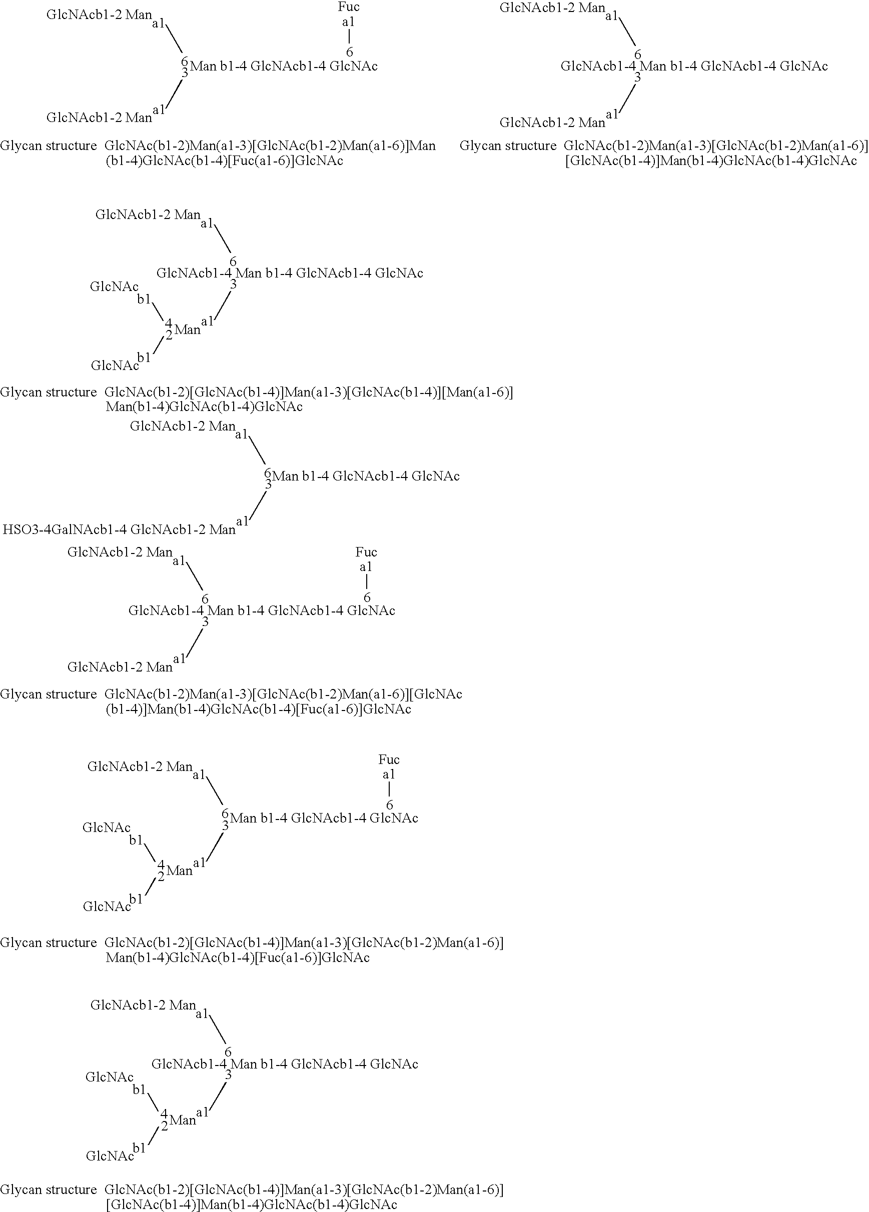

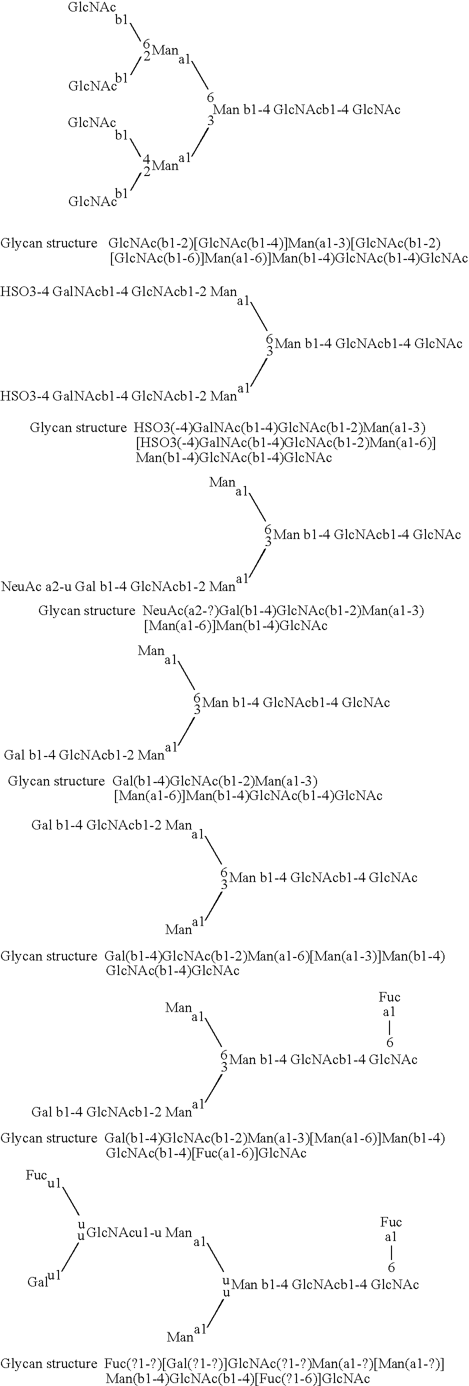

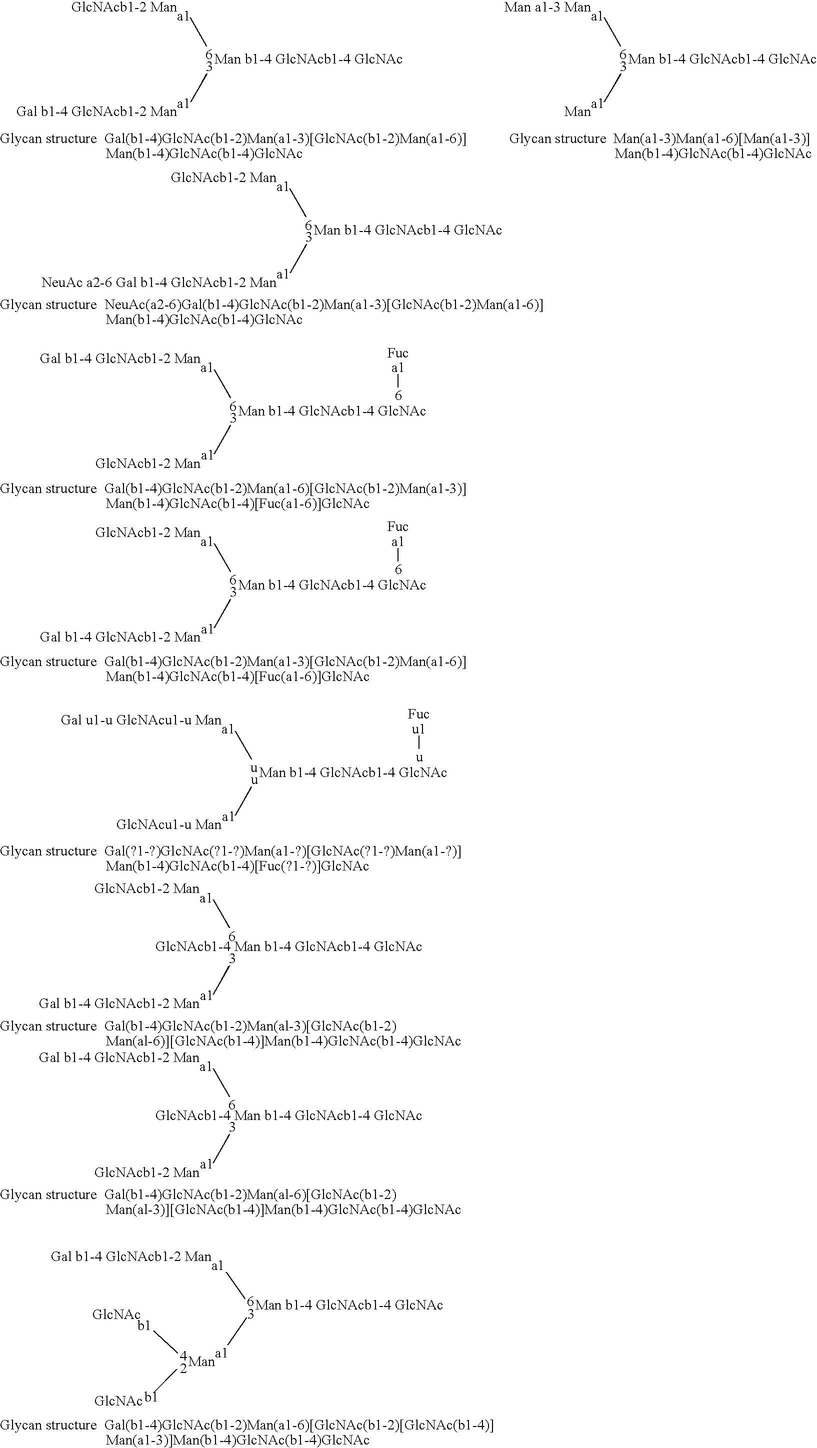

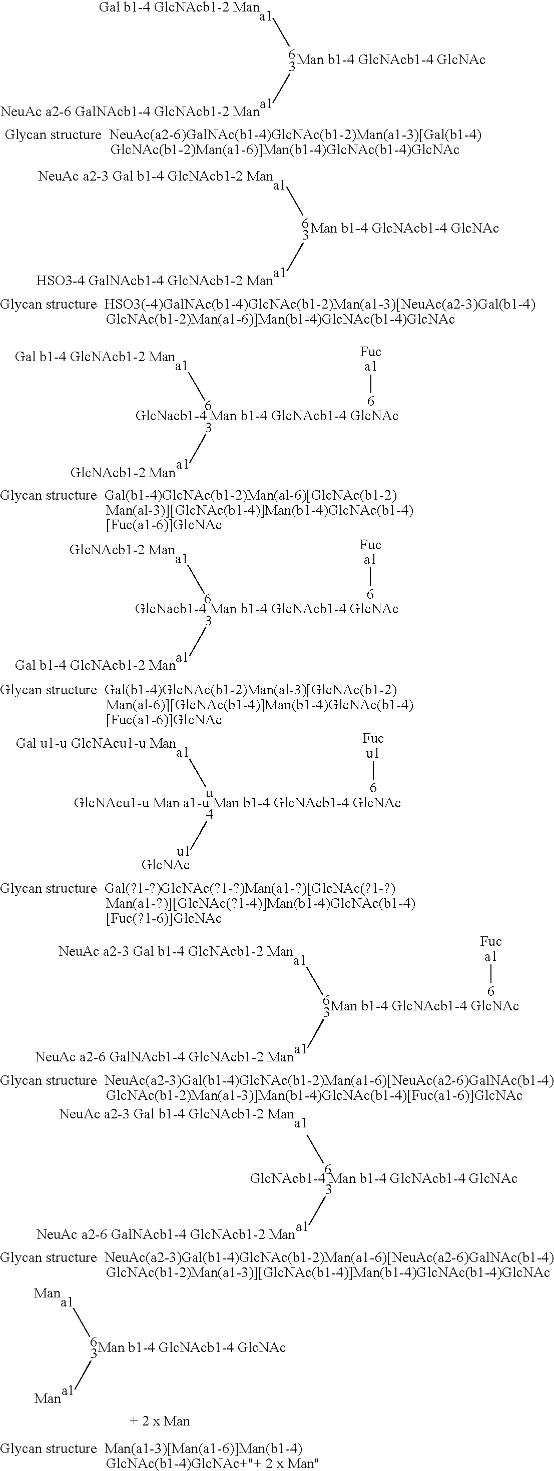

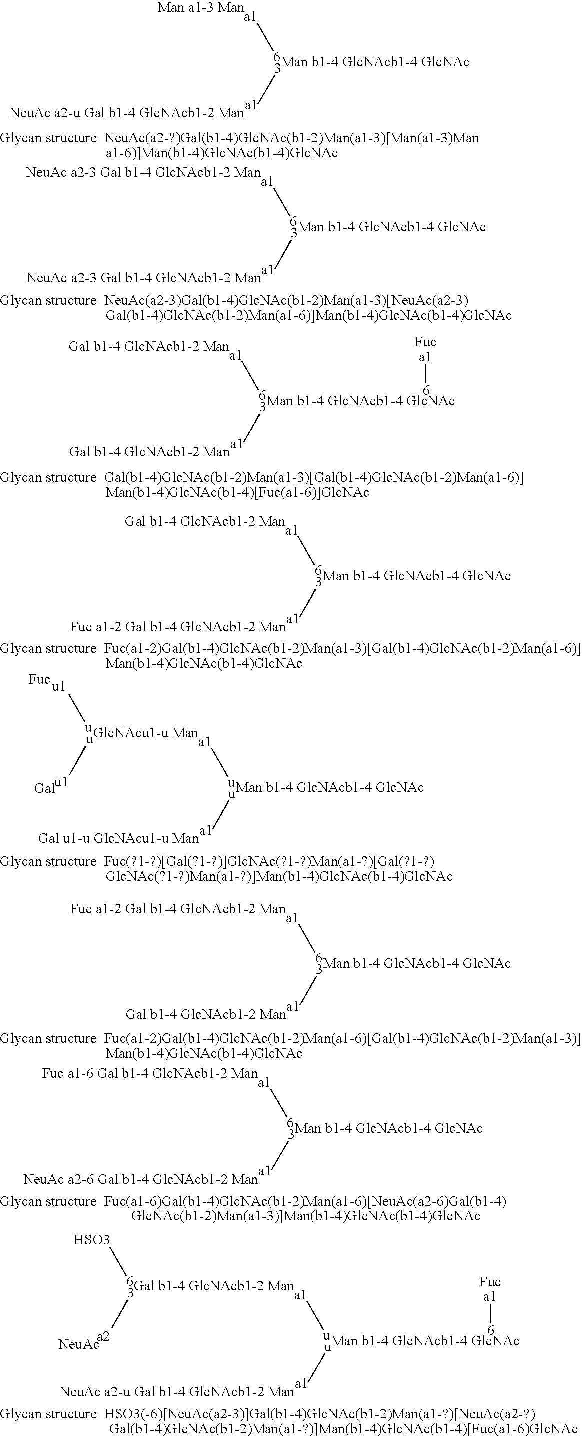

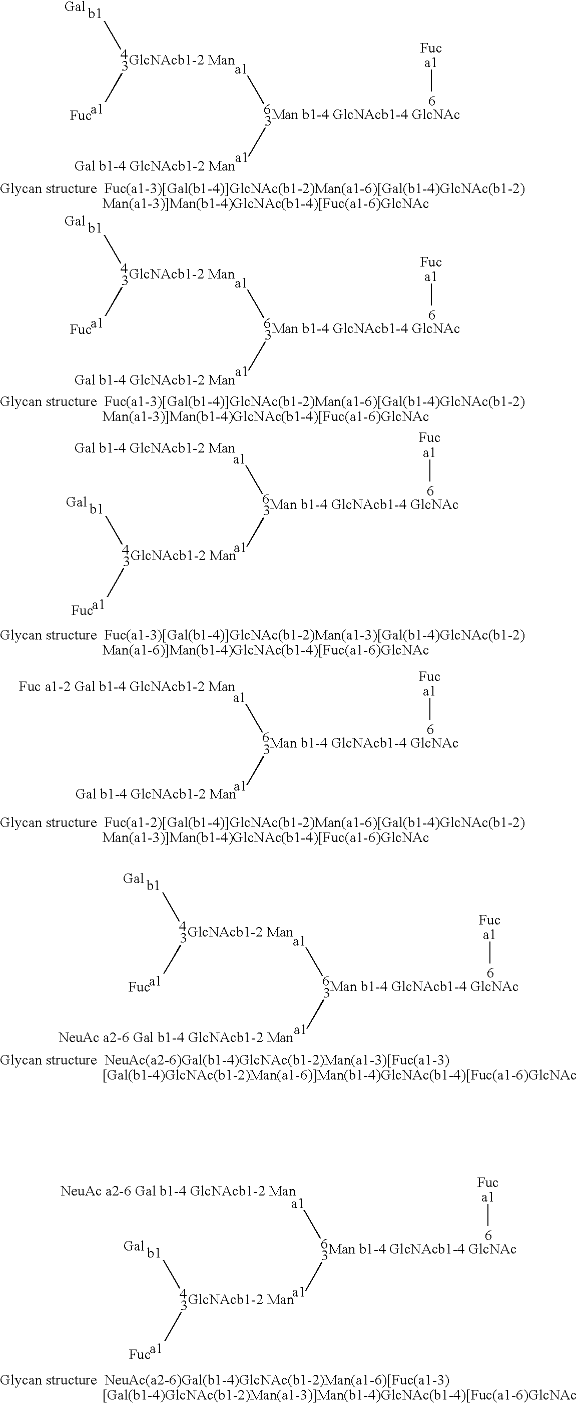

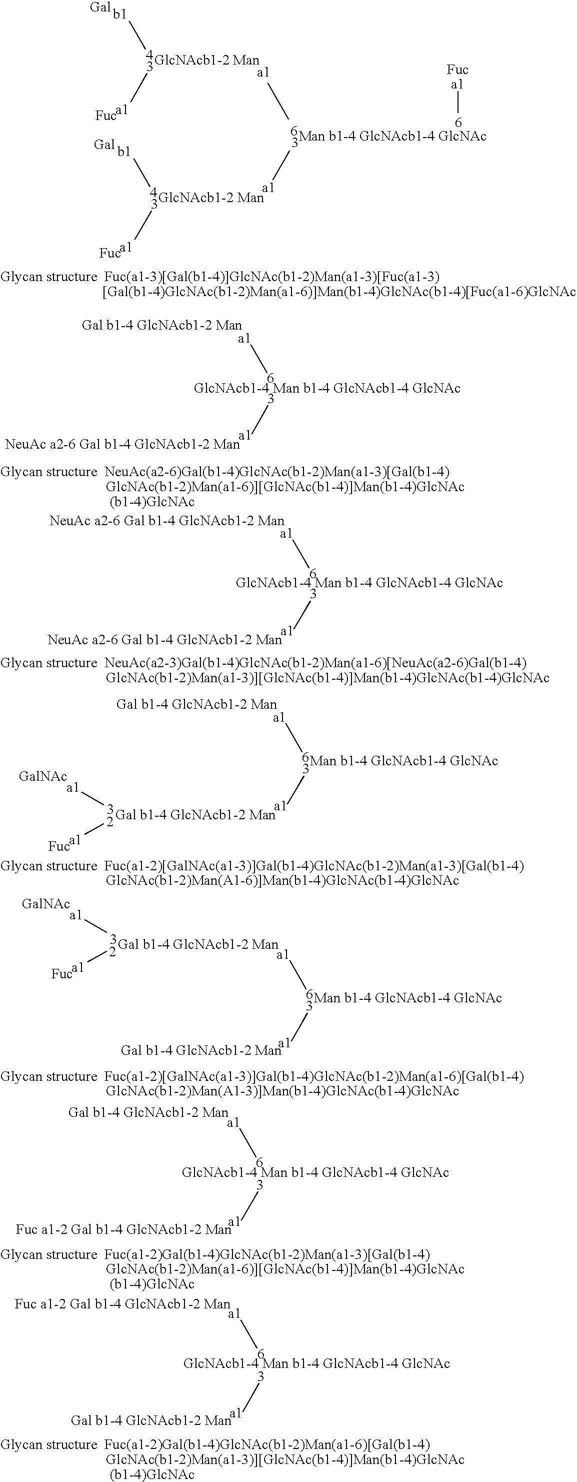

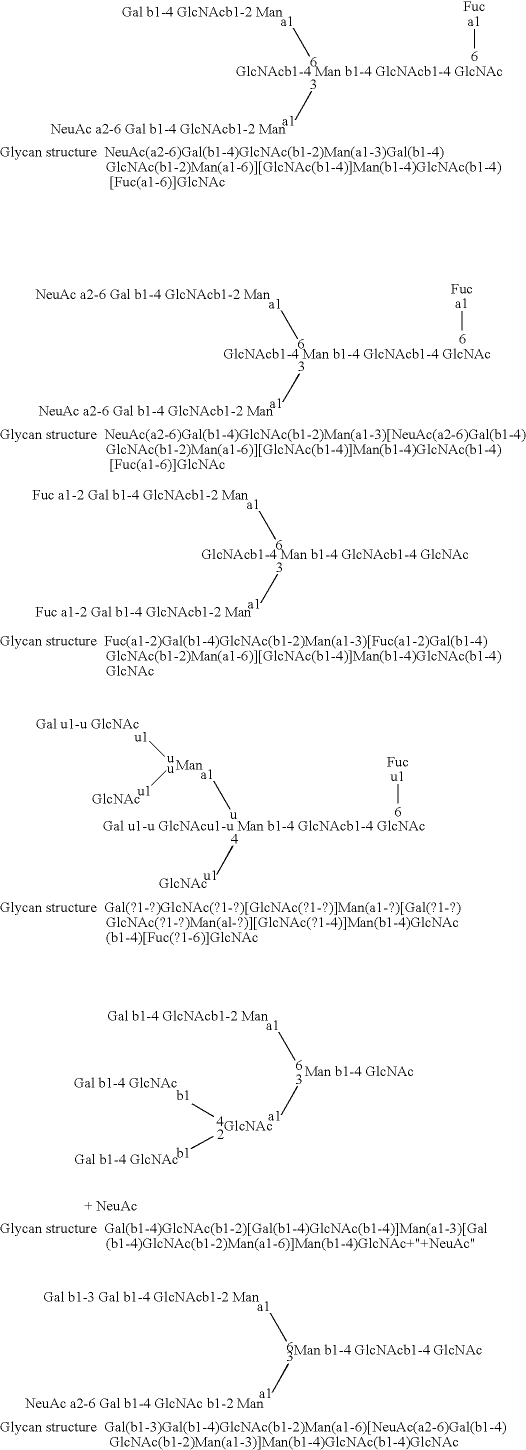

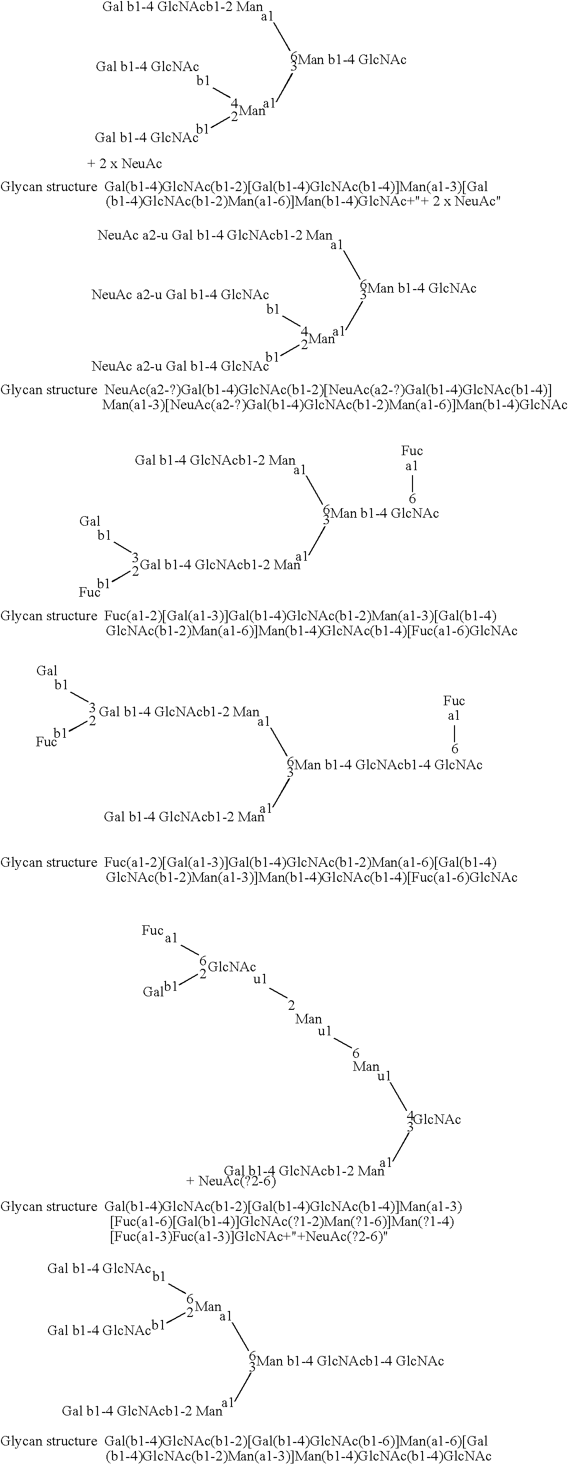

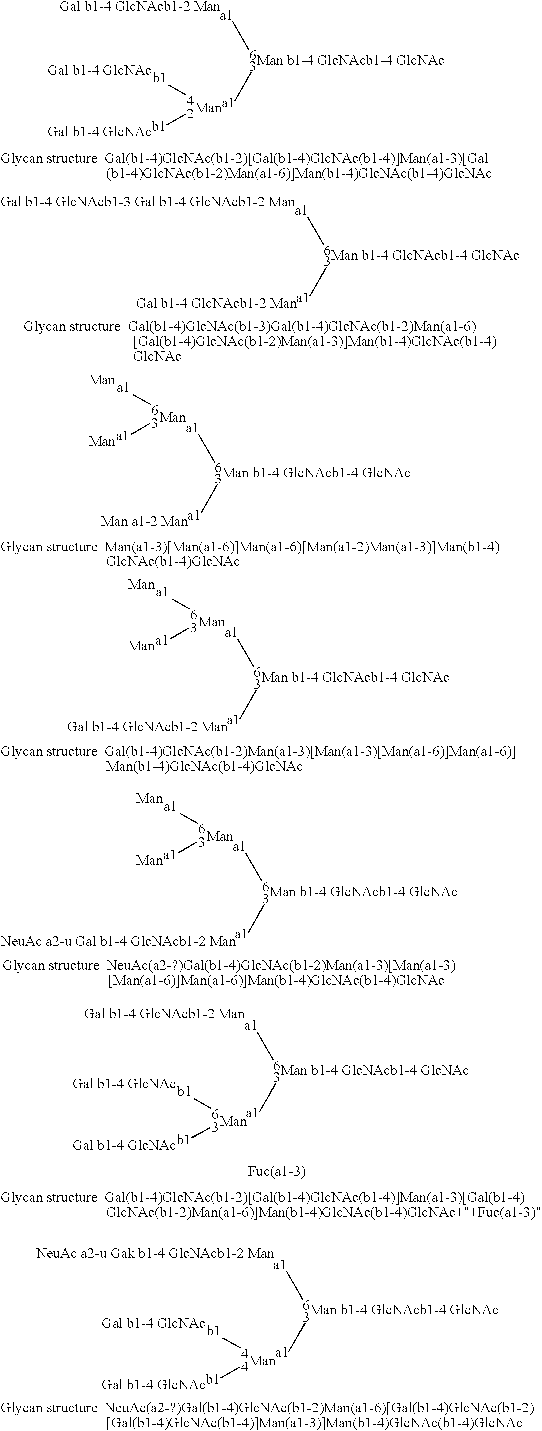

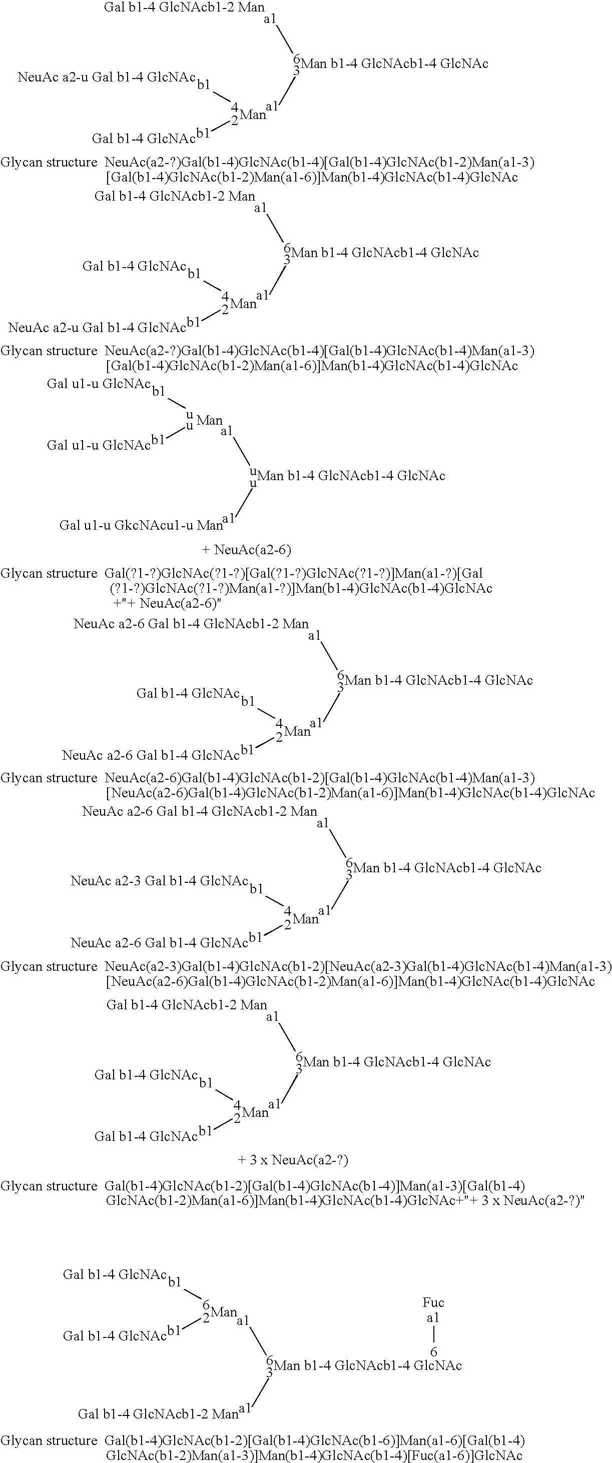

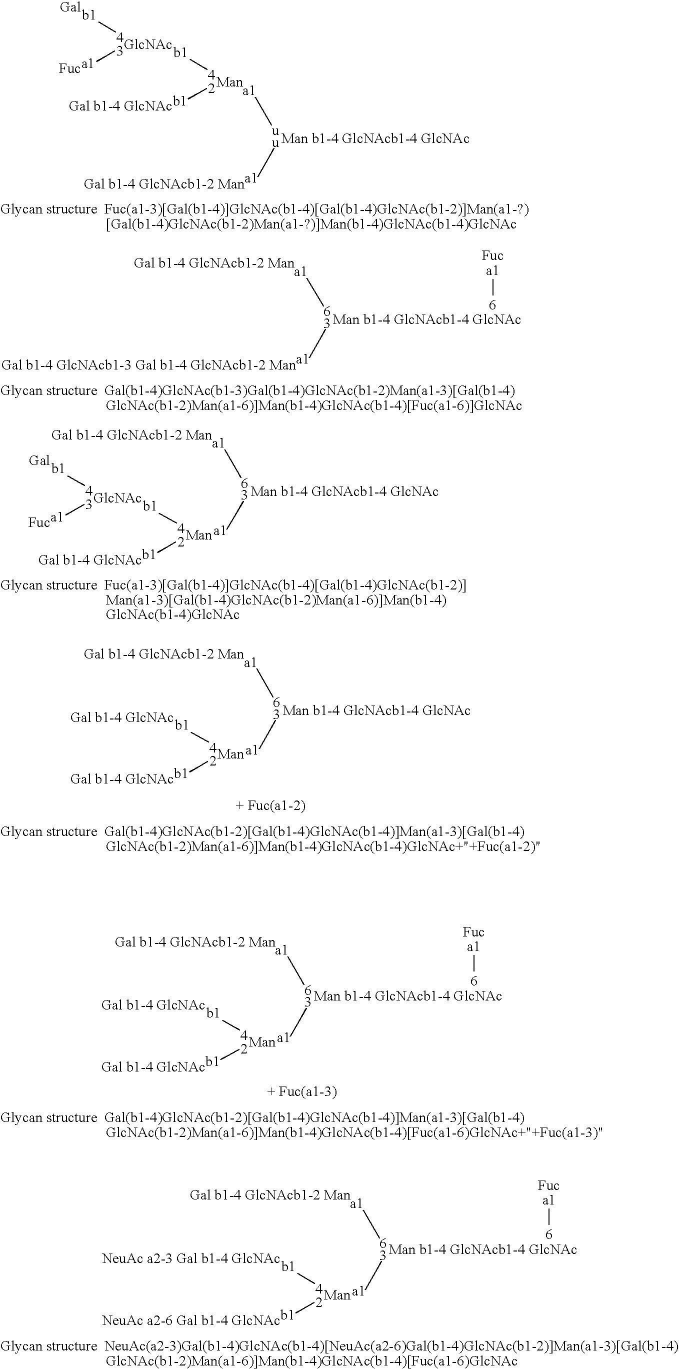

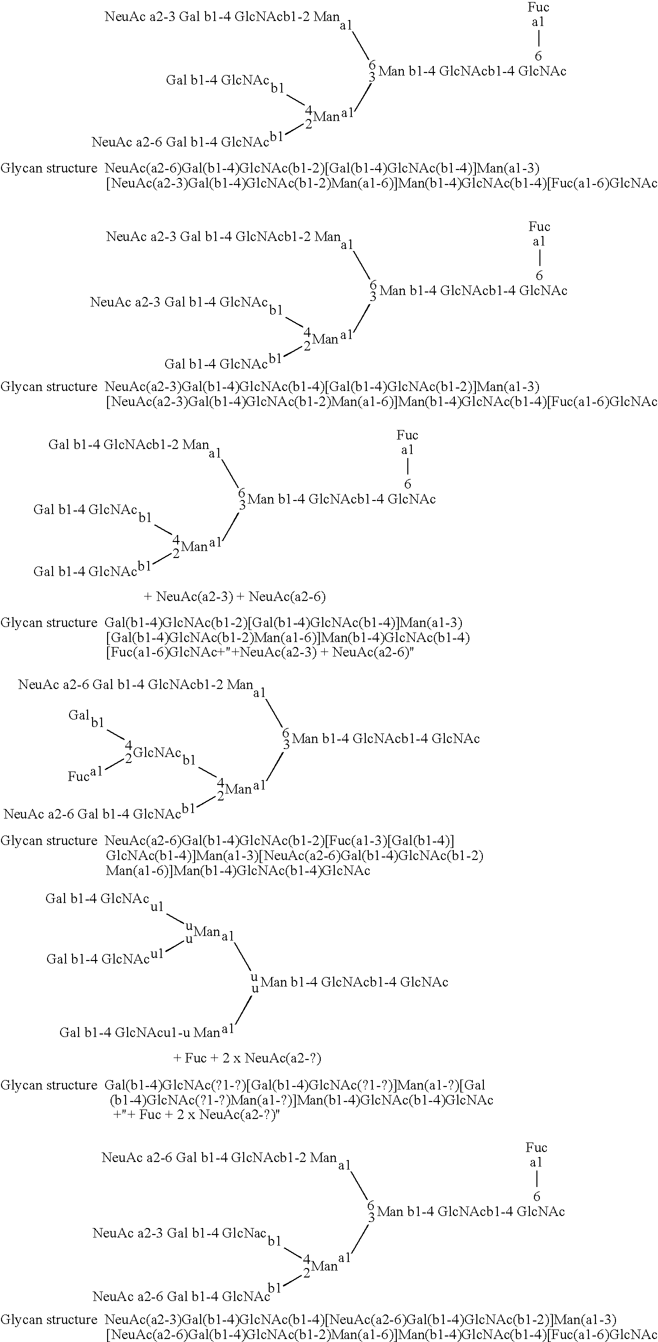

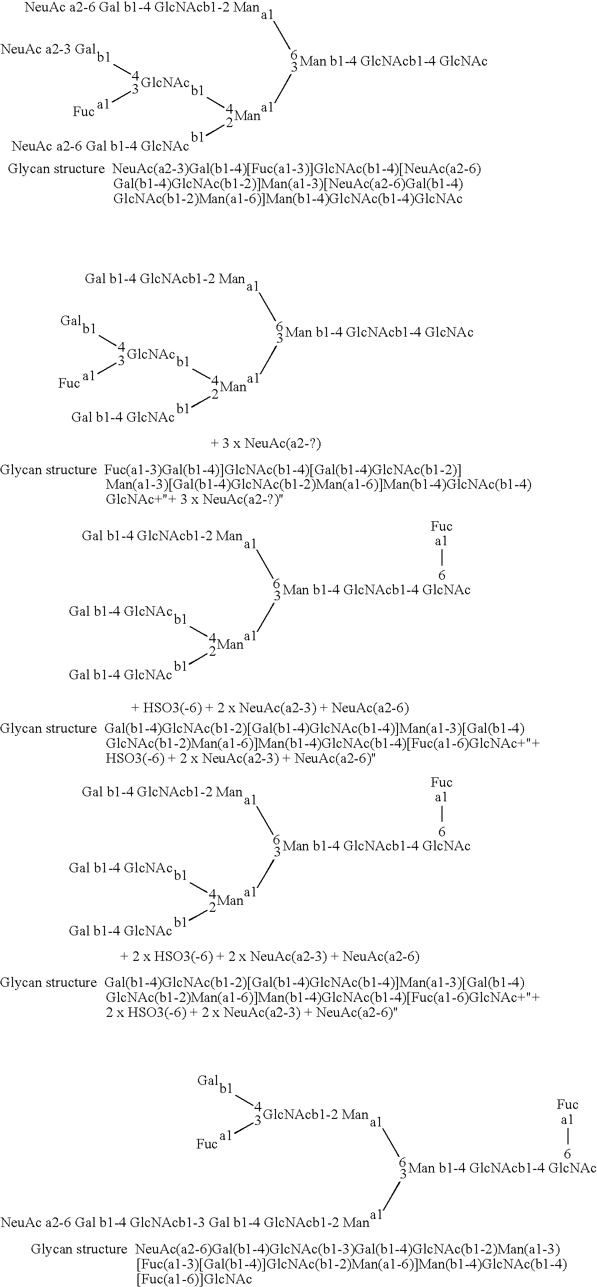

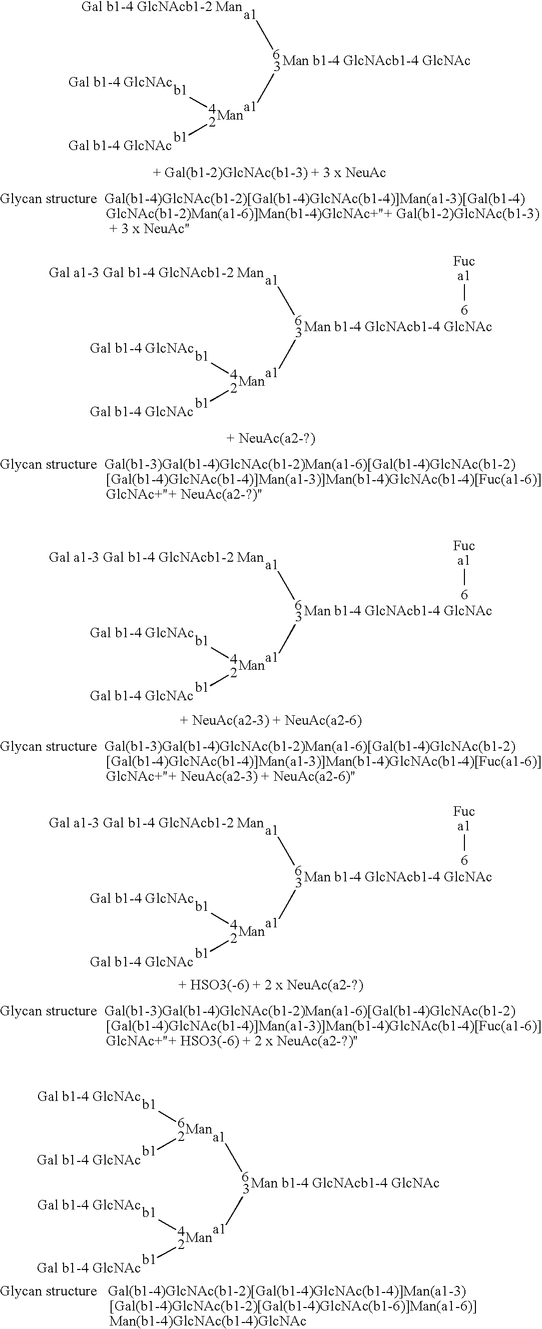

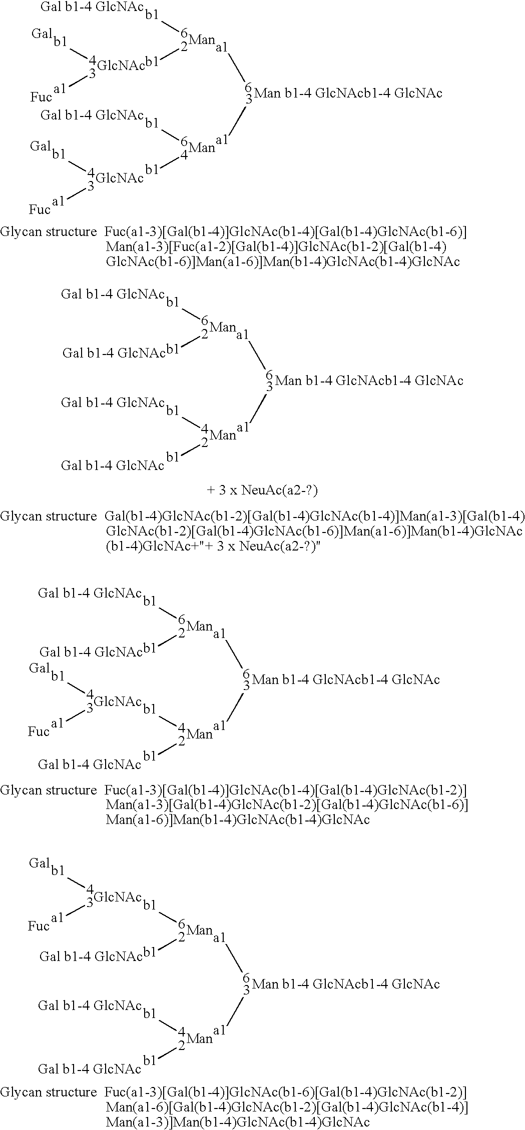

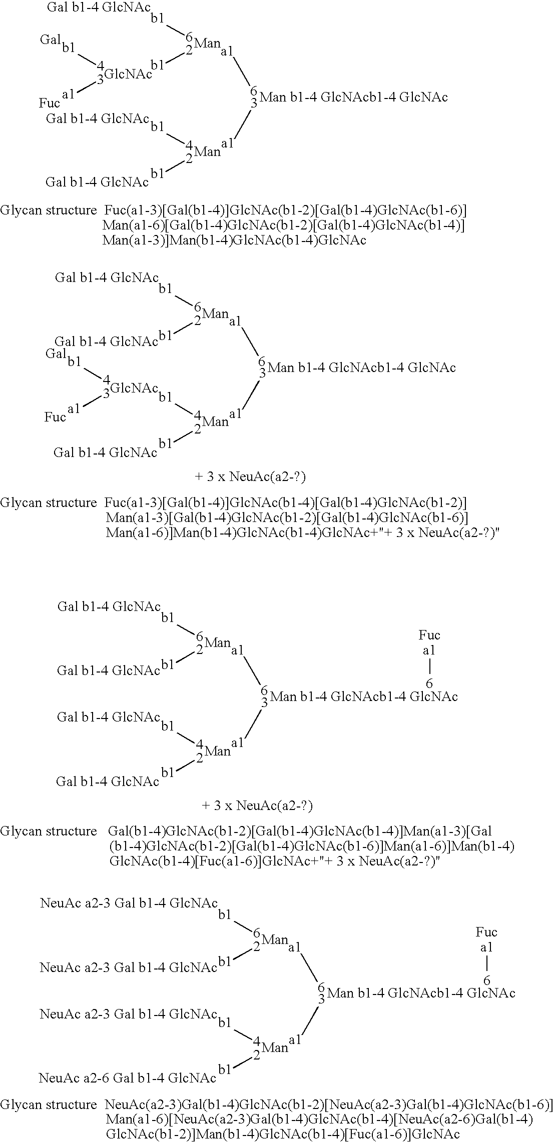

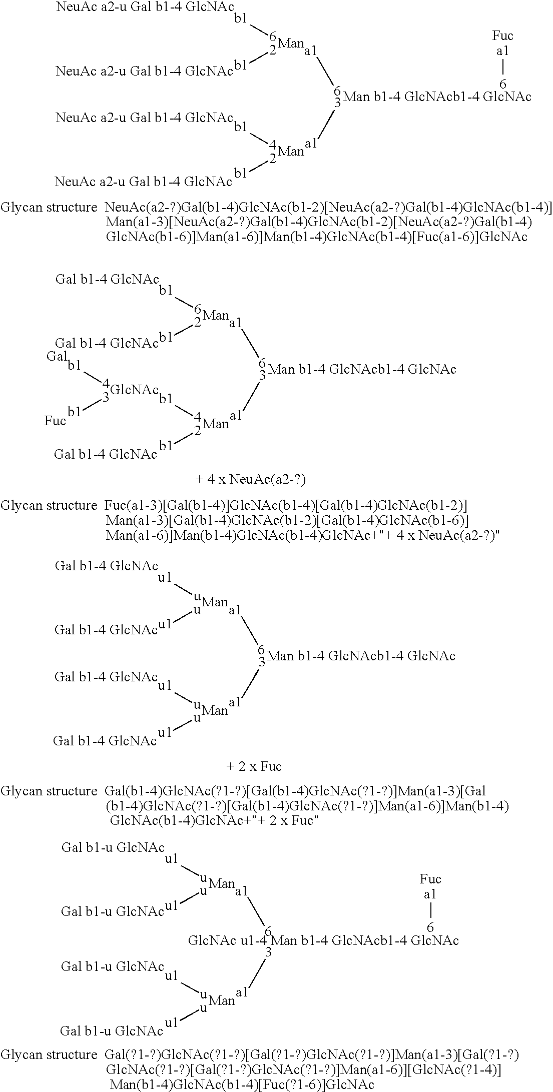

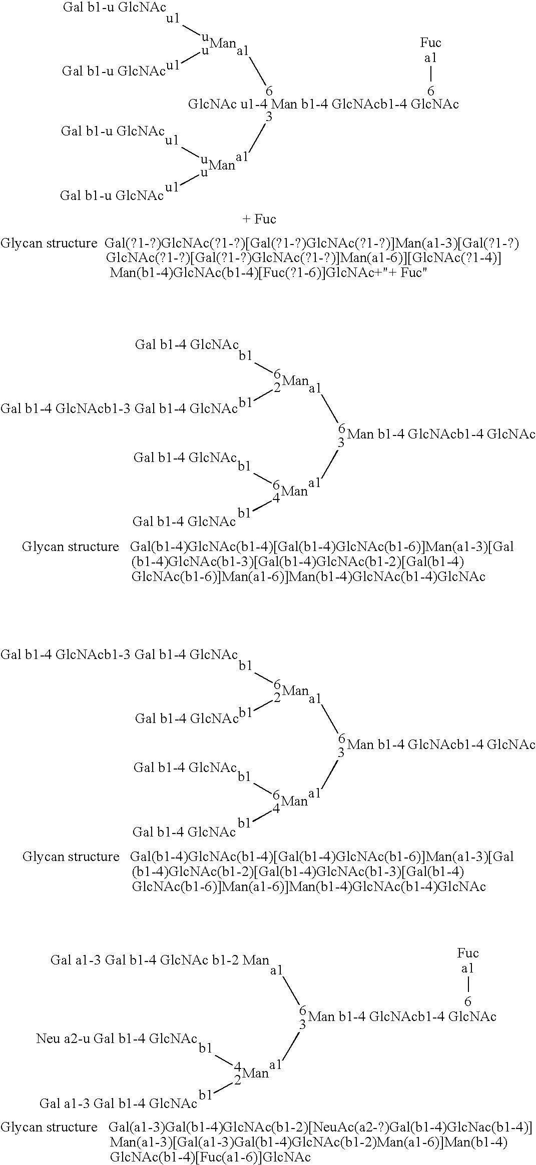

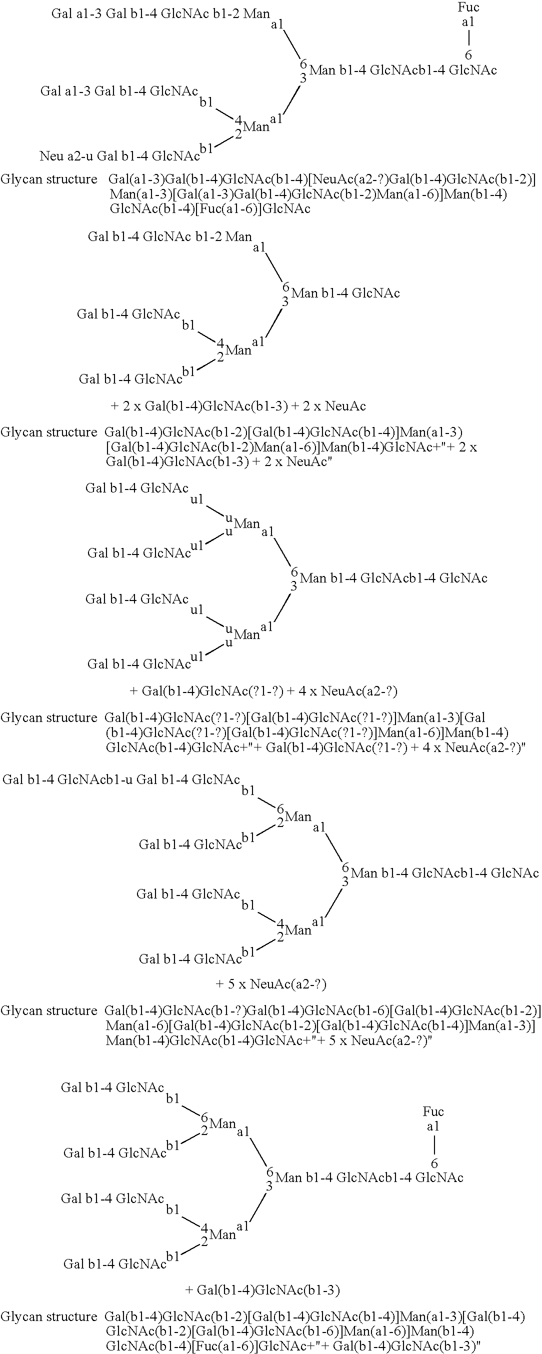

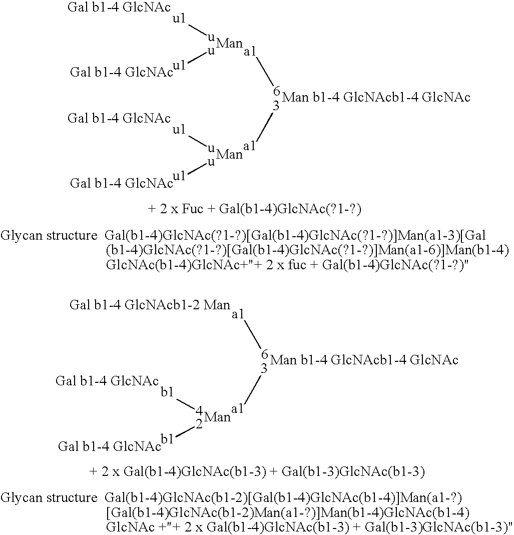

In one embodiment, the protein or chimeric molecule of the present invention contains at least one of the following structures in the O-linked fraction (P₂₀). In these representations, “u” or “?” represents that the anomeric configuration is either a or b, and/or the linkage position is 2, 3, 4, and/or 6.

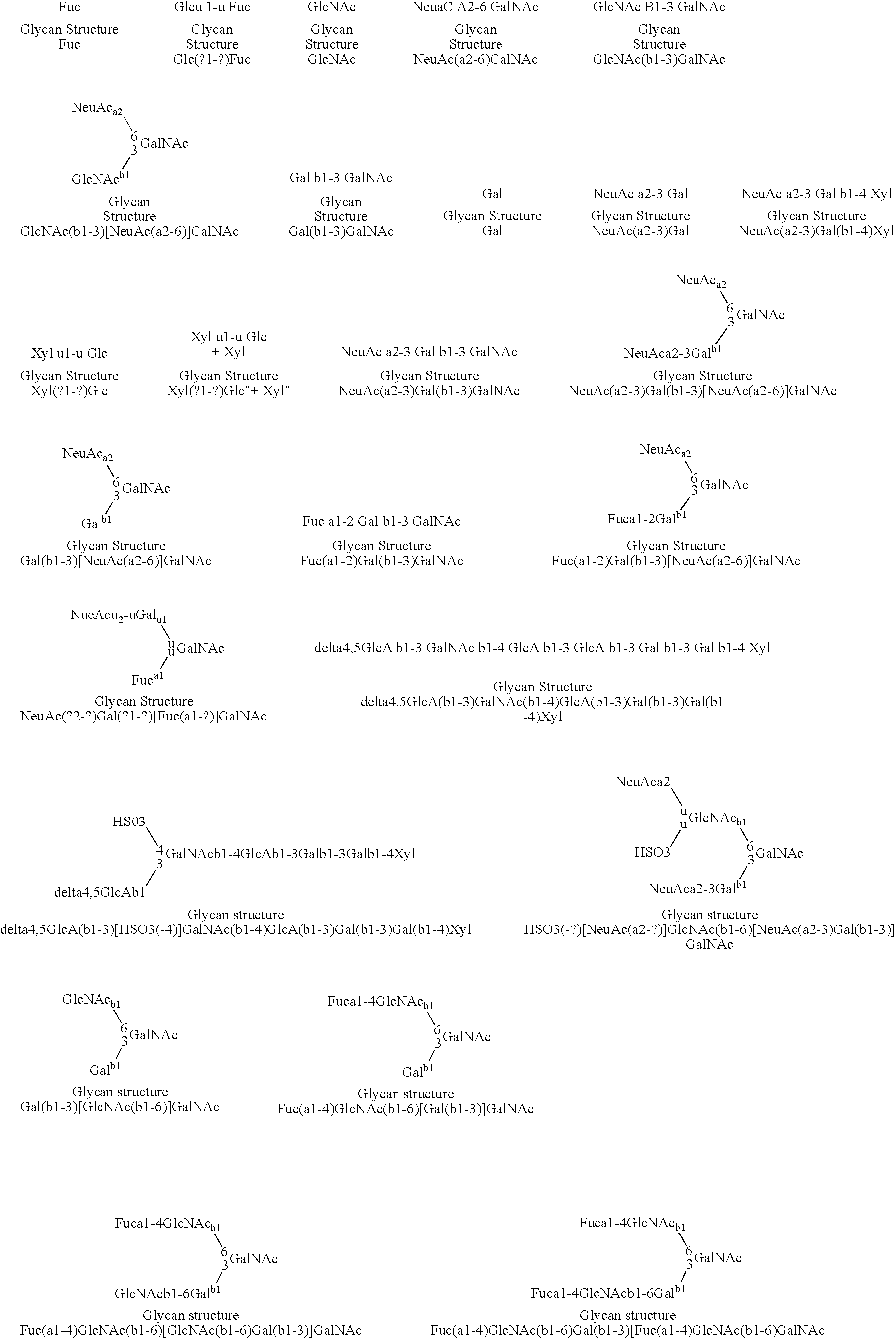

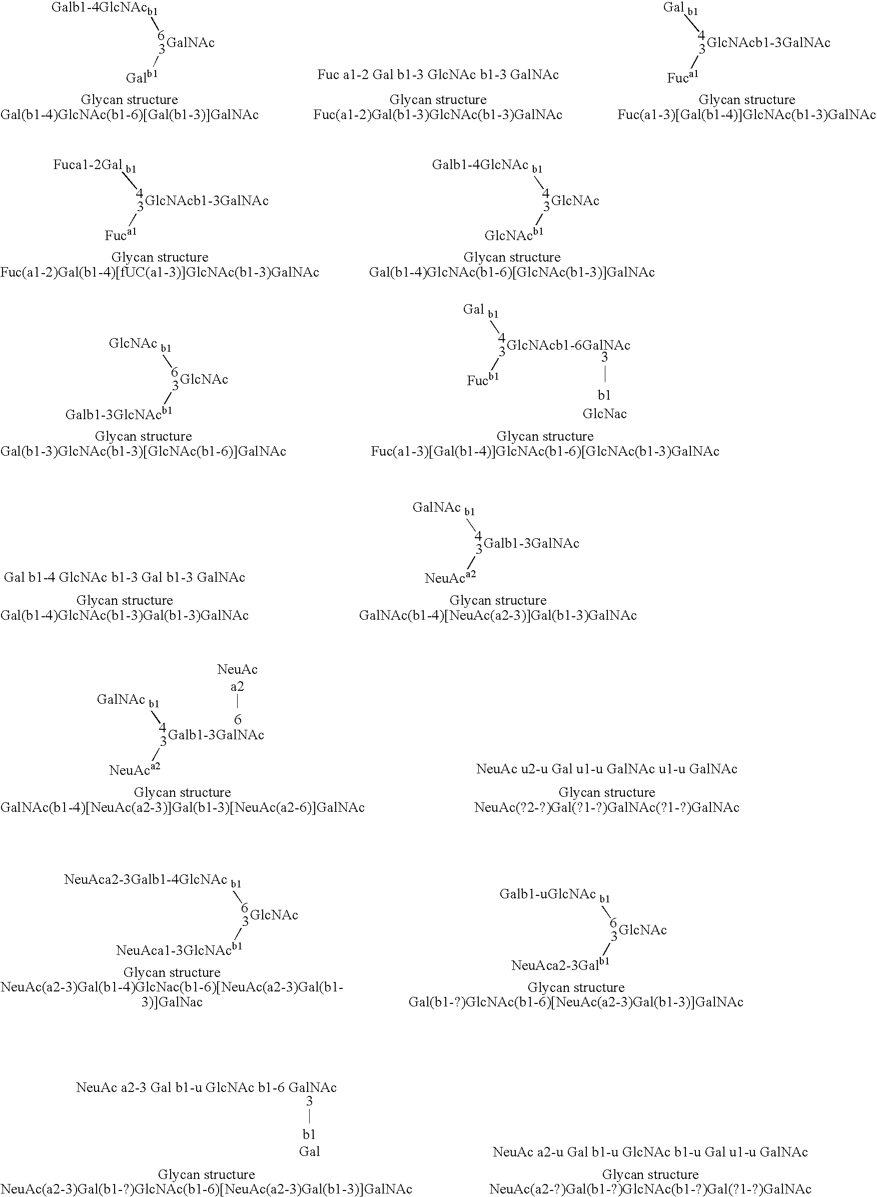

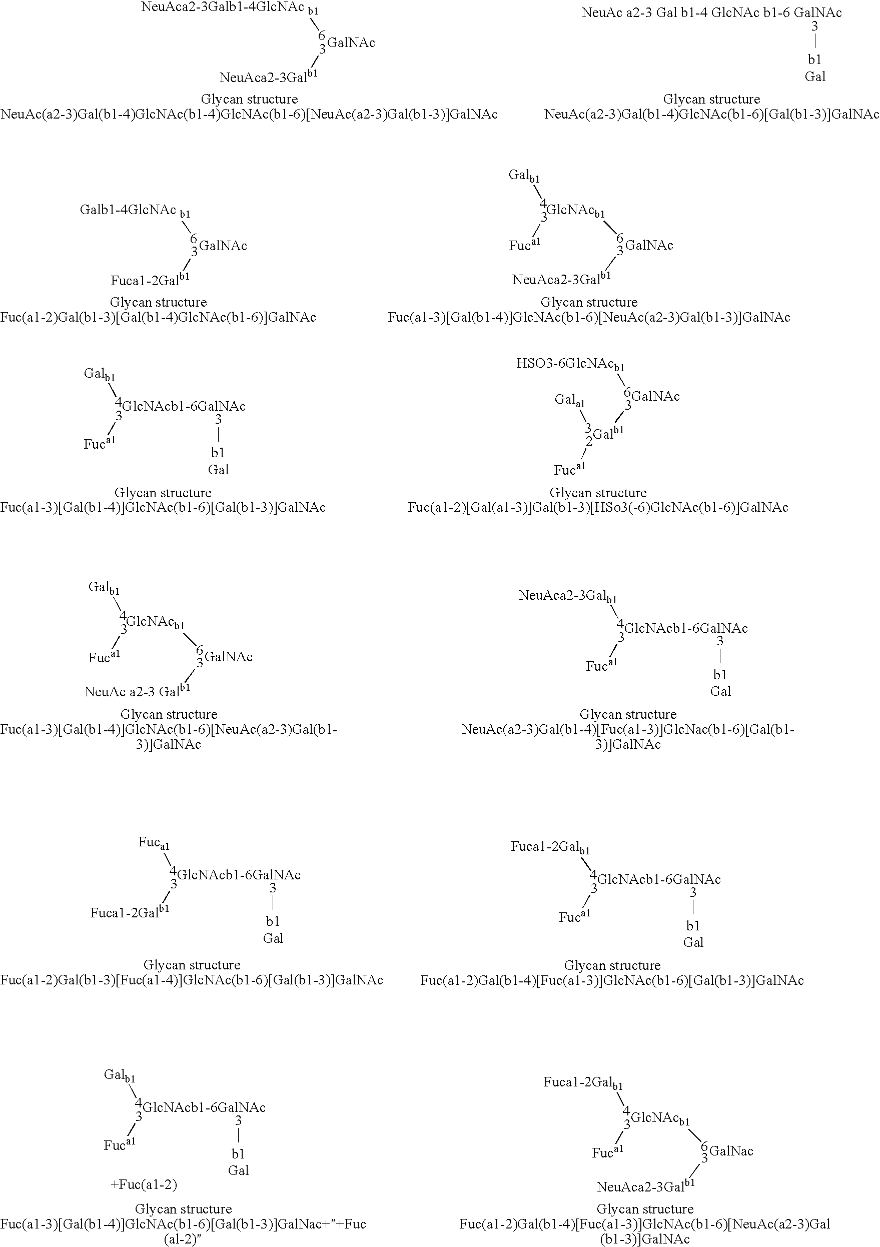

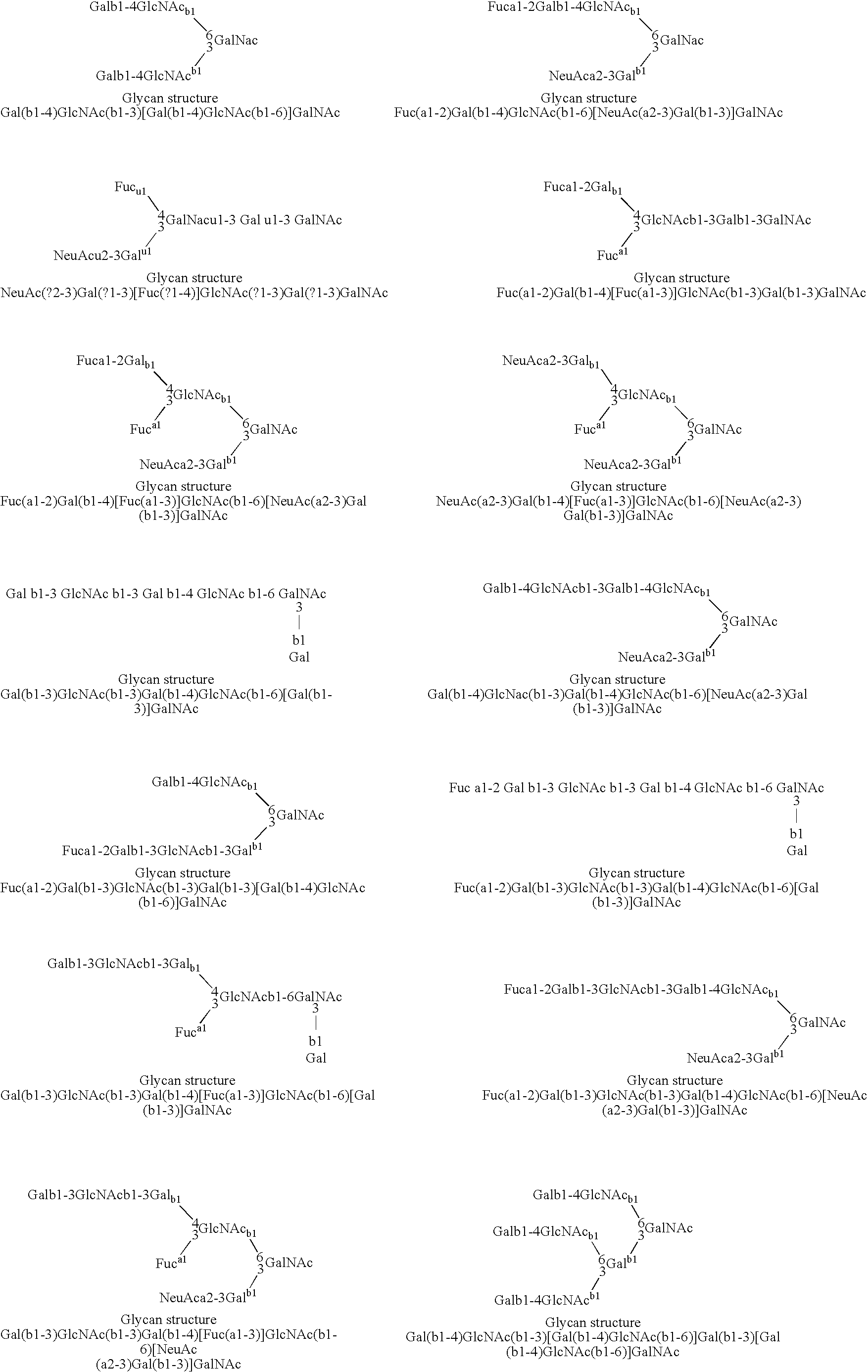

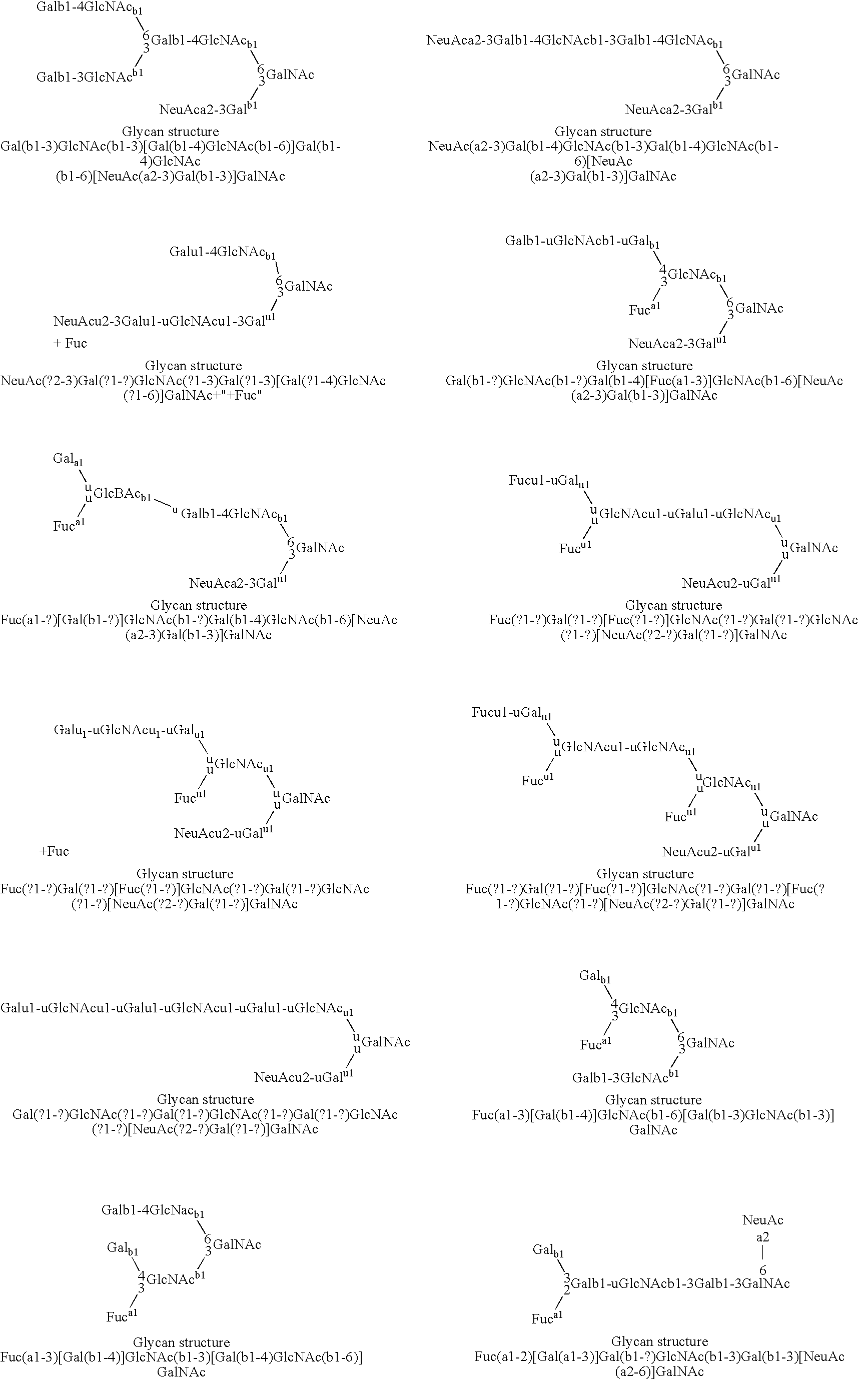

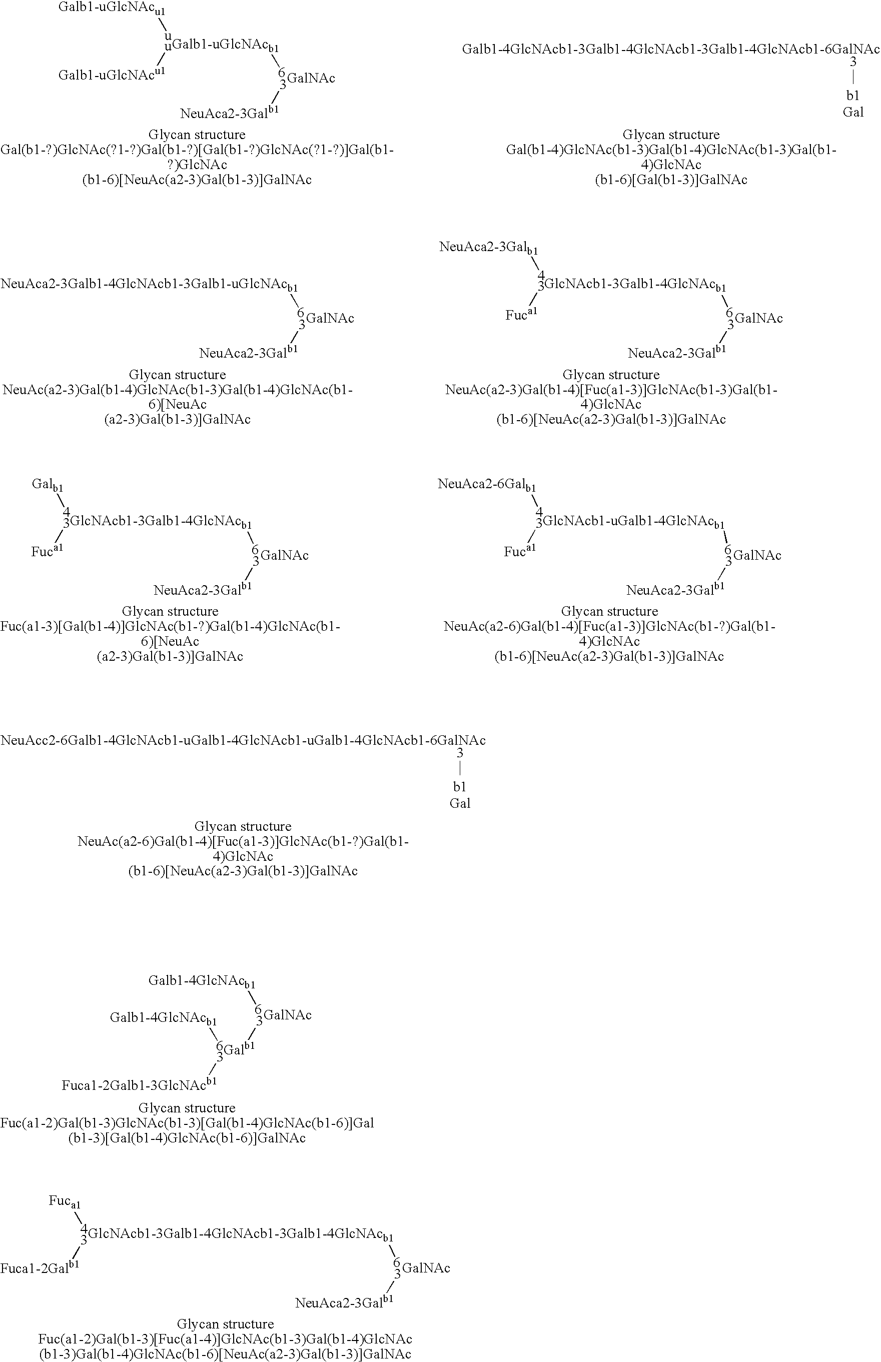

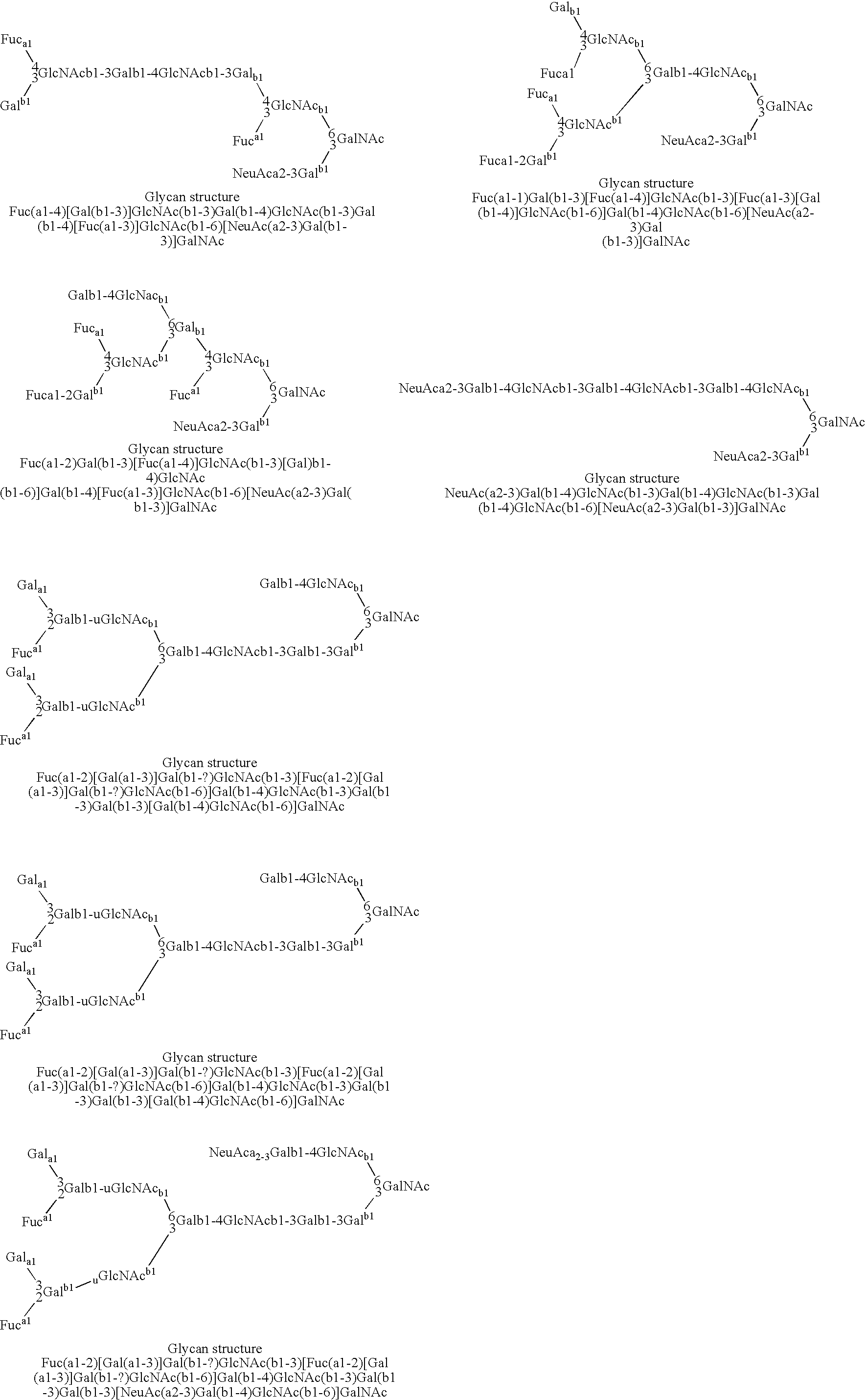

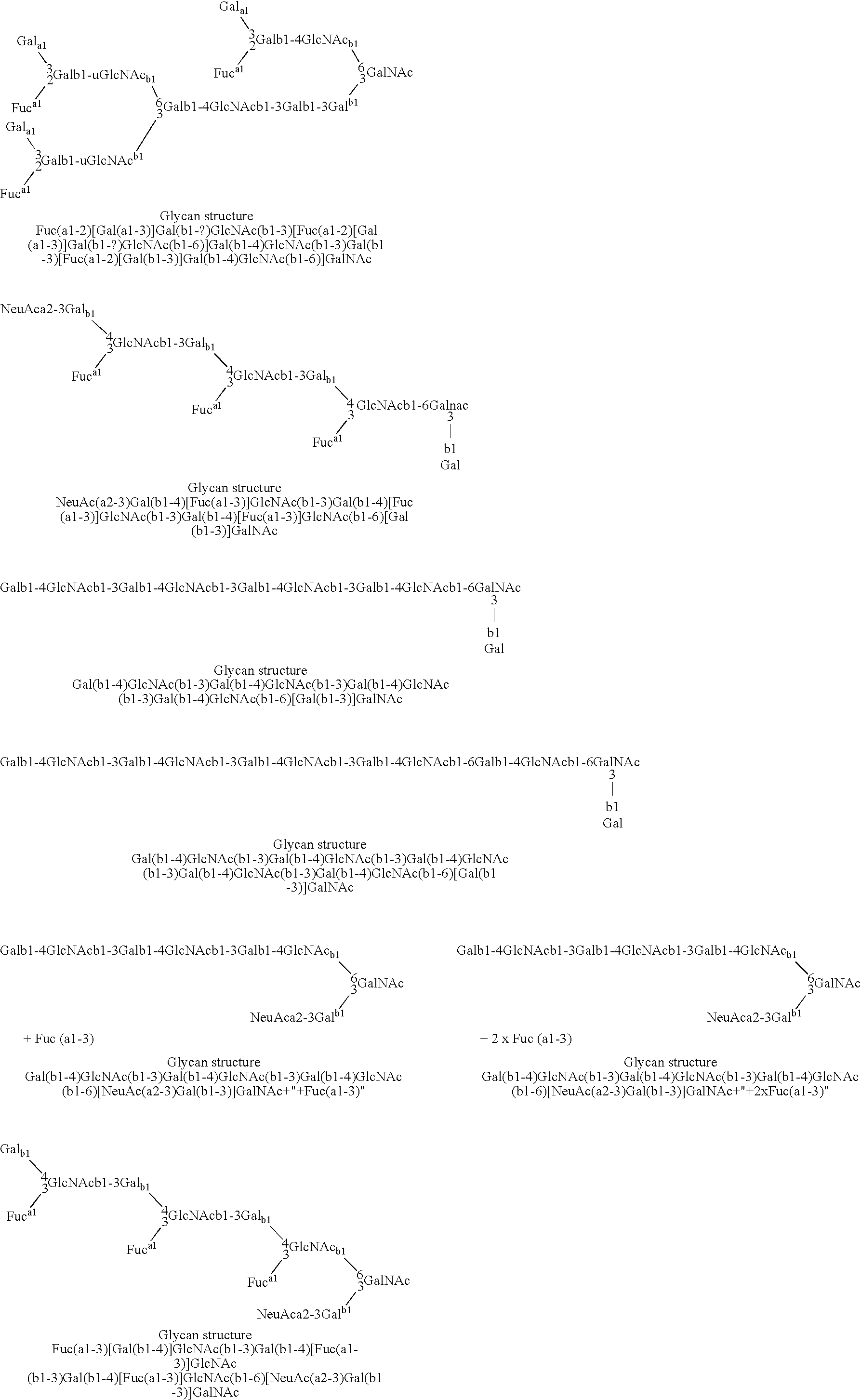

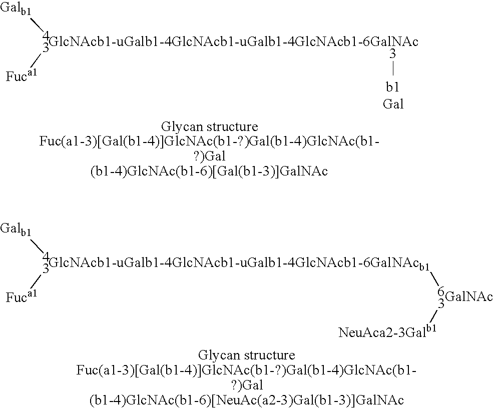

The physiochemical form of the protein or chimeric molecule of the present invention may be achieved by modifying the host cell by a variety of ways known in the art, including but not limited to the introduction of one or more transgene into the host cell that encodes an enzyme or enzymes that will produce the desired physiochemical form. Such transgenes include various types of sialyltransferases, such as ST3Gal1, ST3Gal2, ST3Gal3, ST3Gal4, ST3Gal5, ST3Gal6, ST6Gal1, ST6Gal2, ST6GalNAc1, ST6GalNAc2, ST6GalNAc3, ST6GalNAc4, ST6GalNAc5, ST8Sia1, ST8Sia2, ST8Sia3, ST8Sia4, ST8Sia5, ST8Sia6; galactosyltransferases, such as GalT1, GalT2; fucosyltransferases such as FUT1, FUT2, FUT3, FUT4, FUT5, FUT6, FUT7, FUT8, FUT9, FUT10, FUT11; sulfotransferases; GlcNAc transferases such as GNT1, GNT2, GNT3, GNT4, GNT5; antenna-cleaving enzymes and endoglycosidases.

For instance, inefficient terminal sialylation of N-glycan structures that results in reduced serum half-life of an expressed protein such as recombinant human AchE can be ameliorated by the addition of a rat beta-galactoside alpha-2,6-sialyltransferase transgene to HEK 293 cells (J Biochem 336:647-658, 1998; J Biochen 363:619-631, 2002).

Similarly, inefficient formation of particular Lewis x groups such as sialyl Lewis x structures on N-glycan structures that results in reduced ligand binding of an expressed protein such as recombinant human PSGL-1 can be ameliorated by the addition of a fucosyltransferase transgene to HEK 293 cells (Fritz et al. PNAS 95:12283-12288, 1998).

In one embodiment, a protein or chimeric molecule thereof is produced using a human cell line transformed with either α-2,3 or α-2,6 sialytransferase, or both α-2,3 sialytransferase and α-2,6 sialytransferase (“sialylated-protein”). Examples of sialylated-protein include sialylated-GM-CSF, sialylated-GM-CSF-Fc, sialylated-IL-3, sialylated-IL-3-Fc, sialylated-IL-4, sialylated-IL-4-Fc, sialylated-IL-5 and sialylated-IL-5-Fc.

In particular, the sialylated-protein is characterized by a profile of physiochemical parameters (Pₓ) comprising monosaccharide (P₉) and sialic acid contents (P₁₀) of, when normalized to GalNAc, 1 to 0.1-100 NeuNAc; and when normalized to 3 times of mannose 3 to 0.1-100 NeuNAc. Neutral percentage of N-linked oligosaccharides (P₁₃) of the sialylated-protein is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99%. Acidic percentage of N-linked oligosaccharides (P₁₄) of the sialylated-protein is 1 to 100% such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. Neutral percentage of O-linked oligosaccharides (P₁₅) of the sialylated-protein is 0 to 99% such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99%. Acidic percentage of O-linked oligosaccharides (P₁₆) of the sialylated-protein is 1 to 100% such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

The in vivo half-life (T₁₁) of the sialylated-protein is increased in comparison to the half-life of the protein or chimeric molecule of the invention expressed without the transgene.

In one embodiment, the sialylated-protein contains at least one of the structural formulae described herein or at least one of the structural formulae described herein where one or more NeuNAc linkage is a α 2,6 linkage in the N-linked fraction.

In one embodiment, the sialylated-protein contains at least one of the structural formulae described herein or at least one of the structural formulae described herein where one or more NeuNAc linkage is a α 2,6 linkage in the O-linked fraction.

In one embodiment, the protein or chimeric molecule thereof of the invention is produced using a human cell line transformed with FUT3 (“fucosylated-protein”). Examples of fucosylated-protein include fucosylated-GM-CSF, fucosylated-GM-CSF-Fc, fucosylated-IL-3, fucosylated-IL-3-Fc, fucosylated-IL-4, fucosylated-IL-4-Fc, fucosylated-IL-5 and fucosylated-IL-5-Fc.

In particular, the fucosylated-protein is characterized by a profile of physiochemical parameters (Pₓ) comprising monosaccharide (P₉) and sialic acid contents (P₁₀) of, when normalized to GalNAc, 1 to 0.1-100 NeuNAc; and when normalized to 3 times of mannose 3 to 0.1-100 NeuNAc.

In one embodiment, the fucosylated-protein has a higher proportion of structure containing Lewis structures (such as Lewis a, Lewis b, Lewis x or Lewis y) or sialyl Lewis structures (such as sialyl Lewis a or sialyl Lewis x).

In one embodiment, the fucosylated-protein has altered binding affinity to ligands in comparison to the binding affinity of the protein or chimeric molecule of the invention expressed without the transgene.

Using respective forward primer and reverse primer for the protein molecule selected from GM-CSF, IL-3, IL-4 and IL-5, the DNA encoding the relevant protein was amplified from an EST by Polymerase Chain Reaction (PCR) by methods known in the art, for example, according to the method of Invitrogen's PCR Super Mix High Fidelity (Cat. No.:10790-020). The amplicon is digested and ligated into the corresponding restriction enzyme sites of an appropriate vector, for instance, pIRESbleo3, pCMV-SPORT6, pUMCV3, pORF, pORF9, pcDNA3.1/GS, pCEP4, pIRESpuro3, pIRESpuro4, pcDNA3.1/Hygro(+), pcDNA3.1/Hygro(−), pEF6/V5-His. The ligated vector is transformed into an appropriate E. coli host cell, for instance, XLGold, ultracompetant cell (Strategene), XL-Blue, DH5α, DH10B or the like.

For the production of chimeric molecules, the DNA sequence for the Fc domain of an immunoglobulin, such as IgG1, IgG2, IgG3, IgG4, IgGA1, IgGA2, IgGM, IgGE, IgGD is amplified from the EST using the appropriate forward and reverse primers by PCR. The amplicon is cloned into the corresponding restriction enzyme sites of an appropriate vector, for instance, pIRESbleo3, pCMV-SPORT6, pUMCV3, pORF, pORF9, pcDNA3.1/GS, pCEP4, pIRESpuro3, pIRESpuro4, pcDNA3.1/Hygro(+), pcDNA3.1/Hygro(−), pEF6/V5-His. The DNA sequence of relevant protein is amplified and cloned into the corresponding restriction enzyme sites of the respective Fc-vector in frame with the Fc.

In a particular embodiment, the Fc receptor binding region or the complement activating region of the Fc region may be modified recombinantly, comprising one or more amino acid insertions, deletions or substitutions relative to the amino acid sequence of the Fc region. In addition, the receptor binding region or the complement activating region of the Fc region may be modified chemically by changes to its glycosylation pattern, the addition or removal of carbohydrate moieties, the addition of polyunsaturated fatty acid moieties or other lipid based moieties to the amino acid backbone or to any associated co- or post-translational entities. The Fc region may also be in a truncated form, resulting from the cleavage by an enzyme including papain, pepsin or any other site-specific proteases. The Fc region may promote the spontaneous formation by the chimeric protein of a dimer, trimer or higher order multimer that is better capable of binding to its corresponding ligand or receptor.

Diagnostic digests using the appropriate restriction enzymes are performed to identify/isolate bacterial colonies containing the vector bearing the correct gene. Positive colonies are isolated and stored as Glycerol stocks at −70° C. The clone is then expanded to 750 ml of sterile LB broth containing ampicillin (100 μg/ml) at 37° C. with shaking for 16 hours. The plasmid is prepared in accordance with methods known in the art, preferably, in accordance with a Qiagen Endofree Plasmid Mega Kit (Qiagen Mega Prep Kit #12381).

Human host cells suitable for the introduction of the cloned DNA sequence comprising a the protein or chimeric molecule of the present invention include but are not limited to HEK 293 and any derivatives thereof, HEK 293 c18, HEK 293-T, HEK 293 CEN4, HEK 293F, HEK 293FT, HEK 293E, AD-293 (Stratagene), 293A (Invitrogen), Hela cells and any derivatives thereof, HepG2, PA-1 Jurkat, THP-1, HL-60, H9, HuT 78, Hep-2, Hep G2, MRC-5, PER.C6, SKO-007, U266, Y2 (Apollo), WI-38, W1-L2.

The physiochemical form of protein or chimeric molecule of the present invention may be achieved by modifying the host cell by a variety of ways known in the art, including but not limited to the introduction of a transgene into the host cell that encodes an enzyme or enzymes that will produce the desired physiochemical form. The introduction of specific DNA sequences can be used to optimize the integration of the cloned DNA sequence into the host cell genome, the various types of integration including but not limited to site-specific, targeted, direct or enzyme-mediated integration.

The DNA of protein or chimeric molecule thereof can be introduced into suitable host cells by various transfection methods known in the art, for instance, using chemical reagents such as DEAE-dextran, calcium phosphate, artificial liposomes, or by direct microinjection, electroporation, biolistic particle delivery or infection or transfection with viral constructs as described below.

DEAE-dextran is a cationic polymer that associates with negatively charged nucleic acids. An excess of positive charge, contributed by the polymer in the DNA/polymer complex allows the complex to come into closer association with the negatively charged cell membrane. Uptake of the complex is presumably by endocytosis. Other synthetic cationic polymers including polybrene, polyethyleneimine and dendrimers have also been used for transfection.

Calcium phosphate co-precipitation can be used for transient and stable transfection of a variety of cell types. The DNA is mixed with calcium chloride in a controlled manner and added to a buffered saline/phosphate solution and the mixture is incubated at room temperature. A precipitate is generated and is taken up by the cells via endocytosis or phagocytosis.

The most commonly used synthetic lipid component of liposomes for liposome-mediated gene delivery is one which has overall net positive charge at physiological pH. Often the cationic lipid is mixed with a neutral lipid such as L-dioleoyl phosphatidylethanolamine (DOPE). The cationic portion of the lipid molecule associates with the negatively charged nucleic acids, resulting in compaction of the nucleic acid in a liposome/nucleic acid complex. Uptake of the complex is by endocytosis.

Direct microinjection of DNA into cultured cells or nuclei is an effective, although laborious technique, which is not appropriate if a large number of transfected cells are required.

Electroporation utilizes an electric pulse, which generates pores that allow the passage of nucleic acids into the cells. This technique requires fine-tuning and optimization for duration and strength of the pulse for each type of cell used. Commercially available electroporation device includes Amaxa Biosystems' Nucleofector Kits (Amaxa Biosystems, Germany).

This method relies upon high velocity delivery of nucleic acids on microprojectiles to recipient cells.

Infection or transfection with viral or retroviral constructs include the use of retrovirus, such as lentivirus, or DNA viruses, such as adenovirus. The process involves using a viral or retroviral vector to transfer a foreign gene to the host's cells.

In some embodiments, the protein or chimeric molecule thereof is produced by either transient methods or from stably transfected cell lines. Transient transfection is performed using either adherent or suspension cell lines. For adherent cell lines, the cells are grown in serum containing medium (between 2-10% serum) and in medium such as DMEM, DMEM/F12 (JRH). Serum used can be fetal calf serum (FCS), donor calf serum (DCS), new born calf serum (NBCS) or the like. Plasmid vectors are introduced into the cells by standard methods known in the art. In a particular embodiment, the DNA of the protein or chimeric molecule thereof is transfected using DEAE dextran or calcium phosphate precipitation. Following transfection, the cells are switched to an appropriate collection medium (e.g. serum free DMEM/F12) for collection of the expressed protein or chimeric molecule thereof.

Transient expression of the protein or chimeric molecule thereof from suspension cells can be performed by introducing the plasmid vector using the methods outlined above. The suspension cells can be grown in either serum containing medium, or in serum free medium (e.g. Freestyle medium (Invitrogen), CD293 medium (Invitrogen), Excell medium (JRH) or the like). The transfection can be performed in the absence of serum by transfecting in an appropriate media using a suitable transfection method, for instance, lipofectamine in OptiMEM medium.

Transient expression usually results in a peak of expression 2-3 days after transfection. Episomal vectors are replicated within the cell and give sustained expression. Therefore, to obtain large amounts of product, episomal expression vectors are transfected into cells and the cells are expanded. A protein or chimeric molecule thereof is expressed into the medium, which is collected as the cells are expanded over a period of weeks. The expression medium can be serum containing or serum free and the cells can be either adherent or suspension adapted.

Stable clones are obtained by transfection of the expression vector into the cells, then selecting with an appropriate agent, for instance, phleomycin, hygromycin, puromycin, neomycin G418, methotrexate or the like. Stable clones will survive selection as the plasmid contains a resistance gene in addition to the gene encoding the protein or the chimeric molecule. One to two days after introduction of the gene, selection is begun on either the whole population of cells (stable pools) or on cells plated at clonal density. A non-transfected population of cells is also selected to determine the efficacy of cell killing by the selective agent. For adherent cells, the cells are allowed to grow on a tissue culture plate until visible separate clones are obtained. They are then removed from the plate by trypsinization, or physical removal and placed into tissue culture wells (eg, one clone per well of a 96 well plate). For suspension cells, limiting dilution cloning is performed subsequent to selection. The clones are then expanded, then either characterized and/or subjected to a further round of limiting dilution analysis.

Stable clones growing in serum containing medium can be adapted by gradual reduction of serum levels followed by detachment and growth under low serum in suspension. The serum levels are then reduced further until serum free status is achieved. Some growth media allow more rapid adaptation (e.g. a straight swap from serum containing adherent conditions to serum free suspension growth), an example of which is Invitrogen's CD293 media.

Following growth in serum free media, the clones can begin media optimization. The clones are tested for production characteristics, for example, integral viable cell number, in many different growth media until an optimum formulation or formulations are obtained. This may depend on the method of production of the product. For instance, the cells may be expanded in one medium, then additives that enhance expression added prior to product collection.

The over-expressed protein or chimeric molecule may accumulate within host cells. Recovery of intracellular protein involves treatment of the host cells with lysis buffers including but not limited to buffers containing: NP40, Triton X-100, Triton X-114, sodium dodecyl sulfate (SDS), sodium cholate, sodium deoxycholate, CHAPS, CHAPSO, Brij-35, Brij-58, Tween-20, Tween-80, Octylglucoside and Octylthioglucoside. Alternative methods of host cell lysis may include sonication, homogenization, french press treatment and repeated cycles of freeze thawing and treatment of the cells with hypotonic solutions.

The final product can be produced in many different sorts of bioreactors, by way of non-limiting examples, including stirred tank, airlift, packed bed perfusion, microcarriers, hollow fibre, bag technologies, cell factories. The methods may be continuous culture, batch, fed batch or induction. Peptones may be added to low serum cultures to achieve increases in volumetric protein production.

The protein or chimeric molecule of the present invention is purified using a purification strategy specifically tailored for protein or chimeric molecule of the present invention. Purification methods include but are not limited to: tangential flow filtration (TFF); ammonium sulfate precipitation; size exclusion chromatography (SEC); gel filtration chromatography (GFC); affinity chromatography (AFC); Protein A Affinity Purification; Receptor mediated Ligand Chromatography (RMLC); dye ligand chromatography (DLC); ion exchange chromotogaphy (IEC), including anion or cation exchange chromatography (AEC or CEC); reversed-phase chromatography (RPC); hydrophobic interaction chromatography (HIC); metal chelating chromatography (MCC).

TFF is a rapid and efficient method for biomolecule separation and is used for concentrating, desalting, or fractionating samples. TFF can concentrate samples as large as hundreds of litres down to as little as 10 ml. In conjunction with a suitable molecular weight cut off membrane, TFF can separate and isolate biomolecules of differing size and molecular weight (nominal molecular weight cutoff (NMWC) 5 KDa, 10 KDa, 30 KDa, 100 KDa). The process of diafiltration involving dilution of the sample followed by re-concentration can be used to desalt or exchange the sample buffer.

Salting out or ammonium sulfate precipitation is useful for concentrating dilute solutions of proteins. It is also useful for fractionating a mixture of proteins. Increases in the ionic strength of a solution containing protein causes a reduction in the repulsive effect of like charges between protein molecules. It also reduces the forces holding the solvation shell around the protein molecules. When these forces are sufficiently reduced, the protein will precipitate; hydrophobic proteins precipitating at lower salt concentrations than hydrophilic proteins. Fractionation of protein mixtures by the stepwise increase in the ionic strength followed by centrifugation can be a very effective way of partly purifying proteins.

SEC separates proteins by size, based on the flow of the sample through a porous matrix. SEC has the same principle as GFC when it is used to separate molecules in aqueous systems. In SEC, molecules larger than pores of the packing elute with the solvent front first and are completely excluded. Intermediate sizes of molecules, between the completely excluded and the retained, pass through the pores of the matrix according to their sizes. Small molecules which freely pass in and out of the pores are retained. Therefore, different sizes of proteins have different elution volume and retention times. For structurally similar molecules, the larger the molecular sizes, the earlier they elute out. Before running any samples, a standard curve should be established to determine the working limits and reference retention time.

When the protein shapes are the same, molecular weight can be screened in the elutes from the column rapidly by UV absorption, fluorescence or light scattering, according to the packing materials of various pore sizes on the column. Photon correlation spectroscopy (PCS) has been usually performed on static samples and for liquid chromatographic detection. Low angle laser light scattering has also been coupled to chromatographic detection to detect the molecular weights directly, independent of the shapes of the proteins (Carr et al. Anal Biochem 175:492-499, 1988). SEC-HPLC was used to detect hGH degradation and aggregation (Pikal et al. Pharm Res 8:427-436, 1991). It was also used for estimation of contamination in studying β-galactosidase (Yoshioka et al. Pharm Res 10:103-108, 1993).

AFC purifies biological molecules according to specific interactions between their chemical structures and the suitable affinity ligands. The target molecule is adsorbed by a complementary immobilized ligand specifically and reversibly. The ligand can be an inhibitor, substrate, analog or cofactor, or an antibody which can recognize the target molecules specifically. Subsequently, the adsorbed molecules are either eluted by competitive displacement, or by the conformation change through a pH or ionic strength shift.

Protein A Affinity Purification is an example of affinity purification utilising the affinity of certain bacterial proteins that bind generally to antibodies, regardless of the antibody's specificity to antigen. Protein A, Protein G and Protein L are three that have well characterised antibody-binding properties. These proteins have been produced recombinantly and used routinely for affinity purification of key antibody types from a variety of species. A genetically engineered recombinant form of Protein A and G, called Protein A/G, is also available. These antibody-binding proteins can be immobilized to support matrixes. This method has been modified to purify recombinant proteins that have had the Protein A binding region of an antibody (Fc region) linked to the target protein. Binding to the immobilised Protein A molecule is performed under physiological conditions and eluted by change in pH or ionic strength.

RMLC is a special kind of AFC utilising the inherent affinity of a receptor for its cognate target molecule. The receptor molecule is immobilised on a suitable chromatography support matrix via reactive amines, reactive hydrogens, carbonyl, carboxyl or sulfhydryl groups. In one example of RMLC, the receptor-Fc chimera molecule is immobilised on Protein A sepharose beads via affinity of the Fc portion of the receptor to the Protein A. This method has the advantage of immobilising the receptor in an orientation that exposes its ligand-binding site to its cognate cytokine. Adsorption of the target molecule to the receptor is performed under physiological conditions and elution is achieved by change in pH or ionic strength.

DLC is a kind of ALC utilizing the ability of reactive dyes to bind proteins in a selective and reversible manner. The dyes are generally monochlorotriazine compounds. The reactive chloro group allows easy immobilization of the triazine dye to a support matrix, such as Sepharose or agarose, and, more recently, to nylon membranes.

The initial discovery of the ability of these dyes to bind proteins came from the observation that blue dextran (a conjugate of cibacron blue FG-3A), used as a void volume marker on gel filtration columns, could retard the elution of certain proteins. A number of studies have been carried out on the specificity of the dyes for particular proteins, mostly using the prototype cibacron blue dye. The dyes appear to be most effective at binding proteins and enzymes that utilize nucleotide cofactors, such as kinases and dehydrogenases, although other proteins such as serum albumin also bind tightly. It has been proposed that the aromatic triazine dye structure resembles the nucleotide structure of nicotinamide adenine dinucleotide (NAD) and that the dye interacts with the dinucleotide fold in these proteins. In many cases, bound proteins can be eluted from the columns by a substrate or nucleotide cofactor in a competitive fashion, and dyes have been shown to compete for substrate-binding sites in free solution. It seems likely that these dyes can bind proteins by electrostatic and hydrophobic interactions and by more specific “pseudoaffinity” interactions with ligand-binding sites. Enhancing the specificity of dye ligands by modification to further resemble ligands (biomimetic dyes) has been successful in the purification of a number of dehydrogenases and proteases (McGettrick et al. Methods Mol Biol 244:151-7, 2004).

Ion Exchange Chromatography (IEC) purifies proteins using protein retention on columns resulting from the electrostatic interactions between the ion exchange column matrix and the proteins. When the pH of the mobile phase is above the pI of the target protein will be negatively charged and will interact with an anion exchange column (AEC). When the pH of the mobile phase is below the pI of the target protein the protein will be positively charged and a cation exchange column (CEC) should be used. The target proteins are eluted by increasing the concentrations of a counter ion with the same charge as the target molecule.

RPC separates biological molecules according to the hydrophobic interactions between the molecule and a chromatographic support matrix. Ionizable compounds are best analyzed in their neutral form by controlling the pH of the separation. Mobile phase additives, such as trifluoroacetic acid, increase protein hydrophobicity by forming ion pairs which strongly adsorb to the stationary phase. By changing the polarity of the mobile phase, the biological molecules are eluted from the chromatographic support.

HIC is similar to RPC, but with a larger nominal pore size. In HIC, the elution solvent uses an aqueous salt solution, instead of the aqueous or organic mobile phases used in RPC. Also, the order of sample elution is reversed from that obtained from RPC. The surfaces of proteins consist of hydrophilic residues and hydrophobic “patches”, which are usually located in the interior of the folded proteins to stabilize the proteins. When the hydrophobic patches become exposed to the aqueous environment, they will disrupt the normal solvation properties of the protein, which is thermodynamically unfavorable. In the aqueous mobile phase, the higher the concentrations of inorganic salts (e.g. ammonium sulfate), the higher surface tension, thereby increasing the strength of hydrophobic interactions between the hydrophobic groups of the HIC resin and the proteins, which are adsorbed. However, while descending the salt concentration gradient, the surface tension of the aqueous mobile phase is decreased, thus reducing the hydrophobic interaction, resulting in the proteins desorbing from the hydrophobic groups of the column.

MCC is a technique in which proteins are separated on the basis of their affinity for chelated metal ions. Various metal ions including but not limited to Cu²⁺, Co²⁺, Zn²⁺, Mn²⁺, Mg²⁺ or Ni²⁺ are immobilized on the stationary phase of a chromatographic support via a covalently bound chelating ligand (e.g. iminodiacetic acid). Free coordination sites of the metal ions are used to bind different proteins and peptides. Elution can occur by displacement of the protein with a competitive molecule or by changing the pH. For instance, a lowering of the pH in the buffer results in a reduced binding affinity of the protein-metal ion complex and desorption of the protein. Alternatively, bound proteins can be eluted from the column using a descending pH gradient, in the form of a step gradient or as linear gradient.

The physiochemical form of the protein or chimeric molecule of the present invention may be achieved by chemical and/or enzymatic modification to the expressed molecule in a variety of ways known in the art.

The present invention contemplates chemical or enzymatic coupling of carbohydrates to the peptide chain of a protein or chimeric molecule at a time after the protein or chimeric molecule is expressed and purified. Chemical and/or enzymatic coupling procedures may be used to modify, increase or decrease the number or profile of carbohydrate substituents. Depending on the coupling mode used, the sugar(s) may be attached to (a) amide group of arginine, (b) free carboxyl groups, (c) sulfhydroxyl groups such as those of cysteine, (d) hydroxyl groups such as those of serine, threonine, hydroxylysine or hydroxyproline, (e) aromatic residues such as those of phenylalanine, tyrosine, or tryptophan, (f) the amide group of glutamine, or (g) the amino groups such as those of histidine, arginine or lysine. Additions can be carried out chemically or enzymatically. For example serial addition of sugar units to the protein or chimeric molecule thereof can be performed using appropriate recombinant glycosyltransferases. Glycosyltransferases can also be used to add sugars that have covalently attached substituents. For example, sialic acid with covalently attached polyethylene glycol (PEG) can be transferred by a sialyltransferase to a terminal galactosyl residue to increase molecular size and serum half-life.

The carbohydrate side chain of a protein or chimeric molecule can also be modified chemically or enzymatically to incorporate a variety of functionalities, including phosphate, sulfate, hydroxyl, carboxylate, O-sulfate and N-acetyl groups.

Carbohydrates present on a protein or chimeric molecule thereof may also be removed chemically or enzymatically. Trifluoromethanesulfonic acid or an equivalent compound can be used for chemical deglycosylation. This treatment can result in the cleavage of most or all sugars, except the linking sugar, while leaving the polypeptide intact. Individual sugars or the entire chain can also be removed from a protein or chimeric molecule thereof by a variety of endoglycosidases and exoglycosidases.

The glycan component of a protein or a chimeric molecule may be modified synthetically by treatment with sialidases, or mild acid treatment to remove any residual sialic acids; treatment with exo- or endo-glycosidases to trim down the antennae of N-linked oligosaccharides or shorten O-linked oligosaccharides. It may also be treated with fucosidases or sulfatases to remove side groups such as fucose and sulfate. Pseudo glycan structures such as polyethylene glycol or dextrans may be chemically added to the amino acid backbone, or a glycotransferase cocktail can be used with sugar-dUDP precursors to synthetically add sugar subunits to the glycan.

The present invention contemplates a protein or chimeric molecule thereof chemically or enzymatically coupled to radionuclides. Such protein or chimeric molecule may be selected from the list comprising GM-CSF, GM-CSF-Fc, IL-3, IL-3-Fc, IL-4, IL-4-Fc, IL-5 and IL-5-Fc.

Iodination procedures may be used to attach iodine isotopes (e.g. ¹²³I) to the peptide chain of the protein or chimeric molecule thereof. In particular, the isotope(s) may be attached to a (a) phenolic ring of a tyrosine, or (b) the imidazole ring of a histidine on the peptide chain of the protein or the chimeric molecule thereof. Iodination may be performed using the Chloramine-T, iodine monochloride, triiodide, electrolytic, enzymatic, conjugation, demetallation, iodogen or iodo-bead methods.

Technetium labeling procedures may be used to attach ^{99m}Tc to the protein or chimeric molecule of the present invention using a method known in the art, for instance, by the reduction of ^{99m}TcO₄⁻ with a reducing agent (e.g. stannous chloride) followed by ^{99m}Tc labelling of the protein or the chimeric molecule via a bifunctional chelating agent, for instance, diethylenetriamine pentaacetic acid (DTPA).

The present invention contemplates a protein or chimeric molecule thereof chemically or enzymatically coupled to chemotherapeutic agents. Suitable agents (e.g. zoledronic acid) may be conjugated to the protein or the chimeric molecule thereof using methods known in the art, for instance, by a N-hydroxysulfosuccinimide enhanced carbodiimide-mediated coupling reaction.

The present invention contemplates a protein or chimeric molecule thereof chemically or enzymatically coupled to toxins. Suitable toxins, including melittin, various toxin, truncated pseudomonas exotoxin, ricin, gelonin and diptheria toxin may be conjugated to the protein or the chimeric molecule using a method known in the art, for instance, by maleimide or carbodiimide coupling chemistry.

An isolated protein or chimeric molecule thereof described herein may be delivered to the subject by any means that produces contact of the isolated protein or the chimeric molecule with the target receptor or ligand in the subject. In a particular embodiment, a protein or chimeric molecule thereof is delivered to the subject as a “pharmaceutical composition”.

In another aspect, the present invention contemplates a pharmaceutical composition comprising one or more isolated proteins or chimeric protein molecules as hereinbefore described together with a pharmaceutically acceptable carrier or diluent.

Composition forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of surfactants. The preventions of the action of microorganisms can be brought about by various anti-bacterial and anti-fungal agents, for example, parabens, chlorobutanol, phenol, sorbic acid, thirmerosal and the like. In many cases, it will be favorable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization. In the case of sterile powders for the preparation of sterile injectable solutions, suitable methods of preparation include vacuum drying and the freeze-drying technique which yield a powder of active ingredient plus any additionally desired ingredient.

When the active agent is suitably protected, it may be orally administered, for example, with an inert diluent or with an assimilable edible carrier, or it may be enclosed in hard or soft shell gelatin capsule, or it may be compressed into tablets, or it may be incorporated directly with the food of the diet or administered via breast milk. For oral therapeutic administration, the active ingredient may be incorporated with excipients and used in the form of ingestible tablets, buccal tablets, troches, capsules, elixirs, suspensions, syrups, wafers and the like. Such compositions and preparations should contain at least 1% by weight of active agent. The percentage of the compositions and preparations may, of course, be varied and may conveniently be between about 5 to about 80% of the weight of the unit. The amount of active agent in such therapeutically useful compositions is such that a suitable dosage will be obtained. In a particular embodiment, compositions or preparations according to the present invention are prepared so that an oral dosage unit form contains between about 0.1 μg and 200 mg of modulator. Alternative dosage amounts include from about 1 μg to about 1000 mg and from about 10 μg to about 500 mg. These dosages may be per individual or per kg body weight. Administration may be per hour, day, week, month or year.

The tablets, troches, pills, capsules and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention also contemplates topical formulations. In a topical composition, the active agent may be suspended within a cream or lotion or wax or other liquid solution such that topical application of the cream or lotion or wax or liquid solution results in the introduction of the active agent to a biological surface in the subject. The term “biological surface” as used herein, contemplates any surface on or within the organism. Examples of “biological surfaces” to which the topical compositions of the present invention may be applied include any epithelial surface such as the skin, respiratory tract, gastrointestinal tract and genitourinary tract.

In addition to traditional cream, emulsion, patch or spray formulations, the agents of the present invention may also be delivered topically and/or transdermally using a range of iontophoric or poration based methodologies.

“Iontophoresis” is predicated on the ability of an electric current to cause charged particles to move. A pair of adjacent electrodes placed on the skin set up an electrical potential between the skin and the capillaries below. At the positive electrode, positively charged drug molecules are driven away from the skin's surface toward the capillaries. Conversely, negatively charged drug molecules would be forced through the skin at the negative electrode. Because the current can be literally switched on and off and modified, iontophoretic delivery enables rapid onset and offset, and drug delivery is highly controllable and programmable.

Poration technologies, use high-frequency pulses of energy, in a variety of forms (such as radio frequency radiation, laser, heat or sound) to temporarily disrupt the stratum corneum, the layer of skin that stops many drug molecules crossing into the bloodstream. It is important to note that unlike iontophoresis, the energy used in poration technologies is not used to transport the drug across the skin, but facilitates its movement. Poration provides a “window” through which drug substances can pass much more readily and rapidly than they would normally.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the modulator; their use in the pharmaceutical compositions is contemplated. Supplementary active compounds can also be incorporated into the compositions.

In one embodiment, the pharmaceutical composition of the present invention can be used either alone or in conjunction with other drugs or therapies in the same manner as the protein or chimeric molecule thereof expressed by non-human cell line, such as, a protein or chimeric molecule expressed by E. coli, yeast, or CHO, for treatment alone or in conjunction with another drug for conditions including A-Beta-Lipoproteinemia, A-V, A Beta-2-Microglobulin Amyloidosis, A-T, A1AD, A1AT, Aagenaes, Aarskog syndrome, Aarskog-Scott Syndrome, Aase-smith syndrome, Aase Syndrome, AAT, Abderhalden-Kaufmann-Lignac Syndrome, Abdominal Muscle Deficiency Syndrome, Abdominal Wall Defect, Abdominal Epilepsy, Abdominal Migraine, Abductor Spasmodic Dysphonia, Abductor Spastic Dysphonia, Abercrombie Syndrome, blepharon-Macrostomia Syndrome, ABS, Absence of HPRT, Absence of Corpus Callosum Schinzel Typ, Absence Defect of Limbs Scalp and Skull, Absence of Menstruation Primar, Absence of HGPRT, Absorptive Hyperoxaluriaor Enteric, Abt-Letterer-Siwe Disease, ACADL, ACADM Deficiency, ACADM, ACADS, Acanthocytosis-Neurologic Disorder, Acanthocytosis, Acantholysis Bullosa, Acanthosis Nigricans, Acanthosis Bullosa, Acanthosis Nigricans With Insulin Resistance Type A, Acanthosis Nigricans With Insulin Resistance Type B, Acanthotic Nevus, Acatalasemia, Acatalasia, ACC, Accessory Atrioventricular Pathways, Accessory Atrioventricular Pathways, Acephaly, ACF with Cardiac Defects, Achalasia, Achard-Thiers Syndrome, ACHARD (Marfan variant), Achard's syndrome, Acholuric Jaundice, Achondrogenesis, Achondrogenesis Type IV, Achondrogenesis Type III, Achondroplasia, Achondroplasia Tarda, Achondroplastic Dwarfism, Achoo Syndrome, Achromat, Achromatope, Achromatopic, Achromatopsia, Achromic Nevi, Acid Ceramidase Deficiency, Acid Maltase Deficiency, Acid Beta-glucosidase Deficiency, Acidemia Methylmalonic, Acidemia Propionic, Acidemia with Episodic Ataxia and Weakness, Acidosis, Aclasis Tarsoepiphyseal, ACM, Acoustic Neurilemoma, Acoustic Neuroma, ACPS with Leg Hypoplasia, ACPS II, ACPS IV, ACPS III, Acquired Aphasia with Convulsive Disorder, Acquired Brown Syndrome, Acquired Epileptic Aphasia, Acquired Factor XIII Deficiency, Acquired Form of ACC (caused by infection while still in womb), Acquired Hyperoxaluria, Acquired Hypogammaglobulinemia, Acquired Immunodeficiency Syndrome (AIDS), Acquired Iron Overload, Acquired Lipodystrophy, Acquired Partial Lipodystrophy, Acquired Wandering Spleen, ACR, Acral Dysostosis with Facial and Genital Abnormalities, Acro Renal, Acrocallosal Syndrome Schinzel Type, Acrocephalosyndactyly, Acrocephalosyndactyly Type I, Acrocephalosyndactyly Type I Subtype I, Acrocephalopolysyndactyly Type II, Acrocephalopolysyndactyly Type III, Acrocephalopolysyndactyly Type IV, Acrocephalosyndactyly V (ACS5 or ACS V) Subtype I, Acrocephaly Skull Asymmetry and Mild Syndactyly, Acrocephaly, Acrochondrohyperplasia, Acrodermatitis Enteropathica, Acrodysostosis, Acrodystrophic Neuropathy, Acrofacial Dysostosis Nager Type, Acrofacial Dysostosis Postaxial Type, Acrofacial Dysostosis Type Genee-Wiedep, Acrogeria Familial, Acromegaly, Acromelalgia Hereditary, Acromesomelic Dysplasia, Acromesomelic Dwarfism, Acromicric Skeletal Dysplasia, Acromicric Dysplasia, Acroosteolysis with Osteoporosis and Changes in Skull and Mandible, Acroosteolysis, Acroparesthesia, ACS I, ACS Type II, ACS Type III, ACS, ACS3, ACTH Deficiency, Action Myoclonus, Acute Brachial Neuritis Syndrome, Acute Brachial Radiculitis Syndrome, Acute Cerebral Gaucher Disease, Acute Cholangitis, Acute Disseminated Encephalomyeloradiculopathy, Acute Disseminated Histiocytosis-X, Acute Hemorrhagic Polioencephalitis, Acute Idiopathic Polyneuritis, Acute Immune-Mediation Polyneuritis, Acute Infantile Pelizaeus-Merzbacher Brain Sclerosis, Acute Intermittant Porphyria, Acute Porphyrias, Acute Sarcoidosis, Acute Shoulder Neuritis, Acute Toxic Epidermolysis, Acyl-CoA Dehydrogenase Deficiency Long-Chain, Acyl-CoA Dehydrogenase Deficiency Short-Chain, Acyl-CoA Dihydroxyacetone Acyltransferase, Acyl-coenzyme A Oxidase Deficiency, ADA, ADA Deficiency, Adam Complex, Adamantiades-Behcet's Syndrome, Adamantinoma, Adams Oliver Syndrome, Adaptive Colitis, ADD combined type, ADD, Addison Disease with Cerebral Sclerosis, Addison's Anemia, Addison's Disease, Addison-Biermer Anemia, Addison-Schilder Disease, Addisonian Pernicious Anemia, Adducted Thumbs-Mental Retardation, Adductor Spasmodic Dysphonia, Adductor Spastic Dysphonia, Adenoma Associated Virilism of Older Women, Adenomatosis of the Colon and Rectum, Adenomatous polyposis of the Colon, Adenomatous Polyposis Familial, Adenosine Deaminase Deficiency, Adenylosuccinase deficiency, ADHD predominantly hyperactive-impulsive type, ADHD predominantly inattentive type, ADHD, Adhesive Arachnoiditis, Adie Syndrome, Adie's Syndrome, Adie's Tonic Pupil, Adie's Pupil, Adipogenital Retinitis Pigmentosa Polydactyl), Adipogenital-Retinitis Pigmentosa Syndrome, Adiposa Dolorosa, Adiposis Dolorosa, Adiposogenital Dystrophy, Adolescent Cystinosis, ADPKD, Adrenal Cortex Adenoma, Adrenal Disease, Adrenal Hyperfunction resulting from Pituitary ACTH Excess, Adrenal Hypoplasia, Adrenal Insufficiency, Adrenal Neoplasm, Adrenal Virilism, Adreno-Retinitis Pigmentosa-Polydactyly Syndrome, Adrenocortical Insufficiency, Adrenocortical Hypofunction, Adrenocorticotropic Hormone Deficiency Isolated, Adrenogenital Syndrome, Adrenoleukodystrophy, Adrenomyeloneuropathy, Adreno-Retinitis Pigmentosa-Polydactyly Syndrome, Adult Cystinosis, Adult Dermatomyositis, Adult Hypophosphatasia, Adult Macula Lutea Retinae Degeneration, Adult Onset ALD, Adult-Onset Ceroidosis, Adult Onset Medullary Cystic Disease, Adult Onset Pernicious Anemia, Adult Onset Schindler Disease, Adult-Onset Subacute Necrotizing Encephalomyelopathy, Adult Polycystic Kidney Disease, Adult Onset Medullary Cystic Disease, Adynlosuccinate Lyase Deficiency, AE, AEC Syndrome, AFD, Afibrinogenemia, African Siderosis, AGA, Aganglionic Megacolon, Age Related Macular Degeneration, Agenesis of Commissura Magna Cerebri, Agenesis of Corpus Callosum, Agenesis of Corpus Callosum-Infantile Spasms-Ocular Anomalies, Agenesis of Corpus Callosum and Chorioretinal Abnormality, Agenesis of Corpus Callosum-Chorioretinitis Abnormality, Aggressive mastocytosis, Agnosis Primary, AGR Triad, AGU, Agyria, Agyria-pachygria-band spectrum, AHC, AHD, AHDS, AHF Deficiency, AHG Deficiency, AHO, Ahumada Del Castillo, Aicardi Syndrome, AIED, AIMP, AlP, AIS, Akinetic Seizure, ALA-D Porphyria, Alactasia, Alagille Syndrome, Aland Island Eye Disease (X-Linked), Alaninuria, Albers-Schonberg Disease, Albinism, Albinismus, Albinoidism, Albright Hereditary Osteodystrophy, Alcaptonuria, Alcohol-Related Birth Defects, Alcoholic Embryopathy, Alcoholic Liver Cirrohsis, Ald, ALD, ALD, Aldosterone, Aldosteronism With Normal Blood Pressure, Aldrich Syndrome, Alexander's Disease, Alexanders Disease, Algodystrophy, Algoneurodystrophy, Alkaptonuria, Alkaptonuric Ochronosis, Alkyl DHAP synthase deficiency, Allan-Herndon-Dudley Syndrome, Allan-Herndon Syndrome, Allan-Herndon-Dudley Mental Retardation, Allergic Granulomatous Antitis, Allergic Granulomatous Angiitis of Cronkhite-Canada, Alobar Holoprosencephaly, Alopecia Areata, Alopecia Celsi, Alopecia Cicatrisata, Alopecia Circumscripta, Alopecia-Poliosis-Uveitis-Vitiligo-Deafness-Cutaneous-Uveo-O, Alopecia Seminuniversalis, Alopecia Totalis, Alopecia Universalis, Alpers Disease, Alpers Diffuse Degeneration of Cerebral Gray Matter with Hepatic Cirrhosis, Alpers Progressive Infantile Poliodystrophy, Alpha-1-Antitrypsin Deficiency, Alpha-1 4 Glucosidase Deficiency, Alpha-Galactosidase A Deficiency, Alpha-Galactosidase B Deficiency, Alpha High-Density Lipoprotein Deficieny, Alpha-L-Fucosidase Deficiency Fucosidosis Type 3, Alpha-GalNAc Deficiency Schindler Type, Alphalipoproteinemia, Alpha Mannosidosis, Alpha-N-Acetylgalactosaminidase Deficiency Schindler Type, Alpha-NAGA Deficiency Schindler Type, Alpha-Neuraminidase Deficiency, Alpha-Thalassemia/mental retardation syndrome non-deletion type, Alphalipoproteinemia, Alport Syndrome, ALS, Alstroem's Syndrome, Alstroem, Alstrom Syndrome, Alternating Hemiplegia Syndrome, Alternating Hemiplegia of Childhood, Alzheimer's Disease, Amaurotic Familial Idiocy, Amaurotic Familial Idiocy Adult, Amaurotic Familial Infantile Idiocy, Ambiguous Genitalia, AMC, AMD, Ameloblastoma, Amelogenesis Imperfecta, Amenorrhea-Galactorrhea Nonpuerperal, Amenorrhea-Galactorrhea-FSH Decrease Syndrome, Amenorrhea, Amino Acid Disorders, Aminoaciduria-Osteomalacia-Hyperphosphaturia Syndrome, AMN, Amniocentesis, Amniotic Bands, Amniotic Band Syndrome, Amniotic Band Disruption Complex, Amniotic Band Sequence, Amniotic Rupture Sequence, Amputation Congenital, AMS, Amsterdam Dwarf Syndrome de Lange, Amylo-1 6-Glucosidase Deficiency, Amyloid Arthropathy of Chronic Hemodialysis, Amyloid Corneal Dystrophy, Amyloid Polyneuropathy, Amyloidosis, Amyloidosis of Familial Mediterranean Fever, Amylopectinosis, Amyoplasia Congenita, Amyotrophic Lateral Sclerosis, Amyotrophic Lateral Sclerosis, Amyotrophic Lateral Sclerosis-Polyglucosan Bodies, AN, AN 1, AN 2, Anal Atresia, Anal Membrane, Anal Rectal Malformations, Anal Stenosis, Analine 60 Amyloidosis, Analphalipoproteinemia, Analrectal, Analrectal, Anaplastic Astrocytoma, Andersen Disease, Anderson-Fabry Disease, Andersen Glycogenosis, Anderson-Warburg Syndrome, Andre Syndrome, Andre Syndrome Type II, Androgen Insensitivity, Androgen Insensitivity Syndrome Partial, Androgen Insensitivity Syndrome Partial, Androgenic Steroids, Anemia Autoimmune Hemolytic, Anemia Blackfan Diamond, Anemia, Congenital, Triphalangeal Thumb Syndrome, Anemia Hemolytic Cold Antibody, Anemia Hemolytic with PGK Deficiency, Anemia Pernicious, Anencephaly, Angelman Syndrome, Angio-Osteohypertrophy Syndrome, Angiofollicular Lymph Node Hyperplasia, Angiohemophilia, Angiokeratoma Corporis, Angiokeratoma Corporis Diffusum, Angiokeratoma Diffuse, Angiomatosis Retina, Angiomatous Lymphoid, Angioneurotic Edema Hereditary, Anhidrotic Ectodermal Dysplasia, Anhidrotic X-Linked Ectodermal Dysplasias, Aniridia, Aniridia-Ambiguous Genitalia-Mental Retardation, Aniridia Associated with Mental Retardation, Aniridia-Cerebellar Ataxia-Mental Deficiency, Aniridia Partial-Cerebellar Ataxia-Mental Retardation, Aniridia Partial-Cerebellar Ataxia-Oligophrenia, Aniridia Type I, Aniridia Type II, Aniridia-Wilms' Tumor Association, Aniridia-Wilms' Tumor-Gonadoblastoma, Ankyloblepharon-Ectodermal Defects-Cleft Lip/Palate, Ankylosing Spondylitis, Annular groves, Anodontia, Anodontia Vera, Anomalous Trichromasy, Anomalous Dysplasia of Dentin, Coronal Dentin Dysplasia, Anomic Aphasia, Anophthalmia, Anorectal, Anorectal Malformations, Anosmia, Anterior Bowing of the Legs with Dwarfism, Anterior Membrane Corneal Dystrophy, Anti-Convulsant Syndrome, Anti-Epstein-Barr Virus Nuclear Antigen (EBNA) Antibody Deficiency, Antibody Deficiency, Antibody Deficiency with near normal Immunoglobulins, Antihemophilic Factor Deficiency, Antihemophilic Globulin Deficiency, Antiphospholipid Syndrome, Antiphospholipid Antibody Syndrome, Antithrombin III Deficiency, Antithrombin III Deficiency Classical (Type I), Antitrypsin Deficiency, Antley-Bixler Syndrome, Antoni's Palsy, Anxietas Tibialis, Aorta Arch Syndrome, Aortic and Mitral Atresia with Hypoplasic Left Heart Syndrome, Aortic Stenosis, Aparoschisis, APC, APECED Syndrome, Apert Syndrome, Aperts, Aphasia, Aplasia Axialis Extracorticales Congenital, Aplasia Cutis Congenita, Aplasia Cutis Congenita with Terminal Transverse Limb Defects, Aplastic Anemia, Aplastic Anemia with Congenital Anomalies, APLS, Apnea, Appalachian Type Amyloidosis, Apple Peel Syndrome, Apraxia, Apraxia Buccofacial, Apraxia Constructional, Apraxia Ideational, Apraxia Ideokinetic, Apraxia Ideomotor, Apraxia Motor, Apraxia Oculomotor, APS, Arachnitis, Arachnodactyly Contractural Beals Type, Arachnodactyl), Arachnoid Cysts, Arachnoiditis Ossificans, Arachnoiditis, Aran-Duchenne, Aran-Duchenne Muscular Atrophy, Aregenerative Anemia, Arginase Deficiency, Argininemia, Arginino Succinase Deficiency, Argininosuccinase Deficiency, Argininosuccinate Lyase Deficiency, Argininosuccinic Acid Lyase-ASL, Argininosuccinic Acid Synthetase Deficiency, Argininosuccinic Aciduria, Argonz-Del Castillo Syndrome, Arhinencephaly, Armenian Syndrome, Amold-Chiari Malformation, Amold-Chiari Syndrome, ARPKD, Arrhythmic Myoclonus, Arrhythmogenic Right Ventricular Dysplasia, Arteriohepatic Dysplasia, Arteriovenous Malformation, Arteriovenous Malformation of the Brain, Arteritis Giant Cell, Arthritis, Arthritis Urethritica, Arthro-Dento-Osteodysplasia, Arthro-Opthalmopathy, Arthrochalasis Multiplex Congenita, Arthrogryposis Multiplex Congenita, Arthrogryposis Multiplex Congenita, Distal, Type IIA, ARVD, Arylsulfatase-B Deficiency, AS, ASA Deficiency, Ascending Paralysis, ASD, Atrioseptal Defects, ASH, Ashermans Syndrome, Ashkenazi Type Amyloidosis, ASL Deficiency, Aspartylglucosaminuria, Aspartylglycosaminuria, Asperger's Syndrome, Asperger's Type Autism, Asphyxiating Thoracic Dysplasia, Asplenia Syndrome, ASS Deficiency, Asthma, Astrocytoma Grade I (Benign), Astrocytoma Grade II (Benign), Asymmetric Crying Facies with Cardiac Defects, Asymmetrical septal hypertrophy, Asymptomatic Callosal Agenesis, AT, AT III Deficiency, AT III Variant IA, AT III Variant Ib, AT 3, Ataxia, Ataxia Telangiectasia, Ataxia with Lactic Acidosis Type II, Ataxia Cerebral Palsy, Ataxiadynamia, Ataxiophemia, ATD, Athetoid Cerebral Palsy, Atopic Eczema, Atresia of Esophagus with or without Tracheoesophageal Fistula, Atrial Septal Defects, Atrial Septal Defect Primum, Atrial and Septal and Small Ventricular Septal Defect, Atrial Flutter, Atrial Fibrillation, Atriodigital Dysplasia, Atrioseptal Defects, Atrioventricular Block, Atrioventricular Canal Defect, Atrioventricular Septal Defect, Atrophia Bulborum Hereditaria, Atrophic Beriberi, Atrophy Olivopontocerebellar, Attention Deficit Disorder, Attention Deficit Hyperactivity Disorder, Attentuated Adenomatous Polyposis Coli, Atypical Amyloidosis, Atypical Hyperphenylalaninemia, Auditory Canal Atresia, Auriculotemporal Syndrome, Autism, Autism Asperger's Type, Autism Dementia Ataxia and Loss of Purposeful Hand Use, Autism Infantile Autism, Autoimmune Addison's Disease, Autoimmune Hemolytic Anemia, Autoimmune Hepatitis, Autoimmune-Polyendocrinopathy-Candidias, Autoimmune Polyglandular Disease Type I, Autosomal Dominant Albinism, Autosomal Dominant Compelling Helioophthalmic Outburst Syndrome, Autosomal Dominant Desmin Distal myopathy with Late Onset, Autosomal Dominant EDS, Autosomal Dominant Emery-Dreifuss Muscular Dystrophy, Autosomal Dominant Keratoconus, Autosomal Dominant Pelizaeus-Merzbacher Brain Sclerosis, Autosomal Dominant Polycystic Kidney Disease, Autosomal Dominant Spinocerebellar Degeneration, Autosomal Recessive Agammaglobulinemia, Autosomal Recessive Centronuclear myopathy, Autosomal Recessive Conradi-Hunernann Syndrome, Autosomal Recessive EDS, Autosomal Recessive Emery-Dreifuss Muscular Dystrophy, Autosomal Recessive Forms of Ocular Albinism, Autosomal Recessive Inheritance Agenesis of Corpus Callosum, Autosomal Recessive Keratoconus, Autosomal Recessive Polycystic Kidney Disease, Autosomal Recessive Severe Combined Immunodeficiency, AV, AVM, AVSD, AWTA, Axilla Abscess, Axonal Neuropathy Giant, Azorean Neurologic Disease, B-K Mole Syndrome, Babinski-Froelich Syndrome, BADS, Baillarger's Syndrome, Balkan Disease, Baller-Gerold Syndrome, Ballooning Mitral Valve, Balo Disease Concentric Sclerosis, Baltic Myoclonus Epilepsy, Bannayan-Zonana syndrome (BZS), Bannayan-Riley-Ruvalcaba syndrome, Banti's Disease, Bardet-Biedl Syndrome, Bare Lymphocyte Syndrome, Barlow's syndrome, Barraquer-Simons Disease, Barrett Esophagus, Barrett Ulcer, Barth Syndrome, Bartter's Syndrome, Basal Cell Nevus Syndrome, Basedow Disease, Bassen-Kornzweig Syndrome, Batten Disease, Batten-Mayou Syndrome, Batten-Spielmeyer-Vogt's Disease, Batten Turner Syndrome, Batten Turner Type Congenital myopathy, Batten-Vogt Syndrome, BBB Syndrome, BBB Syndrome (Opitz), BBB Syndrome, BBBG Syndrome, BCKD Deficiency, BD, BDLS, BE, Beals Syndrome, Beals Syndrome, Beals-Hecht Syndrome, Bean Syndrome, BEB, Bechterew Syndrome, Becker Disease, Becker Muscular Dystrophy, Becker Nevus, Beckwith Wiedemann Syndrome, Beckwith-Syndrome, Begnez-Cesar's Syndrome, Behcet's syndrome, Behcet's Disease, Behr 1, Behr 2, Bell's Palsy, Benign Acanthosis Nigricans, Benign Astrocytoma, Benign Cranial Nerve Tumors, Benign Cystinosis, Benign Essential Blepharospasm, Benign Essential Tremor, Benign Familial Hematuria, Benign Focal Amyotrophy, Benign Focal Amyotrophy of ALS, Benign Hydrocephalus, Benign Hypermobility Syndrome, Benign Keratosis Nigricans, Benign Paroxysmal Peritonitis, Benign Recurrent Hematuria, Benign Recurrent Intrahepatic Cholestasis, Benign Spinal Muscular Atrophy with Hypertrophy of the Calves, Benign Symmetrical Lipomatosis, Benign Tumors of the Central Nervous System, Berardinelli-Seip Syndrome, Berger's Disease, Beriberi, Berman Syndrome, Bemard-Horner Syndrome, Bemard-Soulier Syndrome, Besnier Prurigo, Best Disease, Beta-Alanine-Pyruvate Aminotransferase, Beta-Galactosidase Deficiency Morquio Syndrome, Beta-Glucuronidase Deficiency, Beta Oxidation Defects, Beta Thalassemia Major, Beta Thalassemia Minor, Betalipoprotein Deficiency, Bethlem myopathy, Beuren Syndrome, BH4 Deficiency, Biber-Haab-Dimmer Corneal Dystrophy, Bicuspid Aortic Valve, Biedl-Bardet, Bifid Cranium, Bifunctional Enzyme Deficiency, Bilateral Acoustic Neurofibromatosis, Bilateral Acoustic Neuroma, Bilateral Right-Sidedness Sequence, Bilateral Renal Agenesis, Bilateral Temporal Lobe Disorder, Bilious Attacks, Bilirubin Glucuronosyltransferase Deficiency Type I, Binder Syndrome, Binswanger's Disease, Binswanger's Encephalopathy, Biotimidase deficiency, Bird-Headed Dwarfism Seckel Type, Birth Defects, Birthmark, Bitemporal Forceps Marks Syndrome, Biventricular Fibrosis, Bjornstad Syndrome, B-K Mole Syndrome, Black Locks-Albinism-Deafness of Sensoneural Type (BADS), Blackfan-Diamond Anemia, Blennorrheal Idiopathic Arthritis, Blepharophimosis, Ptosis, Epicanthus Inversus Syndrome, Blepharospasm, Blepharospasm Benign Essential, Blepharospasm Oromandibular Dystonia, Blessig Cysts, BLFS, Blindness, Bloch-Siemens Incontinentia Pigmenti Melanoblastosis Cutis Linearis, Bloch-Siemens-Sulzberger Syndrome, Bloch-Sulzberger Syndrome, Blood types, Blood type A, Blood type B, Blood type AB, Blood type O, Bloom Syndrome, Bloom-Torre-Mackacek Syndrome, Blue Rubber Bleb Nevus, Blue Baby, Blue Diaper Syndrome, BMD, BOD, BOFS, Bone Tumor-Epidennoid Cyst-Polyposis, Bonnet-Dechaume-Blanc Syndrome, Bonnevie-Ulrich Syndrome, Book Syndrome, BOR Syndrome, BORJ, Borjeson Syndrome, Borjeson-Forssman-Lehmann Syndrome, Bowen Syndrome, Bowen-Conradi Syndrome, Bowen-Conradi Hutterite, Bowen-Conradi Type Hutterite Syndrome, Bowman's Layer, BPEI, BPES, Brachial Neuritis, Brachial Neuritis Syndrome, Brachial Plexus Neuritis, Brachial-Plexus-Neuropathy, Brachiocephalic Ischemia, Brachmaim-de Lange Syndrome, Brachycephaly, Brachymorphic Type Congenital, Bradycardia, Brain Injury due to perinatal asphyxia, Brain Tumors, Brain Tumors Benign, Brain Tumors Malignant, Branched Chain Alpha-Ketoacid Dehydrogenase Deficiency, Branched Chain Ketonuria I, Brancher Deficiency, Branchio-Oculo-Facial Syndrome, Branchio-Oto-Renal Dysplasia, Branchio-Oto-Renal Syndrome, Branchiooculofacial Syndrome, Branchiootic Syndrome, Brandt Syndrome, Brandywine Type Dentinogenesis Imperfecta, Brandywine type Dentinogenesis Imperfecta, Breast Cancer, BRIC Syndrome, Brittle Bone Disease, Broad Beta Disease, Broad Thumb Syndrome, Broad Thumbs and Great Toes Characteristic Facies and Mental Retardation, Broad Thumb-Hallux, Broca's Aphasia, Brocq-Duhring Disease, Bronze Diabetes, Bronze Schilder's Disease, Brown Albinism, Brown Enamel Hereditary, Brown-Sequard Syndrome, Brown Syndrome, BRRS, Brueghel Syndrome, Bruton's Agammaglobulinemia Common, BS, BSS, Buchanan's Syndrome, Budd's Syndrome, Budd-Chiari Syndrome, Buerger-Gruetz Syndrome, Bulbospinal Muscular Atrophy-X-linked, Bulldog Syndrome, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, BWS, Byler's Disease, C Syndrome, C1 Esterase Inhibitor Dysfunction Type II Angioedema, C1-INH, C1 Esterase Inhibitor Deficiency Type I Angioedema, C1NH, Cacchi-Ricci Disease, CAD, CADASIL, CAH, Calcaneal Valgus, Calcaneovalgus, Calcium Pyrophosphate Dihydrate Deposits, Callosal Agenesis and Ocular Abnormalities, Calves-Hypertrophy of Spinal Muscular Atrophy, Campomelic Dysplasia, Campomelic Dwarfism, Campomelic Syndrome, Camptodactyly-Cleft Palate-Clubfoot, Camptodactyly-Limited Jaw Excursion, Camptomelic Dwarfism, Camptomelic Syndrome, Camptomelic Syndrome Long-Limb Type, Camurati-Engelmann Disease, Canada-Cronkhite Disease, Canavan disease, Canavan's Disease Included, Canavan's Leukodystrophy, Cancer, Cancer Family Syndrome Lynch Type, Cantrell Syndrome, Cantrell-Haller-Ravich Syndrome, Cantrell Pentalogy, Carbamyl Phosphate Synthetase Deficiency, Carbohydrate Deficient Glycoprotein Syndrome, Carbohydrate-Deficient Glycoprotein Syndrome Type Ia, Carbohydrate-Induced Hyperlipemia, Carbohydrate Intolerance of Glucose Galactose, Carbon Dioxide Acidosis, Carboxylase Deficiency Multiple, Cardiac-Limb Syndrome, Cardio-auditory Syndrome, Cardioauditory Syndrome of Jervell and Lange-Nielsen, Cardiocutaneous Syndrome, Cardio-facial-cutaneous syndrome, Cardiofacial Syndrome Cayler Type, Cardiomegalia Glycogenica Diffusa, Cardiomyopathic Lentiginosis, Cardio myopathy, Cardio myopathy Associated with Desmin Storage myopathy, Cardio myopathy Due to Desmin Defect, Cardio myopathy-Neutropenia Syndrome, Cardio myopathy-Neutropenia Syndrome Lethal Infantile Cardio myopathy, Cardiopathic Amyloidosis, Cardiospasm, Cardocardiac Syndrome, Carnitine-Acylcarnitine Translocase Deficiency, Carnitine Deficiency and Disorders, Carnitine Deficiency Primary, Carnitine Deficiency Secondary, Carnitine Deficiency Secondary to MCAD Deficiency, Carnitine Deficiency Syndrome, Carnitine Palmitoyl Transferase I & II (CPT I & II), Carnitine Palmitoyltransferase Deficiency, Carnitine Palmitoyltransferase Deficiency Type 1, Carnitine Palmitoyltransferase Deficiency Type 2 benign classical muscular form included severe infantile form included, Carnitine Transport Defect (Primary Carnitine Deficiency), Carnosinase Deficiency, Carnosinemia, Caroli Disease, Carpenter syndrome, Carpenter's, Cartilage-Hair Hypoplasia, Castleman's Disease, Castleman's Disease Hyaline Vascular Type, Castleman's Disease Plasma Cell Type, Castleman Tumor, Cat Eye Syndrome, Cat's Cry Syndrome, Catalayse deficiency, Cataract-Dental Syndrome, Cataract X-Linked with Hutchinsonian Teeth, Catecholamine hormones, Catel-Manzke Syndrome, Catel-Manzke Type Palatodigital Syndrome, Caudal Dysplasia, Caudal Dysplasia Sequence, Caudal Regression Syndrome, Causalgia Syndrome Major, Cavernomas, Cavernous Angioma, Cavernous Hemangioma, Cavernous Lymphangioma, Cavernous Malformations, Cayler Syndrome, Cazenave's Vitiligo, CBGD, CBPS, CCA, CCD, CCHS, CCM Syndrome, CCMS, CCO, CD, CDG1a, CDG1A, CDGS Type Ia, CDGS, CDI, CdLS, Celiac Disease, Celiac sprue, Celiac Sprue-Dermatitis, Cellular Immunodeficiency with Purine Nucleoside Phosphorylase Deficiency, Celsus' Vitiligo, Central Apnea, Central Core Disease, Central Diabetes Insipidus, Central Form Neurofibromatosis, Central Hypoventilation, Central Sleep Apnea, Centrifugal Lipodystrophy, Centronuclear myopathy, CEP, Cephalocele, Cephalothoracic Lipodystrophy, Ceramide Trihexosidase Deficiency, Cerebellar Agenesis, Cerebellar Aplasia, Cerebellar Hemiagenesis, Cerebellar Hypoplasia, Cerebellar Vermis Aplasia, Cerebellar Vermis Agenesis-Hypernea-Episodic Eye Moves-Ataxia-Retardation, Cerebellar Syndrome, Cerebellarparenchymal Disorder IV, Cerebellomedullary Malformation Syndrome, Cerebello-Oculocutaneous Telangiectasia, Cerebelloparenchymal Disorder IV Familial, Cerebellopontine Angle Tumor, Cerebral Arachnoiditis, Cerebral Autosomal Dominant Arteriopathy with Subcortical Infarcts and Leukodystrophy, Cerebral Beriberi, Cerebral Diplegia, Cerebral Gigantism, Cerebral Ischemia, Cerebral Malformations Vascular, Cerebral Palsy, Cerebro-Oculorenal Dystrophy, Cerebro-Oculo-Facio-Skeletal Syndrome, Cerebrocostomandibular syndrome, Cerebrohepatorenal Syndrome, Cerebromacular Degeneration, Cerebromuscular Dystrophy Fukuyama Type, Cerebroocular Dysgenesis, Cerebroocular Dysplasia-Muscular Dystrophy Syndrome, Cerebrooculofacioskeletal Syndrome, Cerebroretinal Arteriovenous Aneurysm, Cerebroside Lipidosis, Cerebrosidosis, Cerebrotendinous Xanthomatosis, Cerebrovascular Ferrocalcinosis, Ceroid-Lipofuscinosis Adult form, Cervical Dystonia, Cervical Dystonia, Cervico-Oculo-Acoustic Syndrome, Cervical Spinal Stenosis, Cervical Vertebral Fusion, CES, CF, CFC syndrome, CFIDS, CFND, CGD, CGF, Chalasodermia Generalized, Chanarin Dorfman Disease, Chanarin Dorfman Syndrome, Chanarin Dorfman Ichthyosis Syndrome, Chandler's Syndrome, Charcot's Disease, Charcot-Marie-Tooth, Charcot-Marie-Tooth Disease, Charcot-Marie-Tooth Disease Variant, Charcot-Marie-Tooth-Roussy-Levy Disease, CHARGE Association, Charge Syndrome, CHARGE Syndrome, Chaund's Ectodermal Dysplasias, Chediak-Higashi Syndrome, Chediak-Steinbrinck-Higashi Syndrome, Cheilitis Granulomatosa, Cheiloschisis, Chemke Syndrome, Cheney Syndrome, Cherry Red Spot and Myoclonus Syndrome, CHF, CHH, Chiari's Disease, Chiari Malformation I, Chiari Malformation, Chiari Type I (Chiari Malformation I), Chiari Type II (Chiari Malformation II), Chiari I Syndrome, Chiari-Budd Syndrome, Chiari-Frommel Syndrome, Chiari Malformation II, CHILD Syndrome, CHILD Ichthyosis Syndrome, CHILD Syndrome Ichthyosis, Childhood Adrenoleukodystrophy, Childhood Dermatomyositis, Childhood-onset Dystonia, Childhood Cyclic Vomiting, Childhood Giant Axonal Neuropathy, Childhood Hypophosphatasia, Childhood Muscular Dystrophy, CHN, Cholestasis, Cholestasis Hereditary Norwegian Type, Cholestasis Intrahepatic, Cholestasis Neonatal, Cholestasis of Oral Contraceptive Users, Cholestasis with Peripheral Pulmonary Stenosis, Cholestasis of Pregnancy, Cholesterol Desmolase Deficiency, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Choreoacanthocytosis, Chorionic VIIIi Sampling, Chorioretinal Anomalies, Chorioretinal Anomalies with ACC, Chorireninal Coloboma-Joubert Syndrome, Choroidal Sclerosis, Choroideremia, Chotzen Syndrome, Christ-Siemens-Touraine Syndrome, Christ-Siemans-Touraine Syndrome, Christmas Disease, Christmas Tree Syndrome, Chromosome 3 Deletion of Distal 3p, Chromosome 3 Distal 3p Monosomy, Chromosome 3-Distal 3q2 Duplication, Chromosome 3-Distal 3q2 Trisomy, Chromosome 3 Monosomy 3p2, Chromosome 3q Partial Duplication Syndrome, Chromosome 3q, Partial Trisomy Syndrome, Chromosome 3-Trisomy 3q2, Chromosome 4 Deletion 4q31-qter Syndrome, Chromosome 4 Deletion 4q32-qter Syndrome, Chromosome 4 Deletion 4q33-qter Syndrome, Chromosome 4 Long Arm Deletion, Chromosome 4 Long Arm Deletion, Chromosome 4 Monosomy 4q, Chromosome 4-Monosomy 4q, Chromosome 4 Monosomy Distal 4q, Chromosome 4 Partial Deletion 4p, Chromosome 4, Partial Deletion of the Short Arm, Chromosome 4 Partial Monosomy of Distal 4q, Chromosome 4 Partial Monosomy 4p, Chromosome 4 Partial Trisomy 4 (q25-qter), Chromosome 4 Partial Trisomy 4 (q26 or q27-qter), Chromosome 4 Partial Trisomy 4 (q31 or 32-qter), Chromosome 4 Partial Trisomy 4p, Chromosome 4 Partial Trisomies 4q2 and 4q3, Chromosome 4 Partial Trisomy Distal 4, Chromosome 4 Ring, Chromosome 4 4q Terminal Deletion Syndrome, Chromosome 4q-Syndrome, Chromosome 4q-Syndrome, Chromosome 4 Trisomy 4, Chromosome 4 Trisomy 4p, Chromosome 4 XY/47 XXY (Mosiac), Chromosome 5 Monosomy 5p, Chromosome 5, Partial Deletion of the Short Arm Syndrome, Chromosome 5 Trisomy 5p, Chromosome 5 Trisomy 5p Complete (5p11-pter), Chromosome 5 Trisomy 5p Partial (5p13 or 14-pter), Chromosome 5p-Syndrome, Chromosome 6 Partial Trisomy 6q, Chromosome 6 Ring, Chromosome 6 Trisomy 6q2, Chromosome 7 Monosomy 7p2, Chromosome 7 Partial Deletion of Short Arm (7p2-), Chromosome 7 Terminal 7p Deletion [del (7) (p21-p22)], Chromosome 8 Monosomy 8p2, Chromosome 8 Monosomy 8p21-pter, Chromosome 8 Partial Deletion (short arm), Chromosome 8 Partial Monosomy 8p2, Chromosome 9 Complete Trisomy 9P, Chromosome 9 Partial Deletion of Short Arm, Chromosome 9 Partial Monosomy 9p, Chromosome 9 Partial Monosomy 9p22, Chromosome 9 Partial Monosomy 9p22-pter, Chromosome 9 Partial Trisomy 9P Included, Chromosome 9 Ring, Chromosome 9 Tetrasomy 9p, Chromosome 9 Tetrasomy 9p Mosaicism, Chromosome 9 Trisomy 9p (Multiple Variants), Chromosome 9 Trisomy 9 (pter-p21 to q32) Included, Chromosome 9 Trisomy Mosaic, Chromosome 9 Trisomy Mosaic, Chromosome 10 Distal Trisomy 10q, Chromosome 10 Monosomy, Chromosome 10 Monosomy 10p, Chromosome 10, Partial Deletion (short arm), Chromosome 10, 10p-Partial, Chromosome 10 Partial Trisomy 10q24-qter, Chromosome 10 Trisomy 10q2, Partial Monosomy of Long Arm of Chromosome 11, Chromosome 11 Partial Monosomy 11q, Chromosome 11 Partial Trisomy, Chromosome 11 Partial Trisomy 11q13-qter, Chromosome 11 Partial Trisomy 11q21-qter, Chromosome 11 Partial Trisomy 11q23-qter, Chromosome 11q, Partial Trisomy, Chromosome 12 Isochromosome 12p Mosaic, Chromosome 13 Partial Monosomy 13q, Chromosome 13, Partial Monosomy of the Long Arm, Chromosome 14 Ring, Chromosome 14 Trisomy, Chromosome 15 Distal Trisomy 15q, Chromosome r15, Chromosome 15 Ring, Chromosome 15 Trisomy 15q2, Chromosome 15q, Partial Duplication Syndrome, Chromosome 17 Interstitial Deletion 17p, Chromosome 18 Long Arm Deletion Syndrome, Chromosome 18 Monosomy 18p, Chromosome 18 Monosomy 18Q, Chromosome 18 Ring, Chromosome 18 Tetrasomy 18p, Chromosome 18q-Syndrome, Chromosome 21 Mosaic 21 Syndrome, Chromosome 21 Ring, Chromosome 21 Translocation 21 Syndrome, Chromosome 22 Inverted Duplication (22pter-22q11), Chromosome 22 Partial Trisomy (22pter-22q11), Chromosome 22 Ring, Chromosome 22 Trisomy Mosaic, Chromosome 48 XXYY, Chromosome 48 XXXY, Chromosome r15, Chromosomal Triplication, Chromosome Triplication, Chromosome Triploidy Syndrome, Chromosome X, Chromosome XXY, Chronic Acholuric Jaundice, Chronic Adhesive Arachnoiditis, Chronic Adrenocortical Insufficiency, Chronic Cavernositis, Chronic Congenital Aregenerative Anemia, Chronic Dysphagocytosis, Chronic Familial Granulomatosis, Chronic Familial Icterus, Chronic Fatigue Immune Dysfunction Syndrome (CFIDS), Chronic Granulomatous Disease, Chronic Guillain-Barre Syndrome, Chronic Idiopathic Jaundice, Chronic Idiopathic Polyneuritis (CIP), Chronic Inflammatory Demyelinating Polyneuropathy, Chronic Inflammatory Demyelinating Polyradiculoneuropathy, Chronic Motor Tic, Chronic Mucocutaneous Candidiasis, Chronic Multiple Tics, Chronic Non-Specific Ulcerative Colitis, Chronic Obliterative Cholangitis, Chronic Peptic Ulcer and Esophagitis Syndrome, Chronic Progressive Chorea, Chronic Progressive External Opthalmoplegia Syndrome, Chronic Progressive External Opthalmoplegia and myopathy, Chronic Progressive External Opthalmoplegia with Ragged Red Fibers, Chronic Relapsing Polyneuropathy, Chronic Sarcoidosis, Chronic Spasmodic Dysphonia, Chronic Vomiting in Childhood, CHS, Churg-Strauss Syndrome, Cicatricial Pemphigoid, CIP, Cirrhosis Congenital Pigmentary, Cirrhosis, Cistinuria, Citrullinemia, CJD, Classic Schindler Disease, Classic Type Pfeiffer Syndrome, Classical Maple Syrup Urine Disease, Classical Hemophilia, Classical Form Cockayne Syndrome Type I (Type A), Classical Leigh's Disease, Classical Phenylketonuria, Classical X-Linked Pelizaeus-Merzbacher Brain Sclerosis, CLE, Cleft Lip/Palate Mucous Cysts Lower Lip PP Digital and Genital Anomalies, Cleft Lip-Palate Blepharophimosis Lagopthalmos and Hypertelorism, Cleft Lip/Palate with Abnormal Thumbs and Microcephaly, Cleft palate-joint contractures-dandy walker malformations, Cleft Palate and Cleft Lip, Cleidocranial Dysplasia w/ Micrognathia, Absent Thumbs, & Distal Aphalangia, Cleidocranial Dysostosis, Cleidocranial Dysplasia, Click murmur syndrome, CLN1, Clonic Spasmodic, Cloustons Syndrome, Clubfoot, CMDI, CMM, CMT, CMTC, CMTX, COA Syndrome, Coarctation of the aorta, Coats' Disease, Cobblestone dysplasia, Cochin Jewish Disorder, Cockayne Syndrome, COD-MD Syndrome, COD, Coffin Lowry Syndrome, Coffin Syndrome, Coffin Siris Syndrome, COFS Syndrome, Cogan Corneal Dystrophy, Cogan Reese Syndrome, Cohen Syndrome, Cold Agglutinin Disease, Cold Antibody Disease, Cold Antibody Hemolytic Anemia, Colitis Ulcerative, Colitis Gravis, Colitis Ulcerative Chronic Non-Specific Ulcerative Colitis, Collodion Baby, Coloboma Heart Defects Atresia of the Choanae Retardation of Growth and Development Genital and Urinary Anomalies and Ear Anomalies, Coloboma, Colonic Neurosis, Color blindness, Colour blindness, Colpocephaly, Columnar-Like Esophagus, Combined Cone-Rod Degeneration, Combined Immunodeficiency with Immunoglobulins, Combined Mesoectodermal Dysplasia, Common Variable Hypogammaglobulinemia, Common Variable Immunodeficiency, Common Ventricle, Communicating Hydrocephalus, Complete Absense of Hypoxanthine-Guanine Phosphoribosyltranferase, Complete Atrioventricular Septal Defect, Complement Component 1 Inhibitor Deficiciency, Complement Component C1 Regulatory Component Deficiency, Complete Heart Block, Complex Carbohydrate Intolerance, Complex Regional Pain Syndrome, Complex V ATP Synthase Deficiency, Complex I, Complex I NADH dehydrogenase deficiency, Complex II, Complex II Succinate dehydrogenase deficiency, Complex III, Complex III Ubiquinone-cytochrome c oxidoreductase deficiency, Complex IV, Complex IV Cytochrome c oxidase deficiency, Complex IV Deficiency, Complex V, Concussive Brain Injury, Cone-Rod Degeneration, Cone-Rod Degeneration Progressive, Cone Dystrophy, Cone-Rod Dystrophy, Confluent Reticular Papillomatosis, Congenital with low PK Kinetics, Congenital Absence of Abdominal Muscles, Congenital Absence of the Thymus and Parathyroids, Congenital Achromia, Congenital Addison's Disease, Congenital Adrenal Hyperplasia, Congenital Adreneal Hyperplasia, Congenital Afibrinogenemia, Congenital Alveolar Hypoventilation, Congenital Anemia of Newborn, Congenital Bilateral Persylvian Syndrome, Congenital Brown Syndrome, Congenital Cardiovascular Defects, Congenital Central Hypoventilation Syndrome, Congenital Cerebral Palsy, Congenital Cervical Synostosis, Congenital Clasped Thumb with Mental Retardation, Congenital Contractural Arachnodactyl), Congenital Contractures Multiple with Arachnodactyl), Congenital Cyanosis, Congenital Defect of the Skull and Scalp, Congenital Dilatation of Intrahepatic Bile Duct, Congenital Dysmyelinating Neuropathy, Congenital Dysphagocytosis, Congenital Dysplastic Angiectasia, Congenital Erythropoietic Porphyria, Congenital Factor XIII Deficiency, Congenital Failure of Autonomic Control of Respiration, Congenital Familial Nonhemolytic Jaundice Type I, Congenital Familial Protracted Diarrhea, Congenital Form Cockayne Syndrome Type II (Type B), Congenital Generalized Fibromatosis, Congenital German Measles, Congenital Giant Axonal Neuropathy, Congenital Heart Block, Congenital Heart Defects, Congenital Hemidysplasia with Ichthyosis Erythroderma and Limb Defects, Congenital Hemolytic Jaundice, Congenital Hemolytic Anemia, Congenital Hepatic Fibrosis, Congenital Hereditary Corneal Dystrophy, Congenital Hereditary Lymphedema, Congenital Hyperchondroplasia, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Keratoconus, Congenital Lactic Acidosis, Congenital Lactose Intolerance, Congenital Lipodystrophy, Congenital Liver Cirrhosis, Congenital Lobar Emphysema, Congenital Localized Emphysema, Congenital Macroglossia, Congenital Medullary Stenosis, Congenital Megacolon, Congenital Melanocytic Nevus, Congenital Mesodermal Dysmorphodystrophy, Congenital Mesodermal Dystrophy, Congenital Microvillus Atrophy, Congenital Multiple Arthrogryposis, Congenital Myotonic Dystrophy, Congenital Neuropathy caused by Hypomyelination, Congenital Pancytopenia, Congenital Pernicious Anemia, Congenital Pernicious Anemia due to Defect of Intrinsic Factor, Congenital Pernicious Anemia due to Defect of Intrinsic Factor, Congenital Pigmentary Cirrhosis, Congenital Porphyria, Congenital Proximal myopathy Associated with Desmin Storage myopathy, Congenital Pulmonary Emphysema, Congenital Pure Red Cell Anemia, Congenital Pure Red Cell Aplasia, Congenital Retinal Blindness, Congenital Retinal Cyst, Congenital Retinitis Pigmentosa, Congenital Retinoschisis, Congenital Rod Disease, Congenital Rubella Syndrome, Congenital Scalp Defects with Distal Limb Reduction Anomalies, Congenital Sensory Neuropathy, Congenital SMA with arthrogryposis, Congenital Spherocytic Anemia, Congenital Spondyloepiphyseal Dysplasia, Congenital Tethered Cervical Spinal Cord Syndrome, Congenital Tyrosinosis, Congenital Varicella Syndrome, Congenital Vascular Cavernous Malformations, Congenital Vascular Veils in the Retina, Congenital Word Blindness, Congenital Wandering Spleen (Pediatric), Congestive Cardio myopathy, Conical Cornea, Conjugated Hyperbilirubinemia, Conjunctivitis, Conjunctivitis Ligneous, Conjunctivo-Urethro-Synovial Syndrome, Conn's Syndrome, Connective Tissue Disease, Conradi Disease, Conradi Hunermann Syndrome, Constitutional Aplastic Anemia, Constitutional Erythroid Hypoplasia, Constitutional Eczema, Constitutional Liver Dysfunction, Constitutional Thrombopathy, Constricting Bands Congenital, Constrictive Pericarditis with Dwarfism, Continuous Muscle Fiber Activity Syndrome, Contractural Arachnodactyl), Contractures of Feet Muscle Atrophy and Oculomotor Apraxia, Convulsions, Cooley's anemia, Copper Transport Disease, Coproporphyria Porphyria Hepatica, Cor Triatriatum, Cor Triatriatum Sinistrum, Cor Triloculare Biatriatum, Cor Biloculare, Cori Disease, Cornea Dystrophy, Corneal Amyloidosis, Corneal Clouding-Cutis Laxa-Mental Retardation, Corneal Dystrophy, Cornelia de Lange Syndrome, Coronal Dentine Dysplasia, Coronary Artery Disease, Coronary Heart Disease, Corpus Callosum Agenesis, Cortical-Basal Ganglionic Degeneration, Corticalis Deformaris, Cortico-Basal Ganglionic Degeneration (CBGD), Corticobasal Degeneration, Corticosterone Methloxidase Deficiency Type I, Corticosterone Methyloxidase Deficiency Type II, Cortisol, Costello Syndrome, Cot Death, COVESDEM Syndrome, COX, COX Deficiency, COX Deficiency French-Canadian Type, COX Deficiency Infantile Mitochondrial myopathy de Toni-Fanconi-Debre included, COX Deficiency Type Benign Infantile Mitochondrial Myopathy, CP, CPEO, CPEO with myopathy, CPEO with Ragged-Red Fibers, CPPD Familial Form, CPT Deficiency, CPTD, Cranial Arteritis, Cranial Meningoencephalocele, Cranio-Oro-Digital Syndrome, Craniocarpotarsal dystrophy, Craniocele, Craniodigital Syndrome-Mental Retardation Scott Type, Craniofacial Dysostosis, Craniofacial Dysostosis-PD Arteriosus-Hypertrichosis-Hypoplasia of Labia, Craniofrontonasal Dysplasia, Craniometaphyseal Dysplasia, Cranioorodigital Syndrome, Cranioorodigital Syndrome Type II, Craniostenosis Crouzon Type, Craniostenosis, Craniosynostosis-Choanal Atresia-Radial Humeral Synostosis, Craniosynostosis-Hypertrichosis-Facial and Other Anomalies, Craniosynostosis Midfacial Hypoplasia and Foot Abnormalities, Craniosynostosis Primary, Craniosynostosis-Radial Aplasia Syndrome, Craniosynostosis with Radial Defects, Cranium Bifidum, CREST Syndrome, Creutzfeldt Jakob Disease, Cri du Chat Syndrome, Crib Death, Crigler Najjar Syndrome Type I, Crohn's Disease, Cronkhite-Canada Syndrome, Cross Syndrome, Cross' Syndrome, Cross-McKusick-Breen Syndrome, Crouzon, Crouzon Syndrome, Crouzon Craniofacial Dysostosis, Cryoglobulinemia Essential Mixed, Cryptopthalmos-Syndactyly Syndrome, Cryptorchidism-Dwarfism-Subnormal Mentality, Crystalline Corneal Dystrophy of Schnyder, CS, CSD, CSID, CSO, CST Syndrome, Curly Hair-Ankyloblephanon-Nail Dysplasia, Curschmann-Batten-Steinert Syndrome, Curth Macklin Type Ichthyosis Hystric, Curth-Macklin Type, Cushing's, Cushing Syndrome, Cushing's III, Cutaneous Malignant Melanoma Hereditary, Cutaneous Porphyrias, Cutis Laxa, Cutis Laxa-Growth Deficiency Syndrome, Cutis Marmorata Telangiectatica Congenita, CV1, CVID, CVS, Cyclic vomiting syndrome, Cystic Disease of the Renal Medulla, Cystic Hygroma, Cystic Fibrosis, Cystic Lymphangioma, Cystine-Lysine-Arginine-Ornithinuria, Cystine Storage Disease, Cystinosis, Cystinuria, Cystinuria with Dibasic Aminoaciduria, Cystinuria Type I, Cystinuria Type II, Cystinuria Type III, Cysts of the Renal Medulla Congenital, Cytochrome C Oxidase Deficiency, D.C., Dacryosialoadenopathy, Dacryosialoadenopathia, Dalpro, Dalton, Daltonism, Danbolt-Cross Syndrome, Dancing Eyes-Dancing Feet Syndrome, Dandy-Walker Syndrome, Dandy-Walker Cyst, Dandy-Walker Deformity, Dandy Walker Malformation, Danish Cardiac Type Amyloidosis (Type III), Darier Disease, Davidson's Disease, Davies' Disease, DBA, DBS, DC, DD, De Barsy Syndrome, De Barsy-Moens-Diercks Syndrome, de Lange Syndrome, De Morsier Syndrome, De Santis Cacchione Syndrome, de Toni-Fanconi Syndrome, Deafness Congenital and Functional Heart Disease, Deafness-Dwarfism-Retinal Atrophy, Deafness-Functional Heart Disease, Deafness Onychodystrophy Osteodystrophy and Mental Retardation, Deafness and Pili Torti Bjomstad Type, Deafness Sensorineural with Imperforate Anus and Hypoplastic Thumbs, Debrancher Deficiency, Deciduous Skin, Defect of Enterocyte Intrinsic Factor Receptor, Defect in Natural Killer Lymphocytes, Defect of Renal Reabsorption of Carnitine, Deficiency of Glycoprotein Neuraminidase, Deficiency of Mitochondrial Respiratory Chain Complex IV, Deficiency of Platelet Glycoprotein Ib, Deficiency of Von Willebrand Factor Receptor, Deficiency of Short-Chain Acyl-CoA Dehydrogenase (ACADS), Deformity with Mesomelic Dwarfism, Degenerative Chorea, Degenerative Lumbar Spinal Stenosis, Degos Disease, Degos-Kohlmeier Disease, Degos Syndrome, DEH, Dejerine-Roussy Syndrome, Dejerine Sottas Disease, Deletion 9p Syndrome Partial, Deletion 11q Syndrome Partial, Deletion 13q Syndrome Partial, Delleman-Oorthuys Syndrome, Delleman Syndrome, Dementia with Lobar Atrophy and Neuronal Cytoplasmic Inclusions, Demyelinating Disease, DeMyer Syndrome, Dentin Dysplasia Coronal, Dentin Dysplasia Radicular, Dentin Dysplasia Type I, Dentin Dysplasia Type II, Dentinogenesis Imperfecta Brandywine type, Dentinogenesis Imperfecta Shields Type, Dentinogenesis Imperfecta Type III, Dento-Oculo-Osseous Dysplasia, Dentooculocutaneous Syndrome, Denys-Drash Syndrome, Depakene, Depakene™ exposure, Depakote, Depakote Sprinkle, Depigmentation-Gingival Fibromatosis-Microphthalmia, Dercum Disease, Dermatitis Atopic, Dermatitis Exfoliativa, Dermatitis Herpetiformis, Dermatitis Multiformis, Dermatochalasia Generalized, Dermatolysis Generalized, Dermatomegaly, Dermatomyositis sine myositis, Dermatomyositis, Dermatosparaxis, Dermatostomatitis Stevens Johnson Type, Desbuquois Syndrome, Desmin Storage myopathy, Desquamation of Newborn, Deuteranomaly, Developmental Reading Disorder, Developmental Gerstmann Syndrome, Devergie Disease, Devic Disease, Devic Syndrome, Dextrocardia-Bronchiectasis and Sinusitis, Dextrocardia with Situs Inversus, DGS, DGSX Golabi-Rosen Syndrome Included, DH, DHAP alkyl transferase deficiency, DHBS Deficiency, DHOF, DHPR Deficiency, Diabetes Insipidus, Diabetes Insipidus Diabetes Mellitus Optic Atrophy and Deafness, Diabetes Insipidus Neurohypophyseal, Diabetes Insulin Dependent, Diabetes Mellitus, Diabetes Mellitus Addison's Disease Myxedema, Diabetic Acidosis, Diabetic Bearded Woman Syndrome, Diabetic Neuropathy, Diamond-Blackfan Anemia, Diaphragmatic Apnea, Diaphyseal Aclasis, Diastrophic Dwarfism, Diastrophic Dysplasia, Diastrophic Nanism Syndrome, Dicarboxylic Aminoaciduria, Dicarboxylicaciduria Caused by Defect in Beta-Oxidation of Fatty Acids, Dicarboxylicaciduria due to Defect in Beta-Oxidation of Fatty Acids, Dicarboxylicaciduria due to MCADH Deficiency, Dichromasy, Dicker-Opitz, DIDMOAD, Diencephalic Syndrome, Diencephalic Syndrome of Childhood, Diencephalic Syndrome of Emaciation, Dienoyl-CoA Reductase Deficiency, Diffuse Cerebral Degeneration in Infancy, Diffuse Degenerative Cerebral Disease, Diffuse Idiopathic Skeletal Hyperostosis, Diffusum-Glycopeptiduria, DiGeorge Syndrome, Digital-Oro-Cranio Syndrome, Digito-Oto-Palatal Syndrome, Digito-Oto-Palatal Syndrome Type I, Digito-Oto-Palatal Syndrome Type II, Dihydrobiopterin Synthetase Deficiency, Dihydropteridine Reductase Deficiency, Dihydroxyacetonephosphate synthase, Dilated (Congestive) Cardio myopathy, Dimitri Disease, Diplegia of Cerebral Palsy, Diplo-Y Syndrome, Disaccharidase Deficiency, Disaccharide Intolerance I, Discoid Lupus, Discoid Lupus Erythematosus, DISH, Disorder of Cornification, Disorder of Cornification Type I, Disorder of Cornification 4, Disorder of Cornification 6, Disorder of Cornification 8, Disorder of Cornification 9 Netherton's Type, Disorder of Cornification 11 Phytanic Acid Type, Disorder of Cornification 12 (Neutral Lipid Storage Type), Disorder of Cornification 13, Disorder of Cornification 14, Disorder of Cornification 14 Trichothiodystrophy Type, Disorder of Cornification 15 (Keratitis Deafness Type), Disorder of Cornification 16, Disorder of Cornification 18 Erythrokeratodermia Variabilis Type, Disorder of Cornification 19, Disorder of Cornification 20, Disorder of Cornification 24, Displaced Spleen, Disseminated Lupus Erythematosus, Disseminated Neurodermatitis, Disseminated Sclerosis, Distal 11q Monosomy, Distal 11q-Syndrome, Distal Arthrogryposis Multiplex Congenita Type IIA, Distal Arthrogryposis Multiplex Congenita Type IIA, Distal Arthrogryposis Type IIA, Distal Arthrogryposis Type 2A, Distal Duplication 6q, Distal Duplication 10q, Dup(10q) Syndrome, Distal Duplication 15q, Distal Monosomy 9p, Distal Trisomy 6q, Distal Trisomy 10q Syndrome, Distal Trisomy 11q, Divalproex, DJS, DKC, DLE, DLPIII, DM, DMC Syndrome, DMC Disease, DMD, DNS Hereditary, DOC I, DOC 2, DOC 4, DOC 6 (Harlequin Type), DOC 8 Curth-Macklin Type, DOC 11 Phytanic Acid Type, DOC 12 (Neutral Lipid Storage Type), DOC 13, DOC 14, DOC 14 Trichothiodystrophy Type, DOC 15 (Keratitis Deafness Type), DOC 16, DOC 16 Unilateral Hemidysplasia Type, DOC 18, DOC 19, DOC 20, DOC 24, Dohle's Bodies-Myelopathy, Dolichospondylic Dysplasia, Dolichostenomelia, Dolichostenomelia Syndrome, Dominant Type Kenny-Caffe Syndrome, Dominant Type Myotonia Congenita, Donahue Syndrome, Donath-Landsteiner Hemolytic Anemia, Donath-Landsteiner Syndrome, DOOR Syndrome, DOORS Syndrome, Dopa-responsive Dystonia (DRD), Dorfman Chanarin Syndrome, Dowling-Meara Syndrome, Down Syndrome, DR Syndrome, Drash Syndrome, DRD, Dreifuss-Emery Type Muscular Dystrophy with Contractures, Dressler Syndrome, Drifting Spleen, Drug-induced Acanthosis Nigricans, Drug-induced Lupus Erythematosus, Drug-related Adrenal Insufficiency, Drummond's Syndrome, Dry Beriberi, Dry Eye, DTD, Duane's Retraction Syndrome, Duane Syndrome, Duane Syndrome Type IA 1B and 1C, Duane Syndrome Type 2A 2B and 2C, Duane Syndrome Type 3A 3B and 3C, Dubin Johnson Syndrome, Dubowitz Syndrome, Duchenne, Duchenne Muscular Dystrophy, Duchenne's Paralysis, Duhring's Disease, Duncan Disease, Duncan's Disease, Duodenal Atresia, Duodenal Stenosis, Duodenitis, Duplication 4p Syndrome, Duplication 6q Partial, Dupuy's Syndrome, Dupuytren's Contracture, Dutch-Kennedy Syndrome, Dwarfism, Dwarfism Campomelic, Dwarfism Cortical Thickening of the Tubular Bones & Transient Hypocalcemia, Dwarfism Levi's Type, Dwarfism Metatropic, Dwarfism-Onychodysplasia, Dwarfism-Pericarditis, Dwarfism with Renal Atrophy and Deafness, Dwarfism with Rickets, DWM, Dyggve Melchior Clausen Syndrome, Dysautonomia Familial, Dysbetalipoproteinemia Familial, Dyschondrodysplasia with Hemangiomas, Dyschondrosteosis, Dyschromatosis Universalis Hereditaria, Dysencephalia Splanchnocystica, Dyskeratosis Congenita, Dyskeratosis Congenita Autosomal Recessive, Dyskeratosis Congenita Scoggins Type, Dyskeratosis Congenita Syndrome, Dyskeratosis Follicularis Vegetans, Dyslexia, Dysmyelogenic Leukodystrophy, Dysmyelogenic Leukodystrophy-Megalobare, Dysphonia Spastica, Dysplasia Epiphysialis Punctata, Dysplasia Epiphyseal Hemimelica, Dysplasia of Nails With Hypodontia, Dysplasia Cleidocranial, Dysplasia Fibrous, Dysplasia Gigantism SyndromeX-Linked, Dysplasia Osteodental, Dysplastic Nevus Syndrome, Dysplastic Nevus Type, Dyssynergia Cerebellaris Myoclonica, Dyssynergia Esophagus, Dystonia, Dystopia Canthorum, Dystrophia Adiposogenitalis, Dystrophia Endothelialis Cornea, Dystrophia Mesodermalis, Dystrophic Epidermolysis Bullosa, Dystrophy, Asphyxiating Thoracic, Dystrophy Myotonic, E-D Syndrome, Eagle-Barrett Syndrome, Eales Retinopathy, Eales Disease, Ear Anomalies-Contractures-Dysplasia of Bone with Kyphoscoliosis, Ear Patella Short Stature Syndrome, Early Constraint Defects, Early Hypercalcemia Syndrome with Elfin Facie, Early-onset Dystonia, Eaton Lambert Syndrome, EB, Ebstein's anomaly, EBV Susceptibility (EBVS), EBVS, ECD, ECPSG, Ectodermal Dysplasias, Ectodermal Dysplasia Anhidrotic with Cleft Lip and Cleft Palate, Ectodermal Dysplasia-Exocrine Pancreatic Insufficiency, Ectodermal Dysplasia Rapp-Hodgkin type, Ectodermal and Mesodermal Dysplasia Congenital, Ectodermal and Mesodermal Dysplasia with Osseous Involvement, Ectodermosis Erosiva Pluriorificialis, Ectopia Lentis, Ectopia Vesicae, Ectopic ACTH Syndrome, Ectopic Adrenocorticotropic Hormone Syndrome, Ectopic Anus, Ectrodactilia of the Hand, Ectrodactyl), Ectrodactyly-Ectodermal Dysplasia-Clefting Syndrome, Ectrodactyly Ectodermal Dysplasias Clefting Syndrome, Ectrodactyly Ectodermal Dysplasia Cleft Lip/Cleft Palate, Eczema, Eczema-Thrombocytopenia-Immunodeficiency Syndrome, EDA, EDMD, EDS, EDS Arterial-Ecchymotic Type, EDS Arthrochalasia, EDS Classic Severe Form, EDS Dysfibronectinemic, EDS Gravis Type, EDS Hypermobility, EDS Kyphoscoliotic, EDS Kyphoscoliosis, EDS Mitis Type, EDS Ocular-Scoliotic, EDS Progeroid, EDS Periodontosis, EDS Vascular, EEC Syndrome, EFE, EHBA, EHK, Ehlers Danlos Syndrome, Ehlers-Danlos syndrome, Ehlers Danlos IX, Eisenmenger Complex, Eisenmenger's complex, Eisenmenger Disease, Eisenmenger Reaction, Eisenmenger Syndrome, Ekbom Syndrome, Ekman-Lobstein Disease, Ektrodactyly of the Hand, EKV, Elastin fiber disorders, Elastorrhexis Generalized, Elastosis Dystrophica Syndrome, Elective Mutism (obsolete), Elective Mutism, Electrocardiogram (ECG or EKG), Electron Transfer Flavoprotein (ETF) Dehydrogenase Deficiency: (GAII & MADD), Electrophysiologic study (EPS), Elephant Nails From Birth, Elephantiasis Congenita Angiomatosa, Hemangiectatic Hypertrophy, Elfin Facies with Hypercalcemia, Ellis-van Creveld Syndrome, Ellis Van Creveld Syndrome, Embryoma Kidney, Embryonal Adenomyosarcoma Kidney, Embryonal Carcinosarcoma Kidney, Embryonal Mixed Tumor Kidney, EMC, Emery Dreyfus Muscular Dystrophy, Emery-Dreifuss Muscular Dystrophy, Emery-Dreifuss Syndrome, EMF, EMG Syndrome, Empty Sella Syndrome, Encephalitis Periaxialis Diffusa, Encephalitis Periaxialis Concentrica, Encephalocele, Encephalofacial Angiomatosis, Encephalopathy, Encephalotrigeminal Angiomatosis, Enchondromatosis with Multiple Cavernous Hemangiomas, Endemic Polyneuritis, Endocardial Cushion Defect, Endocardial Cushion Defects, Endocardial Dysplasia, Endocardial Fibroelastosis (EFE), Endogenous Hypertriglyceridemia, Endolymphatic Hydrops, Endometrial Growths, Endometriosis, Endomyocardial Fibrosis, Endothelial Corneal Dystrophy Congenital, Endothelial Epithelial Corneal Dystrophy, Endothelium, Engelmann Disease, Enlarged Tongue, Enterocolitis, Enterocyte Cobalamin Malabsorption, Eosinophia Syndrome, Eosinophilic Cellulitis, Eosinophilic Fasciitis, Eosinophilic Granuloma, Eosinophilic Syndrome, Epidermal Nevus Syndrome, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Epilepsia Procursiva, Epilepsy, Epinephrine, Epiphyseal Changes and High Myopia, Epiphyseal Osteochondroma Benign, Epiphysealis Hemimelica Dysplasia, Episodic-Abnormal Eye Movement, Epithelial Basement Membrane Corneal Dystrophy, Epithelial Corneal Dystrophy of Meesmann Juvenile, Epitheliomatosis Multiplex with Nevus, Epithelium, Epival, EPS, Epstein-Barr Virus-Induced Lymphoproliferative Disease in Males, Erb-Goldflam syndrome, Erdheim Chester Disease, Erythema Multiforme Exudativum, Erythema Polymorphe Stevens Johnson Type, Erythroblastophthisis, Erythroblastosis Fetalis, Erythroblastosis Neonatorum, Erythroblastotic Anemia of Childhood, Erythrocyte Phosphoglycerate Kinase Deficiency, Erythrogenesis Imperfecta, Erythrokeratodermia Progressiva Symmetrica, Erythrokeratodermia Progressiva Symmetrica Ichthyosis, Erythrokeratodermia Variabilis, Erythrokeratodermia Variabilis Type, Erythrokeratolysis Hiemalis, Erythropoietic Porphyrias, Erythropoietic Porphyria, Escobar Syndrome, Esophageal Atresia, Esophageal Aperistalsis, Esophagitis-Peptic Ulcer, Esophagus Atresia and/or Tracheoesophageal Fistula, Essential Familial Hyperlipemia, Essential Fructosuria, Essential Hematuria, Essential Hemorrhagic Thrombocythemia, Essential Mixed Cryoglobulinemia, Essential Moschowitz Disease, Essential Thrombocythemia, Essential Thrombocytopenia, Essential Thrombocytosis, Essential Tremor, Esterase Inhibitor Deficiency, Estren-Dameshek variant of Fanconi Anemia, Estrogen-related Cholestasis, ET, ETF, Ethylmalonic Adipicaciduria, Eulenburg Disease, pc, EVCS, Exaggerated Startle Reaction, Exencephaly, Exogenous Hypertriglyceridemia, Exomphalos-Macroglossia-Gigantism Syndrom, Exophthalmic Goiter, Expanded Rubella Syndrome, Exstrophy of the Bladder, EXT, External Chondromatosis Syndrome, Extrahepatic Biliary Atresia, Extramedullary Plasmacytoma, Exudative Retinitis, Eye Retraction Syndrome, FA1, FAA, Fabry Disease, FAC, FACB, FACD, FACE, FACF, FACG, FACH, Facial Nerve Palsy, Facial Paralysis, Facial Ectodermal Dysplasias, Facial Ectodermal Dysplasia, Facio-Scapulo-Humeral Dystrophy, Facio-Auriculo-Vertebral Spectrum, Facio-cardio-cutaneous syndrome, Facio-Fronto-Nasal Dysplasia, Faciocutaneoskeletal Syndrome, Faciodigitogenital syndrome, Faciogenital dysplasia, Faciogenitopopliteal Syndrome, Faciopalatoosseous Syndrome, Faciopalatoosseous Syndrome Type II, Facioscapulohumeral muscular dystrophy, Factitious Hypoglycemia, Factor VIII Deficiency, Factor IX Deficiency, Factor XI Deficiency, Factor XII deficiency, Factor XIII Deficiency, Fahr Disease, Fahr's Disease, Failure of Secretion Gastric Intrinsic Factor, Fairbank Disease, Fallot's Tetralogy, Familial Acrogeria, Familial Acromicria, Familial Adenomatous Colon Polyposis, Familial Adenomatous Polyposis with Extraintestinal Manifestations, Familial Alobar Holoprosencephaly, Familial Alpha-Lipoprotein Deficiency, Familial Amyotrophic Chorea with Acanthocytosis, Familial Arrhythmic Myoclonus, Familial Articular Chondrocalcinosis, Familial Atypical Mole-Malignant Melanoma Syndrome, Familial Broad Beta Disease, Familial Calcium Gout, Familial Calcium Pyrophosphate Arthropathy, Familial Chronic Obstructive Lung Disease, Familial Continuous Skin Peeling, Familial Cutaneous Amyloidosis, Familial Dysproteinemia, Familial Emphysema, Familial Enteropathy Microvillus, Familial Foveal Retinoschisis, Familial Hibernation Syndrome, Familial High Cholesterol, Familial Hemochromatosis, Familial High Blood Cholesterol, Familial High-Density Lipoprotein Deficiency, Familial High Serum Cholesterol, Familial Hyperlipidema, Familial Hypoproteinemia with Lymphangietatic Enteropathy, Familial Jaundice, Familial Juvenile Nephronophtisis-Associated Ocular Anomaly, Familial Lichen Amyloidosis (Type IX), Familial Lumbar Stenosis, Familial Lymphedema Praecox, Familial Mediterranean Fever, Familial Multiple Polyposis, Familial Nuchal Bleb, Familial Paroxysmal Polyserositis, Familial Polyposis Coli, Familial Primary Pulmonary Hypertension, Familial Renal Glycosuria, Familial Splenic Anemia, Familial Startle Disease, Familial Visceral Amyloidosis (Type VIII), FAMMM, FANCA, FANCB, FANCC, FANCD, FANCE, Fanconi Panmyelopathy, Fanconi Pancytopenia, Fanconi II, Fanconi's Anemia, Fanconi's Anemia Type I, Fanconi's Anemia Complementation Group, Fanconi's Anemia Complementation Group A, Fanconi's Anemia Complementation Group B, Fanconi's Anemia Complementation Group C, Fanconi's Anemia Complementation Group D, Fanconi's Anemia Complementation Group E, Fanconi's Anemia Complementation Group G, Fanconi's Anemia Complementation Group H, Fanconi's Anemia Estren-Dameshek Variant, FANF, FANG, FANH, FAP, FAPG, Farber's Disease, Farber's Lipogranulomatosis, FAS, Fasting Hypoglycemia, Fat-Induced Hyperlipemia, Fatal Granulomatous Disease of Childhood, Fatty Oxidation Disorders, Fatty Liver with Encephalopathy, FAV, FCH, FCMD, FCS Syndrome, FD, FDH, Febrile Mucocutaneous Syndrome Stevens Johnson Type, Febrile Neutrophilic Dermatosis Acute, Febrile Seizures, Feinberg's syndrome, Feissinger-Leroy-Reiter Syndrome, Female Pseudo-Turner Syndrome, Femoral Dysgenesis Bilateral-Robin Anomaly, Femoral Dysgenesis Bilateral, Femoral Facial Syndrome, Femoral Hypoplasia-Unusual Facies Syndrome, Fetal Alcohol Syndrome, Fetal Anti-Convulsant Syndrome, Fetal Cystic Hygroma, Fetal Effects of Alcohol, Fetal Effects of Chickenpox, Fetal Effects of Thalidomide, Fetal Effects of Varicella Zoster Virus, Fetal Endomyocardial Fibrosis, Fetal Face Syndrome, Fetal Iritis Syndrome, Fetal Transfusion Syndrome, Fetal Valproate Syndrome, Fetal Valproic Acid Exposure Syndrome, Fetal Varicella Infection, Fetal Varicella Zoster Syndrome, FFDD Type II, FG Syndrome, FGDY, FHS, Fibrin Stabilizing Factor Deficiency, Fibrinase Deficiency, Fibrinoid Degeneration of Astrocytes, Fibrinoid Leukodystrophy, Fibrinoligase Deficiency, Fibroblastoma Perineural, Fibrocystic Disease of Pancreas, Fibrodysplasia Ossificans Progressiva, Fibroelastic Endocarditis, Fibromyalgia, Fibromyalgia-Fibromyositis, Fibromyositis, Fibrosing Cholangitis, Fibrositis, Fibrous Ankylosis of Multiple Joints, Fibrous Cavemositis, Fibrous Dysplasia, Fibrous Plaques of the Penis, Fibrous Sclerosis of the Penis, Fickler-Winkler Type, Fiedler Disease, Fifth Digit Syndrome, Filippi Syndrome, Finnish Type Amyloidosis (Type V), First Degree Congenital Heart Block, First and Second Branchial Arch Syndrome, Fischer's Syndrome, Fish Odor Syndrome, Fissured Tongue, Flat Adenoma Syndrome, Flatau-Schilder Disease, Flavin Containing Monooxygenase 2, Floating Beta Disease, Floating-Harbor Syndrome, Floating Spleen, Floppy Infant Syndrome, Floppy Valve Syndrome, Fluent aphasia, FMD, FMF, FMO Adult Liver Form, FMO2, FND, Focal Brain Ischemia, Focal Dermal Dysplasia Syndrome, Focal Dermal Hypoplasia, Focal Dermato-Phalangeal Dysplasia, Focal Dystonia, Focal Epilepsy, Focal Facial Dermal Dysplasia Type II, Focal Neuromyotonia, FODH, Folling Syndrome, Fong Disease, FOP, Forbes Disease, Forbes-Albright Syndrome, Forestier's Disease, Forsius-Eriksson Syndrome (X-Linked), Fothergill Disease, Fountain Syndrome, Foveal Dystrophy Progressive, FPO Syndrome Type II, FPO, Fraccaro Type Achondrogenesis (Type IB), Fragile X syndrome, Franceschetti-Zwalen-Klein Syndrome, Francois Dyscephaly Syndrome, Francois-Neetens Speckled Dystrophy, Flecked Corneal Dystrophy, Fraser Syndrome, FRAXA, FRDA, Fredrickson Type I Hyperlipoproteinemia, Freeman-Sheldon Syndrome, Freire-Maia Syndrome, Frey's Syndrome, Friedreich's Ataxia, Friedreich's Disease, Friedreich's Tabes, FRNS, Froelich's Syndrome, Frommel-Chiari Syndrome, Frommel-Chiari Syndrome Lactation-Uterus Atrophy, Frontodigital Syndrome, Frontofacionasal Dysostosis, Frontofacionasal Dysplasia, Frontonasal Dysplasia, Frontonasal Dysplasia with Coronal Craniosynostosis, Fructose-1-Phosphate Aldolase Deficiency, Fructosemia, Fructosuria, Fryns Syndrome, FSH, FSHD, FSS, Fuchs Dystrophy, Fucosidosis Type 1, Fucosidosis Type 2, Fucosidosis Type 3, Fukuhara Syndrome, Fukuyama Disease, Fukuyama Type Muscular Dystrophy, Fumarylacetoacetase deficiency, Furrowed Tongue, G Syndrome, G6PD Deficiency, G6PD, GA I, GA IIB, GA IIA, GA II, GAII & MADD, Galactorrhea-Amenorrhea Syndrome Nonpuerperal, Galactorrhea-Amenorrhea without Pregnancy, Galactosamine-6-Sulfatase Deficiency, Galactose-1-Phosphate Uridyl Transferase Deficiency, Galactosemia, GALB Deficiency, Galloway-Mowat Syndrome, Galloway Syndrome, GALT Deficiency, Gammaglobulin Deficiency, GAN, Ganglioside Neuraminidase Deficiency, Ganglioside Sialidase Deficiency, Gangliosidosis GM1 Type 1, Gangliosidosis GM2 Type 2, Gangliosidosis Beta Hexosaminidase B Defeciency, Gardner Syndrome, Gargoylism, Garies-Mason Syndrome, Gasser Syndrome, Gastric Intrinsic Factor Failure of Secretion, Enterocyte Cobalamin, Gastrinoma, Gastritis, Gastroesophageal Laceration-Hemorrhage, Gastrointestinal Polyposis and Ectodermal Changes, Gastrointestinal ulcers, Gastroschisis, Gaucher Disease, Gaucher-Schlagenhaufer, Gayet-Wernicke Syndrome, GBS, GCA, GCM Syndrome, GCPS, Gee-Herter Disease, Gee-Thaysen Disease, Gehrig's Disease, Gelineau's Syndrome, Genee-Wiedemann Syndrome, Generalized Dystonia, Generalized Familial Neuromyotonia, Generalized Fibromatosis, Generalized Flexion Epilepsy, Generalized Glycogenosis, Generalized Hyperhidrosis, Generalized Lipofuscinosis, Generalized Myasthenia Gravis, Generalized Myotonia, Generalized Sporadic Neuromytonia, Genetic Disorders, Genital Defects, Genital and Urinary Tract Defects, Gerstmann Syndrome, Gerstmann Tetrad, GHBP, GHD, GHR, Giant Axonal Disease, Giant Axonal Neuropathy, Giant Benign Lymphoma, Giant Cell Glioblastoma Astrocytoma, Giant Cell Arteritis, Giant Cell Disease of the Liver, Giant Cell Hepatitis, Giant Cell of Newborns Cirrhosis, Giant Cyst of the Retina, Giant Lymph Node Hyperplasia, Giant Platelet Syndrome Hereditary, Giant Tongue, gic Macular Dystrophy, Gilbert's Disease, Gilbert Syndrome, Gilbert-Dreyfus Syndrome, Gilbert-Lereboullet Syndrome, Gilford Syndrome, Gilles de la Tourette's syndrome, Gillespie Syndrome, Gingival Fibromatosis-Abnormal Fingers Nails Nose Ear Splenomegaly, GLA Deficiency, GLA, GLB1, Glaucoma, Glioma Retina, Global aphasia, Globoid Leukodystrophy, Glossoptosis Micrognathia and Cleft Palate, Glucocerebrosidase deficiency, Glucocerebrosidosis, Glucose-6-Phosphate Dehydrogenase Deficiency, Glucose-6-Phosphate Transport Defect, Glucose-6-Phospate Translocase Deficiency, Glucose-G-Phosphatase Deficiency, Glucose-Galactose Malabsorption, Glucosyl Ceramide Lipidosis, Glutaric Aciduria I, Glutaric Acidemia I, Glutaric Acidemia II, Glutaric Aciduria II, Glutaric Aciduria Type II, Glutaric Aciduria Type III, Glutaricacidemia I, Glutaricacidemia II, Glutaricaciduria I, Glutaricaciduria II, Glutaricaciduria Type IIA, Glutaricaciduria Type IIB, Glutaryl-CoA Dehydrogenase Deficiency, Glutaurate-Aspartate Transport Defect, Gluten-Sensitive Enteropathy, Glycogen Disease of Muscle Type VII, Glycogen Storage Disease I, Glycogen Storage Disease III, Glycogen Storage Disease IV, Glycogen Storage Disease Type V, Glycogen Storage Disease VI, Glycogen Storage Disease VII, Glycogen Storage Disease VIII, Glycogen Storage Disease Type II, Glycogen Storage Disease-Type II, Glycogenosis, Glycogenosis Type I, Glycogenosis Type IA, Glycogenosis Type IB, Glycogenosis Type II, Glycogenosis Type II, Glycogenosis Type III, Glycogenosis Type IV, Glycogenosis Type V, Glycogenosis Type VI, Glycogenosis Type VII, Glycogenosis Type VIII, Glycolic Aciduria, Glycolipid Lipidosis, GM2 Gangliosidosis Type 1, GM2 Gangliosidosis Type 1, GNPTA, Goitrous Autoimmune Thyroiditis, Goldenhar Syndrome, Goldenhar-Gorlin Syndrome, Goldscheider's Disease, Goltz Syndrome, Goltz-Gorlin Syndrome, Gonadal Dysgenesis 45X, Gonadal Dysgenesis XO, Goniodysgenesis-Hypodontia, Goodman Syndrome, Goodman, Goodpasture Syndrome, Gordon Syndrome, Gorlin's Syndrome, Gorlin-Chaudhry-Moss Syndrome, Gottron Erythrokeratodermia Congenitalis Progressiva Symmetrica, Gottron's Syndrome, Gougerot-Carteaud Syndrome, Grand Mal Epilepsy, Granular Type Corneal Dystrophy, Granulomatous Arteritis, Granulomatous Colitis, Granulomatous Dermatitis with Eosinophilia, Granulomatous Ileitis, Graves Disease, Graves' Hyperthyroidism, Graves' Disease, Greig Cephalopolysyndactyly Syndrome, Groenouw Type I Corneal Dystrophy, Groenouw Type II Corneal Dystrophy, Gronblad-Strandberg Syndrome, Grotton Syndrome, Growth Hormone Receptor Deficiency, Growth Hormone Binding Protein Deficiency, Growth Hormone Deficiency, Growth-Mental Deficiency Syndrome of Myhre, Growth Retardation-Rieger Anomaly, GRS, Gruber Syndrome, GS, GSD6, GSD8, GTS, Guanosine Triphosphate-Cyclohydrolase Deficiency, Guanosine Triphosphate-Cyclohydrolase Deficiency, Guenther Porphyria, Guerin-Stern Syndrome, Guillain-Barré, Guillain-Barre Syndrome, Gunther Disease, H Disease, H. Gottron's Syndrome, Habit Spasms, HAE, Hageman Factor Deficiency, Hageman factor, Haim-Munk Syndrome, Hajdu-Cheney Syndrome, Hajdu Cheney, HAL Deficiency, Hall-Pallister Syndrome, Hallennann-Streiff-Francois syndrome, Hallermann-Streiff Syndrome, Hallervorden-Spatz Disease, Hallervorden-Spatz Syndrome, Hallopeau-Siemens Disease, Hallux Duplication Postaxial Polydactyly and Absence of Corpus Callosum, Halushi-Behcet's Syndrome, Hamartoma of the Lymphatics, Hand-Schueller-Christian Syndrome, HANE, Hanhart Syndrome, Happy Puppet Syndrome, Harada Syndrome, HARD+/−E Syndrome, HARD Syndrome, Hare Lip, Harlequin Fetus, Harlequin Type DOC 6, Harlequin Type Ichthyosis, Harley Syndrome, Harrington Syndrome, Hart Syndrome, Hartnup Disease, Hartnup Disorder, Hartnup Syndrome, Hashimoto's Disease, Hashimoto-Pritzker Syndrome, Hashimoto's Syndrome, Hashimoto's Thyroiditis, Hashimoto-Pritzker Syndrome, Hay Well's Syndrome, Hay-Wells Syndrome of Ectodermal Dysplasia, HCMM, HCP, HCTD, HD, Heart-Hand Syndrome (Holt-Oram Type), Heart Disease, Hecht Syndrome, HED, Heerferdt-Waldenstrom and Lofgren's Syndromes, Hegglin's Disease, Heinrichsbauer Syndrome, Hemangiomas, Hemangioma Familial, Hemangioma-Thrombocytopenia Syndrome, Hemangiomatosis Chondrodystrophica, Hemangiomatous Branchial Clefts-Lip Pseudocleft Syndrome, Hemifacial Microsomia, Hemimegalencephaly, Hemiparesis of Cerebral Palsy, Hemiplegia of Cerebral Palsy, Hemisection of the Spinal Cord, Hemochromatosis, Hemochromatosis Syndrome, Hemodialysis-Related Amyloidosis, Hemoglobin Lepore Syndromes, Hemolytic Anemia of Newborn, Hemolytic Cold Antibody Anemia, Hemolytic Disease of Newborn, Hemolytic-Uremic Syndrome, Hemophilia, Hemophilia A, Hemophilia B, Hemophilia B Factor IX, Hemophilia C, Hemorrhagic Dystrophic Thrombocytopenia, Hemorrhagica Aleukia, Hemosiderosis, Hepatic Fructokinase Deficiency, Hepatic Phosphorylase Kinase Deficiency, Hepatic Porphyria, Hepatic Porphyrias, Hepatic Veno-Occlusive Diseas, Hepatitis C, Hepato-Renal Syndrome, Hepatolenticular Degeneration, Hepatophosphorylase Deficiency, Hepatorenal Glycogenosis, Hepatorenal Syndrome, Hepatorenal Tyrosinemia, Hereditary Acromelalgia, Hereditary Alkaptonuria, Hereditary Amyloidosis, Hereditary Angioedema, Hereditary Areflexic Dystasia, Heredopathia Atactica Polyneuritiformis, Hereditary Ataxia, Hereditary Ataxia Friedrich's Type, Hereditary Benign Acanthosis Nigricans, Hereditary Cerebellar Ataxia, Hereditary Chorea, Hereditary Chronic Progressive Chorea, Hereditary Connective Tissue Disorders, Hereditary Coproporphyria, Hereditary Coproporphyria Porphyria, Hereditary Cutaneous Malignant Melanoma, Hereditary Deafness-Retinitis Pigmentosa, Heritable Disorder of Zinc Deficiency, Hereditary DNS, Hereditary Dystopic Lipidosis, Hereditary Emphysema, Hereditary Fructose Intolerance, Hereditary Hemorrhagic Telangiectasia, Hereditary Hemorrhagic Telangiectasia Type I, Hereditary Hemorrhagic Telangiectasia Type II, Hereditary Hemorrhagic Telangiectasia Type III, Hereditary Hyperuricemia and Choreoathetosis Syndrome, Hereditary Leptocytosis Major, Hereditary Leptocytosis Minor, Hereditary Lymphedema, Hereditary Lymphedema Tarda, Hereditary Lymphedema Type I, Hereditary Lymphedema Type II, Hereditary Motor Sensory Neuropathy, Hereditary Motor Sensory Neuropathy I, Hereditary Motor Sensory Neuropathy Type III, Hereditary Nephritis, Hereditary Nephritis and Nerve Deafness, Hereditary Nephropathic Amyloidosis, Hereditary Nephropathy and Deafness, Hereditary Nonpolyposis Colorectal Cancer, Hereditary Nonpolyposis Colorectal Carcinoma, Hereditary Nonspherocytic Hemolytic Anemia, Hereditary Onychoosteodysplasia, Hereditary Optic Neuroretinopathy, Hereditary Polyposis Coli, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Hereditary Site Specific Cancer, Hereditary Spherocytic Hemolytic Anemia, Hereditary Spherocytosis, Hereditary Tyrosinemia Type 1, Heritable Connective Tissue Disorders, Herlitz Syndrome, Hermans-Herzberg Phakomatosis, Hermansky-Pudlak Syndrome, Hermaphroditism, Herpes Zoster, Herpes Iris Stevens-Johnson Type, Hers Disease, Heterozygous Beta Thalassemia, Hexoaminidase Alpha-Subunit Deficiency (Variant B), Hexoaminidase Alpha-Subunit Deficiency (Variant B), HFA, HFM, HGPS, HH, HHHO, HHRH, HHT, Hiatal Hernia-Microcephaly-Nephrosis Galloway Type, Hidradenitis Suppurativa, Hidrosadenitis Axillaris, Hidrosadenitis Suppurativa, Hidrotic Ectodermal Dysplasias, HIE Syndrome, High Imperforate Anus, High Potassium, High Scapula, HIM, Hirschsprung's Disease, Hirschsprung's Disease Acquired, Hirschsprung Disease Polydactyly of Ulnar & Big Toe and VSD, Hirschsprung Disease with Type D Brachydactyl), Hirsutism, HIS Deficiency, Histidine Ammonia-Lyase (HAL) Deficiency, Histidase Deficiency, Histidinemia, Histiocytosis, Histiocytosis X, HLHS, HLP Type II, HMG, HMI, HMSN I, HNHA, HOCM, Hodgkin Disease, Hodgkin's Disease, Hodgkin's Lymphoma, Hollaender-Simons Disease, Holmes-Adie Syndrome, Holocarboxylase Synthetase Deficiency, Holoprosencephaly, Holoprosencephaly Malformation Complex, Holoprosencephaly Sequence, Holt-Oram Syndrome, Holt-Oram Type Heart-Hand Syndrome, Homocystinemia, Homocystinuria, Homogentisic Acid Oxidase Deficiency, Homogentisic Acidura, Homozygous Alpha-1-Antitrypsin Deficiency, HOOD, Horner Syndrome, Horton's disease, HOS, HOSI, Houston-Harris Type Achrondrogenesis (Type IA), HPS, HRS, HS, HSAN Type I, HSAN Type II, HSAN-III, HSMN, HSMN Type III, HSN I, HSN-III, Huebner-Herter Disease, Hunner's Patch, Hunner's Ulcer, Hunter Syndrome, Hunter-Thompson Type Acromesomelic Dysplasia, Huntington's Chorea, Huntington's Disease, Hurler Disease, Hurler Syndrome, Hurler-Scheie Syndrome, HUS, Hutchinson-Gilford Progeria Syndrome, Hutchinson-Gilford Syndrome, Hutchinson-Weber-Peutz Syndrome, Hufterite Syndrome Bowen-Conradi Type, Hyaline Panneuropathy, Hydranencephaly, Hydrocephalus, Hydrocephalus Agyria and Retinal Dysplasia, Hydrocephalus Internal Dandy-Walker Type, Hydrocephalus Noncommunicating Dandy-Walker Type, Hydrocephaly, Hydronephrosis With Peculiar Facial Expression, Hydroxylase Deficiency, Hygroma Colli, Hyper-IgE Syndrome, Hyper-IgM Syndrome, Hyperaldosteronism, Hyperaldosteronism With Hypokalemic Alkatosis, Hyperaldosteronism Without Hypertension, Hyperammonemia, Hyperammonemia Due to Carbamylphosphate Synthetase Deficiency, Hyperammonemia Due to Ornithine Transcarbamylase Deficiency, Hyperammonemia Type II, Hyper-Beta Carnosinemia, Hyperbilirubinemia I, Hyperbilirubinemia II, Hypercalcemia Familial with Nephrocalcinosis and Indicanuria, Hypercalcemia-Supravalvar Aortic Stenosis, Hypercalciuric Rickets, Hypercapnic acidosis, Hypercatabolic Protein-Losing Enteropathy, Hyperchloremic acidosis, Hypercholesterolemia, Hypercholesterolemia Type IV, Hyperchylomicronemia, Hypercystinuria, Hyperekplexia, Hyperextensible joints, Hyperglobulinemic Purpura, Hyperglycinemia with Ketoacidosis and Lactic Acidosis Propionic Type, Hyperglycinemia Nonketotic, Hypergonadotropic Hypogonadism, Hyperimmunoglobulin E Syndrome, Hyperimmunoglobulin E-Recurrent Infection Syndrome, Hyperimmunoglobulinemia E-Staphylococcal, Hyperkalemia, Hyperkinetic Syndrome, Hyperlipemic Retinitis, Hyperlipidemia I, Hyperlipidemia IV, Hyperlipoproteinemia Type I, Hyperlipoproteinemia Type III, Hyperlipoproteinemia Type IV, Hyperoxaluria, Hyperphalangy-Clinodactyly of Index Finger with Pierre Robin Syndrome, Hyperphenylalanemia, Hyperplastic Epidermolysis Bullosa, Hyperpnea, Hyperpotassemia, Hyperprebeta-Lipoproteinemia, Hyperprolinemia Type I, Hyperprolinemia Type II, Hypersplenism, Hypertelorism with Esophageal Abnormalities and Hypospadias, Hypertelorism-Hypospadias Syndrome, Hypertrophic Cardio myopathy, Hypertrophic Interstitial Neuropathy, Hypertrophic Interstitial Neuritis, Hypertrophic Interstitial Radiculoneuropathy, Hypertrophic Neuropathy of Refsum, Hypertrophic Obstructive Cardio myopathy, Hyperuricemia Choreoathetosis Self-multilation Syndrome, Hyperuricemia-Oligophrenia, Hypervalinemia, Hypocalcified (Hypomineralized) Type, Hypochondrogenesis, Hypochrondroplasia, Hypogammaglobulinemia, Hypogammaglobulinemia Transient of Infancy, Hypogenital Dystrophy with Diabetic Tendency, Hypoglossia-Hypodactylia Syndrome, Hypoglycemia, Exogenous Hypoglycemia, Hypoglycemia with Macroglossia, Hypoglycosylation Syndrome Type 1a, Hypoglycosylation Syndrome Type 1a, Hypogonadism with Anosmia, Hypogonadotropic Hypogonadism and Anosmia, Hypohidrotic Ectodermal Dysplasia, Hypohidrotic Ectodermal Dysplasia Autosomal Dominant type, Hypohidrotic Ectodermal Dysplasias Autorecessive, Hypokalemia, Hypokalemic Alkalosis with Hypercalciuria, Hypokalemic Syndrome, Hypolactasia, Hypomaturation Type (Snow-Capped Teeth), Hypomelanosis of Ito, Hypomelia-Hypotrichosis-Facial Hemangioma Syndrome, Hypomyelination Neuropathy, Hypoparathyroidism, Hypophosphatasia, Hypophosphatemic Rickets with Hypercalcemia, Hypopigmentation, Hypopigmented macular lesion, Hypoplasia of the Depressor Anguli Oris Muscle with Cardiac Defects, Hypoplastic Anemia, Hypoplastic Congenital Anemia, Hypoplastic Chondrodystrophy, Hypoplastic Enamel-Onycholysis-Hypohidrosis, Hypoplastic (Hypoplastic-Explastic) Type, Hypoplastic Left Heart Syndrome, Hypoplastic-Triphalangeal Thumbs, Hypopotassemia Syndrome, Hypospadias-Dysphagia Syndrome, Hyposmia, Hypothalamic Hamartoblastoma Hypopituitarism Imperforate Anus Polydactyl), Hypothalamic Infantilism-Obesity, Hypothyroidism, Hypotonia-Hypomentia-Hypogonadism-Obesity Syndrome, Hypoxanthine-Guanine Phosphoribosyltranferase Defect (Complete Absense of), I-Cell Disease, Iatrogenic Hypoglycemia, IBGC, IBIDS Syndrome, IBM, IBS, IC, I-Cell Disease, ICD, ICE Syndrome Cogan-Reese Type, Icelandic Type Amyloidosis (Type VI), I-Cell Disease, Ichthyosiform Erythroderma Corneal Involvement and Deafness, Ichthyosiform Erythroderma Hair Abnormality Growth and Men, Ichthyosiform Erythroderma with Leukocyte Vacuolation, Ichthyosis, Ichthyosis Congenita, Ichthyosis Congenital with Trichothiodystrophy, Ichthyosis Hystrix, Ichthyosis Hystrix Gravior, Ichthyosis Linearis Circumflexa, Ichthyosis Simplex, Ichthyosis Tay Syndrome, Ichthyosis Vulgaris, Ichthyotic Neutral Lipid Storage Disease, Icteric Leptospirosis, Icterohemorrhagic Leptospirosis, Icterus (Chronic Familial), Icterus Gravis Neonatorum, Icterus Intermittens Juvenalis, Idiopathic Alveolar Hypoventilation, Idiopathic Amyloidosis, Idiopathic Arteritis of Takayasu, Idiopathic Basal Ganglia Calcification (IBGC), Idiopathic Brachial Plexus Neuropathy, Idiopathic Cervical Dystonia, Idiopathic Dilatation of the Pulmonary Artery, Idiopathic Facial Palsy, Idiopathic Familial Hyperlipemia, Idiopathic Hypertrophic Subaortic Stenosis, Idiopathic Hypoproteinemia, Idiopathic Immunoglobulin Deficiency, Idiopathic Neonatal Hepatitis, Idiopathic Non-Specific Ulcerative Colitis, Idiopathic Peripheral Periphlebitis, Idiopathic Pulmonary Fibrosis, Idiopathic Refractory Sideroblastic Anemia, Idiopathic Renal Hematuria, Idiopathic Steatorrhea, Idiopathic Thrombocythemia, Idiopathic Thrombocytopenic Purpura, Idiopathic Thrombocytopenia Purpura (ITP), IDPA, IgA Nephropathy, IHSS, Ileitis, Ileocolitis, Illinois Type Amyloidosis, ILS, 1M, IMD2, IMD5, Immune Defect due to Absence of Thymus, Immune Hemolytic Anemia Paroxysmal Cold, Immunodeficiency with Ataxia Telangiectasia, Immunodeficiency Cellular with Abnormal Immunoglobulin Synthesis, Immunodeficiency Common Variable Unclassifiable, Immunodeficiency with Hyper-IgM, Immunodeficiency with Leukopenia, Immunodeficiency-2, Immunodeficiency-5 (IMD5), Immunoglobulin Deficiency, Imperforate Anus, Imperforate Anus with Hand Foot and Ear Anomalies, Imperforate Nasolacrimal Duct and Premature Aging Syndrome, Impotent Neutrophil Syndrome, Inability To Open Mouth Completely And Short Finger-Flexor, INAD, Inborn Error of Urea Synthesis Arginase Type, Inborn Error of Urea Synthesis Arginino Succinic Type, Inborn Errors of Urea Synthesis Carbamyl Phosphate Type, Inborn Error of Urea Synthesis Citrullinemia Type, Inborn Errors of Urea Synthesis Glutamate Synthetase Type, INCL, Inclusion body myositis, Incomplete Atrioventricular Septal Defect, Incomplete Testicular Feminization, Incontinentia Pigmenti, Incontinenti Pigmenti Achromians, Index Finger Anomaly with Pierre Robin Syndrome, Indiana Type Amyloidosis (Type II), Indolent systemic mastocytosis, Infantile Acquired Aphasia, Infantile Autosomal Recessive Polycystic Kidney Disease, Infantile Beriberi, Infantile Cerebral Ganglioside, Infantile Cerebral Paralysis, Infantile Cystinosis, Infantile Epileptic, Infantile Fanconi Syndrome with Cystinosis, Infantile Finnish Type Neuronal Ceroid Lipofuscinosis, Infantile Gaucher Disease, Infantile Hypoglycemia, Infantile Hypophasphatasia, Infantile Lobar Emphysema, Infantile Myoclonic Encephalopathy, Infantile Myoclonic Encephalopathy and Polymyoclonia, Infantile Myofibromatosis, Infantile Necrotizing Encephalopathy, Infantile Neuronal Ceroid Lipofuscinosis, Infantile Neuroaxonal Dystrophy, Infantile Onset Schindler Disease, Infantile Phytanic Acid Storage Disease, Infantile Refsum Disease (IRD), Infantile Sipoidosis GM-2 Gangliosideosis (Type S), Infantile Sleep Apnea, Infantile Spasms, Infantile Spinal Muscular Atrophy (all types), Infantile Spinal Muscular Atrophy ALS, Infantile Spinal Muscular Atrophy Type I, Infantile Type Neuronal Ceroid Lipofuscinosis, Infectious Jaundice, Inflammatory Bowel Disease, Inflammatory Breast Cancer, Inflammatory Linear Nevus Sebaceous Syndrome, Iniencephaly, Insulin Resistant Acanthosis Nigricans, Insulin Lipodystrophy, Insulin dependent Diabetes, Intention Myoclonus, Intermediate Cystinosis, Intermediate Maple Syrup Urine Disease, Intermittent Ataxia with Pyruvate Dehydrogenase Deficiency, Intermittent Maple Syrup Urine Disease, Internal Hydrocephalus, Interstitial Cystitis, Interstitial Deletion of 4q Included, Intestinal Lipodystrophy, Intestinal Lipophagic Granulomatosis, Intestinal Lymphangiectasia, Intestinal Polyposis I, Intestinal Polyposis II, Intestinal Polyposis III, Intestinal Polyposis-Cutaneous Pigmentation Syndrome, Intestinal Pseudoobstruction with External Opthalmoplegia, Intracranial Neoplasm, Intracranial Tumors, Intracranial Vascular Malformations, Intrauterine Dwarfism, Intrauterine Synechiae, Inverted Smile And Occult Neuropathic Bladder, Iowa Type Amyloidosis (Type IV), IP, IPA, Iridocorneal Endothelial Syndrome, Iridocorneal Endothelial (ICE) Syndrome Cogan-Resse Type, Iridogoniodysgenesis With Somatic Anomalies, Iris Atrophy with Corneal Edema and Glaucoma, Iris Nevus Syndrome, Iron Overload Anemia, Iron Overload Disease, Irritable Bowel Syndrome, Irritable Colon Syndrome, Isaacs Syndrome, Isaacs-Merten Syndrome, Ischemic Cardio myopathy, Isolated Lissencephaly Sequence, Isoleucine 33 Amyloidosis, Isovaleric Acid CoA Dehydrogenase Deficiency, Isovaleric Acidaemia, Isovalericacidemia, Isovaleryl CoA Carboxylase Deficiency, ITO Hypomelanosis, ITO, ITP, IVA, Ivemark Syndrome, Iwanoff Cysts, Jackknife Convulsion, Jackson-Weiss Craniosynostosis, Jackson-Weiss Syndrome, Jacksonian Epilepsy, Jacobsen Syndrome, Jadassohn-Lewandowsky Syndrome, Jaffe-Lichenstein Disease, Jakob's Disease, Jakob-Creutzfeldt Disease, Janeway I, Janeway Dysgammaglobulinemia, Jansen Metaphyseal Dysostosis, Jansen Type Metaphyseal Chondrodysplasia, Jarcho-Levin Syndrome, Jaw-Winking, JBS, JDMS, Jegher's Syndrome, Jejunal Atresia, Jejunitis, Jejunoileitis, Jervell and Lange-Nielsen Syndrome, Jeune Syndrome, JMS, Job Syndrome, Job-Buckley Syndrome, Johanson-Blizzard Syndrome, John Dalton, Johnson-Stevens Disease, Jonston's Alopecia, Joseph's Disease, Joseph's Disease Type I, Joseph's Disease Type II, Joseph's Disease Type III, Joubert Syndrome, Joubert-Bolthauser Syndrome, JRA, Juberg Hayward Syndrome, Juberg-Marsidi Syndrome, Juberg-Marsidi Mental Retardation Syndrome, Jumping Frenchmen, Jumping Frenchmen of Maine, Juvenile Arthritis, Juvenile Autosomal Recessive Polycystic Kidney Disease, Juvenile Cystinosis, Juvenile (Childhood) Dermatomyositis (JDMS), Juvenile Diabetes, Juvenile Gaucher Disease, Juvenile Gout Choreoathetosis and Mental Retardation Syndrome, Juvenile Intestinal Malabsorption of Vit B12, Juvenile Intestinal Malabsorption of Vitamin B12, Juvenile Macular Degeneration, Juvenile Pernicious Anemia, Juvenile Retinoschisis, Juvenile Rheumatoid Arthritis, Juvenile Spinal Muscular Atrophy Included, Juvenile Spinal Muscular Atrophy ALS Included, Juvenile Spinal Muscular Atrophy Type III, Juxta-Articular Adiposis Dolorosa, Juxtaglomerular Hyperplasia, Kabuki Make-Up Syndrome, Kahler Disease, Kallmann Syndrome, Kanner Syndrome, Kanzaki Disease, Kaposi Disease (not Kaposi Sarcoma), Kappa Light Chain Deficiency, Karsch-Neugebauer Syndrome, Kartagener Syndrome-Chronic Sinobronchial Disease and Dextrocardia, Kartagener Triad, Kasabach-Merritt Syndrome, Kast Syndrome, Kawasaki Disease, Kawasaki Syndrome, KBG Syndrome, KD, Kearns-Sayre Disease, Kearns-Sayre Syndrome, Kennedy Disease, Kennedy Syndrome, Kennedy Type Spinal and Bulbar Muscular Atrophy, Kennedy-Stefanis Disease, Kenny Disease, Kenny Syndrome, Kenny Type Tubular Stenosis, Kenny-Caffe Syndrome, Kera. Palmoplant. Con. Pes Planus Ony. Periodon. Arach., Keratitis Ichthyosis Deafness Syndrome, Keratoconus, Keratoconus Posticus Circumscriptus, Keratolysis, Keratolysis Exfoliativa Congenita, Keratolytic Winter Erythema, Keratomalacia, Keratosis Follicularis, Keratosis Follicularis Spinulosa Decalvans, Keratosis Follicularis Spinulosa Decalvans Ichthyosis, Keratosis Nigricans, Keratosis Palmoplantaris with Periodontopathia and Onychogryposis, Keratosis Palmoplantaris Congenital Pes Planus Onychogryposis Periodontosis Arachnodactyl), Keratosis Palmoplantaris Congenital, Pes Planus, Onychogryphosis, Periodontosis, Arachnodactyl), Acroosteolysis, Keratosis Rubra Figurata, Keratosis Seborrheica, Ketoacid Decarboxylase Deficiency, Ketoaciduria, Ketotic Glycinemia, KFS, KID Syndrome, Kidney Agenesis, Kidneys Cystic-Retinal Aplasia Joubert Syndrome, Killian Syndrome, Killian/Teschler-Nicola Syndrome, Kiloh-Nevin syndrome III, Kinky Hair Disease, Kinsbourne Syndrome, Kleeblattschadel Deformity, Kleine-Levin Syndrome, Kleine-Levin Hibernation Syndrome, Klinefelter, Klippel-Feil Syndrome, Klippel-Feil Syndrome Type I, Klippel-Feil Syndrome Type II, Klippel-Feil Syndrome Type III, Klippel Trenaunay Syndrome, Klippel-Trenaunay-Weber Syndrome, Kluver-Bucy Syndrome, KMS, Kniest Dysplasia, Kniest Syndrome, Kobner's Disease, Koebberling-Dunnigan Syndrome, Kohlmeier-Degos Disease, Kok Disease, Korsakoff Psychosis, Korsakoff's Syndrome, Krabbe's Disease Included, Krabbe's Leukodystrophy, Kramer Syndrome, KSS, KTS, KTW Syndrome, Kufs Disease, Kugelberg-Welander Disease, Kugelberg-Welander Syndrome, Kussmaul-Landry Paralysis, KWS, L-3-Hydroxy-Acyl-CoA Dehydrogenase (LCHAD) Deficiency, Laband Syndrome, Labhart-Willi Syndrome, Labyrinthine Syndrome, Labyrinthine Hydrops, Lacrimo-Auriculo-Dento-Digital Syndrome, Lactase Isolated Intolerance, Lactase Deficiency, Lactation-Uterus Atrophy, Lactic Acidosis Leber Hereditary Optic Neuropathy, Lactic and Pyruvate Acidemia with Carbohydrate Sensitivity, Lactic and Pyruvate Acidemia with Episodic Ataxia and Weakness, Lactic and Pyruvate, Lactic acidosis, Lactose Intolerance of Adulthood, Lactose Intolerance, Lactose Intolerance of Childhood, LADD Syndrome, LADD, Lafora Disease Included, Lafora Body Disease, Laki-Lorand Factor Deficiency, LAM, Lambert Type Ichthyosis, Lambert-Eaton Syndrome, Lambert-Eaton Myasthenic Syndrome, Lamellar Recessive Ichthyosis, Lamellar Ichthyosis, Lancereaux-Mathieu-Weil Spirochetosis, Landau-Kleffner Syndrome, Landouzy Dejerine Muscular Dystrophy, Landry Ascending Paralysis, Langer-Salidino Type Achondrogensis (Type II), Langer Giedion Syndrome, Langerhans-Cell Granulomatosis, Langerhans-Cell Histiocytosis (LCH), Large Atrial and Ventricular Defect, Laron Dwarfism, Laron Type Pituitary Dwarfism, Larsen Syndrome, Laryngeal Dystonia, Latah (Observed in Malaysia), Late Infantile Neuroaxonal Dystrophy, Late Infantile Neuroaxonal Dystrophy, Late Onset Cockayne Syndrome Type III (Type C), Late-Onset Dystonia, Late-Onset Immunoglobulin Deficiency, Late Onset Pelizaeus-Merzbacher Brain Sclerosis, Lattice Corneal Dystrophy, Lattice Dystrophy, Launois-Bensaude, Launois-Cleret Syndrome, Laurence Syndrome, Laurence-Moon Syndrome, Laurence-Moon/Bardet-Biedl, Lawrence-Seip Syndrome, LCA, LCAD Deficiency, LCAD, LCAD, LCADH Deficiency, LCH, LCHAD, LCPD, Le Jeune Syndrome, Leband Syndrome, Leber's Amaurosis, Leber's Congenital Amaurosis, Congenital Absence of the Rods and Cones, Leber's Congenital Tapetoretinal Degeneration, Leber's Congenital Tapetoretinal Dysplasia, Leber's Disease, Leber's Optic Atrophy, Leber's Optic Neuropathy, Left Ventricular Fibrosis, Leg Ulcer, Legg-Calve-Perthes Disease, Leigh's Disease, Leigh's Syndrome, Leigh's Syndrome (Subacute Necrotizing Encephalomyelopathy), Leigh Necrotizing Encephalopathy, Lennox-Gastaut Syndrome, Lentigio-Polypose-Digestive Syndrome, Lenz Dysmorphogenetic Syndrome, Lenz Dysplasia, Lenz Microphthalmia Syndrome, Lenz Syndrome, LEOPARD Syndrome, Leprechaunism, Leptomeningeal Angiomatosis, Leptospiral Jaundice, Leri-Weill Disease, Leri-Weil Dyschondrosteosis, Leri-Weil Syndrome, Lermoyez Syndrome, Leroy Disease, Lesch Nyhan Syndrome, Lethal Infantile Cardio myopathy, Lethal Neonatal Dwarfism, Lethal Osteochondrodysplasia, Letterer-Siwe Disease, Leukocytic Anomaly Albinism, Leukocytic Inclusions with Platelet Abnormality, Leukodystrophy, Leukodystrophy with Rosenthal Fibers, Leukoencephalitis Periaxialis Concentric, Levine-Critchley Syndrome, Levulosuria, Levy-Hollister Syndrome, LGMD, LGS, LHON, LIC, Lichen Ruber Acuminatus, Lichen Acuminatus, Lichen Amyloidosis, Lichen Planus, Lichen Psoriasis, Lignac-Debre-Fanconi Syndrome, Lignac-Fanconi Syndrome, Ligneous Conjunctivitis, Limb-Girdle Muscular Dystrophy, Limb Malformations-Dento-Digital Syndrome, Limit Dextrinosis, Linear Nevoid Hypermelanosis, Linear Nevus Sebacous Syndrome, Linear Scleroderma, Linear Sebaceous Nevus Sequence, Linear Sebaceous Nevus Syndrome, Lingua Fissurata, Lingua Plicata, Lingua Scrotalis, Linguofacial Dyskinesia, Lip Pseudocleft-hemangiomatous Branchial Cyst Syndrome, Lipid Granulomatosis, Lipid Histiocytosis, Lipid Kerasin Type, Lipid Storage Disease, Lipid-Storage myopathy Associated with SCAD Deficiency, Lipidosis Ganglioside Infantile, Lipoatrophic Diabetes Mellitus, Lipodystrophy, Lipoid Corneal Dystrophy, Lipoid Hyperplasia-Male Pseudohernaphroditism, Lipomatosis of Pancreas Congenital, Lipomucopolysaccharidosis Type I, Lipomyelomeningocele, Lipoprotein Lipase Deficiency Familial, LIS, LIS1, Lissencephaly 1, Lissencephaly Type I, Lissencephaly variants with agenesis of the corpus callosum cerebellar hypoplasia or other anomalies, Little Disease, Liver Phosphorylase Deficiency, LKS, LM Syndrome, Lobar Atrophy, Lobar Atrophy of the Brain, Lobar Holoprosencephaly, Lobar Tension Emphysema in Infancy, Lobstein Disease (Type I), Lobster Claw Deformity, Localized Epidermolysis Bullosa, Localized Lipodystrophy, Localized Neuritis of the Shoulder Girdle, Loeffler's Disease, Loeffler Endomyocardial Fibrosis with Eosinophilia, Loeffler Fibroplastic Parietal Endocarditis, Loken Syndrome, Loken-Senior Syndrome, Long-Chain 3-hydroxyacyl-CoA Dehydrogenase (LCHAD), Long Chain Acyl CoA Dehydrogenase Deficiency, Long-Chain Acyl-CoA Dehydrogenase (ACADL), Long-Chain Acyl-CoA Dehydrogenase Deficiency, Long QT Syndrome without Deafness, Lou Gehrig's Disease, Lou Gehrig's Disease Included, Louis-Bar Syndrome, Low Blood Sugar, Low-Density Beta Lipoprotein Deficiency, Low Imperforate Anus, Low Potassium Syndrome, Lowe syndrome, Lowe's Syndrome, Lowe-Bickel Syndrome, Lowe-Terry-MacLachlan Syndrome, Lower Back Pain, LS, LTD, Lubs Syndrome, Luft Disease, Lumbar Canal Stenosis, Lumbar Spinal Stenosis, Lumbosacral Spinal Stenosis, Lundborg-Unverricht Disease, Lundborg-Unverricht Disease Included, Lupus, Lupus, Lupus Erythematosus, Luschka-Magendie Foramina Atresia, Lyell Syndrome, Lyelles Syndrome, Lymphadenoid Goiter, Lymphangiectatic Protein-Losing Enteropathy, Lymphangioleiomatosis, Lymphangioleimyomatosis, Lymphangiomas, Lymphatic Malformations, Lynch Syndromes, Lynch Syndrome I, Lynch Syndrome II, Lysosomal Alpha-N-Acetylgalactosaminidase Deficiency Schindler Type, Lysosomal Glycoaminoacid Storage Disease-Angiokeratoma Corporis Diffusum, Lysosomal Glucosidase Deficiency, MAA, Machado Disease, Machado-Joseph Disease, Macrencephaly, Macrocephaly, Macrocephaly Hemihypertrophy, Macrocephaly with Multiple Lipomas and Hemangiomata, Macrocephaly with Pseudopapilledema and Multiple Hemangiomata, Macroglobulinemia, Macroglossia, Macroglossia-Omphalocele-Visceromegaly Syndrome, Macrostomia Ablepheron Syndrome, Macrothrombocytopenia Familial Bernard-Soulier Type, Macula Lutea degeneration, Macular Amyloidosis, Macular Degeneration, Macular Degeneration Disciform, Macular Degeneration Senile, Macular Dystrophy, Macular Type Corneal Dystrophy, MAD, Madelung's Disease, Maffucci Syndrome, Major Epilepsy, Malabsorption, Malabsorption-Ectodermal Dysplasia-Nasal Alar Hypoplasia, Maladie de Roger, Maladie de Tics, Malaria, Male Malformation of Limbs and Kidneys, Male Turner Syndrome, Malignant Acanthosis, Malignant Acanthosis Nigricans, Malignant Astrocytoma, Malignant Atrophic Papulosis, Malignant Fever, Malignant Hyperphenylalaninemia, Malignant Hyperpyrexia, Malignant Hyperthermia, Malignant Melanoma, Malignant Tumors of the Central Nervous System, Mallory-Weiss Laceration, Mallory-Weiss Tear, Mallory-Weiss Syndrome, Mammary Paget's Disease, Mandibular Ameloblastoma, Mandibulofacial Dysostosis, Mannosidosis, Map-Dot-Fingerprint Type Corneal Dystrophy, Maple Syrup Urine Disease, Marble Bones, Marchiafava-Micheli Syndrome, Marcus Gunn Jaw-Winking Syndrome, Marcus Gunn Phenomenon, Marcus Gunn Ptosis with jaw-winking, Marcus Gunn Syndrome, Marcus Gunn (Jaw-Winking) Syndrome, Marcus Gunn Ptosis (with jaw-winking), Marden-Walker Syndrome, Marden-Walker Type Connective Tissue Disorder, Marfan's Abiotrophy, Marfan-Achard syndrome, Marfan Syndrome, Marfan's Syndrome I, Marfan's Variant, Marfanoid Hypermobility Syndrome, Marginal Corneal Dystrophy, Marie's Ataxia, Marie Disease, Marie-Sainton Disease, Marie Strumpell Disease, Marie-Strumpell Spondylitis, Marinesco-Sjogren Syndrome, Marinesco-Sjogren-Gorland Syndrome, Marker X Syndrome, Maroteaux Lamy Syndrome, Maroteaux Type Acromesomelic Dysplasia, Marshall's Ectodermal Dysplasias With Ocular and Hearing Defects, Marshall-Smith Syndrome, Marshall Syndrome, Marshall Type Deafness-Myopia-Cataract-Saddle Nose, Martin-Albright Syndrome, Martin-Bell Syndrome, Martorell Syndrome, MASA Syndrome, Massive Myoclonia, Mast Cell Leukemia, Mastocytosis, Mastocytosis With an Associated Hematologic Disorder, Maumenee Corneal Dystrophy, Maxillary Ameloblastoma, Maxillofacial Dysostosis, Maxillonasal Dysplasia, Maxillonasal Dysplasia Binder Type, Maxillopalpebral Synkinesis, May-Hegglin Anomaly, MCAD Deficiency, MCAD, McArdle Disease, McCune-Albright, MCD, McKusick Type Metaphyseal Chondrodysplasia, MCR, MCTD, Meckel Syndrome, Meckel-Gruber Syndrome, Median Cleft Face Syndrome, Mediterranean Anemia, Medium-Chain Acyl-CoA dehydrogenase (ACADM), Medium Chain Acyl-CoA Dehydrogenase (MCAD) Deficiency, Medium-Chain Acyl-CoA Dehydrogenase Deficiency, Medullary Cystic Disease, Medullary Sponge Kidney, MEF, Megaesophagus, Megalencephaly, Megalencephaly with Hyaline Inclusion, Megalencephaly with Hyaline Panneuropathy, Megaloblastic Anemia, Megaloblastic Anemia of Pregnancy, Megalocornea-Mental Retardation Syndrome, Meier-Gorlin Syndrome, Meige's Lymphedema, Meige's Syndrome, Melanodermic Leukodystrophy, Melanoplakia-Intestinal Polyposis, Melanoplakia-Intestinal Polyposis, MELAS Syndrome, MELAS, Melkersson Syndrome, Melnick-Fraser Syndrome, Melnick-Needles Osteodysplasty, Melnick-Needles Syndrome, Membranous Lipodystrophy, Mendes Da Costa Syndrome, Meniere Disease, Meniere's Disease, Meningeal Capillary Angiomatosis, Menkes Disease, Menke's Syndrome I, Mental Retardation Aphasia Shuffling Gait Adducted Thumbs (MASA), Mental Retardation-Deafness-Skeletal Abnormalities-Coarse Face with Full Lips, Mental Retardation with Hypoplastic 5th Fingernails and Toenails, Mental Retardation with Osteocartilaginous Abnormalities, Mental Retradation-X-linked with Growth Delay-Deafness-Microgenitalism, Menzel Type OPCA, Mermaid Syndrome, MERRF, MERRF Syndrome, Merten-Singleton Syndrome, MES, Mesangial IGA Nephropathy, Mesenteric Lipodystrophy, Mesiodens-Cataract Syndrome, Mesodermal Dysmorphodystrophy, Mesomelic Dwarfism-Madelung Deformity, Metabolic Acidosis, Metachromatic Leukodystrophy, Metatarsus Varus, Metatropic Dwarfism Syndrome, Metatropic Dysplasia, Metatropic Dysplasia I, Metatropic Dysplasia II, Methylmalonic Acidemia, Methylmalonic Aciduria, Meulengracht's Disease, MFD1, MG, MH, MHA, Micrencephaly, Microcephalic Primordial Dwarfism I, Microcephaly, Microcephaly-Hiatal Hernia-Nephrosis Galloway Type, Microcephaly-Hiatal Hernia-Nephrotic Syndrome, Microcystic Corneal Dystrophy, Microcythemia, Microlissencephaly, Microphthalmia, Microphthalmia or Anopthalmos with Associated Anomalies, Micropolygyria With Muscular Dystrophy, Microtia Absent Patellae Micrognathia Syndrome, Microvillus Inclusion Disease, MID, Midsystolic-click-late systolic murmur syndrome, Miescher's Type I Syndrome, Mikulicz Syndrome, Mikulicz-Radecki Syndrome, Mikulicz-Sjogren Syndrome, Mild Autosomal Recessive, Mild Intermediate Maple Syrup Urine Disease, Mild Maple Syrup Urine Disease, Miller Syndrome, Miller-Dieker Syndrome, Miller-Fisher Syndrome, Milroy Disease, Minkowski-Chauffard Syndrome, Minor Epilepsy, Minot-Von Willebrand Disease, Mirror-Image Dextrocardia, Mitochondrial Beta-Oxidation Disorders, Mitrochondrial and Cytosolic, Mitochondrial Cytopathy, Mitochondrial Cytopathy, Kearn-Sayre Type, Mitochondrial Encephalopathy, Mitochondrial Encephalo myopathy Lactic Acidosis and Strokelike Episodes, Mitochondrial myopathy, Mitochondrial myopathy Encephalopathy Lactic Acidosis Stroke-Like Episode, Mitochondrial PEPCK Deficiency, Mitral-valve prolapse, Mixed Apnea, Mixed Connective Tissue Disease, Mixed Hepatic Porphyria, Mixed Non-Fluent Aphasia, Mixed Sleep Apnea, Mixed Tonic and Clonic Torticollis, MJD, MKS, ML I, ML II, ML III, ML IV, ML Disorder Type I, ML Disorder Type II, ML Disorder Type III, ML Disorder Type IV, MLNS, MMR Syndrome, MND, MNGIE, MNS, Mobitz I, Mobitz II, Mobius Syndrome, Moebius Syndrome, Moersch-Woltmann Syndrome, Mohr Syndrome, Monilethrix, Monomodal Visual Amnesia, Mononeuritis Multiplex, Mononeuritis Peripheral, Mononeuropathy Peripheral, Monosomy 3p2, Monosomy 9p Partial, Monosomy 11q Partial, Monosomy 13q Partial, Monosomy 18q Syndrome, Monosomy X, Monostotic Fibrous Dysplasia, Morgagni-Turner-Albright Syndrome, Morphea, Morquio Disease, Morquio Syndrome, Morquio Syndrome A, Morquio Syndrome B, Morquio-Brailsford Syndrome, Morvan Disease, Mosaic Tetrasomy 9p, Motor Neuron Disease, Motor Neuron Syndrome, Motor Neurone Disease, Motoneuron Disease, Motoneurone Disease, Motor System Disease (Focal and Slow), Moya-moya Disease, Moyamoya Disease, MPS, MPS I, MPS I H, MPS1H/S Hurler/Scheie Syndrome, MPS I S Scheie Syndrome, MPS II, MPS IIA, MPS IIB, MPS II-AR Autosomal Recessive Hunter Syndrome, MPS II-XR, MPS II-XR Severe Autosomal Recessive, MPS III, MPS III A B C and D Sanfiloppo A, MPS IV, MPS IV A and B Morquio A, MPS V, MPS VI, MPS VI Severe Intermediate Mild Maroteaux-Lamy, MPS VII, MPS VII Sly Syndrome, MPS VIII, MPS Disorder, MPS Disorder I, MPS Disorder II, MPS Disorder III, MPS Disorder VI, MPS Disorder Type VII, MRS, MS, MSA, MSD, MSL, MSS, MSUD, MSUD, MSUD Type Ib, MSUD Type II, Mucocutaneous Lymph Node Syndrome, Mucolipidosis I, Mucolipidosis II, Mucolipidosis III, Mucolipidosis IV, Mucopolysaccharidosis, Mucopolysaccharidosis I-H, Mucopolysaccharidosis I-S, Mucopolysaccharidosis II, Mucopolysaccharidosis III, Mucopolysaccharidosis IV, Mucopolysaccharidosis VI, Mucopolysaccharidosis VII, Mucopolysaccharidosis Type I, Mucopolysaccharidosis Type II, Mucopolysaccharidosis Type III, Mucopolysaccharidosis Type VII, Mucosis, Mucosulfatidosis, Mucous Colitis, Mucoviscidosis, Mulibrey Dwarfism, Mulibrey Nanism Syndrome, Mullerian Duct Aplasia-Renal Aplasia-Cervicothoracic Somite Dysplasia, Mullerian Duct-Renal-Cervicothoracic-Upper Limb Defects, Mullerian Duct and Renal Agenesis with Upper Limb and Rib Anomalies, Mullerian-Renal-Cervicothoracic Somite Abnormalities, Multi-Infarct Dementia Binswanger's Type, Multicentric Castleman's Disease, Multifocal Eosinophilic Granuloma, Multiple Acyl-CoA Dehydrogenase Deficiency, Multiple Acyl-CoA Dehydrogenase Deficiency/Glutaric Aciduria Type II, Multiple Angiomas and Endochondromas, Multiple Carboxylase Deficiency, Multiple Cartilaginous Enchondroses, Multiple Cartilaginous Exostoses, Multiple Enchondromatosis, Multiple Endocrine Deficiency Syndrome Type II, Multiple Epiphyseal Dysplasia, Multiple Exostoses, Multiple Exostoses Syndrome, Multiple Familial Polyposis, Multiple Lentigines Syndrome, Multiple Myeloma, Multiple Neuritis of the Shoulder Girdle, Multiple Osteochondromatosis, Multiple Peripheral Neuritis, Multiple Polyposis of the Colon, Multiple Pterygium Syndrome, Multiple Sclerosis, Multiple Sulfatase Deficiency, Multiple Symmetric Lipomatosis, Multiple System Atrophy, Multisynostotic Osteodysgenesis, Multisynostotic Osteodysgenesis with Long Bone Fractures, Mulvihill-Smith Syndrome, MURCS Association, Murk Jansen Type Metaphyseal Chondrodysplasia, Muscle Carnitine Deficiency, Muscle Core Disease, Muscle Phosphofructokinase Deficiency, Muscular Central Core Disease, Muscular Dystrophy, Muscular Dystrophy Classic X-linked Recessive, Muscular Dystrophy Congenital With Central Nervous System Involvement, Muscular Dystrophy Congenital Progressive with Mental Retardation, Muscular Dystrophy Facioscapulohumeral, Muscular Rheumatism, Muscular Rigidity—Progressive Spasm, Musculoskeletal Pain Syndrome, Mutilating Acropathy, Mutism, mvp, MVP, MWS, Myasthenia Gravis, Myasthenia Gravis Pseudoparalytica, Myasthenic Syndrome of Lambert-Eaton, Myelinoclastic Diffuse Sclerosis, Myelomatosis, Myhre Syndrome, Myoclonic Astatic Petit Mal Epilepsy, Myoclonic Dystonia, Myoclonic Encephalopathy of Infants, Myoclonic Epilepsy, Myoclonic Epilepsy Hartung Type, Myoclonus Epilepsy Associated with Ragged Red Fibers, Myoclonic Epilepsy and Ragged-Red Fiber Disease, Myoclonic Progressive Familial Epilepsy, Myoclonic Progressive Familial Epilepsy, Myoclonic Seizure, Myoclonus, Myoclonus Epilepsy, Myoencephalopathy Ragged-Red Fiber Disease, Myofibromatosis, Myofibromatosis Congenital, Myogenic Facio-Scapulo-Peroneal Syndrome, Myoneurogastointestinal Disorder and Encephalopathy, Myopathic Arthrogryposis Multiplex Congenita, Myopathic Carnitine Deficiency, Myopathy Central Fibrillar, myopathy Congenital Nonprogressive, myopathy Congenital Nonprogressive with Central Axis, myopathy with Deficiency of Carnitine Palmitoyltransferase, myopathy-Marinesco-Sjogren Syndrome, myopathy-Metabolic Carnitine Palmitoyltransderase Deficiency, myopathy Mitochondrial-Encephalopathy-Lactic Acidosis-Stroke, myopathy with Sarcoplasmic Bodies and Intermediate Filaments, Myophosphorylase Deficiency, Myositis Ossificans Progressiv, Myotonia Atrophica, Myotonia Congenita, Myotonia Congenita Intermittens, Myotonic Dystrophy, Myotonic myopathy Dwarfism Chondrodystrophy Ocular and Facial Anomalies, Myotubular myopathy, Myotubular myopathy X-linked, Myproic Acid, Myriachit (Observed in Siberia), Myxedema, N-Acetylglucosamine-1-Phosphotransferase Deficiency, N-Acetyl Glutamate Synthetase Deficiency, NADH-COQ reductase deficiency, Naegeli Ectodermal Dysplasias, Nager Syndrome, Nager Acrofacial Dysostosis Syndrome, Nager Syndrome, NAGS Deficiency, Nail Dystrophy-Deafness Syndrome, Nail Dysgenesis and Hypodontia, Nail-Patella Syndrome, Nance-Horan Syndrome, Nanocephalic Dwarfism, Nanocephaly, Nanophthalmia, Narcolepsy, Narcoleptic syndrome, NARP, Nasal-fronto-faciodysplasia, Nasal Alar Hypoplasia Hypothyroidism Pancreatic Achylia Congenital Deafness, Nasomaxillary Hypoplasia, Nasu Lipodystrophy, NBIA1, ND, NDI, NDP, Necrotizing Encephalomyelopathy of Leigh's, Necrotizing Respiratory Granulomatosis, Neill-Dingwall Syndrome, Nelson Syndrome, Nemaline myopathy, Neonatal Adrenoleukodystrophy, Neonatal Adrenoleukodystrophy (NALD), Neonatal Adrenoleukodystrophy (ALD), Neonatal Autosomal Recessive Polycystic Kidney Disease, Neonatal Dwarfism, Neonatal Hepatitis, Neonatal Hypoglycemia, Neonatal Lactose Intolerance, Neonatal Lymphedema due to Exudative Enteropathy, Neonatal Necrotizing Enterocolitis, Neonatal Progeroid Syndrome, Neonatal Pseudo-Hydrocephalic Progeroid Syndrome of Wiedemann-Rautenstrauch, Neoplastic Arachnoiditis, Nephroblastom, Nephrogenic Diabetes Insipidus, Nephronophthesis Familial Juvenile, Nephropathic Cystinosis, Nephropathy-Pseudohermaphroditism-Wilms Tumor, Nephrosis-Microcephaly Syndrome, Nephrosis-Neuronal Dysmigration Syndrome, Nephrotic-Glycosuric-Dwarfism-Rickets-Hypophosphatemic Syndrome, Netherton Disease, Netherton Syndrome, Netherton Syndrome Ichthyosis, Nettleship Falls Syndrome (X-Linked), Neu-Laxova Syndrome, Neuhauser Syndrome, Neural-tube defects, Neuralgic Amyotrophy, Neuraminidase Deficiency, Neuraocutaneous melanosis, Neurinoma of the Acoustic Nerve, Neurinoma, Neuroacanthocytosis, Neuroaxonal Dystrophy Schindler Type, Neurodegeneration with brain iron accumulation type 1 (NBIA1), Neurofibroma of the Acoustic Nerve, Neurogenic Arthrogryposis Multiplex Congenita, Neuromyelitis Optica, Neuromyotonia, Neuromyotonia, Focal, Neuromyotonia, Generalized, Familial, Neuromyotonia, Generalized, Sporadic, Neuronal Axonal Dystrophy Schindler Type, Neuronal Ceroid Lipofuscinosis Adult Type, Neuronal Ceroid Lipofuscinosis Juvenile Type, Neuronal Ceroid Lipofuscinosis Type 1, Neuronopathic Acute Gaucher Disease, Neuropathic Amyloidosis, Neuropathic Beriberi, Neuropathy Ataxia and Retinitis Pigmentosa, Neuropathy of Brachialpelxus Syndrome, Neuropathy Hereditary Sensory Type I, Neuropathy Hereditary Sensory Type II, Neuropsychiatric Porphyria, Neutral Lipid Storage Disease, Nevii, Nevoid Basal Cell Carcinoma Syndrome, Nevus, Nevus Cavernosus, Nevus Comedonicus, Nevus Depigmentosus, Nevus Sebaceous of Jadassohn, Nezelof's Syndrome, Nezelof's Thymic Aplasia, Nezelof Type Severe Combined Immunodeficiency, NF, NF1, NF2, NF-1, NF-2, NHS, Niemann Pick Disease, Nieman Pick disease Type A (acute neuronopathic form), Nieman Pick disease Type B, Nieman Pick Disease Type C (chronic neuronopathic form), Nieman Pick disease Type D (Nova Scotia variant), Nieman Pick disease Type E, Nieman Pick disease Type F (sea-blue histiocyte disease), Night Blindness, Nigrospinodentatal Degeneration, Niikawakuroki Syndrome, NLS, NM, Noack Syndrome Type I, Nocturnal Myoclonus Hereditary Essential Myoclonus, Nodular Cornea Degeneration, Non-Bullous CIE, Non-Bullous Congenital Ichthyosiform Erythroderma, Non-Communicating Hydrocephalus, Non-Deletion Type Alpha-Thalassemia/Mental Retardation syndrome, Non-Ketonic Hyperglycinemia Type I (NKHI), Non-Ketotic Hyperglycinemia, Non-Lipid Reticuloendotheliosis, Non-Neuronopathic Chronic Adult Gaucher Disease, Non-Scarring Epidermolysis Bullosa, Nonarteriosclerotic Cerebral Calcifications, Nonarticular Rheumatism, Noncerebral, Juvenile Gaucher Disease, Nondiabetic Glycosuria, Nonischemic Cardio myopathy, Nonketotic Hypoglycemia and Carnitine Deficiency due to MCAD Deficiency, Nonketotic Hypoglycemia Caused by Deficiency of Acyl-CoA Dehydrogenase, Nonketotic Glycinemia, Norme's Syndrome, Norme-Milroy-Meige Syndrome, Nonopalescent Opalescent Dentine, Nonpuerperal Galactorrhea-Amenorrhea, Nonsecretory Myeloma, Nonspherocytic Hemolytic Anemia, Nontropical Sprue, Noonan Syndrome, Norepinephrine, Normal Pressure Hydrocephalus, Norman-Roberts Syndrome, Norrbottnian Gaucher Disease, Norrie Disease, Norwegian Type Hereditary Cholestasis, NPD, NPS, NS, NSA, Nuchal Dystonia Dementia Syndrome, Nutritional Neuropathy, Nyhan Syndrome, OAV Spectrum, Obstructive Apnea, Obstructive Hydrocephalus, Obstructive Sleep Apnea, OCC Syndrome, Occlusive Thromboaortopathy, OCCS, Occult Intracranial Vascular Malformations, Occult Spinal Dysraphism Sequence, Ochoa Syndrome, Ochronosis, Ochronotic Arthritis, OCR, OCRL, Octocephaly, Ocular Albinism, Ocular Herpes, Ocular Myasthenia Gravis, Oculo-Auriculo-Vertebral Dysplasia, Oculo-Auriculo-Vertebral Spectrum, Oculo-Bucco-Genital Syndrome, Oculocerebral Syndrome with Hypopigmentation, Oculocerebrocutaneous Syndrome, Oculo-Cerebro-Renal, Oculocerebrorenal Dystrophy, Oculocerebrorenal Syndrome, Oculocraniosomatic Syndrome (obsolete), Oculocutaneous Albinism, Oculocutaneous Albinism Chediak-Higashi Type, Oculo-Dento-Digital Dysplasia, Oculodentodigital Syndrome, Oculo-Dento-Osseous Dysplasia, Oculo Gastrointestinal Muscular Dystrophy, Oculo Gastrointestinal Muscular Dystrophy, Oculomandibulodyscephaly with hypotrichosis, Oculomandibulofacial Syndrome, Oculomotor with Congenital Contractures and Muscle Atrophy, Oculosympathetic Palsy, ODD Syndrome, ODOD, Odontogenic Tumor, Odontotrichomelic Syndrome, OFD, OFD Syndrome, Ohio Type Amyloidosis (Type VII), OI, OI Congenita, OI Tarda, Oldfield Syndrome, Oligohydramnios Sequence, Oligophrenia Micropthalmos, Oligophrenic Polydystrophy, Olivopontocerebellar Atrophy, Olivopontocerebellar Atrophy with Dementia and Extrapyramidal Signs, Olivopontocerebellar Atrophy with Retinal Degeneration, Olivopontocerebellar Atrophy I, Olivopontocerebellar Atrophy II, Olivopontocerebellar Atrophy III, Olivopontocerebellar Atrophy IV, Olivopontocerebellar Atrophy V, Ollier Disease, Ollier Osteochondromatosis, Omphalocele-Visceromegaly-Macroglossia Syndrome, Ondine's Curse, Onion-Bulb Neuropathy, Onion Bulb Polyneuropathy, Onychoosteodysplasia, Onychotrichodysplasia with Neutropenia, OPCA, OPCA I, OPCA II, OPCA III, OPCA IV, OPCA V, OPD Syndrome, OPD Syndrome Type I, OPD Syndrome Type II, OPD I Syndrome, OPD II Syndrome, Opthalmoarthropathy, Opthalmoplegia-Intestinal Pseudoobstruction, Opthalmoplegia, Pigmentary Degeneration of the Retina and Cadio myopathy, Opthalmoplegia Plus Syndrome, Opthalmoplegia Syndrome, Opitz BBB Syndrome, Opitz BBB/G Compound Syndrome, Opitz BBBG Syndrome, Opitz-Frias Syndrome, Opitz G Syndrome, Opitz G/BBB Syndrome, Opitz Hypertelorism-Hypospadias Syndrome, Opitz-Kaveggia Syndrome, Opitz Oculogenitolaryngeal Syndrome, Opitz Trigonocephaly Syndrome, Opitz Syndrome, Opsoclonus, Opsoclonus-Myoclonus, Opthalmoneuromyelitis, Optic Atrophy Polyneuropathy and Deafness, Optic Neuroencephalomyelopathy, Optic Neuromyelitis, Opticomyelitis, Optochiasmatic Arachnoiditis, Oral-Facial Clefts, Oral-facial Dyskinesia, Oral Facial Dystonia, Oral-Facial-Digital Syndrome, Oral-Facial-Digital Syndrome Type I, Oral-Facial-Digital Syndrome I, Oral-Facial-Digital Syndrome II, Oral-Facial-Digital Syndrome III, Oral-Facial-Digital Syndrome IV, Orbital Cyst with Cerebral and Focal Dermal Malformations, Ornithine Carbamyl Transferase Deficiency, Ornithine Transcarbamylase Deficiency, Orocraniodigital Syndrome, Orofaciodigital Syndrome, Oromandibular Dystonia, Orthostatic Hypotension, Osler-Weber-Rendu disease, Osseous-Oculo-Dento Dysplasia, Osseous-Oculo-Dento Dysplasia, Osteitis deformans, Osteochondrodystrophy Deformans, Osteochondroplasia, Osteodysplasty of Melnick and Needles, Osteogenesis Imperfect, Osteogenesis Imperfecta, Osteogenesis Imperfecta Congenita, Osteogenesis Imperfecta Tarda, Osteohypertrophic Nevus Flammeus, Osteopathia Hyperostotica Scleroticans Multiplex Infantalis, Osteopathia Hyperostotica Scleroticans Multiplex Infantalis, Osteopathyrosis, Osteopetrosis, Osteopetrosis Autosomal Dominant Adult Type, Osteopetrosis Autosomal Recessive Malignant Infantile Type, Osteopetrosis Mild Autosomal Recessive Intermediate Typ, Osteosclerosis Fragilis Generalisata, Osteosclerotic Myeloma, Ostium Primum Defect (endocardial cushion defects included), Ostium Secundum Defect, OTC Deficiency, Oto Palato Digital Syndrome, Oto-Palato-Digital Syndrome Type I, Oto-Palatal-Digital Syndrome Type II, Otodental Dysplasia, Otopalatodigital Syndrome, Otopalataldigital Syndrome Type II, Oudtshoorn Skin, Ovarian Dwarfism Turner Type, Ovary Aplasia Turner Type, OWR, Oxalosis, Oxidase deficiency, Oxycephaly, Oxycephaly-Acrocephaly, P-V, PA, PAC, Pachyonychia Ichtyosiforme, Pachyonychia Congenita with Natal Teeth, Pachyonychia Congenita, Pachyonychia Congenita Keratosis Disseminata Circumscripta (follicularis), Pachyonychia Congenita Jadassohn-Lewandowsky Type, PAF with MSA, Paget's Disease, Paget's Disease of Bone, Paget's Disease of the Breast, Paget's Disease of the Nipple, Paget's Disease of the Nipple and Areola, Pagon Syndrome, Painful Opthalmoplegia, PAIS, Palatal Myoclonus, Palato-Oto-Digital Syndrome, Palatal-Oto-Digital Syndrome Type I, Palatal-Oto-Digital Syndrome Type II, Pallister Syndrome, Pallister-Hall Syndrome, Pallister-Killian Mosaic Syndrome, Pallister Mosaic Aneuploidy, Pallister Mosaic Syndrome, Pallister Mosaic Syndrome Tetrasomy 12p, Pallister-W Syndrome, Palmoplantar Hyperkeratosis and Alopecia, Palsy, Pancreatic Fibrosis, Pancreatic Insufficiency and Bone Marrow Dysfunction, Pancreatic Ulcerogenic Tumor Syndrome, Panmyelophthisis, Panmyelopathy, Pantothenate kinase associated neurodegeneration (PKAN), Papillon-Lefevre Syndrome, Papillotonic Psuedotabes, Paralysis Periodica Paramyotonica, Paralytic Beriberi, Paralytic Brachial Neuritis, Paramedian Lower Lip Pits-Popliteal Pyerygium Syndrome, Paramedian Diencephalic Syndrome, Paramyeloidosis, Paramyoclonus Multiple, Paramyotonia Congenita, Paramyotonia Congenita of Von Eulenburg, Parkinson's disease, Paroxysmal Atrial Tachycardia, Paroxysmal Cold Hemoglobinuria, Paroxysmal Dystonia, Paroxysmal Dystonia Choreathetosis, Paroxysmal Kinesigenic Dystonia, Paroxysmal Nocturnal Hemoglobinuria, Paroxysmal Normal Hemoglobinuria, Paroxysmal Sleep, Parrot Syndrome, Parry Disease, Parry-Romberg Syndrome, Parsonage-Turner Syndrome, Partial Androgen Insensitivity Syndrome, Partial Deletion of the Short Arm of Chromosome 4, Partial Deletion of the Short Arm of Chromosome 5, Partial Deletion of Short Arm of Chromosome 9, Partial Duplication 3q Syndrome, Partial Duplication 15q Syndrome, Partial Facial Palsy With Urinary Abnormalities, Partial Gigantism of Hands and Feet-Nevi-Hemihypertrophy-Macrocephaly, Partial Lipodystrophy, Partial Monosomy of Long Arm of Chromosome 11, Partial Monosomy of the Long Arm of Chromosome 13, Partial Spinal Sensory Syndrome, Partial Trisomy 11q, Partington Syndrome, PAT, Patent Ductus Arteriosus, Pathological Myoclonus, Pauciarticular-Onset Juvenile Arthritis, Paulitis, PBC, PBS, PC Deficiency, PC Deficiency Group A, PC Deficiency Group B, PC, Eulenburg Disease, PCC Deficiency, PCH, PCLD, PCT, PD, PDA, PDH Deficiency, Pearson Syndrome Pyruvate Carboxylase Deficiency, Pediatric Obstructive Sleep Apnea, Peeling Skin Syndrome, Pelizaeus-Merzbacher Disease, Pelizaeus-Merzbacher Brain Sclerosis, Pellagra-Cerebellar Ataxia-Renal Aminoaciduria Syndrome, Pelvic Pain Syndrome, Pemphigus Vulgaris, Pena Shokeir II Syndrome, Pena Shokeir Syndrome Type II, Penile Fibromatosis, Penile Fibrosis, Penile Induration, Penta X Syndrome, Pentalogy of Cantrell, Pentalogy Syndrome, Pentasomy X, PEPCK Deficiency, Pepper Syndrome, Perheentupa Syndrome, Periarticular Fibrositis, Pericardial Constriction with Growth Failure, Pericollagen Amyloidosis, Perinatal Polycystic Kidney Diseases, Perineal Anus, Periodic Amyloid Syndrome, Periodic Peritonitis Syndrome, Periodic Somnolence and Morbid Hunger, Periodic Syndrome, Peripheral Cystoid Degeneration of the Retina, Peripheral Dysostosis-Nasal Hypoplasia-Mental Retardation, Peripheral Neuritis, Peripheral Neuropathy, Peritoneopericardial Diaphragmatic Hernia, Pernicious Anemia, Peromelia with Micrognathia, Peroneal Muscular Atrophy, Peroneal Nerve Palsy, Peroutka Sneeze, Peroxisomal Acyl-CoA Oxidase, Peroxisomal Beta-Oxidation Disorders, Peroxisomal Bifunctional Enzyme, Peroxisomal Thiolase, Peroxisomal Thiolase Deficiency, Persistent Truncus Arteriosus, Perthes Disease, Petit Mal Epilepsy, Petit Mal Variant, Peutz-Jeghers Syndrome, Peutz-Touraine Syndrome, Peyronie Disease, Pfeiffer, Pfeiffer Syndrome Type I, PGA I, PGA II, PGA III, PGK, PH Type I, PH Type I, Pharyngeal Pouch Syndrome, PHD Short-Chain Acyl-CoA Dehydrogenase Deficiency, Phenylalanine Hydroxylase Deficiency, Phenylalaninemia, Phenylketonuria, Phenylpyruvic Oligophrenia, Phocomelia, Phocomelia Syndrome, Phosphoenolpyruvate Carboxykinase Deficiency, Phosphofructokinase Deficiency, Phosphoglycerate Kinase Deficiency, Phosphoglycerokinase, Phosphorylase 6 Kinase Deficiency, Phosphorylase Deficiency Glycogen Storage Disease, Phosphorylase Kinase Deficiency of Liver, Photic Sneeze Reflex, Photic Sneezing, Phototherapeutic keratectomy, PHS, Physicist John Dalton, Phytanic Acid Storage Disease, Pi Phenotype ZZ, PI, Pick Disease of the Brain, Pick's Disease, Pickwickian Syndrome, Pierre Robin Anomalad, Pierre Robin Complex, Pierre Robin Sequence, Pierre Robin Syndrome, Pierre Robin Syndrome with Hyperphalangy and Clinodactyl), Pierre-Marie's Disease, Pigmentary Degeneration of Globus Pallidus Substantia Nigra Red Nucleus, Pili Torti and Nerve Deafness, Pili Torti-Sensorineural Hearing Loss, Pituitary Dwarfism II, Pituitary Tumor after Adrenalectomy, Pityriasis Pilaris, Pityriasis Rubra Pilaris, PJS, PKAN, PKD, PKD1, PKD2, PKD3, PKU, PKU1, Plagiocephaly, Plasma Cell Myeloma, Plasma Cell Leukemia, Plasma Thromboplastin Component Deficiency, Plasma Transglutaminase Deficiency, Plastic Induration Corpora Cavemosa, Plastic Induration of the Penis, PLD, Plicated Tongue, PLS, PMD, Pneumorenal Syndrome, PNH, PNM, PNP Deficiency, POD, POH, Poikiloderma Atrophicans and Cataract, Poikiloderma Congenitale, Poland Anomaly, Poland Sequence, Poland Syndactyl), Poland Syndrome, Poliodystrophia Cerebri Progressiva, Polyarthritis Enterica, Polyarteritis Nodosa, Polyarticular-Onset Juvenile Arthritis Type I, Polyarticular-Onset Juvenile Arthritis Type II, Polyarticular-Onset Juvenile Arthritis Types I and II, Polychondritis, Polycystic Kidney Disease, Polycystic Kidney Disease Medullary Type, Polycystic Liver Disease, Polycystic Ovary Disease, Polycystic Renal Diseases, Polydactyly-Joubert Syndrome, Polydysplastic Epidermolysis Bullosa, Polydystrophia Oligophrenia, Polydystrophic Dwarfism, Polyglandular Autoimmune Syndrome Type III, Polyglandular Autoimmune Syndrome Type II, Polyglandular Autoimmune Syndrome Type I, Polyglandular Autoimmune Syndrome Type II, Polyglandular Deficiency Syndrome Type II, Polyglandular Syndromes, Polymorphic Macula Lutea Degeneration, Polymorphic Macular Degeneration, Polymorphism of Platelet Glycoprotien Ib, Polymorphous Corneal Dystrophy Hereditary, Polymyalgia Rheumatica, Polymyositis and Dermatomyositis, Primary Agammaglobulinemia, Polyneuritis Peripheral, Polyneuropathy-Deafness-Optic Atrophy, Polyneuropathy Peripheral, Polyneuropathy and Polyradiculoneuropathy, Polyostotic Fibrous Dysplasia, Polyostotic Sclerosing Histiocytosis, Polyposis Familial, Polyposis Gardner Type, Polyposis Hamartomatous Intestinal, Polyposis-Osteomatosis-Epidermoid Cyst Syndrome, Polyposis Skin Pigmentation Alopecia and Fingernail Changes, Polyps and Spots Syndrome, Polyserositis Recurrent, Polysomy Y, Polysyndactyl with Peculiar Skull Shape, Polysyndactyly-Dysmorphic Craniofacies Greig Type, Pompe Disease, Pompe Disease, Popliteal Pterygium Syndrome, Porcupine Man, Porencephaly, Porencephaly, Porphobilinogen deaminase (PBG-D), Porphyria, Porphyria Acute Intermittent, Porphyria ALA-D, Porphyria Cutanea Tarda, Porphyria Cutanea Tarda Hereditaria, Porphyria Cutanea Tarda Symptomatica, Porphyria Hepatica Variegate, Porphyria Swedish Type, Porphyria Variegate, Porphyriam Acute Intermittent, Porphyrins, Porrigo Decalvans, Port Wine Stains, Portuguese Type Amyloidosis, Post-Infective Polyneuritis, Postanoxic Intention Myoclonus, Postaxial Acrofacial Dysostosis, Postaxial Polydactyl), Postencephalitic Intention Myoclonus, Posterior Corneal Dystrophy Hereditary, Posterior Thalamic Syndrome, Postmyelographic Arachnoiditis, Postnatal Cerebral Palsy, Postoperative Cholestasis, Postpartum Galactorrhea-Amenorrhea Syndrome, Postpartum Hypopituitarism, Postpartum Panhypopituitary Syndrome, Postpartum Panhypopituitarism, Postpartum Pituitary Necrosis, Postural Hypotension, Potassium-Losing Nephritis, Potassium Loss Syndrome, Potter Type I Infantile Polycystic Kidney Diseases, Potter Type III Polycystic Kidney Disease, PPH, PPS, Prader-Willi Syndrome, Prader-Labhart-Willi Fancone Syndrome, Prealbumin Tyr-77 Amyloidosis, Preexcitation Syndrome, Pregnenolone Deficiency, Premature Atrial Contractions, Premature Senility Syndrome, Premature Supraventricular Contractions, Premature Ventricular Complexes, Prenatal or Connatal Neuroaxonal Dystrophy, Presenile Dementia, Presenile Macula Lutea Retinae Degeneration, Primary Adrenal Insufficiency, Primary Agammaglobulinemias, Primary Aldosteronism, Primary Alveolar Hypoventilation, Primary Amyloidosis, Primary Anemia, Primary Beriberi, Primary Biliary, Primary Biliary Cirrhosis, Primary Brown Syndrome, Primary Carnitine Deficiency, Primary Central Hypoventilation Syndrome, Primary Ciliary Dyskinesia Kartagener Type, Primary Cutaneous Amyloidosis, Primary Dystonia, Primary Failure Adrenocortical Insufficiency, Primary Familial Hypoplasia of the Maxilla, Primary Hemochromatosis, Primary Hyperhidrosis, Primary Hyperoxaluria [Type I], Primary Hyperoxaluria Type 1 (PH1), Primary Hyperoxaluria Type 1, Primary Hyperoxaluria Type II, Primary Hyperoxaluria Type III, Primary Hypogonadism, Primary Intestinal Lymphangiectasia, Primary Lateral Sclerosis, Primary Nonhereditary Amyloidosis, Primary Obliterative Pulmonary Vascular Disease, Primary Progressive Multiple Sclerosis, Primary Pulmonary Hypertension, Primary Reading Disability, Primary Renal Glycosuria, Primary Sclerosing Cholangitis, Primary Thrombocythemia, Primary Tumors of Central Nervous System, Primary Visual Agnosia, Proctocolitis Idiopathic, Proctocolitis Idiopathic, Progeria of Adulthood, Progeria of Childhood, Progeroid Nanism, Progeriod Short Stature with Pigmented Nevi, Progeroid Syndrome of De Barsy, Progressive Autonomic Failure with Multiple System Atrophy, Progressive Bulbar Palsy, Progressive Bulbar Palsy Included, Progressive Cardiomyopathic Lentiginosis, Progressive Cerebellar Ataxia Familial, Progressive Cerebral Poliodystrophy, Progressive Choroidal Atrophy, Progressive Diaphyseal Dysplasia, Progressive Facial Hemiatrophy, Progressive Familial Myoclonic Epilepsy, Progressive Hemifacial Atrophy, Progressive Hypoerythemia, Progressive Infantile Poliodystrophy, Progressive Lenticular Degeneration, Progressive Lipodystrophy, Progressive Muscular Dystrophy of Childhood, Progressive Myoclonic Epilepsy, Progressive Osseous Heteroplasia, Progressive Pallid Degeneration Syndrome, Progressive Spinobulbar Muscular Atrophy, Progressive Supranuclear Palsy, Progressive Systemic Sclerosis, Progressive Tapetochoroidal Dystrophy, Proline Oxidase Deficiency, Propionic Acidemia, Propionic Acidemia Type I (PCCA Deficiency), Propionic Acidemia Type II (PCCB Deficiency), Propionyl CoA Carboxylase Deficiency, Protanomaly, Protanopia, Protein-Losing Enteropathy Secondary to Congestive Heart Failure, Proteus Syndrome, Proximal Deletion of 4q Included, PRP, PRS, Prune Belly Syndrome, PS, Pseudo-Hurler Polydystrophy, Pseudo-Polydystrophy, Pseudoacanthosis Nigricans, Pseudoachondroplasia, Pseudocholinesterase Deficiency, Pseudogout Familial, Pseudohemophilia, Pseudohermaphroditism, Pseudohermaphroditism-Nephron Disorder-Wilm's Tumor, Pseudohypertrophic Muscular Dystrophy, Pseudohypoparathyroidism, Pseudohypophosphatasia, Pseudopolydystrophy, Pseudothalidomide Syndrome, Pseudoxanthoma Elasticum, Psoriasis, Psorospermosis Follicularis, PSP, PSS, Psychomotor Convulsion, Psychomotor Epilepsy, Psychomotor Equivalent Epilepsy, PTC Deficiency, Pterygium, Pterygium Colli Syndrome, Pterygium Universale, Pterygolymphangiectasia, Pulmonary Atresia, Pulmonary Lymphangiomyomatosis, Pulmonary Stenosis, Pulmonic Stenosis-Ventricular Septal Defect, Pulp Stones, Pulpal Dysplasia, Pulseless Disease, Pure Alymphocytosis, Pure Cutaneous Histiocytosis, Purine Nucleoside Phosphorylase Deficiency, Purpura Hemorrhagica, Purtilo Syndrome, PXE, PXE Dominant Type, PXE Recessive Type, Pycnodysostosis, Pyknodysostosis, Pyknoepilepsy, Pyroglutamic Aciduria, Pyroglutamicaciduria, Pyrroline Carboxylate Dehydrogenase Deficiency, Pyruvate Carboxylase Deficiency, Pyruvate Carboxylase Deficiency Group A, Pyruvate Carboxylase Deficiency Group B, Pyruvate Dehydrogenase Deficiency, Pyruvate Kinase Deficiency, q25-qter, q26 or q27-qter, q31 or 32-qter, QT Prolongation with Extracellular Hypohypocalcinemia, QT Prolongation without Congenital Deafness, QT Prolonged with Congenital Deafness, Quadriparesis of Cerebral Palsy, Quadriplegia of Cerebral Palsy, Quantal Squander, Quantal Squander, r4, r6, r14, r18, r21, r22, Rachischisis Posterior, Radial Aplasia-Amegakaryocytic Thrombocytopenia, Radial Aplasia-Thrombocytopenia Syndrome, Radial Nerve Palsy, Radicular Neuropathy Sensory, Radicular Neuropathy Sensory Recessive, Radicular Dentin Dysplasia, Rapid-onset Dystonia-parkinsonism, Rapp-Hodgkin Syndrome, Rapp-Hodgkin (hypohidrotic) Ectodermal Dysplasia syndrome, Rapp-Hodgkin Hypohidrotic Ectodermal Dysplasias, Rare hereditary ataxia with polyneuritic changes and deafness caused by a defect in the enzyme phytanic acid hydroxylase, Rautenstrauch-Wiedemann Syndrome, Rautenstrauch-Wiedemann Type Neonatal Progeria, Raynaud's Phenomenon, RDP, Reactive Functional Hypoglycemia, Reactive Hypoglycemia Secondary to Mild Diabetes, Recessive Type Kenny-Caffe Syndrome, Recklin Recessive Type Myotonia Congenita, Recklinghausen Disease, Rectoperineal Fistula, Recurrent Vomiting, Reflex Neurovascular Dystrophy, Reflex Sympathetic Dystrophy Syndrome, Refractive Errors, Refractory Anemia, Refrigeration Palsy, Refsum Disease, Refsum's Disease, Regional Enteritis, Reid-Barlow's syndrome, Reifenstein Syndrome, Reiger Anomaly-Growth Retardation, Reiger Syndrome, Reimann Periodic Disease, Reimann's Syndrome, Reis-Bucklers Corneal Dystrophy, Reiter's Syndrome, Relapsing Guillain-Barre Syndrome, Relapsing-Remitting Multiple Sclerosis, Renal Agenesis, Renal Dysplasia-Blindness Hereditary, Renal Dysplasia-Retinal Aplasia Loken-Senior Type, Renal Glycosuria, Renal Glycosuria Type A, Renal Glycosuria Type B, Renal Glycosuria Type O, Renal-Oculocerebrodystrophy, Renal-Retinal Dysplasia with Medullary Cystic Disease, Renal-Retinal Dystrophy Familial, Renal-Retinal Syndrome, Rendu-Osler-Weber Syndrome, Respiratory Acidosis, Respiratory Chain Disorders, Respiratory Myoclonus, Restless Legs Syndrome, Restrictive Cardio myopathy, Retention Hyperlipemia, Rethore Syndrome (obsolete), Reticular Dysgenesis, Retinal Aplastic-Cystic Kidneys-Joubert Syndrome, Retinal Cone Degeneration, Retinal Cone Dystrophy, Retinal Cone-Rod Dystrophy, Retinitis Pigmentosa, Retinitis Pigmentosa and Congenital Deafness, Retinoblastoma, Retinol Deficiency, Retinoschisis, Retinoschisis Juvenile, Retraction Syndrome, Retrobulbar Neuropathy, Retrolenticular Syndrome, Rett Syndrome, Reverse Coarction, Reye Syndrome, Reye's Syndrome, RGS, Rh Blood Factors, Rh Disease, Rh Factor Incompatibility, Rh Incompatibility, Rhesus Incompatibility, Rheumatic Fever, Rheumatoid Arthritis, Rheumatoid Myositis, Rhinosinusogenic Cerebral Arachnoiditis, Rhizomelic Chondrodysplasia Punctata (RCDP), Acatalasemia, Classical Refsum disease, RHS, Rhythmical Myoclonus, Rib Gap Defects with Micrognathia, Ribbing Disease (obsolete), Ribbing Disease, Richner-Hanhart Syndrome, Rieger Syndrome, Rieter's Syndrome, Right Ventricular Fibrosis, Riley-Day Syndrome, Riley-Smith syndrome, Ring Chromosome 14, Ring Chromosome 18, Ring 4, Ring 4 Chromosome, Ring 6, Ring 6 Chromosome, Ring 9, Ring 9 Chromosome R9, Ring 14, Ring 15, Ring 15 Chromosome (mosaic pattern), Ring 18, Ring Chromosome 18, Ring 21, Ring 21 Chromosome, Ring 22, Ring 22 Chromosome, Ritter Disease, Ritter-Lyell Syndrome, RLS, RMSS, Roberts SC-Phocomelia Syndrome, Roberts Syndrome, Roberts Tetraphocomelia Syndrome, Robertson's Ectodermal Dysplasias, Robin Anomalad, Robin Sequence, Robin Syndrome, Robinow Dwarfism, Robinow Syndrome, Robinow Syndrome Dominant Form, Robinow Syndrome Recessive Form, Rod myopathy, Roger Disease, Rokitansky's Disease, Romano-Ward Syndrome, Romberg Syndrome, Rootless Teeth, Rosenberg-Chutorian Syndrome, Rosewater Syndrome, Rosselli-Gulienatti Syndrome, Rothmund-Thomson Syndrome, Roussy-Levy Syndrome, RP, RS X-Linked, RS, RSDS, RSH Syndrome, RSS, RSTS, RTS, Rubella Congenital, Rubinstein Syndrome, Rubinstein-Taybi Syndrome, Rubinstein Taybi Broad Thumb-Hallux syndrome, Rufous Albinism, Ruhr's Syndrome, Russell's Diencephalic Cachexia, Russell's Syndrome, Russell Syndrome, Russell-Silver Dwarfism, Russell-Silver Syndrome, Russell-Silver Syndrome X-linked, Ruvalcaba-Myhre-Smith syndrome (RMSS), Ruvalcaba Syndrome, Ruvalcaba Type Osseous Dysplasia with Mental Retardation, Sacral Regression, Sacral Agenesis Congenital, SAE, Saethre-Chotzen Syndrome, Sakati, Sakati Syndrome, Sakati-Nyhan Syndrome, Salaam Spasms, Salivosudoriparous Syndrome, Salzman Nodular Corneal Dystrophy, Sandhoff Disease, Sanfilippo Syndrome, Sanfilippo Type A, Sanfilippo Type B, Santavuori Disease, Santavuori-Haltia Disease, Sarcoid of Boeck, Sarcoidosis, Sathre-chotzen, Saturday Night Palsy, SBMA, SC Phocomelia Syndrome, SC Syndrome, SCA 3, SCAD Deficiency, SCAD Deficiency Adult-Onset Localized, SCAD Deficiency Congenital Generalized, SCAD, SCADH Deficiency, Scalded Skin Syndrome, Scalp Defect Congenital, Scaphocephaly, Scapula Elevata, Scapuloperoneal myopathy, Scapuloperoneal Muscular Dystrophy, Scapuloperoneal Syndrome Myopathic Type, Scarring Bullosa, SCHAD, Schaumann's Disease, Scheie Syndrome, Schereshevkii-Turner Syndrome, Schilder Disease, Schilder Encephalitis, Schilder's Disease, Schindler Disease Type I (Infantile Onset), Schindler Disease Infantile Onset, Schindler Disease, Schindler Disease Type II (Adult Onset), Schinzel Syndrome, Schinzel-Giedion Syndrome, Schinzel Acrocallosal Syndrome, Schinzel-Giedion Midface-Retraction Syndrome, Schizencephaly, Schizophrenia, Schmid Type Metaphyseal Chondrodysplasia, Schmid Metaphyseal Dysostosis, Schmid-Fraccaro Syndrome, Schmidt Syndrome, Schopf-Schultz-Passarge Syndrome, Schueller-Christian Disease, Schut-Haymaker Type, Schwartz-Jampel-Aberfeld Syndrome, Schwartz-Jampel Syndrome Types 1A and 1B, Schwartz-Jampel Syndrome, Schwartz-Jampel Syndrome Type 2, SCID, Scleroderma, Sclerosis Familial Progressive Systemic, Sclerosis Diffuse Familial Brain, Sciatic Nerve Crush, Scott Craniodigital Syndrome With Mental Retardation, Scrotal Tongue, SCS, SD, SDS, SDYS, Seasonal Conjunctivitis, Sebaceous Nevus Syndrome, Sebaceous nevus, Seborrheic Keratosis, Seborrheic Warts, Seckel Syndrome, Seckel Type Dwarfism, Second Degree Congenital Heart Block, Secondary Amyloidosis, Secondary Blepharospasm, Secondary Non-tropical Sprue, Secondary Brown Syndrome, Secondary Beriberi, Secondary Generalized Amyloidosis, Secondary Dystonia, Secretory Component Deficiency, Secretory IgA Deficiency, SED Tarda, SED Congenital, SEDC, Segmental linear achromic nevus, Segmental Dystonia, Segmental Myoclonus, Seip Syndrome, Seitelberger Disease, Seizures, Selective Deficiency of IgG Subclasses, Selective Mutism, Selective Deficiency of IgG Subclass, Selective IgM Deficiency, Selective Mutism, Selective IgA Deficiency, Self-Healing Histiocytosis, Semilobar Holoprosencephaly, Seminiferous Tubule Dysgenesis, Senile Retinoschisis, Senile Warts, Senior-Loken Syndrome, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Septic Progressive Granulomatosis, Septo-Optic Dysplasia, Serous Circumscribed Meningitis, Serum Protease Inhibitor Deficiency, Serum Camosinase Deficiency, Setleis Syndrome, Severe Combined Immunodeficiency, Severe Combined Immunodeficiency with Adenosine Deaminase Deficiency, Severe Combined Immunodeficiency (SCID), Sex Reversal, Sexual Infantilism, SGB Syndrome, Sheehan Syndrome, Shields Type Dentinogenesis Imperfecta, Shingles, varicella-zoster virus, Ship Beriberi, SHORT Syndrome, Short Arm 18 Deletion Syndrome, Short Chain Acyl CoA Dehydrogenase Deficiency, Short Chain Acyl-CoA Dehydrogenase (SCAD) Deficiency, Short Stature and Facial Telangiectasis, Short Stature Facial/Skeletal Anomalies-Retardation-Macrodontia, Short Stature-Hyperextensibility-Rieger Anomaly-Teething Delay, Short Stature-Onychodysplasia, Short Stature Telangiectatic Erythema of the Face, SHORT Syndrome, Shoshin Beriberi, Shoulder girdle syndrome, Shprintzen-Goldberg Syndrome, Shulman Syndrome, Shwachman-Bodian Syndrome, Shwachman-Diamond Syndrome, Shwachman Syndrome, Shwachman-Diamond-Oski Syndrome, Shwachmann Syndrome, Shy Drager Syndrome, Shy-Magee Syndrome, SI Deficiency, Sialidase Deficiency, Sialidosis Type I Juvenile, Sialidosis Type II Infantile, Sialidosis, Sialolipidosis, Sick Sinus Syndrome, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Sideroblastic Anemias, Sideroblastic Anemia, Sideroblastosis, SIDS, Siegel-Cattan-Mamou Syndrome, Siemens-Bloch type Pigmented Dermatosis, Siemens Syndrome, Siewerling-Creutzfeldt Disease, Siewert Syndrome, Silver Syndrome, Silver-Russell Dwarfism, Silver-Russell Syndrome, Simmond's Disease, Simons Syndrome, Simplex Epidermolysis Bullosa, Simpson Dysmorphia Syndrome, Simpson-Golabi-Behmel Syndrome, Sinding-Larsen-Johansson Disease, Singleton-Merten Syndrome, Sinus Arrhythmia, Sinus Venosus, Sinus tachycardia, Sirenomelia Sequence, Sirenomelus, Situs Inversus Bronchiectasis and Sinusitis, SJA Syndrome, Sjogren Larsson Syndrome Ichthyosis, Sjogren Syndrome, Sjogren's Syndrome, SJS, Skeletal dysplasia, Skeletal Dysplasia Weismann Netter Stuhl Type, Skin Peeling Syndrome, Skin Neoplasms, Skull Asymmetry and Mild Retardation, Skull Asymmetry and Mild Syndactyl), SLE, Sleep Epilepsy, Sleep Apnea, SLO, Sly Syndrome, SMA, SMA Infantile Acute Form, SMA I, SMA III, SMA type I, SMA type II, SMA type III, SMA3, SMAX1, SMCR, Smith Lemli Opitz Syndrome, Smith Magenis Syndrome, Smith-Magenis Chromosome Region, Smith-McCort Dwarfism, Smith-Opitz-Inbom Syndrome, Smith Disease, Smoldering Myeloma, SMS, SNE, Sneezing From Light Exposure, Sodium valproate, Solitary Plasmacytoma of Bone, Sorsby Disease, Sotos Syndrome, Souques-Charcot Syndrome, South African Genetic Porphyria, Spasmodic Dysphonia, Spasmodic Torticollis, Spasmodic Wryneck, Spastic Cerebral Palsy, Spastic Colon, Spastic Dysphonia, Spastic Paraplegia, SPD Calcinosis, Specific Antibody Deficiency with Normal Immunoglobulins, Specific Reading Disability, SPH2, Spherocytic Anemia, Spherocytosis, Spherophakia-Brachymorphia Syndrome, Sphingomyelin Lipidosis, Sphingomyelinase Deficiency, Spider fingers, Spielmeyer-Vogt Disease, Spielmeyer-Vogt-Batten Syndrome, Spina Bifida, Spina Bifida Aperta, Spinal Arachnoiditis, Spinal Arteriovenous Malformation, Spinal Ataxia Hereditofamilial, Spinal and Bulbar Muscular Atrophy, Spinal Cord Crush, Spinal Diffuse Idiopathic Skeletal Hyperostosis, Spinal DISH, Spinal Muscular Atrophy, Spinal Muscular Atrophy All Types, Spinal Muscular Atrophy Type ALS, Spinal Muscular Atrophy-Hypertrophy of the Calves, Spinal Muscular Atrophy Type I, Spinal Muscular Atrophy Type III, Spinal Muscular Atrophy type 3, Spinal Muscular Atrophy-Hypertrophy of the Calves, Spinal Ossifying Arachnoiditis, Spinal Stenosis, Spino Cerebellar Ataxia, Spinocerebellar Atrophy Type I, Spinocerebellar Ataxia Type I (SCAI), Spinocerebellar Ataxia Type II (SCAII), Spinocerebellar Ataxia Type III (SCAIII), Spinocerebellar Ataxia Type III (SCA 3), Spinocerebellar Ataxia Type IV (SCAIV), Spinocerebellar Ataxia Type V (SCAV), Spinocerebellar Ataxia Type VI (SCAVI), Spinocerebellar Ataxia Type VII (SCAVII), Spirochetal Jaundice, Splenic Agenesis Syndrome, Splenic Ptosis, Splenoptosis, Split Hand Deformity-Mandibulofacial Dysostosis, Split Hand Deformity, Spondyloarthritis, Spondylocostal Dysplasia—Type I, Spondyloepiphyseal Dysplasia Tarda, Spondylothoracic Dysplasia, Spondylotic Caudal Radiculopathy, Sponge Kidney, Spongioblastoma Multiforme, Spontaneous Hypoglycemia, Sprengel Deformity, Spring Ophthalmia, SRS, ST, Stale Fish Syndrome, Staphyloccal Scalded Skin Syndrome, Stargardt's Disease, Startle Disease, Status Epilepticus, Steele-Richardson-Olszewski Syndrome, Steely Hair Disease, Stein-Leventhal Syndrome, Steinert Disease, Stengel's Syndrome, Stengel-Batten-Mayou-Spielmeyer-Vogt-Stock Disease, Stenosing Cholangitis, Stenosis of the Lumbar Vertebral Canal, Stenosis, Steroid Sulfatase Deficiency, Stevanovic's Ectodermal Dysplasias, Stevens Johnson Syndrome, STGD, Stickler Syndrome, Stiff-Man Syndrome, Stiff Person Syndrome, Still's Disease, Stilling-Turk-Duane Syndrome, Stillís Disease, Stimulus-Sensitive Myoclonus, Stone Man Syndrome, Stone Man, Streeter Anomaly, Striatonigral Degeneration Autosomal Dominant Type, Striopallidodentate Calcinosis, Stroma, Descemet's Membrane, Stromal Corneal Dystrophy, Struma Lymphomatosa, Sturge-Kalischer-Weber Syndrome, Sturge Weber Syndrome, Sturge-Weber Phakomatosis, Subacute Necrotizing Encephalomyelopathy, Subacute Spongiform Encephalopathy, Subacute Necrotizing Encephalopathy, Subacute Sarcoidosis, Subacute Neuronopathic, Subaortic Stenosis, Subcortical Arteriosclerotic Encephalopathy, Subendocardial Sclerosis, Succinylcholine Sensitivity, Sucrase-Isomaltase Deficiency Congenital, Sucrose-Isomaltose Malabsorption Congenital, Sucrose Intolerance Congenital, Sudanophilic Leukodystrophy ADL, Sudanophilic Leukodystrophy Pelizaeus-Merzbacher Type, Sudanophilic Leukodystrophy Included, Sudden Infant Death Syndrome, Sudeck's Atrophy, Sugio-Kajii Syndrome, Summerskill Syndrome, Summit Acrocephalosyndactyl), Summitt's Acrocephalosyndactyl), Summitt Syndrome, Superior Oblique Tendon Sheath Syndrome, Suprarenal glands, Supravalvular Aortic Stenosis, Supraventricular tachycardia, Surdicardiac Syndrome, Surdocardiac Syndrome, SVT, Sweat Gland Abscess, Sweating Gustatory Syndrome, Sweet Syndrome, Swiss Cheese Cartilage Syndrome, Syndactylic Oxycephaly, Syndactyly Type I with Microcephaly and Mental Retardation, Syndromatic Hepatic Ductular Hypoplasia, Syringomyelia, Systemic Aleukemic Reticuloendotheliosis, Systemic Amyloidosis, Systemic Carnitine Deficiency, Systemic Elastorrhexis, Systemic Lupus Erythematosus, Systemic Mast Cell Disease, Systemic Mastocytosis, Systemic-Onset Juvenile Arthritis, Systemic Sclerosis, Systopic Spleen, T-Lymphocyte Deficiency, Tachyalimentation Hypoglycemia, Tachycardia, Takahara syndrome, Takayasu Disease, Takayasu Arteritis, Talipes Calcaneus, Talipes Equinovarus, Talipes Equinus, Talipes Varus, Talipes Valgus, Tandem Spinal Stenosis, Tangier Disease, Tapetoretinal Degeneration, TAR Syndrome, Tardive Dystonia, Tardive Muscular Dystrophy, Tardive Dyskinesia, Tardive Oral Dyskinesia, Tardive Dystonia, Tardy Ulnar Palsy, Target Cell Anemia, Tarsomegaly, Tarui Disease, TAS Midline Defects Included, TAS Midline Defect, Tay Sachs Sphingolipidosis, Tay Sachs Disease, Tay Syndrome Ichthyosis, Tay Sachs Sphingolipidosis, Tay Syndrome Ichthyosis, Taybi Syndrome Type I, Taybi Syndrome, TCD, TCOF1, TCS, TD, TDO Syndrome, TDO-I, TDO-II, TDO-III, Telangiectasis, Telecanthus with Associated Abnormalities, Telecanthus-Hypospadias Syndrome, Temporal Lobe Epilepsy, Temporal Arteritis/Giant Cell Arteritis, Temporal Arteritis, TEN, Tendon Sheath Adherence Superior Obliqu, Tension Myalgia, Terminal Deletion of 4q Included, Terrian Corneal Dystrophy, Teschler-Nicola/Killian Syndrome, Tethered Spinal Cord Syndrome, Tethered Cord Malformation Sequence, Tethered Cord Syndrome, Tethered Cervical Spinal Cord Syndrome, Tetrahydrobiopterin Deficiencies, Tetrahydrobiopterin Deficiencies, Tetralogy of Fallot, Tetraphocomelia-Thrombocytopenia Syndrome, Tetrasomy Short Arm of Chromosome 9, Tetrasomy 9p, Tetrasomy Short Arm of Chromosome 18, Thalamic Syndrome, Thalamic Pain Syndrome, Thalamic Hyperesthetic Anesthesia, Thalassemia Intermedia, Thalassemia Minor, Thalassemia Major, Thiamine Deficiency, Thiamine-Responsive Maple Syrup Urine Disease, Thin-Basement-Membrane Nephropathy, Thiolase deficiency, RCDP, Acyl-CoA dihydroxyacetonephosphate acyltransferase, Third and Fourth Pharyngeal Pouch Syndrome, Third Degree Congenital (Complete) Heart Block, Thomsen Disease, Thoracic-Pelvic-Phalangeal Dystrophy, Thoracic Spinal Canal, Thoracoabdominal Syndrome, Thoracoabdominal Ectopia Cordis Syndrome, Three M Syndrome, Three-M Slender-Boned Nanism, Thrombasthenia of Glanzmann and Naegeli, Thrombocythemia Essential, Thrombocytopenia-Absent Radius Syndrome, Thrombocytopenia-Hemangioma Syndrome, Thrombocytopenia-Absent Radii Syndrome, Thrombophilia Hereditary Due to AT III, Thrombotic Thrombocytopenic Purpura, Thromboulcerative Colitis, Thymic Dysplasia with Normal Immunoglobulins, Thymic Agenesis, Thymic Aplasia DiGeorge Type, Thymic Hypoplasia Agammaglobulinemias Primary Included, Thymic Hypoplasia DiGeorge Type, Thymus Congenital Aplasia, Tic Douloureux, Tics, Tinel's syndrome, Tolosa Hunt Syndrome, Tonic Spasmodic Torticollis, Tonic Pupil Syndrome, Tooth and Nail Syndrome, Torch Infection, TORCH Syndrome, Torsion Dystonia, Torticollis, Total Lipodystrophy, Total anomalous pulmonary venous connection, Touraine's Aphthosis, Tourette Syndrome, Tourette's disorder, Townes-Brocks Syndrome, Townes Syndrome, Toxic Paralytic Anemia, Toxic Epidermal Necrolysis, Toxopachyosteose Diaphysaire Tibio-Peroniere, Toxopachyosteose, Toxoplasmosis Other Agents Rubella Cytomegalovirus Herpes Simplex, Tracheoesophageal Fistula with or without Esophageal Atresia, Tracheoesophageal Fistula, Transient neonatal myasthenia gravis, Transitional Atrioventricular Septal Defect, Transposition of the great arteries, Transtelephonic Monitoring, Transthyretin Methionine-30 Amyloidosis (Type I), Trapezoidocephaly-Multiple Synostosis Syndrome, Treacher Collins Syndrome, Treacher Collins-Franceschetti Syndrome 1, Trevor Disease, Triatrial Heart, Tricho-Dento-Osseous Syndrome, Trichodento Osseous Syndrome, Trichopoliodystrophy, Trichorhinophalangeal Syndrome, Trichorhinophalangeal Syndrome, Tricuspid atresia, Trifunctional Protein Deficiency, Trigeminal Neuralgia, Triglyceride Storage Disease Impaired Long-Chain Fatty Acid Oxidation, Trigonitis, Trigonocephaly, Trigonocephaly Syndrome, Trigonocephaly “C” Syndrome, Trimethylaminuria, Triphalangeal Thumbs-Hypoplastic Distal Phalanges-Onychodystrophy, Triphalangeal Thumb Syndrome, Triple Symptom Complex of Behcet, Triple X Syndrome, Triplo X Syndrome, Triploid Syndrome, Triploidy, Triploidy Syndrome, Trismus-Pseudocamptodactyly Syndrome, Trisomy, Trisomy G Syndrome, Trisomy X, Trisomy 6q Partial, Trisomy 6q Syndrome Partial, Trisomy 9 Mosaic, Trisomy 9P Syndrome (Partial) Included, Trisomy 11q Partial, Trisomy 14 Mosaic, Trisomy 14 Mosaicism Syndrome, Trisomy 21 Syndrome, Trisomy 22 Mosaic, Trisomy 22 Mosaicism Syndrome, TRPS, TRPS1, TRPS2, TRPS3, True Hermaphroditism, Truncus arteriosus, Tryptophan Malabsorption, Tryptophan Pyrrolase Deficiency, TS, TTP, TTTS, Tuberous Sclerosis, Tubular Ectasia, Turcot Syndrome, Turner Syndrome, Turner-Kieser Syndrome, Turner Phenotype with Normal Chromosomes (Karyotype), Turner-Varny Syndrome, Turricephaly, Twin-Twin Transfusion Syndrome, Twin-to-Twin Transfusion Syndrome, Type A, Type B, Type AB, Type O, Type I Diabetes, Type I Familial Incomplete Male, Type I Familial Incomplete Male Pseudohermaphroditism, Type I Gaucher Disease, Type I (PCCA Deficiency), Type I Tyrosinemia, Type II Gaucher Disease, Type II Histiocytosis, Type II (PCCB Deficiency), Type II Tyrosinnemia, Type IIA Distal Arthrogryposis Multiplex Congenita, Type III Gaucher Disease, Type III Tyrosinemia, Type III Dentinogenesis Imperfecta, Typical Retinoschisis, Tyrosinase Negative Albinism (Type I), Tyrosinase Positive Albinism (Type II), Tyrosinemia type 1 acute form, Tyrosinemia type 1 chronic form, Tyrosinosis, UCE, Ulcerative Colitis, Ulcerative Colitis Chronic Non-Specific, Ulnar-Mammary Syndrome, Ulnar-Mammary Syndrome of Pallister, Ulnar Nerve Palsy, UMS, Unclassified FODs, Unconjugated Benign Bilirubinemiav, Underactivity of Parathyroid, Unilateral Ichthyosiform Erythroderma with Ipsilateral Malformations Limb, Unilateral Chondromatosis, Unilateral Defect of Pectoralis Muscle and Syndactyly of the Hand, Unilateral Hemidysplasia Type, Unilateral Megalencephaly, Unilateral Partial Lipodystrophy, Unilateral Renal Agenesis, Unstable Colon, Unverricht Disease, Unverricht-Lundborg Disease, Unverricht-Lundborg-Laf Disease, Unverricht Syndrome, Upper Limb—Cardiovascular Syndrome (Holt-Oram), Upper Motor Neuron Disease, Upper Airway Apnea, Urea Cycle Defects or Disorders, Urea Cycle Disorder Arginase Type, Urea Cycle Disorder Arginino Succinase Type, Urea Cycle Disorders Carbamyl Phosphate Synthetase Type, Urea Cycle Disorder Citrullinemia Type, Urea Cycle Disorders N-Acrtyl Glutamate Synthetase Typ, Urea Cycle Disorder OTC Type, Urethral Syndrome, Urethro-Oculo-Articular Syndrome, Uridine Diphosphate Glucuronosyltransferase Severe Def. Type I, Urinary Tract Defects, Urofacial Syndrome, Uroporphyrinogen III cosynthase, Urticaria pigmentosa, Usher Syndrome, Usher Type I, Usher Type II, Usher Type III, Usher Type IV, Uterine Synechiae, Uoporphyrinogen I-synthase, Uveitis, Uveomeningitis Syndrome, V-CJD, VACTEL Association, VACTERL Association, VACTERL Syndrome, Valgus Calcaneus, Valine Transaminase Deficiency, Valinemia, Valproic Acid, Valproate acid exposure, Valproic acid exposure, Valproic acid, Van Buren's Disease, Van der Hoeve-Habertsma-Waardenburg-Gauldi Syndrome, Variable Onset Immunoglobulin Deficiency Dysgammaglobulinemia, Variant Creutzfeldt-Jakob Disease (V-CJD), Varicella Embryopathy, Variegate Porphyria, Vascular Birthmarks, Vascular Dementia Binswanger's Type, Vascular Erectile Tumor, Vascular Hemophilia, Vascular Malformations, Vascular Malformations of the Brain, Vasculitis, Vasomotor Ataxia, Vasopressin-Resistant Diabetes Insipidus, Vasopressin-Sensitive Diabetes Insipidus, VATER Association, Vcf syndrome, Vcfs, Velocardiofacial Syndrome, VeloCardioFacial Syndrome, Venereal Arthritis, Venous Malformations, Ventricular Fibrillation, Ventricular Septal Defects, Congenital Ventricular Defects, Ventricular Septal Defect, Ventricular Tachycardia, Venual Malformations, VEOHD, Vermis Aplasia, Vermis Cerebellar Agenesis, Vernal Keratoconjunctivitis, Verruca, Vertebral Anal Tracheoesophageal Esophageal Radial, Vertebral Ankylosing Hyperostosis, Very Early Onset Huntington's Disease, Very Long Chain Acyl-CoA Dehydrogenase (VLCAD) Deficiency, Vestibular Schwannoma, Vestibular Schwannoma Neurofibromatosis, Vestibulocerebellar, Virchow's Oxycephaly, Visceral Xanthogranulomatosis, Visceral Xantho-Granulomatosis, Visceral myopathy-External Opthalmoplegia, Visceromegaly-Umbilical Hemia-Macroglossia Syndrome, Visual Amnesia, Vitamin A Deficiency, Vitamin B-1 Deficiency, Vitelline Macular Dystrophy, Vitiligo, Vitiligo Capitis, Vitreoretinal Dystrophy, VKC, VKH Syndrome, VLCAD, Vogt Syndrome, Vogt Cephalosyndactyl), Vogt Koyanagi Harada Syndrome, Von Bechterew-Strumpell Syndrome, Von Eulenburg Paramyotonia Congenita, Von Frey's Syndrome, Von Gierke Disease, Von Hippel-Lindau Syndrome, Von Mikulicz Syndrome, Von Recklinghausen Disease, Von Willebrandt Disease, VP, Vrolik Disease (Type II), VSD, Vulgaris Type Disorder of Cornification, Vulgaris Type Ichthyosis, W Syndrome, Waardenburg Syndrome, Waardenburg-Klein Syndrome, Waardenburg Syndrome Type I (WS1), Waardenburg Syndrome Type II (WS2), Waardenburg Syndrome Type IIA (WS2A), Waardenburg Syndrome Type IIB (WS2B), Waardenburg Syndrome Type III (WS3), Waardenburg Syndrome Type IV (WS4), Waelsch's Syndrome, WAGR Complex, WAGR Syndrome, Waldenstroem's Macroglobulinemia, Waldenstrom's Purpura, Waldenstrom's Syndrome, Waldmann Disease, Walker-Warburg Syndrome, Wandering Spleen, Warburg Syndrome, Warm Antibody Hemolytic Anemia, Warm Reacting Antibody Disease, Wartenberg Syndrome, WAS, Water on the Brain, Watson Syndrome, Watson-Alagille Syndrome, Waterhouse-Friderichsen syndrome, Waxy Disease, WBS, Weaver Syndrome, Weaver-Smith Syndrome, Weber-Cockayne Disease, Wegener's Granulomatosis, Weil Disease, Weil Syndrome, Weill-Marchesani, Weill-Marchesani Syndrome, Weill-Reyes Syndrome, Weismann-Netter-Stuhl Syndrome, Weissenbacher-Zweymuller Syndrome, Wells Syndrome, Wenckebach, Werdnig-Hoffman Disease, Werdnig-Hoffman Paralysis, Werlhof's Disease, Werner Syndrome, Wernicke's (C) I Syndrome, Wernicke's aphasia, Wernicke-Korsakoff Syndrome, West Syndrome, Wet Beriberi, WHCR, Whipple's Disease, Whipple Disease, Whistling face syndrome, Whistling Face-Windmill Vane Hand Syndrome, White-Darier Disease, Whitnall-Norman Syndrome, Whorled nevoid hypermelanosis, WHS, Wieacker Syndrome, Wieacher Syndrome, Wieacker-Wolff Syndrome, Wiedmann-Beckwith Syndrome, Wiedemann-Rautenstrauch Syndrome, Wildervanck Syndrome, Willebrand-Juergens Disease, Willi-Prader Syndrome, Williams Syndrome, Williams-Beuren Syndrome, Wilms' Tumor, Wilms' Tumor-Aniridia-Gonadoblastoma-Mental Retardation Syndrome, Wilms Tumor Aniridia Gonadoblastoma Mental Retardation, Wilms' Tumor-Aniridia-Genitourinary Anomalies-Mental Retardation Syndrome, Wilms Tumor-Pseudohermaphroditism-Nephropathy, Wilms Tumor and Pseudohermaphroditism, Wilms Tumor-Pseuodohermaphroditism-Glomerulopathy, Wilson's Disease, Winchester Syndrome, Winchester-Grossman Syndrome, Wiskott-Aldrich Syndrome, Wiskott-Aldrich Type Immunodeficiency, Witkop Ectodermal Dysplasias, Witkop Tooth-Nail Syndrome, Wittmaack-Ekbom Syndrome, WM Syndrome, WMS, WNS, Wohlfart-Disease, Wohlfart-Kugelberg-Welander Disease, Wolf Syndrome, Wolf-Hirschhorn Chromosome Region (WHCR), Wolf-Hirschhom Syndrome, Wolff-Parkinson-White Syndrome, Wolfram Syndrome, Wolman Disease (Lysomal Acid Lypase Deficiency), Woody Guthrie's Disease, WPW Syndrome, Writer's Cramp, WS, WSS, WWS, Wybumn-Mason Syndrome, X-Linked Addison's Disease, X-linked Adrenoleukodystrophy (X-ALD), X-linked Adult Onset Spinobulbar Muscular Atrophy, X-linked Adult Spinal Muscular Atrophy, X-Linked Agammaglobulinemia with Growth Hormone Deficiency, X-Linked Agammaglobulinemia, Lymphoproliferate X-Linked Syndrome, X-linked Cardio myopathy and Neutropenia, X-Linked Centronuclear myopathy, X-linked Copper Deficiency, X-linked Copper Malabsorption, X-Linked Dominant Conradi-Hunermann Syndrome, X-Linked Dominant Inheritance Agenesis of Corpus Callosum, X-Linked Dystonia-parkinsonism, X Linked Ichthyosis, X-Linked Infantile Agammaglobulinemia, X-Linked Infantile Nectrotizing Encephalopathy, X-linked Juvenile Retinoschisis, X-linked Lissencephaly, X-linked Lymphoproliferative Syndrome, X-linked Mental Retardation-Clasped Thumb Syndrome, X-Linked Mental Retardation with Hypotonia, X-linked Mental Retardation and Macroorchidism, X-Linked Progressive Combined Variable Immunodeficiency, X-Linked Recessive Conradi-Hunermann Syndrome, X-Linked Recessive Severe Combined Immunodeficiency, X-Linked Retinoschisis, X-linked Spondyloepiphyseal Dysplasia, Xanthine Oxidase Deficiency (Xanthinuria Deficiency, Hereditary), Xanthinuria Deficiency, Hereditary (Xanthine Oxidase Deficiency), Xanthogranulomatosis Generalized, Xanthoma Tuberosum, Xeroderma Pigmentosum, Xerodemia Pigmentosum Dominant Type, Xeroderma Pigmentosum Type A I XPA Classical Form, Xeroderma Pigmentosum Type B II XPB, Xeroderma Pigmentosum Type E V XPE, Xeroderma Pigmentosum Type C III XPC, Xeroderma Pigmentosum Type D IV XPD, Xeroderma Pigmentosum Type F VI XPF, Xeroderma Pigmentosum Type G VII XPG, Xeroderma Pigmentosum Variant Type XP-V, Xeroderma-Talipes- and Enamel Defect, Xerodermic Idiocy, Xerophthalmia, Xerotic Keratitis, XLP, XO Syndrome, XP, XX Male Syndrome, Sex Reversal, XXXXX Syndrome, XXY Syndrome, XYY Syndrome, XYY Chromosome Pattern, Yellow Mutant Albinism, Yellow Nail Syndrome, YKL, Young Female Arteritis, Yunis-Varon Syndrome, YY Syndrome, Z-E Syndrome, Z- and -Protease Inhibitor Deficiency, Zellweger Syndrome, Zellweger cerebro-hepato-renal syndrome, ZES, Ziehen-Oppenheim Disease (Torsion Dystonia), Zimmermann-Laband Syndrome, Zinc Deficiency Congenital, Zinsser-Cole-Engman Syndrome, ZLS, Zollinger-Ellison Syndrome.

In another embodiment, the pharmaceutical composition comprising an isolated GM-CSF or chimeric molecule thereof can be used, alone or in conjunction with other biologics, drugs or therapies (e.g. surgery, radiotherapy, bone marrow transplantation, peripheral stem cell transplantation; conventional chemotherapy, combined chemotherapy including cisplatin, streptozocin, doxorubicin, fluorouracil, mitomycin C, busulfan, oblimersen, etoposide, vincristine, cyclophosphamide, gemcitabine, pacitaxel, taxol, cytabarine; antivirals including didanosine, AZT, ribavirin, viramidine, zidovudine, dideoycytidine; other therapies including antibiotics, cytokines, growth factor, small molecule pharmaceuticals) for several types of leukaemia, including acute myeloid leukaemia and acute lymphoblastic leukaemia, and various pathologies associated with anti-cancer chemotherapy, such as leukopenia, (a reduction in leukocytes); neutropenia, including febrile neutropenia in patients receiving chemotherapy for acute myeloid leukaemia or in other high risk patients, neutropenia after consolidation chemotherapy for acute myeloid leukaemia and in myelodysplastic syndrome, induced neutropenia in HIV patients with Kaposi sarcoma; facilitating engraftment in bone marrow or peripheral stem cell transplants; assisting failed or delayed bone marrow engraftment; accelerating myeloid recovery after autologous bone marrow or peripheral stem cell (peripheral blood progenitor cell, PBPC) transplantation (for example, in patients with non-Hodgkin's lymphoma); Hodgkin's disease; and increasing the mobilisation and yield of PBPC to be collected for transplantation.

In yet another embodiment, the pharmaceutical composition comprising an isolated GM-CSF or chimeric molecule thereof can be used, alone or in conjunction with other biologics, drugs or therapies as an adjunct to increase chemotherapy dose-intensity; to provide a priming effect in chemotherapy (i.e. to alter cell cycle kinetics to increase cytotoxicity); to act as a hematopoietic protectant in chemotherapy; to reverse neutropenia in combined radio- and chemotherapy; to prevent or treat mucosal damage (mucositis), which occurs as a side effect of chemotherapy; to reduce severity of fungal infections in leukemia patients; as vaccine adjuvant for the contribution of maturation of dendritic cells and antigen presenting cells; as adjuvant for several vaccines including hepatitis B and influenza; as an adjunct to treat bacterial and fungal infections in preterm infants with sepsis; as an adjuvant to prevent infections in organ transplantion patients who are immunosuppressed; to inhibit HIV replication, to facilitate increased CD4 counts and decreased infections in AIDS patients; as a tumour vaccine in order to enhance immune response to tumour antigen epitope peptides in melanoma; to promote wound repair, such as non-healing leg ulcers; to stimulate arteriogenesis, for instance, in peripheral artery disease; to stimulate of axonal regeneration in nervous system injury; to protect the brain following stroke; to treat pulmonary alveolar proteinosis; and to reduce of graft versus host disease, for instance, in stem cell transplantation.

In another embodiment, the pharmaceutical composition comprising an isolated IL-3 or chimeric molecule thereof can be used, alone or in conjunction with other biologics, drugs or therapies, to treat various diseases or conditions including but not limited to bone marrow disorders such as myelodysplastic syndromes (MDS), heterogeneous related clonal hematopoietic disorders, diseases characterized by a bone marrow disorder in morphology and maturation (dysmyelopoiesis) resulting in peripheral blood cytopenias, anemia, leukopenia and thrombocytopenia; Diamond-Blackfan anemia (DBA); transplantation to augment bone marrow transplant engraftment after high dose chemotherapy treatment for haematological malignancies such as leukocyte malignancies; non-Hodgkin's lymphoma; breast cancer; gene transfer therapy; HIV related cytopenia and myelosuppression; thrombopenias; secondary hematopoietic insufficiencies; certain types of anaemia such as aplastic anemia and follicular small cleaved-cell lymphoma (FSCCL); parasitic infections such as the Trichinella spiralis worm; lung carcinoma tumorigenicity; viral infections such as herpes virus.

In another embodiment, the pharmaceutical composition comprising an isolated IL-4 or chimeric molecule thereof can be used, alone or in conjunction with other biologics, drugs or therapies, in the prevention or treatment of diseases including but not limited to: autoimmune disease, for instance, psoriasis, by promoting TH2 cell induction, Grave's hyperthyroidism, autoimmune diabetes, immune-demyelinating neuropathies (e.g. multiple sclerosis), and Crohn's disease; to treat allergic diseases, for instance, as support therapy in allograft transplantation to promote tissue survival (e.g. skin, heart transplants), reversal or prevention of delayed type hypersensitivity reactions and inflammatory bowel diseases; to treat inflammation due to infection (e.g. gastritis due to Helicobacter pylori); glomerulonephritis; Heymann Nephritis, membranous nephropathy, periodontal disease, arthritis and rheumatoid arthritis; to prevent leukocyte trafficking in pleuritis; to restore cellular immunity after dysregulated inflammatory response (e.g. due to burns and other injuries); to regulate sleeping behaviours (e.g. in the treatment of sleep disorders).

In another embodiment, the pharmaceutical composition comprising an isolated IL-5 or chimeric molecule thereof can be used, alone or in conjunction with other biologics, drugs or therapies, in the treatment of diseases associated with, but not limited to the containment of parasitic worms such as filaria, other parasites and in cancers such acute myelogenous leukemia (AML).

However, the pharmaceutical composition of the present invention has higher pharmaceutical efficacy, increased thermal stability, increased serum half-life or higher solubility in the bloodstream when compared with the protein or chimeric molecule thereof expressed in non-human cell lines. The present invention also shows reduced risks for immune-related clearance or related side effects. Because of these improved properties, the composition of the present invention can be administered at a lower frequency than a protein or chimeric molecule expressed in non-human cell lines. Decreased frequency of administration is anticipated to enhance patient compliance resulting in improved treatment outcomes. The quality of life of the patient is also elevated.

Accordingly, in one embodiment, the pharmaceutical composition of the present invention can be administered in a therapeutically effective amount to patients in the same way a protein or chimeric molecule expressed in non-human cell lines is administered. The therapeutic amount is that amount of the composition necessary for the desired in vivo activity. The exact amount of composition administered is a matter of preference subject to such factors as the exact type of condition being treated, the condition of the patient being treated and the other ingredients in the composition. The pharmaceutical compositions containing the isoforms of the protein or chimeric molecule of the present invention may be formulated at a strength effective for administration by various means to a human patient experiencing one or more of the above disease conditions. Average therapeutically effective amounts of the composition may vary. Effective doses are anticipated to range from 0.1 ng/kg body weight to 20 μg/kg body weight; or based upon the recommendations and prescription of a qualified physician.

The present invention further extends to uses of the isolated protein or the chimeric molecule comprising at least part of the protein or chimeric molecule thereof and a composition comprising same in a variety of therapeutic and/or diagnostic applications.

More particularly, the present invention extends to a method of treating or preventing a condition in a mammalian subject, wherein the condition can be ameliorated by increasing the amount or activity of the protein or chimeric molecule of the present invention, the method comprising administering to said mammalian subject an effective amount of an isolated protein, a chimeric molecule comprising the protein, a fragment or an extracellular domain thereof or a composition comprising the isolated protein or the chimeric molecule.

The present invention is further described by the following non-limiting examples.

### Example 1

### (a) Production of a pIRESbleo3-Fc Construct

The DNA sequence encoding the Fc domain of human IgG1 was amplified from EST cDNA library (Clone ID 6277773, Invitrogen) by Polymerase Chain Reaction (PCR), using forward primer (SEQ ID NO:21) and reverse primer (SEQ ID NO:22) incorporating restriction enzyme sites BamHI and BstXI respectively. This amplicon was cloned into the corresponding enzyme sites of pIRESbleo3 (Cat. No. 6989-1, BD Biosciences) to produce the construct pIRESbleo3-Fc. Digestion of pIRESbleo3-Fc with BamHI and BstX1 released an expected size insert of 780 bp as determined by gel electrophoresis.

### (b) Production of a DNA Construct Expressing a Protein

The DNA sequence encoding the protein was amplified from an EST cDNA library by PCR, using forward primer and reverse primers that incorporated restriction enzyme sites according to Table 8. After amplification, the amplicon was digested with suitable restriction enzymes and cloned into an expression vector as per Table 8, to produce the vector-Protein construct. Suitable restriction enzymes were used to digest the vector containing the DNA sequence encoding the Protein to release the expected size fragments as shown in Table 8. Vector-Protein constructs were sequenced to confirm the integrity of the cloning procedures as herein described.

TABLE 8
Protein-Fc and relevant cloning information

| | | | | Restriction | | |
|---|---|---|---|---|---|---|
| | | Forward | Reverse | Enzyme | | Size |
| Protein | cDNA Source | Primer | Primer | sites | Vector | (bp) |
| GM-CSF | pUMCV1-GM- | SEQ ID | SEQ ID | EcoRV, | pIRESbleo3 | 447 |
| | CSF, Aldevron | NO: 23 | NO: 24 | BamH1 | (Cat. No. 6989-1, | |
| | | | | | BD Biosciences) | |
| IL-3 | RNA extracted | SEQ ID | SEQ ID | BamHI, | pIRESbleo3 | 491 |
| | from stimulated | NO: 33 | NO: 34 | BamHI | (Cat. No. 6989-1, | |
| | Jurkat cells | | | | BD Biosciences) | |
| IL-4 | pUMCV-IL-4 | SEQ ID | SEQ ID | NotI, | pIRESbleo3 | 481 |
| | Aldevron | NO: 49 | NO: 50 | BamHI | (Cat. No. 6989-1, | |
| | | | | | BD Biosciences) | |
| IL-5 | IL-5 pBR322, | SEQ ID | SEQ ID | EcoRI, | pIRESbleo3 | 437 |
| | ATCC | NO: 59 | NO: 60 | BamHI | (Cat. No. 6989-1, | |
| | | | | | BD Biosciences) | |

### (c) Preparation of Megaprep Vector-Protein

750 ml of sterile LB broth containing ampicillin (100 μg/ml) was inoculated with 750 μl of overnight culture of E. Coli transformed with vector-Protein. The culture was incubated at 37° C. with shaking for 16 hours. Plasmid was prepared in accordance with a Qiagen Endofree Plasmid Mega Kit (Qiagen Mega Prep Kit # 12381).

Alternatively, the nucleotide sequence of the Protein that was cloned into the vector (such as pIRESbleo3), can be amplified with primers that incorporate restriction sites allowing the cloning of the DNA sequence encoding the Protein upstream of the Fc nucleotide sequence in a vector-Fc (such as pIRESbleo3-Fc), such that the Protein and the Fc nucleotide sequences are fused in-frame directly or by a linker.

### Example 2

### (a) Production and Purification of Human Cell Expressed GM-CSF

### (i) Production of GM-CSF of the Present Invention

At day 0, five 500 cm² tissue culture dishes (Corning) were seeded with 3×10⁷ cells of a transformed embryonal human kidney cell line, for example HEK 293, HEK 293 c18, HEK 293T, 293 CEN4, HEK 293F, HEK 293FT, HEK 293E, AD-293 (Stratagene) 293A (Invitrogen). Cells were seeded in 90 ml per plate of Dulbecco's Modified Eagle's Medium/Ham's Nutrient Mixture F12 (DMEM/F12) (JRH Biosciences), the medium being supplemented with 10% (v/v) heat-inactivated fetal calf serum (FCS, JRH Biosciences), 10 mM HEPES (Sigma), 4 mM L-glutamine (Ameresco) and 1% (v/v) Penicillin-Streptomycin (JRH Biosciences). Alternatively, 10% (v/v) heat-inactivated donor calf serum (DCS, JRH Biosciences) was used in place of FCS without HEPES.

At day 1, transfection was performed using calcium phosphate. Before transfection, the medium in each plate was replaced with 120 ml of fresh DMEM/F12 with the above-mentioned supplements. Calcium phosphate/DNA precipitate was prepared by adding 1200 μg of pIRESbleo (Invitrogen) plasmid DNA harboring the gene for human GM-CSF and 3000 or 3720 μl 2.5M CaCl₂ in sterile H₂O to a final volume of 30 ml (solution A). Solution A was added drop-wise to 30 ml of 2×HEPES Buffered Saline (HBS) (solution B) with a 10 ml pipette. During the course of addition, bubbles were gently blown through solution B. The mixture was incubated at 25° C. for 20 minutes and vortexed. Alternatively, the mixture was incubated without vortexing. 12 ml of the mixture was added drop-wise to each plate. After 4 hours the medium containing the transfection medium was removed and 100 ml per plate of DMEM/F12 supplemented with 10% (v/v) heat-inactivated donor calf serum (DCS, JRH Biosciences), 10 mM HEPES, 4 mM L-glutamine, 1% (v/v) Penicillin-Streptomycin and 3.5 mM HCl to pH 7 was added and incubated at 37° C. overnight.

At day 2, the DMEM/F12 medium was removed, 100 mM PMSF (1% (v/v)) and 500 mM EDTA (1% (v/v)) were added to the collected medium. The contents in the plates were washed twice with 50 ml of serum-free DMEM/F12 medium per plate and 100 ml of fresh serum-free DMEM/F12 medium was added to each plate (DMEM/F12 medium supplemented with 40 mM N-acetyl-D-mannosamine, 10 mM L-Glutamine, 4.1 g/L Mannose, 1% (v/v) Penicillin-Streptomycin and ITS solution (5 mg/L bovine insulin, 5 mg/L partially iron saturated human transferrin and 5 μg/ml selenium) (Sigma). Alternatively, the supplement contained 40 mM N-acetyl-D-mannosamine, 7 mM L-Glutamine, 0.5 g/L Mannose and 1% (v/v) Penicillin-Streptomycin. The plates were incubated at 37° C.

At day 3, the serum-free DMEM/F12 medium was collected and 100 ml fresh serum-free DMEM/F12 medium was added to each plate, supplemented as indicated above for day 2. 100 mM PMSF (1% (v/v)) and 500 mM EDTA (1% (v/v)) were added to the collected medium and the mixture was stored at 4° C.

At day 4, the serum-free DMEM/F12 medium in the plates was collected. 100 mM PMSF (1% (v/v)) and 500 mM EDTA (1% (v/v)) was added to the collected medium. 5 ml of the mixture was taken for ELISA and the rest of the mixture was combined with the day 3 serum free collection. The combined collections were adjusted to pH 6 by the addition of a one tenth volume of 200 mM MES/50 mM MgCl₂ pH6 before particulate removal using a 0.45 mm low-protein binding filter (Durapore, Millipore). The mixture was either stored at −70° C. or used immediately.

### (ii) Purification of GM-CSF of the Present Invention

The process of Dye-ligand chromatography (DLC) was used as the primary step in the purification of GM-CSF. A library of immobilised reactive dye was used to screen GM-CSF for efficient binding and release in a batch purification microtitre format. Suitable dye-protein combinations were then tested in a small scale column format.

In small scale purification 5 ml samples of thawed cell culture supernatant were passed through 0.5 ml dye-ligand columns at a pH of 6. In this optimisation step optimal dye bead-cytokine and pH combinations were selected for maximal recovery in fractions for up scaling in bulk DLC.

For bulk scale DLC reactive dye number 78 High (Zymatrix) was selected as the reactive dye with the best binding and elution properties for GM-CSF. The filtered cell culture supernatant was passed under gravity flow over 4.0 ml or 8.0 ml column bodies (Alltech, Extract Clean Filter columns) with 3 ml or 6 ml respectively of DLC resin pre-equilibrated to pH 6 with 50 mM MES/5 mM MgCl₂. The bulk flow through sample was stored at 4° C. until ELISA results confirmed that the purification was successful. The column was washed with Buffer A (20 mM MES/5 mM MgCl₂ pH 6) until fractions were free of protein as estimated by colourmetric protein assay (Biorad protein assay). GM-CSF was eluted using three Elution Buffers in the following order.

### Elute 1: Buffer C (50 mM Tris-Cl/10 mM EDTA pH 8)

### Elute 2: EN1.0 (50 mM Tris-Cl/10 mM EDTA/1.0 M NaCl pH 8)

### Elute 3: EN2.0 (50 mM Tris-Cl/10 mM EDTA/2.0 M NaCl pH 8)

The eluted fractions were assayed by silver stained SDS PAGE using 4-20% Tris-Glycine gels (Invitrogen) and monitered by GM-CSF ELISA (R&D Systems Duoset). GM-CSF was found to elute in Buffer EN1.0. It was estimated by SDS PAGE analysis that 90% of the contaminating proteins were removed in this primary purification step. DLC Fractions containing GM-CSF were pooled for size exclusion chromatography.

Size exclusion chromatography (SEC) was performed on the combined DLC fractions using Superdex 75 preparative grade 16/70 (Pharmacia, Uppsala, Sweden) column. An isocratic flow of 1% Ammonium Bicarbonatewas used at a flow rate of 1 ml/min. Alternatively, a Sephadex 200 preparative grade (Pharmacia, Uppsala, Sweden) column was used with 50 mM MES buffer (pH 6.5) at a flow rate of 1.5 ml/min. Total run time was 120 min with peaks eluting between 40 and 100 minutes for the Superdex column, or between 20-100 minutes for the Sephadex column. The eluted fractions were assayed by silver stained SDS PAGE using 4-20% Tris-Glycine gels (Invitrogen). GM-CSF was found have an elution time of between 55 and 65 minutes for the Superdex column and an elution time of approximately 52 minutes for the Sephadex column.

GM-CSF purified using the Sephadex column was further purified by passing the selected fractions over an anion exchange column (Uno Q, Bio-Rad Laboratories) pre-equilibrated to pH 6.5 with 50 mM MES (Sigma). The bound GM-CSF was then eluted from the column with a linear gradient from 50 mM MES pH 6.5 to 50 mM MES pH 6.5 containing 1 M NaCl. Fractions containing the cytokine were desalted into PBS using a HiPrep 26/10 fast desalting column (Pharmacia).

The purified GM-CSF was found to have an apparent MW of 14-32 kDa and to be 100% pure as assessed by silver stained SDS PAGE. Fractions containing GM-CSF were pooled and concentrated using a centrifugal filter device (Amicon Ultra, Millipore) to a volume of less than 2 ml. The purified GM-CSF was found to have a concentration of 417 ug/ml as determined by absorption at 280 μm using a molar extinction co-efficient of 14230 M⁻¹ cm⁻¹.

### (b) Production and Purification of Human Cell Expressed IL-3

### (i) Production of IL-3 of the Present Invention

At day 0, five 500 cm² tissue culture dishes (Corning) were seeded with 3×10⁷ cells of a transformed embryonal human kidney cell line, for example HEK 293, HEK 293 c18, HEK 293T, 293 CEN4, HEK 293F, HEK 293FT, HEK 293E, AD-293 (Stratagene), or 293A (Invitrogen). Cells were seeded in 90 ml per plate of Dulbecco's Modified Eagle's Medium/Ham's Nutrient Mixture F12 (DMEM/F12) (JRH Biosciences), the medium being supplemented with 10% (v/v) heat-inactivated foetal calf serum (FCS, JRH Biosciences), 4 mM L-glutamine (Amresco) and 1% (v/v) Penicillin-Streptomycin (Penicillin G 5000 U/ml, Streptomycin Sulphate 5000 μg/ml) (JRH Biosciences). Alternatively, 10% (v/v) heat-inactivated donor calf serum (DCS, JRH Biosciences) was used in place of FCS without HEPES.

At day 1, transfection was performed using calcium phosphate. Before transfection, the medium in each plate was replaced with 120 ml of fresh DMEM/F12 with the above-mentioned supplements. Calcium phosphate/DNA precipitate was prepared by adding 1200 μg of pIRESbleo3 (Invitrogen) plasmid DNA harbouring the gene for human IL-3 and 3000 or 3720 μl CaCl₂ (2.5 M) in sterile H₂O to a final volume of 30 ml (solution A). Solution A was added drop-wise to 30 ml of 2×HEPES Buffered Saline (HBS) (solution B) with a 10 ml pipette. During the course of addition, bubbles were gently blown through solution B. The mixture was incubated at 25° C. for 20 minutes and vortexed. Alternatively, the mixture was incubated without vortexing. 12 ml of the mixture was added drop-wise to each plate. The plates were incubated at 37° C. and 5% CO₂ overnight.

At day 2, the cell culture supernatant was discarded. The contents in the plates were washed twice with 50 ml of DMEM/F12 medium per plate and 100 ml of fresh serum-free DMEM/F12 medium, supplemented with 4 mM L-glutamine and 1% (v/v) Penicillin-Streptomycin, was added to each plate. Alternatively, the supplement contained 40 mM N-acetyl-D-mannosamine, 7 mM L-Glutamine, 0.5 g/L Mannose and 1% (v/v) Penicillin-Streptomycin. The plates were incubated at 37° C. and 5% CO₂ overnight.

At day 3, the cell culture supernatant was collected and 100 ml fresh serum-free DMEM/F12 medium, supplemented with 4 mM L-glutamine, and 1% (v/v) Penicillin-Streptomycin, was added to each plate, supplemented as indicated above for day 2. The plates were incubated at 37° C. and 5% CO₂ overnight. 100 mM PMSF (1% (v/v)) and 500 mM EDTA (1% (v/v)) were added to the collected cell culture supernatant and the mixture was stored at 4° C.

At day 4, the cell culture supernatant was collected. 100 mM PMSF (1% (v/v)) and 500 mM EDTA (1% (v/v)) were added to the collected cell culture supernatant and combined with the day 3 collection before particulate removal using a 0.45 μm low-protein binding filter (Durapore, Millipore). The mixture was either stored at −70° C. or used immediately.

### (ii) Purification of IL-3 of the Present Invention

One litre of filtered cell culture supernatant was concentrated using a tangential flow filtration (TFF) device (Pelicon XL, Ultracell, Millipore). The sample was pumped at 150 ml/min across 150 cm² of regenerated cellulose membrane, with a nominal molecular weight cut-off of 5 KDa until the sample had concentrated down to a volume of 40 or 50 ml. The concentrated sample was diafiltered by the addition of an equal volume of buffer, 50 mM MES pH 5.6, followed by another concentration down to 40 ml. This diafiltration step was repeated 2-3 times with a final concentration to 80 ml. The concentrated diafiltered sample was then filtered through a 0.45 μm low-protein binding filter (Durapore, Millipore).

Purification of IL-3 was achieved by passing the concentrated cell culture supernatant from the TFF over a Cation Exchange column (Bio-Rad Laboratories, Uno S12) pre-equilibrated to pH 5.6 with 50 mM MES pH 5.6 (Sigma). Alternatively, the bound IL-3 was then eluted from the column with a linear gradient from 50 mM MES pH 5.6 to 50 mM MES pH 5.6 containing 1 M NaCl. The resulting fractions were analysed for apparent molecular weight and level of purity by ELISA and ID SDS PAGE using 4-20% gradient Tris-Glycine gels (Invitrogen) and quantitated by anti-IL-3 ELISA (R & D Systems). Fractions containing IL-3 were concentrated approximately 50 fold separately using a centrifugal filter device (Amicon Ultra, Millipore) with a nominal molecular weight cut off of 5 KDa.

Further purification of the IL-3 was achieved by performing size exclusion chromatography using a Superdex 75 preparative grade 16/70 (Pharmacia, Uppsala, Sweden) column. An isocratic flow of 1% Ammonium Bicarbonate was used at a flow rate of 0.5 ml/min. Alternatively, an isocratic flow of 1× Dulbecco's Phosphate Buffered Saline (DPBS Modified) (JRH Biosciences) buffer (pH 7.3) was used at a flow rate of 1.5 ml/min. Total run time for the Ammonium Bicarbonate buffer was 240 min with peaks eluting between 70 and 190 minutes. For the DPBS Modified buffer the total run time was 120 minutes with peaks eluting between 20 and 100 minutes. Eluted fractions were assayed by silver stained SDS PAGE using 4-20% Tris-Glycine gels (Invitrogen) and by anti-IL-3 ELISA (R & D Systems) IL-3 eluted between 90 and 120 minutes using the Ammonium Bicarbonate buffer and at approximately 53 minutes using the DPBS Modified buffer.

The purified IL-3 was found to have an apparent MW between 16 to 32 kDa and to be at least 99% pure as assessed by silver stained SDS PAGE. Fractions containing IL-3 were pooled and concentrated using a centrifugal filter device (Amicon Ultra, Millipore) to a volume of less than 2 ml. The purified IL-3 was found to have a concentration of 931 ug/ml as determined by absorption at 280 nm using a molar extinction co-efficient of 12615 M⁻¹ cm⁻¹.

### (c) Production and Purification of Human Cell Expressed IL-4

### (i) Production of IL-4 of the Present Invention

At day 0, five 500 cm² tissue culture dishes (Corning) were seeded with 3×10⁷ cells of a transformed embryonal human kidney cell line, for example HEK 293, HEK 293 c18, HEK 293T, 293 CEN4, HEK 293F, HEK 293FT, HEK 293E, AD-293 (Stratagene) 293A (Invitrogen). Cells were seeded in 90 ml per plate of Dulbecco's Modified Eagle's Medium/Ham's Nutrient Mixture F12 (DMEM/F12) (JRH Biosciences), the medium being supplemented with 10% (v/v) heat-inactivated fetal calf serum (FCS, JRH Biosciences), 10 mM HEPES (Sigma), 4 mM L-glutamine (Ameresco) and 1% (v/v) Penicillin-Streptomycin (JRH Biosciences). Alternatively, 10% (v/v) heat-inactivated donor calf serum (DCS, JRH Biosciences) was used in place of FCS without HEPES.

At day 1, transfection was performed using calcium phosphate. Before transfection, the medium in each plate was replaced with 120 ml of fresh DMEM/F12 with the above-mentioned supplements. Calcium phosphate/DNA precipitate was prepared by adding 1200 μg of pIRESbleo (Invitrogen) plasmid DNA harboring the gene for human IL-4 and 3000 or 3720 μl 2.5M CaCl₂ in sterile H₂O to a final volume of 30 ml (solution A). Solution A was added drop-wise to 30 ml of 2×HEPES Buffered Saline (HBS) (solution B) with a 10 ml pipette. During the course of addition, bubbles were gently blown through solution B. The mixture was incubated at 25° C. for 20 minutes and vortexed. Alternatively, the mixture was incubated without vortexing. 12 ml of the mixture was added drop-wise to each plate. After 4 hours the medium containing the transfection medium was removed and 100 ml per plate of DMEM/F12 supplemented with 10% (v/v) heat-inactivated donor calf serum (DCS, JRH Biosciences), 10 mM HEPES, 4 mM L-glutamine, 1% (v/v) Penicillin-Streptomycin and 3.5 mM HCl to pH 7 was added and incubated at 37° C. overnight.

At day 2, the DMEM/F12 medium was removed, 100 mM PMSF (1% (v/v)) and 500 mM EDTA (1% (v/v)) were added to the collected medium. The contents in the plates were washed twice with 50 ml of serum-free DMEM/F12 medium per plate and 100 ml of fresh serum-free DMEM/F12 medium was added to each plate (DMEM/F12 medium supplemented with 40 mM N-acetyl-D-mannosamine, 10 mM L-Glutamine, 4.1 g/L Mannose, 1% (v/v) Penicillin-Streptomycin and ITS solution (5 mg/L bovine insulin, 5 mg/L partially iron saturated human transferrin and 5 μg/ml selenium) (Sigma). Alternatively, the supplement contained 40 mM N-acetyl-D-mannosamine, 7 mM L-Glutamine, 0.5 g/L Mannose and 1% (v/v) Penicillin-Streptomycin. The plates were incubated at 37° C.

At day 3, the serum-free DMEM/F12 medium was collected and 100 ml fresh serum-free DMEM/F12 medium was added to each plate, supplemented as indicated above for day 2. 100 mM PMSF (1% (v/v)) and 500 mM EDTA (1% (v/v)) were added to the collected medium and the mixture was stored at 4° C.

At day 4, the serum-free DMEM/F12 medium in the plates was collected. 100 mM PMSF (1% (v/v)) and 500 mM EDTA (1% (v/v)) was added to the collected medium. 5 ml of the mixture was taken for ELISA and the rest of the mixture was combined with the day 3 serum free collection. The combined collections were adjusted to pH 6 by the addition of a one tenth volume of 200 mM MES/50 mM MgCl₂ pH6 before particulate removal using a 0.45 mm low-protein binding filter (Durapore, Millipore). The mixture was either stored at −70° C. or used immediately.

### (b) Purification of IL-4 of the Present Invention

The process of Dye-ligand chromatography (DLC) was used as the primary step in the purification of IL-4. A library of immobilised reactive dye was used to screen IL-4 for efficient binding and release in a batch purification microtitre format. Suitable dye-protein combinations were then tested in a small-scale column format.

In small-scale purification 5 ml samples of thawed cell culture supernatant was passed through 0.5 ml dye-ligand columns at a pH of 6. In this optimisation step optimal dye bead-cytokine and pH combinations were selected for maximal recovery in fractions for up scaling in bulk DLC.

For bulk scale DLC reactive dye number 17 High (Zymatrix) was selected as the reactive dye with the best binding and elution properties for IL-4 The filtered cell culture supernatant was passed under gravity flow over 4.0 ml or 8.0 ml column bodies (Alltech, Extract Clean Filter columns) with 3 ml or 6 ml respectively of DLC resin pre-equilibrated to pH 6 with 50 mM MES/5 mM MgCl₂. The bulk flow through sample was stored at 4° C. until ELISA results confirmed that the purification was successful. The column was washed with Buffer A (20 mM MES/5 mM MgCl₂ pH 6) until fractions were free of protein as estimated by colourmetric protein assay (Biorad protein assay). IL-4 was eluted using three Elution Buffers in the following order.

### Elute 1: Buffer C (50 mM Tris-Cl/10 mM EDTA pH 8)

### Elute 2: EN1.0 (50 mM Tris-Cl/10 mM EDTA/1.0 M NaCl pH 8)

### Elute 3: EN2.0 (50 mM Tris-Cl/10 mM EDTA/2.0 M NaCl pH 8)

The eluted fractions were assayed by silver stained SDS PAGE using 4-20% Tris-Glycine gels (Invitrogen) and monitered by IL-4 ELISA (R&D Systems Duoset). IL-4 was found to elute in Buffer 2 and Buffer 3. It was estimated by SDS PAGE analysis that 90% of the contaminating proteins were removed in this primary purification step. DLC Fractions containing IL-4 were pooled and concentrated to less than 2 ml using a centrifugal filter device (Amicon Ultra, Millipore) for size exclusion chromatography.

Size exclusion chromatography (SEC) was performed on the combined DLC fractions using Superdex 75 preparative grade 16/70 (Pharmacia, Uppsala, Sweden) column. An isocratic flow of 1% Ammonium Bicarbonate was used at a flow rate of 1 ml/min. Total run time was 120 min with peaks eluting between 40 and 100 minutes. Alternatively, a Sephadex 200 preparative grade column (Pharmacia, Uppsala, Sweden) was used with an isocratic flow of 50 mM MES buffer (pH 5.6) at a flow rate of 1.5 ml/min. In this case, IL-4 eluted between 20-100 minutes. The eluted fractions were assayed by silver stained SDS PAGE using 4-20% Tris-Glycine gels (Invitrogen) and monitered by IL-4 ELISA (R&D Systems Duoset) For the Superdex column SEC, IL-4 was found to elute between approximately 65 and 70 minutes, whereas IL-4 was found to elute at approximately 75 minutes for the Sephadex column SEC.

The purified IL-4 was found to have an apparent MW of approximately 14 kDa and to be at least 95% pure as assessed by silver stained SDS PAGE. Fractions containing IL-4 were pooled and concentrated using a centrifugal filter device (Amicon Ultra, Millipore) to a volume of less than 2 ml. The purified IL-4 was found to have a concentration of 507 ug/ml as determined by absorption at 280 μm using a molar extinction co-efficient of 8855 M⁻¹ cm⁻¹.

### (d) Production and Purification of Human Cell Expressed IL-5

### (i) Production of IL-5 of the Present Invention

At day 0, five 500 cm² tissue culture dishes (Corning) were seeded with 3×10⁷ cells of a transformed embryonal human kidney cell line, for example HEK 293, HEK 293 c18, HEK 293T, 293 CEN4, HEK 293F, HEK 293FT, HEK 293E, AD-293 (Stratagene) 293A (Invitrogen). Cells were seeded in 90 ml per plate of Dulbecco's Modified Eagle's Medium/Ham's Nutrient Mixture F12 (DMEM/F12) (JRH Biosciences), the medium being supplemented with 10% (v/v) heat-inactivated fetal calf serum (FCS, JRH Biosciences), 10 mM HEPES (Sigma), 4 mM L-glutamine (Ameresco) and 1% (v/v) Penicillin-Streptomycin (JRH Biosciences). Alternatively, 10% (v/v) heat-inactivated donor calf serum (DCS, JRH Biosciences) was used in place of FCS without HEPES.

At day 1, transfection was performed using calcium phosphate. Before transfection, the medium in each plate was replaced with 120 ml of fresh DMEM/F12 with the above-mentioned supplements. Calcium phosphate/DNA precipitate was prepared by adding 1200 μg of pIRESbleo (Invitrogen) plasmid DNA harboring the gene for human IL-5 and 3000 or 3720 μl 2.5M CaCl₂ in sterile H₂O to a final volume of 30 ml (solution A). Solution A was added drop-wise to 30 ml of 2×HEPES Buffered Saline (HBS) (solution B) with a 10 ml pipette. During the course of addition, bubbles were gently blown through solution B. The mixture was incubated at 25° C. for 20 minutes and vortexed. Alternatively, the mixture was incubated without vortexing. 12 ml of the mixture was added drop-wise to each plate. After 4 hours the medium containing the transfection medium was removed and 100 ml per plate of DMEM/F12 supplemented with 10% (v/v) heat-inactivated donor calf serum (DCS, JRH Biosciences), 10 mM HEPES, 4 mM L-glutamine, 1% (v/v) Penicillin-Streptomycin and 3.5 mM HCl to pH 7 was added and incubated at 37° C. overnight.

At day 2, the DMEM/F12 medium was removed, 100 mM PMSF (1% (v/v)) and 500 mM EDTA (1% (v/v)) were added to the collected medium. The contents in the plates were washed twice with 50 ml of serum-free DMEM/F12 medium per plate and 100 ml of fresh serum-free DMEM/F12 medium was added to each plate (DMEM/F12 medium supplemented with 40 mM N-acetyl-D-mannosamine, 10 mM L-Glutamine, 4.1 g/L Mannose, 1% (v/v) Penicillin-Streptomycin and ITS solution (5 mg/L bovine insulin, 5 mg/L partially iron saturated human transferrin and 5 μg/ml selenium) (Sigma). Alternatively, the supplement contained 40 mM N-acetyl-D-mannosamine, 7 mM L-Glutamine, 0.5 g/L Mannose and 1% (v/v) Penicillin-Streptomycin. The plates were incubated at 37° C.

At day 3, the serum-free DMEM/F12 medium was collected and 100 ml fresh serum-free DMEM/F12 medium was added to each plate, supplemented as indicated above for day 2. 100 mM PMSF (1% (v/v)) and 500 mM EDTA (1% (v/v)) were added to the collected medium and the mixture was stored at 4° C.

At day 4, the serum-free DMEM/F12 medium in the plates was collected. 100 mM PMSF (1% (v/v)) and 500 mM EDTA (1% (v/v)) was added to the collected medium. 5 ml of the mixture was taken for ELISA and the rest of the mixture was combined with the day 3 serum free collection. The combined collections were adjusted to pH 6 by the addition of a one tenth volume of 200 mM MES/50 mM MgCl₂ pH6 before particulate removal using a 0.45 mm low-protein binding filter (Durapore, Millipore). The mixture was either stored at −70° C. or used immediately.

### (ii) Purification of IL-5 of the Present Invention

One litre of filtered cell culture supernatant was concentrated 20 fold using a tangential flow filtration (TFF) device (Pelicon XL, Ultracell, Millipore). The sample was pumped at 150 ml/min across 150 cm² of regenerated cellulose membrane, with a nominal molecular weight cut-off of 5 KDa until the sample had concentrated down to a volume of 50 ml. The concentrated sample was diafiltered by the addition of an equal volume of 20 mM Tris-Cl pH 8.5 followed by another concentration down to 50 ml. Alternatively, the concentrated sample was diafiltered by the addition of an equal volume of 50 mM HEPES pH 8 followed by another concentration down to 50 ml. This diafiltration step was repeated twice (in the case of Tris buffer) or 3 times (in the case of HEPES buffer) with a final concentration to 50 ml.

Purification of IL-5 by anion exchange chromatography (AEC) was achieved by passing the concentrated cell culture supernatant from the TFF over a Macroprep-Q column (Bio-Rad Laboratories), pre-equilibrated to 20 mM Tris-Cl pH 8.5 (Sigma). The bound IL-5 was then eluted from the column with a gradient from 20 mM Tris-Cl pH 8.5 to 20 mM Tris-Cl pH 8.5 containing 1 M NaCl. Alternatively, a Uno S12 column (Bio-Rad Laboratories), pre-equilibrated to 50 mM HEPES pH 8 (Sigma) was used for AEC. The bound IL-5 was then eluted from the column with a gradient from 50 mM HEPES pH 8 to 50 mM HEPES containing 1 M NaCl. The resulting fractions were analysed for apparent molecular weight and level of purity by silver stained 1D SDS PAGE using 4-20% gradient Tris-Glycine gels (Invitrogen) and quantified by IL-5 ELISA (R & D Systems).

The fractions containing IL-5 were further purified by performing size exclusion chromatography (SEC) using a Superdex 75 preparative grade 16/70 preparative grade (Pharmacia, Uppsala, Sweden) column. For some experiments, fractions were concentrated to approximately 2 ml using a centrifugal filter device (Amicon Ultra, Millipore) prior to SEC. For SEC, an isocratic flow of 1% Ammonium Bicarbonate was used at a flow rate of 1 ml/min. Total run time was 120 min with peaks eluting between 40 and 100 minutes. Alternatively, a Superdex 75 preparative grade 16/70 preparative grade (Pharmacia, Uppsala, Sweden) was used with an isocratic flow of 50 mM 1× Dulbecco's Phosphate Buffered Saline (DPBS Modified) (JRH Biosciences) at a flow rate of 1.5 ml/min. In this case, IL-5 eluted between 20-100 minutes.

The eluted fractions were assayed by silver stained SDS PAGE using 4-20% Tris-Glycine gels (Invitrogen) or in combination with an IL-5 ELISA (R & D Systems). IL-5 was found to elute between 50 and 65 minutes into the run for the Ammonium Bicarbonate buffer, whereas IL-5 was found to elute 45 minutes into the run for the DPBS Modified buffer. Fractions containing IL-5 were either concentrated to less than 2 ml using a centrifugal filter device (Amicon Ultra, Millipore) or desalted into PBS using a HiPrep 26/10 fast desalting column (Pharmacia, Uppsala, Sweden).

The purified IL-5 was found to have an apparent MW of between 16 and 36 kDa and to be at least 99% pure as assessed by silver stained SDS PAGE using 4-20% Tris-Glycine gels (Invitrogen). The final concentration of the IL-5 was determined either by absorption at 280 nm. The final concentration of the IL-5 was found to be 206 ug/ml as determined by absorption at 280 nm using a molar extinction co-efficient of 8605 M⁻¹ cm⁻¹.

### Example 3

### (a) Characterization of GM-CSF of the Present Invention

### (i) Two-Dimensional Polyacrylamide Electrophoresis

The collected sample from Example 2(a) was buffer exchanged by dialysis or desalting column (Pharmacia HR 10/10 Fast Desalting Column) into re-purified (18 MOhm) water and dried using a SpeedVac concentrator. Alternatively the collected sample underwent TCA or acetone precipitation using methods known in the art. The sample was then re-dissolved into 240 μl MSD buffer (5M urea, 2M thiourea, 65 mM DTT, 2% (w/v) CHAPS, 2% (w/v) sulfobetaine 3-10, 0.2% (v/v) carrier ampholytes, 40 mM Tris, 0.002% (w/v) bromophenol blue, water) and centrifuged at 15000 g for 8 minutes.

Isoelectric focusing (IEF) was performed using either precast 11 cm or precast 17 cm gel pH 3-10 immobilised pH gradient IEF strips (BioRad). The IEF strips were re-hydrated in the sample in a sealed tube at room temperature for at least 6 hours. The IEF strips were placed into the focusing chamber and covered with paraffin oil. IEF was carried out at 100 V for 1 h, 200V for 1 h, 600V for 2 h, 1000 V for 2 h, 2000 V for 2 h, 3500 V for 12 h and 100 V for up to 12 h in the case of 11 cm strips or for 85 kV hours in the case of 17 cm strips (using the same V ramp up procedure).

Following isoelectric focusing the strips were reduced and alkylated before being applied to a second dimension gel. The strips were incubated in 1× Tris/HCl pH 8.8, 6M urea, 2% (w/v) SDS, 2% (v/v) glycerol, 5 mM tributylphosphine (TBP), 2.5% (v/v) acrylamide solution for at least 20 minutes.

The 11 cm strips were separated on the second dimension by Criterion pre poured (11×8 cm; 1 mm thick) 10-20% Tris glycine gradient gels (BioRad). 17 cm strips were separated on 17×17 cm, 1.5 mm thick, self poured 10-20% Tris glycine gradient gels. Precision or Kaleidoscope molecular weight markers (BioRad) were also applied to the gel. The strip was set into place using 0.5% Agarose containing bromophenol blue as a tracking dye.

The SDS-PAGE was run using either a Criterion or Protean II electrophoresis system (BioRad) (200 V for 1 h (until the buffer front was about to run off the end of the gel) for 11 cm gels and 15 mA constant current per gel for 21 h for 17 cm gels). The buffer used was 192 mM glycine, 0.1% (w/v) SDS, 24.8 mM Tris base at pH 8.3.

The completed second dimension gels were fixed for 30 minutes—overnight in 10% methanol (MeOH) and 7% acetic acid (Hac). The gel was then stained using Sypro Ruby gel stain (BioRad) for at least 3 h and destained with 10% MeOH and 7% HAc for at least 30 minutes. Alternatively after fixing the gels were stained using Deep Purple fluorescent stain. The gels were incubated in 300 mM Na₂CO₃, 35 mM NaHCO₃ for 2×30 min, then incubated in 1:200 dilution Deep Purple stain for at least 1 h in the dark. The gels were then destained by 2×15 minute incubations in 10% MeOH, 7% HAc. In both cases the gel was imaged using a FX laser densitometer (BioRad) and the appropriate filter.

### (ii) Analysis of Two-Dimensional Electrophoresis Protein Maps Using Image Analysis Software

ImageJ (http://rsb.info.nih.gov/ij/) was used to analyse the relative intensities of the protein spots on the gel. Densitometry was performed on the spots within a selected area of the gel and a background subtraction was conducted using the appropriate region of the gel lacking protein spots. A volume integration was performed on each protein spot of interest. Relative percentage intensities were calculated for each protein spot and by normalising the combined value of the intensities of all spots to 100%, the intensity of each protein spot relative to the other spots in the gel was determined.

The molecular weights of the respective spots were determined by measuring the respective distance of the spots from the base of the gel and comparing the distance shown by Precision or Kaleidoscope molecular weight markers that were also applied to the gel. A 4^{th} order polynomial and exponential function was fitted to the precision markers to interpolate protein spot locations respectively. In this way, the molecular weights of the respective spots could be accurately determined.

The charge of the isoforms (pKa values) were determined by measuring the respective distance of the spots from the left side of the gel using ImageJ. Since the relationship between the pI values of the strip and the physical distance of the gel is linear, the pI values corresponding to the different pKa values of the isoform spots were readily determined.

The major protein spots in the gel correspond to isoforms of GM-CSF. The low intensity spots may be GM-CSF or low level contaminants, however, these cannot be confirmed by PMF due to the low intensity. Examination of the gel revealed that GM-CSF of the present invention contains 10 to 30 isoforms. Table 9 shows key properties of these isoforms: the pI values (±1.0), the apparent molecular weights (±20%), and the relative intensities (±20% of the actual value or ±2% of the total, whichever is larger). The values listed correspond to the intensity weighted center within the selected area of gel containing the spot and hence, are only reflective of the pI and molecular weight of the protein at one particular reading within the selected area of the gel. Taking into consideration the inherent variability of size and position of protein spots within 2D gels, the pI values for the molecule are determined to range from about 2-7 based on the values listed in Table 9; and the apparent molecular weights of the molecule are determined to range from 16-40 kDa based on the values listed in Table 9.

TABLE 9
Molecular weights and pI values of isoforms of GM-CSF

| | | | Relative Intensity (%) |
|---|---|---|---|
| | Isoelectric Point | Molecular Weight | (Normalized Value) |
| Spot No | (pI) (±1.0) | (kDa) (±20%) | (±20% or ±2% of total) |
| 2 | 3.68 | 33.42 | 9.56 |
| 3 | 3.94 | 32.80 | 13.27 |
| 4 | 4.15 | 34.17 | 10.89 |
| 5 | 4.37 | 34.76 | 9.35 |
| 6 | 4.60 | 35.36 | 8.88 |
| 7 | 4.86 | 36.17 | 9.65 |
| 8 | 5.16 | 36.03 | 8.75 |
| 9 | 3.71 | 29.57 | 1.93 |
| 10 | 4.62 | 22.49 | 4.12 |
| 11 | 5.01 | 23.32 | 1.98 |
| 12 | 4.98 | 21.40 | 2.18 |
| 13 | 5.23 | 23.71 | 1.75 |
| 14 | 5.23 | 21.96 | 2.18 |
| 15 | 5.22 | 20.35 | 1.44 |
| 16 | 5.43 | 22.40 | 2.64 |
| 17 | 5.43 | 20.43 | 2.00 |
| 18 | 5.63 | 22.18 | 1.68 |
| 19 | 5.63 | 20.39 | 1.40 |
| 20 | 4.54 | 17.15 | 0.65 |
| 21 | 4.72 | 17.78 | 0.88 |
| 22 | 5.11 | 15.78 | 0.87 |
| 23 | 5.34 | 14.76 | 2.55 |
| 24 | 5.65 | 15.35 | 1.41 |

### (ii) One-Dimensional Polyacrylamide Electrophoresis

The sample collected from Example 2(a) was dried and then re-solubilised into 60 μl of 1D sample buffer (10% glycerol, 0.1% SDS, 10 mM DTT, 63 mM tris-HCl) and heated at 100° C. for 5 minutes. For PNGaseF treatment, a 30 μL aliquot of the sample was taken and NP40 added to a final concentration of 0.5%. 5 μL of PNGaseF was added and the sample was incubated at 37° C. for 3 hours. For glycosidase cocktail treatment of the sample, an aliquot was taken and NP40 is added to a final concentration of 0.5%. 1 μL of PNGase F, and 1 μL each of Sialidase A (neuramidase), O-Glycanase, β (1-4)-Galactosidase and β-N-Acetylglucosaminidase was added. Treated and untreated samples were incubated at 37° C. for 3 hours. Treated and untreated samples were run on a pre-cast Tris gel, for example, a Tris 4-20% gradient gel (BioRad) or Tris HCl gradient gel (Invitrogen). Precision molecular weight markers (BioRad catalogue number 161-0363) were also applied to the gel. Criterion 4-20% or 18% gels were used for 1D SDS-PAGE (BioRad catalogue numbers: 345-0033 or 345-0024).

The SDS-PAGE was run using either a Mini Protean II or a Criterion electrophoresis system (BioRad) at 200 V for approximately 1 h or until the buffer front was about to run off the end of the gel. The buffer used was 192 mM glycine, 0.1% (w/v) SDS, 24.8 mM Tris base at pH 8.3.

The completed gels were fixed for at least 30 minutes in 10% MeOH and 7% HAc. The gel was then stained using Sypro Ruby gel stain (BioRad) for at least 3 h and destained with 10% MeOH and 7% HAc for at least 30 minutes. Alternatively the gels were stained using Deep Purple (Amersham) as per the manufacturers instructions. The gel was imaged using a FX laser densitometer (BioRad) and the appropriate filter.

The apparent molecular weight of the GM-CSF (as observed by SDS-PAGE) following the release of N-linked oligosaccharides (by PNGase treatment) was between 12 and 30 kDa. The apparent molecular weight of the GM-CSF (as observed by SDS-PAGE) following the release of N-linked and O-linked oligosaccharides (by glycosidase treatment) was between 11 and 25 kDa.

(iii) N-Terminal Sequencing

Protein bands are cut from the gel prepared above (either from a two-dimensional gel or a one-dimensional gel) and are placed into a 0.5 ml tube and 100 ml extraction buffer is added (100 mM Sodium acetate, 0.1% SDS, 50 mM DTT pH 5.5). The gel slices are incubated at 37° C. for 16 h with shaking. The supernatant is applied to a ProSorb membrane (ABI) as per the manufacturers instruction and sequenced using an automated 494 Protein Sequencer (Applied Biosystems) as per the manufacturers instructions. The sequence generated is used to confirm the identity of the protein.

### (iv) Peptide Mass Fingerprinting

Protein bands were cut from the gel prepared above (either from a two-dimensional gel or a one-dimensional gel) and washed with 25 μl of wash buffer (50% acetonitrile in 50 mM NH₄HCO₃). The gel pieces were left at room temperature for at least 1 hour and dried by vacuum centrifugation for 30 minutes. The gel pieces and 12 μl of trypsin solution (20 μg trypsin, 1200 μl NH₄HCO₃) was placed in each sample well and incubated at 4° C. for 1 hour. The remaining trypsin solution was removed and 20 μl 50 mM NH₄HCO₃ was added. The mixture was incubated overnight at 37° C. with gentle shaking. The peptide samples were concentrated and desalted using C18 Zip-Tips (Millipore, Bedford, Mass.) or pre-fabricated micro-columns containing Poros R2 (Perseptive Biosystems, Framingham, Mass.) chromatography resin. Bound peptides were eluted in 0.8 μl of matrix solution (α-cyano-4-hydroxy cinnamic acid (Sigma), 8 mg/ml in 70% acetonitrile/1% formic acid) directly onto a target plate. Peptide mass fingerprints of tryptic peptides were generated by matrix-assisted laser desorption/ionisation time-of-flight mass spectrometry (MALDI-TOF MS) using a Perseptive Biosystems Voyager DE-STR. Spectra were obtained in reflectron mode using an accelerating voltage of 20 kV. Mass calibration was performed using trypsin autolysis peaks, 2211.11 Da and 842.51 Da as internal standards. Data generated from peptide mass fingerprinting (PMF) was used to confirm the identity of the protein. Searches (primarily of Homo sapien (Human) and mammalian entries) were performed in databases such the SWISS-PROT and TrEMBL, via the program PeptIdent (www.expasy.ch/tools/peptident.html). Identification parametres included peptide mass tolerance of 0.1 Da, a maximum of one missed tryptic cleavage per peptide, and the methionine sulfoxide and cysteine-acrylamide modifications. Identifications were based on the number of matching peptide masses and the total percentage of the amino acid sequence that those peptides covered, in comparison to other database entries. Generally, a peptide match with at least 30% total sequence coverage was required for confidence in identification, but very low and high mass proteins, and those resulting from protein fragmentation, may not always meet this criterion, therefore requiring further identification.

Where inconclusive or no protein identification could be obtained from MALDI-TOF PMF analysis, the remaining peptide mixture or the identical spot cut from a replicate gel was subjected to tryptic digest and analysed by electrospray ionization tandem MS (ESI-MS/MS). For ESI-MS/MS, peptides were eluted from Poros R2 micro-columns in 1-2 μl of 70% acetonitrile, 1% formic acid directly into borosilicate nanoelectrospray needles (Micromass, Manchester, UK). Tandem MS was performed using a Q-T of hybrid quadrupole/orthogonal-acceleration TOF mass spectrometer (Micromass). Nanoelectrospray needles containing the sample were mounted in the source and stable flow obtained using capillary voltages of 900-1200V. Precursor ion scans were performed to detect mass to charge ratio (m/z) values for peptides within the mixture. The m/z of each individual precursor ion was selected for fragmentation and collided with argon gas using collision energies of 18-30 eV. Fragment ions (corresponding to the loss of amino acids from the precursor peptide) were recorded and processed using MassLynx Version 3.4 (Micromass). Amino acid sequences were deduced by the mass differences between y- or b-ion ‘ladder’ series using the program MassSeq (Micromass) and confirmed by manual interpretation. Peptide sequences were then used to search the NCBI and TrEMBL databases using the program BLASTP “short nearly exact matches”. A minimum of two matching peptides were required to provide confidence in a given identification.

The identity of the gels spots were confirmed to be GM-CSF.

Further, an observed IDa shift in the masses of tryptic peptides indicated the asparagine residues (N) of 2 NX(S/T/C) motifs found in the theoretical amino acid sequence of human GM-CSF were modified to aspartic acid (D), consistent with the known ability of PNGase F to induce an N to D residue modification upon removal of associated N-linked oligosaccharides. Hence, the confirmed sites of N-glycosylation of the GM-CSF of the present invention are N-44 and N-54 (when numbered from the start of the signal sequence).

### (b) Characterization of IL-3 of the Present Invention

### (i) Two-Dimensional Polyacrylamide Electrophoresis

The sample collected from Example 2(b) was treated and analysed as described above in Example 3(a)(i). The major protein spots in the gel correspond to isoforms of IL-3. The low intensity spots may be IL-3 or low level contaminants, however, these cannot be confirmed by PMF due to the low intensity. Examination of the gel revealed that IL-3 of the present invention contains 5 to 15 isoforms. Table 10 shows key properties of these isoforms: the pI values (±1.0), the apparent molecular weights (±20%), and the relative intensities (±20% of the actual value or ±2% of the total, whichever is larger). The values listed correspond to the intensity weighted center within the selected area of gel containing the spot and hence, are only reflective of the pI and molecular weight of the protein at one particular reading within the selected area of the gel. Taking into consideration the inherent variability of size and position of protein spots within 2D gels, the pI values for the molecule are determined to range from about 3.5 to 7.5 based on the values listed in Table 10; and the apparent molecular weights of the molecule are determined to range from 15 to 35 kDa based on the values listed in Table 10.

TABLE 10
Molecular weights and pI values of isoforms of IL-3

| | | | Relative Intensity (%) |
|---|---|---|---|
| | | | (Normalized Value) |
| | Isoelectric Point (pI) | Molecular Weight | (±20% or ±2% of the |
| Spot No | (±1.0) | (KDa) (±20%) | total) |
| 2 | 5.91 | 21.22 | 1.14 |
| 3 | 5.39 | 20.00 | 1.24 |
| 4 | 5.87 | 20.16 | 2.49 |
| 5 | 5.30 | 19.06 | 1.59 |
| 6 | 5.85 | 18.99 | 3.05 |
| 7 | 5.30 | 17.97 | 4.81 |
| 8 | 5.56 | 17.70 | 6.51 |
| 9 | 5.87 | 17.70 | 5.35 |
| 10 | 6.12 | 17.42 | 3.30 |
| 11 | 5.28 | 16.64 | 3.21 |
| 12 | 5.87 | 16.71 | 3.94 |
| 13 | 5.41 | 15.75 | 1.62 |
| 14 | 5.37 | 14.89 | 1.43 |
| 15 | 6.32 | 19.28 | 0.47 |
| 16 | 6.52 | 19.15 | 2.75 |
| 17 | 6.73 | 19.63 | 6.53 |
| 18 | 6.88 | 19.42 | 3.10 |
| 19 | 6.30 | 17.45 | 2.64 |
| 20 | 6.51 | 17.29 | 4.09 |
| 21 | 6.73 | 17.29 | 5.93 |
| 22 | 6.86 | 17.42 | 2.79 |
| 23 | 6.51 | 15.88 | 5.66 |
| 24 | 6.73 | 15.97 | 5.80 |
| 25 | 6.96 | 16.44 | 1.87 |
| 26 | 6.84 | 15.96 | 1.89 |
| 27 | 6.51 | 14.81 | 2.42 |
| 28 | 5.93 | 12.85 | 1.53 |
| 29 | 6.49 | 13.66 | 3.29 |
| 30 | 6.73 | 13.52 | 0.86 |
| 31 | 6.50 | 12.34 | 5.67 |
| 32 | 6.59 | 12.33 | 3.07 |

### (ii) One-Dimensional Polyacrylamide Electrophoresis

The collected sample from Example 2(b) was treated as described above in Example 3(a)(ii). The apparent molecular weight of the IL-3 (as observed by SDS-PAGE) following the release of N-linked oligosaccharides (by PNGase treatment) was between 10 and 25 kDa.

(iii) N-Terminal Sequencing of Proteins

N-terminal sequencing of the IL-3 of the present invention is performed as described above in Example 3(a)(iii).

### (iv) Peptide Mass Fingerprinting

Peptide mass fingerprinting of the IL-3 of the present invention was performed as described above in Example 3(a)(iv).

The identity of the gel spots were confirmed to be IL-3.

### (c) Characterization of IL-4 of the Present Invention

### (i) Two-Dimensional Polyacrylamide Electrophoresis

The sample collected from Example 2(c) is treated and analysed as described above in Example 3(a)(i).

The major protein spots in the gel correspond to isoforms of IL-4. Examination of the gel revealed that IL-4 of the present invention contains 1 to 3 isoforms. Tables 11 and 12 shows key properties of these isoforms: the pI values (±1.0), the apparent molecular weights (±20%), and the relative intensities (±20% of the actual value or ±2% of the total, whichever is larger). The values listed correspond to the intensity weighted center within the selected area of gel containing the spot and hence, are only reflective of the pI and molecular weight of the protein at one particular reading within the selected area of the gel. Taking into consideration the inherent variability of size and position of protein spots within 2D gels, the pI values for the molecule are determined to range from about 8 to 11 based on the values listed in Tables 11 and 12; and the apparent molecular weights of the molecule are determined to range from 15-20 kDa based on the values listed in Tables 11 and 12.

TABLE 11
Molecular weights and pI values of isoforms of IL-4

| | | | Relative Intensity (%) |
|---|---|---|---|
| | | Molecular | (Normalized Value) |
| | Isoelectric Point | Weight (kDa) | (±20% or ±2% of the |
| Spot Number | (pI) (±1.0) | (±20%) | total) |
| 2 | 9.45 | 14.87 | 13.54 |
| 3 | 9.67 | 15.17 | 55.38 |
| 4 | 9.89 | 15.25 | 31.09 |

TABLE 12
Molecular weights and pI values of isoforms of IL-4

| | | | Relative Intensity (%) |
|---|---|---|---|
| | | Molecular | (Normalized Value) |
| | Isoelectric Point | Weight | (±20% or ±2% of the |
| Spot Number | (pI) (±1.0) | (kDa) (±20%) | total) |
| 2 | 9.75 | 17.56 | 95.65 |
| 3 | 9.69 | 10.95 | 2.55 |
| 4 | 9.81 | 11.09 | 1.79 |

### (ii) One-Dimensional Polyacrylamide Electrophoresis

The sample collected from Example 2(c) was treated as described above in Example 3(a)(ii). The apparent molecular weight of the IL-4 (as observed by SDS-PAGE) following the release of N-linked oligosaccharides (by PNGase treatment) was between 10 and 20 kDa. The apparent molecular weight of the IL-4 (as observed by SDS-PAGE) following the release of N-linked oligosaccharides (by PNGase treatment) and O-linked oligosaccharides (by glycosidase cocktail) was between 10 and 18 kDa.

(iii) N-Terminal Sequencing of Proteins

N-terminal sequencing of the IL-4 of the present invention is performed as described above in Example 3(a)(iii).

### (iv) Peptide Mass Fingerprinting

Peptide mass fingerprinting of the IL-4 of the present invention was performed as described above in Example 3(a)(iv).

The identity of the gel spots were confirmed to be IL-4.

An observed 1 Da shift in the masses of tryptic peptides indicated the asparagine residues (N) of a NX(S/T/C) motif in the theoretical amino acid sequence of human IL-4 was modified to aspartic acid (D). Hence, a confirmed site of N-glycosylation of the IL-4 of the present invention is N-62 (when numbered from the start of the signal sequence).

### (v) Detection of Disulfide Bonds

IL-4 of the present invention was treated with trypsin (enzyme:protein=1:50) in 50 mM NH₄HCO₃ at 37° C. overnight. Half of the mixture was evaporated to dryness and re-dissolved in water. An aliquot of this solution equivalent to 0.5 μg protein was injected into an Agilent XCT Ultra LC-MS equipped with a C18 column (Zorbax C18, 150×0.5 mm, 5u) using acetonitrile/water as mobile phase modified with 0.1% formic acid. Peptides were eluted by a gradient from 0% to 90% acetonitrile in 45 min at a flow rate of 12 μl/min. The mass spectrometer was set in positive mode using Data-dependent Auto MSn. Peptides that were linked to each other by disulfide bonds were detected by MS precursor scanning of their combined MW with a loss of 2H and their structures were confirmed by subsequent MS/MS. The presence of disulfide bonds was further confirmed by treatment of the tryptic digest (1 ug in 25 L 0.1M NH₄HCO₃) with dithiothreitol (DTT, 0.1M, 3 μl) at room temperature in dark for 1 h, followed by a treatment with iodoacetamide (0.5M, 3 μl) under the same conditions. Disappearance of the disulfide-linked peptides provides further evidence for the existence of disulfides.

Alternatively, if disulfide bonds cannot be seen with the above-described method and the protein of the present invention is known to be glycosylated, the method is repeated after enzymatic deglycosylation (either PNGase F or glycosidase cocktail as required), as described above in Example 3(a)(ii).

Masses corresponding to the dipeptides 27-36 and 151-153, 46-61 and 89-99, and 67-71 and 113-126 were found showing that IL-4 of the present invention contains 3 disulfide bonds between Cys-27 and Cys-151, Cys-48 and Cys-89, and Cys-70 and Cys-123. These masses were not present after reduction and alkylation of the peptide sample.

### (d) Characterization of IL-5 of the Present Invention

### (i) Two-Dimensional Polyacrylamide Electrophoresis

The sample collected from Example 2(d) is treated and analysed as described above in Example 3(a)(i).

The major protein spots on the two gels correspond to isoforms of IL-5. The low intensity spots may be IL-5 or low level contaminants, however, there cannot be confirmed by PF due to the low intensity. Examination of the gel revealed that IL-5 of the present invention contains 15 to 25 isoforms. Tables 13 and 14 show key properties of these isoforms: the pI values (±1.0), the apparent molecular weights (±20%), and the relative intensities (±20% of the actual value or ±2% of the total, whichever is larger). The values listed correspond to the intensity weighted center within the selected area of gel containing the spot and hence, are only reflective of the pI and molecular weight of the protein at one particular reading within the selected area of the gel. Taking into consideration the inherent variability of spot size and position within 2D gels, the pI values for the molecule are determined to range from about 4-9 based on the values listed in Tables 13 and 14; and the apparent molecular weights of the molecule are determined to range from 15-25 kDa based on the values listed in Tables 13 and 14.

TABLE 13
Molecular weights and pI values of isoforms of IL-5

| | | Molecular | Relative Intensity (%) |
|---|---|---|---|
| Spot | Isoelectric Point | Weight | (Normalized Value) |
| Number | (pI) (±1.0) | (kDa) (±20%) | (±20% or ±2% of the total) |
| 2 | 4.788 | 18.023 | 1.371 |
| 3 | 5.153 | 19.566 | 0.633 |
| 4 | 5.156 | 18.186 | 1.196 |
| 5 | 5.379 | 19.131 | 1.730 |
| 6 | 5.486 | 18.322 | 3.831 |
| 7 | 5.704 | 21.023 | 0.910 |
| 8 | 6.051 | 20.921 | 1.063 |
| 9 | 6.464 | 20.510 | 0.845 |
| 10 | 5.994 | 16.171 | 2.441 |
| 11 | 6.347 | 16.172 | 0.947 |
| 12 | 6.346 | 15.432 | 0.613 |
| 13 | 6.491 | 13.798 | 0.478 |
| 14 | 5.720 | 18.286 | 16.824 |
| 15 | 6.062 | 18.155 | 22.948 |
| 16 | 6.449 | 17.884 | 20.839 |
| 17 | 6.867 | 17.443 | 14.944 |
| 18 | 7.339 | 17.542 | 4.812 |
| 19 | 7.749 | 17.500 | 3.576 |

TABLE 14
Molecular weights and pI values of isoforms of IL-5

| | | Molecular | Relative Intensity (%) |
|---|---|---|---|
| Spot | Isoelectric Point | Weight | (Normalized Value) |
| Number | (pI) (±1.0) | (kDa) (±20%) | (±20% or ±2% of the total) |
| 2 | 3.41 | 19.78 | 4.88 |
| 3 | 3.70 | 20.15 | 3.37 |
| 4 | 4.14 | 20.31 | 9.39 |
| 5 | 4.77 | 21.22 | 12.02 |
| 6 | 5.22 | 20.91 | 22.38 |
| 7 | 5.51 | 20.24 | 5.24 |
| 8 | 5.66 | 20.25 | 13.42 |
| 9 | 5.80 | 20.37 | 12.16 |
| 10 | 5.95 | 20.22 | 2.08 |
| 11 | 6.13 | 20.77 | 5.68 |
| 12 | 6.26 | 20.75 | 4.18 |
| 13 | 6.51 | 20.58 | 1.17 |
| 14 | 5.94 | 18.50 | 1.49 |
| 15 | 6.11 | 18.73 | 2.55 |

### (ii) One-Dimensional Polyacrylamide Electrophoresis

The collected sample from Example 2(d) was treated as described above in Example 3(a)(ii). The apparent molecular weight of the IL-5 (as observed by SDS-PAGE) following the release of N-linked oligosaccharides (by PNGase treatment) resulted in 2 bands: one between 11 and 16 kDa and the second between 15 and 25 kDa. The apparent molecular weight of the IL-5 (as observed by SDS-PAGE) following the release of N-linked oligosaccharides (by PNGase treatment) and O-linked oligosaccharides (by glycosidase cocktail) resulted in 2 bands: one between 10 and 15 kDa and the second between 15-22 kDa.

(iii) N-Terminal Sequencing

N-terminal sequencing of the IL-5 of the present invention is performed as described above in Example 3(a)(iii).

### (iv) Peptide Mass Fingerprinting

Peptide mass fingerprinting of the IL-5 of the present invention was performed as described above in Example 3(a)(iv).

The identity of the gel spots were confirmed to be IL-5.

### Example 4

### (a) Analysis of Amino Acid, Monosaccharide, Oligosaccharide, Phosphate, Sulphate and Isoform Composition of GM-CSF of the Present Invention

### (i) Preparation of Samples for Amino Acid, Monosaccharide, Oligosaccharide, Phosphate and Sulfate Analysis

For characterisation of monosaccharide and oligosaccharide glycosylation and phosphate and sulfate post-translational modifications, the saccharides were first removed from the polypeptide backbone by hydrolytic or enzymatic means. The sample buffer components were also removed and exchanged with water to avoid inhibition of the hydrolysis and enzymatic reactions before analysis began. A solution of purified GM-CSF in PBS was dialysed extensively against 4 litres of deionised ultrafiltered water (18 MOhm) for four days with two changes per day using a regenerated cellulose dialysis membrane (Spectrapore) with a nominal molecular weight cut-off (NMWC) of 5 KDa. After dialysis the solution was dried using a Savant Speed Vac (New York, USA). The dried down sample was then resuspended in 2 ml of deionised ultrafiltered water (18 MOhm) and divided into aliquots for the various analyses.

### (ii) Analysis of Amino Acid Composition by the Gas Phase Hydrolysis Method

Amino acids in the samples were analysed using precolumn derivatisation with 6-aminoquinolyl-N-hydroxysuccinimidyl carbamate (AQC). The stable fluorescent amino acid derivatives were separated and quantified by reversed phase (C18) HPLC. The procedure employed was based on the Waters AccQTag amino acid analysis methodology.

Three 100 μl samples of the GM-CSF preparation were taken and dried in a Speed Vac. The dried samples were then hydrolysed for 24 hours at 110° C. After hydrolysis the samples were dried again before derivatisation as follows. The dried samples were re-dissolved in 10 μL of an internal amino acid standard (α-aminobutyric acid, AABA), 35 μL of borate buffer was added followed by 15 μL of AQC derivatising reagent. The reaction mix was heated at 50° C. for 12 minutes in a heating block. The derivatised amino acid sample was transferred to the autosampler of a HPLC system consisting of a Waters Alliance 2695 Separation Module, a Waters 474 Fluorescence Detector and a Waters 2487 Dual λ Absorbance Detector in series. The control and analysis software was Waters Empower Pro Module (Waters Corporation, Milford. MA, USA). The samples were passed over a Waters AccQTag column (15 cm×3.9 mm ID) using chromatographic parameters (i.e. suitable eluents and gradient flows) known in the art.

(iii) Analysis of Neutral and Amino Monosaccharide Composition

Two 100 μl samples of the GM-CSF preparation were taken and treated in two different ways to liberate monosaccharides. Each treatment was performed in triplicate.

1. Hydrolysed with 2 M trifluoroacetic acid (TFA) heated to 100° C. for four hours to release neutral sugars (galactose, glucose, fucose and mannose).
2. Hydrolysed with 4 M HCl heated to 100° C. for four hours to release amino sugars (N-acetyl-galactosamine, N-acetyl-glucosamine).

All of the hydrolysates were lyophilised using a Speed Vac system, redissolved in 200 μl water containing 0.8 nmols of internal standard. For neutral and amino sugars the internal standard was 2-deoxy-glucose. The samples were then centrifuged at 10,000 g for 30 minutes to remove protein debris. The supernatant was transferred to a fresh tube and analysed by high pH anion exchange chromatography using a Dionex LC 50 system with a GP₅₀ pump and an ED50 pulsed amperometric detector (Dionex Ltd). Analysis of neutral and amino sugars was performed using a Dionex CarboPac PA-20 column. Elution was performed with an isocratic hydroxide concentration of 10 mM over 20 minutes. This was achieved with the Dionex EG50 eluent generation system.

### (iv) Analysis of Acidic Monosaccharide Composition

A 100 μl sample of the GM-CSF preparation was taken and treated in the following way to liberate sialic acid monosaccharides. The treatment was performed in triplicate.

The sample was hydrolysed with 0.1 M TFA at 80° C. for 40 minutes to release N-Acetyl and N-Glycolyl neuraminic acid. The hydrolysates were lyophilised using a Speed Vac, redissolved in 200 μl water containing 0.8 nmols of internal standard. For sialic acid analysis the internal standard was lactobionic acid. Samples were then centrifuged at 10,000 g for 30 minutes to remove protein debris. The supernatant was transferred to a fresh tube and analysed by high pH anion exchange chromatography using a Dionex LC 50 system with a GP50 pump and an ED50 pulsed amperometric detector. Analysis of sialic acids was performed using a Dionex CarboPac PA1 using chromatographic parameters (i.e. suitable eluents and gradient flows) known in the art.

### (v) Analysis of Oligosaccharide Composition.

For analysis of oligosaccharide composition two 300 μl samples are taken in triplicate and treated in one of the following ways.

Release of N-linked oligosaccharides is achieved with the enzyme peptide-N4-(N-acetyl-β-D-glucosaminyl) Asparagine Amidase (PNGase). A ⅕^{th} volume of denaturation solution (2% SDS (Sigma)/1 M β-mercaptoethanol (Sigma)) is added to the sample. The sample is heated to 100° C. for 5 minutes. A 1/10^{th} volume of 15% Triton-X100 (Sigma) is added to the sample. The sample is mixed gently and allowed to cool to room temperature. 25 Units of PNGase (Sigma) is added and incubated overnight at 37° C.

Release of O-linked oligosaccharides is achieved by the process of β-elimination. A ½ volume of 4M sodium borohydride (freshly made) (Sigma) solution is added to the sample. A ½ volume of 0.4 M NaOH (BDH, HPLC grade) is added to the sample. The sample is incubated at 50° C. for 16 hours. The sample is cooled on ice and a ½ volume of 0.4 M acetic acid (Sigma) is added to the sample.

Both the N-linked and O-linked samples are further processed to remove buffer components using a Carbo Pac graphitised carbon SPE column. The column equilibration and elution conditions are as follows. The column is pre-equilibrated with 1 column volume of 80% v/v acetonitrile (Sigma) followed by two column volumes of H₂O. The sample is loaded under gravity flow and the column washed with two column volumes of H₂O. To elute neutral oligosaccharides 2 ml of 50% v/v acetonitrile is applied to the column. To elute acidic oligosaccharides 2 ml of 50% v/v acetonitrile/0.1% v/v formic acid is applied to the column. Any remaining oligosaccharides are eluted by the addition of 2 ml of 80% v/v acetonitrile/0.1% v/v formic acid.

Individual fractions from the SPE columns containing the neutral or acidic N-linked oligosaccharides and the neutral or acidic O-linked oligosaccharides are dried down to completion using a Speed Vac. The samples are redissolved in 200 μl water and analysed by high pH anion exchange chromatography using a Dionex LC 20 system with a GP50 pump and an ED50 pulsed amperometric detector. Analysis of neutral and acidic oligosaccharides is performed using a CarboPac PA100 column and chromatographic parameters (i.e. suitable eluents and gradient flows) known in the art.

### (vi) Analysis of Sulfate and Phosphate Composition

Sulfate/phosphate analysis is performed essentially by the method described by Harrison and Packer (Harrison and Packer Methods Mol Biol 125:211-216, 2000).

A 100 μl sample of the GM-CSF preparation is taken for sulfate/phosphate analysis and hydrolysed in 4 M HCl at 100° C. for four hours. The HCl is removed by drying the samples in a Speed Vac system. Samples are then redissolved into 200 μl H₂O. 24 μl of sample is injected onto a Dionex LC 50 system with a GP50 pump and a ED50 conductivity detector. Separation is performed by a Dionex IonPac AS11 Anion exchange column using chromatographic parameters (i.e. suitable eluents and gradient flows) known in the art. The hydroxide ions are neutralised using a Dionex Self Regenerating Anion Micromembrane Suppressor (SRMS-1) and the SO₄ and PO₄ ions detected using a conductivity detector.

(vii) Further Separation of Protein Isoforms

Further separation of GM-CSF isoforms is performed using a pellicular anion exchange column. A suitable volume of sample, for example, 24 μl, is separated through a ProPac SAX-10 column (Dionex Ltd) using a Dionex SUMMIT system with UV-Vis detector (Dionex Ltd). Separation is performed using suitable eluents and gradients known in the art. GM-CSF isoforms are found to elute in a pattern of distinct peaks.

(viii) Results

### Amino Acid Composition

The GM-CSF was hydrolysed, derivatised and analysed by reversed phase high performance liquid chromatography as described above to give the following amino acid composition. Results were expressed as amount by weight and the percentage occurrence of each amino acid in the sequence (Table 15).

TABLE 15
Amino Acid Composition

| AA | Run 1 | Run 2 | Run 3 | Average | SD |
|---|---|---|---|---|---|
| D | 8.57 | 8.68 | 8.71 | 8.65 | 0.07 |
| S | 6.72 | 6.78 | 6.35 | 6.61 | 0.23 |
| E | 18.37 | 18.20 | 18.19 | 18.25 | 0.10 |
| G | 5.01 | 5.62 | 5.14 | 5.26 | 0.32 |
| H | 2.11 | 1.68 | 2.21 | 2.00 | 0.28 |
| R | 5.23 | 5.16 | 5.17 | 5.19 | 0.04 |
| T | 8.41 | 8.38 | 8.31 | 8.37 | 0.06 |
| A | 7.92 | 7.88 | 7.87 | 7.89 | 0.03 |
| P | 7.78 | 7.65 | 7.86 | 7.76 | 0.11 |
| Y | 1.54 | 1.34 | 1.47 | 1.45 | 0.10 |
| V | 1.54 | 4.14 | 4.12 | 3.27 | 1.49 |
| M | 2.05 | 2.16 | 2.20 | 2.14 | 0.07 |
| K | 5.45 | 5.32 | 5.30 | 5.36 | 0.08 |
| I | 3.02 | 3.07 | 3.12 | 3.07 | 0.05 |
| L | 10.03 | 10.23 | 10.29 | 10.18 | 0.13 |
| F | 3.66 | 3.72 | 3.70 | 3.69 | 0.03 |
| Total | 97.42 | 100 | 100 | 99.14 | |

### Monosaccharides

The individual monosaccharides were hydrolysed from the amino acid backbone of GM-CSF and analysed by High pH anion exchange chromatography (HP AEC) as described to give the following compositional analysis. Results from the samples are normalised to GalNAc and three times of mannose, respectively (Table 16-18). Table 19 is a summary of results from the three samples.

TABLE 16
Monosaccharide Composition Run 1

| Monosaccharide | Norm. GalNAc | Norm Mannose |
|---|---|---|
| Fucose | 0.19 | 0.11 |
| GalNAc | 1.00 | 0.59 |
| GlcNAc | 10.80 | 6.39 |
| Galactose | 5.36 | 3.17 |
| Glucose | 46.67 | 27.59 |
| Mannose | 5.07 | 3.00 |
| NeuNAC | 2.64 | 1.56 |
| NeuGly | 0 | 0 |

TABLE 17
Monosaccharide Composition Run 2

| Monosaccharide | Norm. GalNAc | Norm Mannose |
|---|---|---|
| Fucose | 1.02 | 1.97 |
| GalNAc | 1.00 | 1.92 |
| GlcNAc | 5.02 | 9.66 |
| Galactose | 2.39 | 4.59 |
| Glucose | 21.69 | 41.74 |
| Mannose | 1.56 | 3.00 |
| NeuNAC | 0.30 | 0.59 |
| NeuGly | 0.00 | 0.00 |

TABLE 18
Monosaccharide Composition Run 3

| Monosaccharide | Norm. GalNAc | Norm Mannose |
|---|---|---|
| Fucose | 0.57 | 0.79 |
| GalNAc | 1.00 | 1.40 |
| GlcNAc | 5.14 | 7.17 |
| Galactose | 1.85 | 2.58 |
| Glucose | 21.26 | 29.69 |
| Mannose | 2.15 | 3.00 |
| NeuNAC | 0.00 | 0.00 |
| NeuGly | 0.00 | 0.00 |

TABLE 19
Monosaccharide Composition

| | |
|---|---|
| Norm. | Norm. |
| GalNAc | Mannose |

| Monosaccharide | Min | Max | Min | Max |
|---|---|---|---|---|
| Fucose | 0.19 | 1.02 | 0.11 | 1.97 |
| GalNAc | 1.00 | 1.00 | 0.59 | 1.92 |
| GlcNAc | 5.02 | 10.80 | 6.39 | 9.66 |
| Galactose | 1.85 | 5.36 | 2.58 | 4.59 |
| Glucose | 21.26 | 46.67 | 27.59 | 41.74 |
| Mannose | 1.56 | 5.07 | 3.00 | 3.00 |
| NeuNAC | 0.00 | 2.64 | 0.00 | 1.56 |
| NeuGly | 0.00 | 0.00 | 0.00 | 0.00 |

Taking into consideration the inherent variability of the above-described chromatographic procedures, the monosaccharide and sialic acid content of the GM-CSF of the present invention, when normalized to GalNAc, is determined to be about 1 to 0.1-1.5 fucose, 1 to 2-12 GlcNAc, 1 to 1.0-6.0 galactose, 1 to 1.0-6.0 mannose and 1 to 0-3.0 NeuNAc; and when normalized to 3 times of mannose, is determined to be about 3 to 0.1-2.5 fucose, 3 to 0.5-2.5 GalNAc, 3 to 5.0-10.0 GlcNAc, 3 to 2.0-5.0 galactose and 3 to 0-3.0 NeuNAc.

The amino acid composition data were combined with the monosaccharide and phosphate and sulfate data to give the content of the various species (Table 20). Taking into consideration the inherent variability of the above-described chromatographic procedures, the percentage acidic monosaccharide content of the GM-CSF of the present invention is determined to range from about 6-11%, the acidic percentage of N-linked oligosaccharide of the GM-CSF of the present invention is determined to range from about 27-41% and the acidic percentage of O-linked oligosaccharide of the GM-CSF of the present invention is determined to range from about 43-66%.

TABLE 20
Calculated content

| | |
|---|---|
| % Acidic monosaccharide | 8.4 |
| Neutral percentage of N-linked oligosaccharide | 67.05 |
| Acidic percentage of N-linked oligosaccharide | 32.95 |
| Neutral percentage of O-linked oligosaccharide | 45.81 |
| Acidic percentage of O-linked oligosaccharide | 54.19 |

### (b) Analysis of Amino Acid, Monosaccharide, Oligosaccharide, Phosphate, Sulfate and Isoform Composition of IL-3 of the Present Invention

### (i) Preparation of Samples for Amino Acid, Monosaccharide, Oligosaccharide, Phosphate, Sulfate and Isoform Analysis

A solution of purified IL-3 in PBS was treated as described above in Example 4(a)(i).

### (ii) Analysis of Amino Acid Composition by the Gas Phase Hydrolysis Method

Samples of the IL-3 preparation were treated as described above in Example 4(a)(ii).

(iii) Analysis of Neutral and Amino Monosaccharide composition

Samples of the IL-3 preparation were treated as described above in Example 4(a)(iii).

### (iv) Analysis of Acidic Monosaccharide Composition

A sample of the IL-3 preparation was treated as described above in Example 4(a)(iv).

### (v) Analysis of Oligosaccharide Composition

Samples of the IL-3 preparation are treated as described above in Example 4(a)(v).

### (vi) Analysis of Sulfate and Phosphate Composition

A sample of the IL-3 preparation is treated as described above in Example 4(a)(vi).

(vii) Further Separation of Protein Isoforms

A sample of the IL-3 preparation is treated as described above in Example 4(a)(vii). IL-3 isoforms are found to elute in a pattern of distinct peaks.

(viii) Results

### Monosaccharides

The individual monosaccharides, phosphate and sulfate was hydrolysed from the amino acid backbone of IL-3 and analysed by High pH anion exchange chromatography (HP AEC) as described above to give the following compositional analysis. Results from the samples are normalised to GalNAc and three times mannose respectively (Table 21-23). Table 24 is a summary of results from the three samples. Note that glucose is a common contaminant and is not normally a component of N- or O-linked oligosacchrides.

TABLE 21
Monosaccharide Composition Run 1

| Monosaccharide | Norm. GalNAc | Norm. Mannose |
|---|---|---|
| Fucose | 5.02 | 15.75 |
| GalNAc | 1.00 | 3.14 |
| GlcNAc | 3.88 | 12.18 |
| Galactose | 1.82 | 5.70 |
| Glucose | 24.18 | 75.84 |
| Mannose | 0.96 | 3.00 |
| NeuNAC | 0.08 | 0.25 |
| NeuGly | 0.00 | 0.00 |

TABLE 22
Monosaccharide Composition Run 2

| Monosaccharide | Norm. GalNAc | Norm. Mannose |
|---|---|---|
| Fucose | 3.20 | 11.84 |
| GalNAc | 1.00 | 3.70 |
| GlcNAc | 3.70 | 13.69 |
| Galactose | 0.90 | 3.32 |
| Glucose | 16.07 | 59.38 |
| Mannose | 0.81 | 3.00 |
| NeuNAC | 0.06 | 0.21 |
| NeuGly | 0.00 | 0.00 |

TABLE 23
Monosaccharide Composition Run 3

| Monosaccharide | Norm. GalNAc | Norm. Mannose |
|---|---|---|
| Fucose | 4.24 | 9.59 |
| GalNAc | 1.00 | 2.26 |
| GlcNAc | 4.07 | 9.19 |
| Galactose | 1.64 | 3.70 |
| Glucose | 20.96 | 47.40 |
| Mannose | 1.33 | 3.00 |
| NeuNAC | 0.05 | 0.12 |
| NeuGly | 0.00 | 0.00 |

TABLE 24
Monosaccharide Composition

| | |
|---|---|
| Norm. | Norm. |
| GalNAc | Mannose |

| Monosaccharide | Min | Max | Min | Max |
|---|---|---|---|---|
| Fucose | 3.20 | 5.02 | 9.59 | 15.75 |
| GalNAc | 1.00 | 1.00 | 2.26 | 3.70 |
| GlcNAc | 3.70 | 4.07 | 9.19 | 13.69 |
| Galactose | 0.90 | 1.82 | 3.32 | 5.70 |
| Glucose | 16.07 | 24.18 | 47.40 | 75.84 |
| Mannose | 0.81 | 1.33 | 3.00 | 3.00 |
| NeuNAC | 0.05 | 0.08 | 0.12 | 0.25 |
| NeuGly | 0.00 | 0.00 | 0.00 | 0.00 |

Taking into consideration the inherent variability of the above-described chromatographic procedures, the monosaccharide and sialic acid content of the IL-3 of the present invention, when normalized to GalNAc, is determined to be about 1 to 2-6 fucose, 1 to 3-5 GlcNAc, 1 to 0.5-2 galactose, 1 to 0.5-2 mannose and 1 to 0-2 NeuNAc; and when normalized to 3 times of mannose, is determined to be about 3 to 5-16 fucose, 3 to 2-4 GalNAc, 3 to 9-14 GlcNAc, 3 to 3-6 galactose and 3 to 0.1-2 NeuAc.

### (c) Analysis of Amino Acid, Monosaccharide, Oligosaccharide, Phosphate, Sulfate and Isoform Composition of IL-4 of the Present Invention

### (i) Preparation of Samples for Amino Acid, Monosaccharide, Oligosaccharide, Phosphate, Sulfate and Isoform Analysis

A solution of purified IL-4 in PBS was treated as described above in Example 4(a)(i).

### (ii) Analysis of Amino Acid Composition by the Gas Phase Hydrolysis Method

Samples of the IL-4 preparation were treated as described above in Example 4(a)(ii).

(iii) Analysis of Neutral and Amino Monosaccharide composition

Samples of the IL-4 preparation were treated as described above in Example 4(a)(iii).

### (iv) Analysis of Acidic Monosaccharide Composition

A sample of the IL-4 preparation was treated as described above in Example 4(a)(iv).

### (v) Analysis of Oligosaccharide Composition

Samples of the IL-4 preparation are treated as described above in Example 4(a)(v).

### (vi) Analysis of Sulfate and Phosphate Composition

A sample of the IL-4 preparation is treated as described above in Example 4(a)(vi).

(vii) Further Separation of Protein Isoforms

A sample of the IL-4 preparation is treated as described above in Example 4(a)(vii). IL-4 isoforms are found to elute in a pattern of distinct peaks.

(viii) Results

The individual monosaccharides, phosphate and sulfate was hydrolysed from the amino acid backbone of IL-4 and analysed by High pH anion exchange chromatography (HP AEC) and combined with the amino acid composition data to give the content of the various species as a percent by weight (Table 25). Glucose is a common contaminant and is not normally found in N- or O-linked oligosaccharides. Taking into consideration the inherent variability of the above-described chromatographic procedures, the acidic percentage of N-linked oligosaccharide of the IL-4 of the present invention is determined to range from 0-30%.

TABLE 25
Calculated content

| | |
|---|---|
| Neutral % of N-linked oligosaccharide | 90 |
| Acidic % of N-linked oligosacchharide | 10 |

### (d) Analysis of Amino Acid, Monosaccharide, Oligosaccharide, Phosphate, Sulfate and Isoform Composition of IL-5 of the Present Invention

### (i) Preparation of Samples for Amino Acid, Monosaccharide, Oligosaccharide, Phosphate, Sulfate and Isoform Analysis

A solution of purified IL-5 in PBS was treated as described above in Example 4(a)(i).

### (ii) Analysis of Amino Acid Composition by the Gas Phase Hydrolysis Method

Samples of the IL-5 preparation were treated as described above in Example 4(a)(ii).

(iii) Analysis of Neutral and Amino Monosaccharide composition

Samples of the IL-5 preparation were treated as described above in Example 4(a)(iii).

### (iv) Analysis of Acidic Monosaccharide Composition

A sample of the IL-5 preparation was treated as described above in Example 4(a)(iv).

### (v) Analysis of Oligosaccharide Composition

Samples of the IL-5 preparation are treated as described above in Example 4(a)(v).

### (vi) Analysis of Sulfate and Phosphate Composition

A sample of the IL-5 preparation was treated as described above in Example 4(a)(vi).

(vii) Further Separation of Protein Isoforms

A sample of the IL-5 preparation is treated as described above in Example 4(a)(vii). IL-5 isoforms are found to elute in a pattern of distinct peaks.

(viii) Results

### Amino Acid Composition

The IL-5 was hydrolysed, derivatised and analysed by reversed phase high performance liquid chromatography as described to give the following amino acid composition (Table 26). Results are expressed as amount by weight and the percentage occurrence of each amino acid in the sequence (including standard deviation (SD)). Glycine is a known contaminant in amino acid analysis that can artificially alter the amino acid composition. With this taken into account, the results are comparable to the theoretical values, except we note that alanine (A) and aspartic acid (D) levels, are higher than expected.

TABLE 26
Amino Acid Composition

| AA | Run 1 | Run 2 | Run 3 | Average | SD |
|---|---|---|---|---|---|
| D | 8.33 | 8.77 | 8.55 | 8.55 | 0.22 |
| S | 5.01 | 4.59 | 4.58 | 4.73 | 0.25 |
| E | 16.99 | 16.95 | 16.89 | 16.94 | 0.05 |
| G | 7.11 | 5.96 | 5.89 | 6.32 | 0.68 |
| H | 2.09 | 2.00 | 2.06 | 2.05 | 0.04 |
| R | 5.26 | 4.95 | 4.94 | 5.05 | 0.18 |
| T | 8.46 | 8.27 | 8.29 | 8.34 | 0.11 |
| A | 5.17 | 5.35 | 5.10 | 5.21 | 0.13 |
| P | 4.09 | 4.15 | 4.13 | 4.12 | 0.03 |
| Y | 1.37 | 1.69 | 1.67 | 1.58 | 0.18 |
| V | 5.99 | 5.97 | 6.01 | 5.99 | 0.02 |
| M | 0.09 | 0.53 | 0.54 | 0.39 | 0.26 |
| K | 8.37 | 8.37 | 8.57 | 8.43 | 0.12 |
| I | 6.84 | 7.11 | 7.20 | 7.05 | 0.19 |
| L | 11.99 | 12.42 | 12.64 | 12.35 | 0.33 |
| F | 2.85 | 2.91 | 2.94 | 2.90 | 0.04 |
| Total | 100.00 | 100.00 | 100.00 | 100.00 | |

### Monosaccharides and Sulfate

The individual monosaccharides, and sulfate was hydrolysed from the amino acid backbone of IL-5 and analysed by High pH anion exchange chromatography (HLP AEC) as described to give the following compositional analysis. Results from the samples are normalised to GalNAc and three times mannose respectively (Table 27-29). Table 30 is a summary of results from the three samples. Glucose is a common contaminant and is not normally a component of N- or O-linked oligosaccharides. We also note that there appeared to be a contaminant that co-eluted with the front edge of the galactose peak, thus artificially raising the galactose level, and that the level of GlcNAc seems high.

TABLE 27
Monosaccharide Composition Run 1

| Monosaccharide | nmol/nmol protein | Norm. GalNAc | Norm Mannose |
|---|---|---|---|
| Fucose | 0.00 | 0.00 | 0.00 |
| GalNAc | 2.06 | 1.00 | 2.56 |
| GlcNAc | 8.49 | 4.12 | 10.56 |
| Galactose | 3.90 | 1.89 | 4.85 |
| Glucose | 4.13 | 2.00 | 5.14 |
| Mannose | 2.41 | 1.17 | 3.00 |
| NeuNAC | 0.23 | 0.11 | 0.29 |
| NeuGly | 0.00 | 0.00 | 0.00 |
| SO₄ | 4.77 | 9.25 | 23.72 |

TABLE 28
Monosaccharide Composition Run 2

| Monosaccharide | nmol/nmol protein | Norm. GalNAc | Norm Mannose |
|---|---|---|---|
| Fucose | 0.72 | 0.33 | 0.87 |
| GalNAc | 2.19 | 1.00 | 2.65 |
| GlcNAc | 9.13 | 4.17 | 11.02 |
| Galactose | 3.75 | 1.71 | 4.53 |
| Glucose | 4.22 | 1.93 | 5.09 |
| Mannose | 2.48 | 1.13 | 3.00 |
| NeuNAC | 0.26 | 0.12 | 0.32 |
| NeuGly | 0.00 | 0.00 | 0.00 |
| SO₄ | 3.02 | 5.29 | 13.99 |

TABLE 29
Monosaccharide Composition Run 3

| Monosaccharide | nmol/nmol protein | Norm. GalNAc | Norm Mannose |
|---|---|---|---|
| Fucose | 0.72 | 0.32 | 0.87 |
| GalNAc | 2.28 | 1.00 | 2.50 |
| GlcNAc | 9.32 | 4.09 | 10.21 |
| Galactose | 3.77 | 1.65 | 4.13 |
| Glucose | 4.84 | 2.13 | 5.31 |
| Mannose | 2.74 | 1.20 | 3.00 |
| NeuNAC | 0.27 | 0.12 | 0.30 |
| NeuGly | 0.00 | 0.00 | 0.00 |
| SO₄ | 4.03 | 7.07 | 17.65 |

TABLE 30
Monosaccharide Composition

| | nmol/nmol protein | | Norm. GalNAc | | Norm Mannose | |
|---|---|---|---|---|---|---|
| Monosaccharide | Min | Max | Min | Max | Min | Max |
| Fucose | 0.00 | 0.72 | 0.00 | 0.33 | 0.00 | 0.87 |
| GalNAc | 2.06 | 2.28 | 1.00 | 1.00 | 2.50 | 2.65 |
| GlcNAc | 8.49 | 9.32 | 4.09 | 4.17 | 10.21 | 11.02 |
| Galactose | 3.75 | 3.90 | 1.65 | 1.89 | 4.13 | 4.85 |
| Glucose | 4.13 | 4.84 | 1.93 | 2.13 | 5.09 | 5.31 |
| Mannose | 2.41 | 2.74 | 1.13 | 1.20 | 3.00 | 3.00 |
| NeuNAC | 0.23 | 0.27 | 0.11 | 0.12 | 0.29 | 0.32 |
| NeuGly | 0.00 | 0.00 | 0.00 | 0.00 | 0.00 | 0.00 |
| SO₄ | 3.02 | 4.77 | 5.29 | 9.25 | 13.99 | 23.72 |

The amino acid composition data were combined with the monosaccharide and phosphate and sulfate data to give the content of the various species (Table 31). Taking into consideration the inherent variability of the above-described chromatographic procedures, the monosaccharide, sialic acid and sulfate content of the IL-5 of the present invention, when normalized to GalNAc, is determined to be about 1 to 0-0.5 fucose, 1 to 2-4.5 GlcNAc, 1 to 1-2 galactose, 1 to 1-2 mannose, 1 to 0.1-1 NeuNAc and 1 to 5-10 sulfate; and when normalized to 3 times of mannose, is determined to be about 3 to 0-1 fucose, 3 to 2-3 GalNAc, 3 to 3-12 GlcNAc, 3 to 2-5 galactose, 3 to 0.2-1 NeuNAc and 3 to 12-24 sulfate.

Further, taking into consideration the inherent variability of the above-described chromatographic procedures, the sialic acidic as a percentage of the monosaccharide content of the IL-5 of the present invention is determined to range from about 2-10%, the sulfation as a percentage of the monosaccharide content of the IL-5 of the present invention is determined to range from about 10-25%, the acidic percentage of N-linked oligosaccharide of the IL-5 of the present invention is determined to range from about 25-50% and the acidic percentage of O-linked oligosaccharide of the IL-5 of the present invention is determined to range from about 0-40%.

TABLE 31
Calculated Content

| | |
|---|---|
| Sialic acid as a percentage of the monosaccharide content | 2.6 |
| Sulfation as a percentage of the monosaccharide content | 17.7 |
| Acidic % of N-linked oligosaccharide | 36.5 |
| Acidic % of O-linked oligosaccharide | 8.2 |

### Example 5

### Glyco Mass Fingerprinting

The protein of the present invention is separated using 2D gel electrophoretic techniques as in Example 3 and blotted onto polyvinyl difluorethane (PVDF) membrane. The spots are stained using one of a standard array of protein stains (Colloidal Coomassie Blue, Sypro Ruby or Deep Purple), and the isoform relative amounts quantified using densitometry algorithms. The individual spots are excised and treated with an array of deglycosylating enzymes and/or chemical means, as appropriate, to remove the oligosaccharides present according to methods described in this document. Once the oligosaccharides are removed, they are separated and analysed on a liquid chromatography-electrospray mass spectrometry system (LC-MS) using a graphitised carbon column and organic solvent (MeCN) gradient elution system. The generated peak profile that is generated is a “fingerprint” of the oligosaccharides present on the isoform. Furthermore, the mass spectrometry system simultaneously generates information on the mass of each of the sugars present in the sample which is used to identify their structure through pattern matching with the GlycoSuite database. In addition, individual mass peaks can be fragmented multiple times to give MSⁿ spectra. These fragments allow structural prediction using methods known in the art, for example, by the use of the GlycosidlQ software package.

The above separation, deglycosylation and analysis procedures are repeated using a corresponding protein expressed in a non-human cell system, e.g. E. coli, yeast or CHO cells and the respective glyco mass fingerprints are found to be significantly different. At a structural level, such a result indicates different patterns of glycan structures present on the protein of the present invention and the corresponding non-human cell expressed protein.

### Example 6

### Fluorophore Assisted Carbohydrate Electrophoresis

Oligosaccharide profiles of the target molecule are derived using the fluorophore assisted carbohydrate electrophoresis protocols (FACE protocols). The oligosaccharides from the target cytokine are hydrolysed from the amino acid backbone using ammonium hydroxide and subsequently labelled using the fluorophore 8-aminonaphthalene-1,3,6-trisulfonic acid (ANTS). Polyacrylamide gel electrophoresis is used to separate the species and standards used to identify an oligosaccharide profile that is typical of the target molecule. Further, the oligosaccharides are identified using matrix assisted laser desorption and ionisation—time of flight mass spectrometry (MALDI-TOF) relying on the fluorophore and a specific matrix to ionise each sugar. The mass of each sugar is determined and potential structures identified using the GlycoSuite database. The potential sugar structures are further characterised by tandem mass spectrometric techniques, which allows partial or complete characterisation of the oligosaccharides present and their relative amounts. Further, the process is repeated using the isoforms identified by 2D gel electrophoresis to generate a profile of the oligosaccharides present on each of the isoforms isolated.

### Example 7

### QCM and SPR

The binding characteristics and activity of the target molecule is determined using either quartz crystal microbalance (QCM) or surface plasmon resonance (SPR). In both cases a suitable receptor for the molecule is bound to a wafer using the chemistry described by the manufacturer. The target molecule is dissolved into a suitable biological buffer and allowed to interact with the receptor on the chip by passing the buffer over it. Changes in the total protein mass on the surface of the wafer are measured either by change of oscillation frequency (in the case of QCM) or changes in the light scattering qualities of the chip (in the case of SPR). The chip is then treated with the biological buffer alone to observe the release of the target molecule back into solution. The rate at which the receptors reach saturation and complete disassociation is then used to calculate the binding curve of the target molecule.

### Example 8

### Generation of a Transgenic Host Cell Line

### (a) Transgenic Host Cell Line with alpha-2,6-sialyltransferase

The cDNA coding for alpha-2,6-sialyltransferase (alpha 2,6ST) is amplified by PCR from poly(A)-primed cDNA. The PCR product is ligated into a suitable vector, for instance pIRESpuro4 or pCEP4, to generate an alpha 2,6ST plasmid. The cloned cDNA is sequenced and its identity verified by comparison with the published alpha-2,6ST cDNA sequence. DNA sequencing is performed using known methods.

Mammalian host cells, including cell clones of the same lineage that express high levels of target molecule (cell line-target molecule) are transfected with the alpha 2,6ST plasmid, which also carries an antibiotic resistance marker. Selection of stably transfected cells is performed by incubation of the cells in the presence of the antibiotic; colonies of antibiotic-resistant cells that appear subsequent to transfection are pooled and examined for intracellular alpha 2,6ST activity. To isolate individual cell clones expressing alpha 2,6ST, cell pools are cloned by a limiting dilution process as described by Kronman (Gene 121: 295-304, 1992). Individual cell clones are chosen at random, cells expanded and clones tested for alpha 2,6ST activity.

Cell pellets are washed, resuspended in lysis buffer and left on ice prior to sonication. The cell lysate is centrifuged and the clear supernatant is assayed for protein concentration (via known methods) and sialyltransferase activity. Sialyltransferase activity is assayed by known methods, for example the method detailed by Datta et al. (J Biol Chem 270:1497-1500, 1995).

Expressed target molecule is purified from high-expressing alpha 2,6ST cell line-target molecule cells and subjected to in vitro and/or in vivo half-life bioassays (see Example 10). Target molecule from high-expressing alpha 2,6ST cell displays an increased in vitro and/or vivo half-life in comparison to target molecule derived from the same parent cell line without any subsequent transgene manuipulation or target molecule derived from other cell lines.

### (b) Transgenic Host Cell Line with Fucosyltransferase

The cDNA coding for a fucosyltransferase (FT) such as FUT1, FUT2, FUT3, FUT4, FUT5, FUT6, FUT7, FUT8, FUT9, FUT10, FUT11 is amplified by PCR from poly(A)-primed cDNA. The PCR product is ligated into a suitable vector, for instance pIRESpuro4 or pCEP4, to generate an alpha 2,6ST plasmid. The cloned cDNA is sequenced and its identity verified by comparison with the published FT cDNA sequence. DNA sequencing is performed using known methods.

Human host cells, including cell clones of the same lineage that express high levels of target molecule (cell line-target molecule) are transfected with the FT plasmid, which also carries an antibiotic resistance marker. Selection of stably transfected cells is performed by incubation of the cells in the presence of the antibiotic; colonies of antibiotic-resistant cells that appear subsequent to transfection are pooled and examined for intracellular FT activity. To isolate individual cell clones expressing FT, cell pools are cloned by a limiting dilution process as described by Kronman 1992 supra; Individual cell clones are chosen at random, cells expanded and clones tested for FT activity.

Cell pellets are washed, resuspended in lysis buffer and left on ice prior to sonication. The cell lysate is centrifuged and the clear supernatant is assayed for protein concentration (via known methods) and FT activity. FT activity is assayed by known methods, for example the method detailed by Mas et al. (Glycobiology 8(6):605-13, 1998).

Expressed target molecule is purified from high-expressing FT cell line-target molecule cells. A Lewis x-specific antibody, such as L5 and a sialyl Lewis x-specific antibody such as KM93, HECA493, 2H5 or CSLEX are used to test the presence of Lewis x or sialyl Lewis x structures according to methods known in the art, for example, as detailed in Lucka et al. (Glycobiology 15(1):87, 2005). Alternatively, the presence of Lewis x or sialyl Lewis x structures may be detected by treating the sample with appropriate glycosidases and detecting the effect of the glycosidases on parameters such as mass using MS or retention time using HPLC. Glyco mass fingerprinting, as described in Example 5, may also be employed to predict the presence of Lewis x or sialyl Lewis x structures.

### Example 9

### Differential Gene Expression

Differences in gene expression can be analyzed using a target cell line of the target molecule. The target cells are grown to the appropriate density and treated with a range of concentration of target molecule or buffer control for a number of hours, for instance, 72 hours.

At various time points RNA is harvested, purified, and reverse transcribed according to Affymetrix protocols. Labelled cRNA (e.g. biotin labelled) is then prepared and hybridised to expression arrays e.g. U133 GeneChips. Following washing and signal amplification, the GeneChips are scanned using a GeneChip scanner (Affymetrix) and the hybridisation intensities and fold change information at various time points is obtained using GeneChip software (Affymetrix).

The target molecule induces unique gene expression and results in different mRNA profiles upon comparison with profiles induced by cytokines or receptors produced from different sources e.g. E. coli, yeast or CHO cells.

### Example 10

### Determining the Half-Life of the Target Molecule of the Present Invention

The half-life of the target molecule is determined in an in vitro system. Composition containing target molecule is mixed into human serum/plasma and incubated at a particular temperature for a particular time (e.g. 37 degrees for 4 h, 12 h etc). The amount of target molecule remaining after this treatment is determined by ELISA methods or dot blot methods known in the art. The biological activity of the remaining target molecule is determined by performing a suitable bioassay chosen by a person skilled in the relevant art. The serum chosen may be from a variety of human blood groups (eg A, B, AB, O etc.).

The half-life of target molecule is also determined in an in vivo system. Composition containing target molecule is labelled by a radioactive tracer (or other means) and injected intravenously, subcutaneously, retro-orbitally, intramuscularly or intraperitonally into the species of choice for the study, for instance, mouse, rat, pig, primate or human. Blood samples are taken at time points after injection and assayed for the presence of target molecule (either by ELISA methods, dot blot methods or by trichloroacetic acid (TCA)-precipitable label e.g. radioactive counts). A comparison composition consisting of target molecule produced from other sources eg E. coli, yeast, or CHO cells can be run as a control.

### Example 11

### In Vivo Studies Using the Target Molecule of the Present Invention

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

Preferably to account for the psychological effects of receiving treatments, the trial is conducted in a double-blinded fashion. Volunteers are randomly assigned to placebo or target molecule treatment groups. Furthermore, the relevant clinicans are blinded as to the treatment regime administered to a given subject to prevent from being biased in their post-treatment observations. Using this randomization approach, each volunteer has the same chance of being given either the new treatment or the placebo.

Volunteers receive either the target molecule or placebo for an appropriate period with biological parameters associated with the indicated disease state or condition being measured at the beginning (baseline measurements before any treatment), end (after the final treatment), and at regular intervals during the study period. Such measurements include the levels of target molecule in body fluids, tissues or organs compared to pre-treatment levels. Other measurements include, but are not limited to, indices of the disease state or condition being treated, body weight, blood pressure, serum titers of pharmacologic indicators of disease such as specific disease indicators or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are adults aged 18 to 65 years and roughly an equal number of males and females participate in the study. Volunteers with certain characteristics are equally distributed for placebo and target molecule treatment. In general, the volunteers treated with placebo have little or no response to treatment, whereas the volunteers treated with the target molecule show positive trends in their disease state or condition index at the conclusion of the study.

### Example 12

### (a) Comparing the Bioactivities of GM-CSF of the Present Invention and GM-CSF Expressed Using Non-Human Systems

GM-CSF has been reported to induce proliferation in TF-1 cells. In a 48-well plate, 30000 TF-1 cells/ml were treated with 0-10 ng/ml GM-CSF for 9 days at 37° C. Cell numbers were then measured using a flow cytometry analysis. For the flow cytometry analysis, viable cells are counted using a FACScan flow cytometer (Becton Dickinson Immunocytometry Systems, San Jose, Calif.) using published methods (Dedov et al. Apoptosis 8:399-406, 2003). Data analysis is performed with CellQuest Software.

The above assay was repeated using two different human GM-CSF molecules (R & D Systems Cat # 215-GM; WHO #88/646), both expressed in E. coli.

ED50 was calculated after curve fitting the absorbance and the GM-CSF molecule concentration values using a 4-parameter equation.

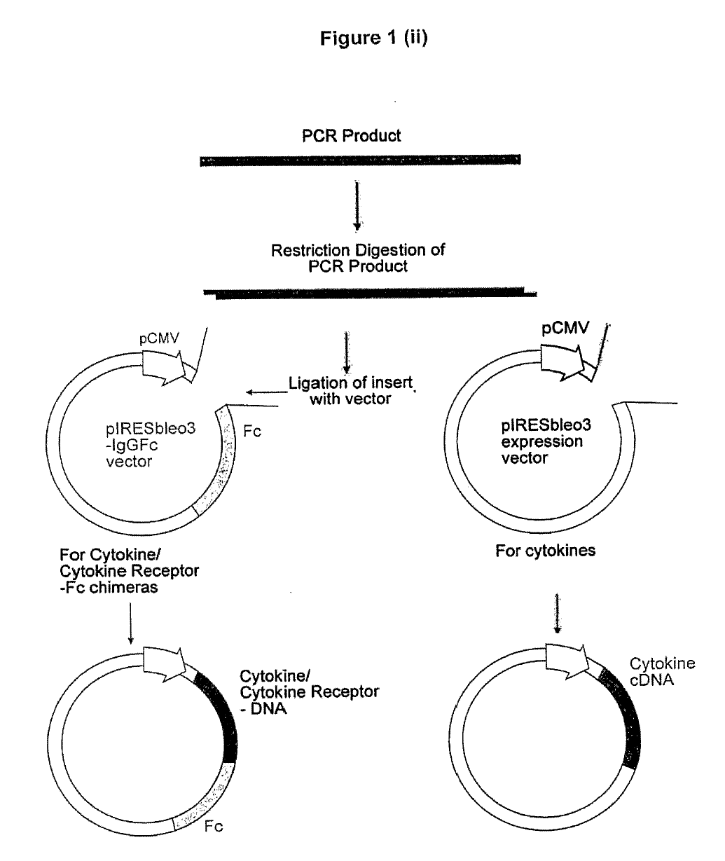

The ED50 of GM-CSF of the present invention was approximately 0.05-0.08 ng/ml, compared with the ED50 of the R & D Systems GM-CSF (0.4-0.6 ng/ml) and WHO GM-CSF (0.4-0.6 ng/ml), both expressed in E. coli (FIG. 2). Thus, the GM-CSF of the present invention displayed an 5-12 fold greater proliferative activity than a human GM-CSF expressed in E. coli.

### (b) Comparing the Serum Stability GM-CSF of the Present Invention and GM-CSF Expressed Using Non-Human Systems

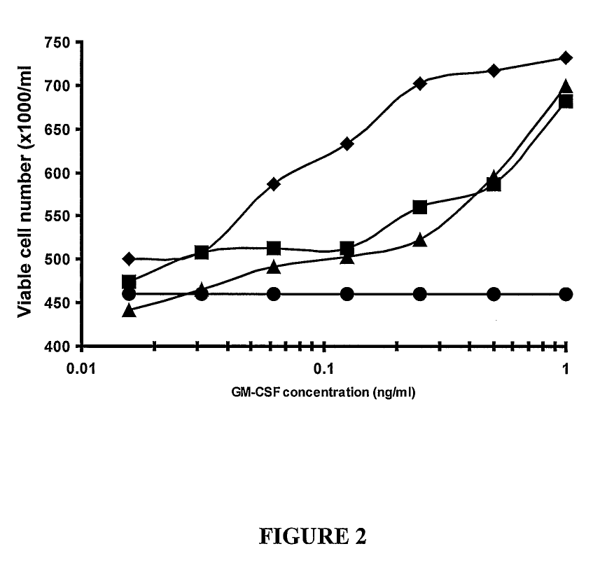

GM-CSF of the present invention and GM-CSF produced in E. coli (R&D Systems) were incubated in fetal calf serum for 24 hours, and the loss in product was determined by a commercially available ELISA (R&D Systems DuoSet) according to the manufacturer's protocol. GM-CSF bioactivity was measured as described above in Example 12(a). The level of loss of products were similar for both human cells and E. coli produced GM-CSF (results not shown). However, GM-CSF of the present invention had retained a higher bioactivity after 24 hours incubation in serum when compared with GM-CSF produced from E. coli (FIG. 3).

### (c) Differentiation and Proliferation of Granulocyte and Macrophage Colonies

GM-CSF has been reported to mediate the differentiation and proliferation of granulocyte and macrophage colonies. 200 TF-1 cells were plated in 35 mm culture dishes in triplicate and treated with GM-CSF of the present invention or human GM-CSF expressed from E. coli. The cells were incubated for 14 days at 37° C. and 5% CO₂ after which colony counts were performed. The colonies were counted using a microscope and a 100 mm culture dish marked with the scoring grid. A colony consisted of at least 40 cells.

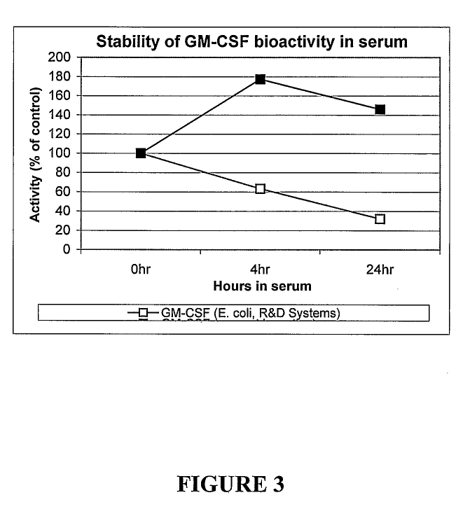

In a colony forming assay using TF-1 cells, GM-CSF of the present invention produced 1.5-2 fold more colonies (FIG. 4) over a range of concentrations, [1.25, 2.5, 5 ng/ml (p<0.01)] than human GM-CSF expressed from E. coli.

### (d) Comparing the Bioactivities of IL-3 of the Present Invention and IL-3 Expressed using Non-Human Systems

IL-3 has been reported to induce proliferation in M-NFS-60 cells. In a 48-well plate, 30000 M-NFS-60 cells/ml were treated with 0-1 ng/ml IL-3 of the present invention for 4 days at 37° C. Cell numbers were then measured using a flow cytometry analysis. For the flow cytometry analysis, viable cells are counted using a FACScan flow cytometer (Becton Dickinson Immunocytometry Systems, San Jose, Calif.) using published methods (Dedov et al., Apoptosis 8:399-406, 2003). Data analysis is performed with CellQuest Software.

The above assay was repeated using two different human IL-3 molecules (R & D Systems Cat # 203-IL; WHO #91/510), both expressed in E. coli.

ED50 was calculated after curve fitting the absorbance and the IL-3 molecule concentration values using a 4-parameter equation.

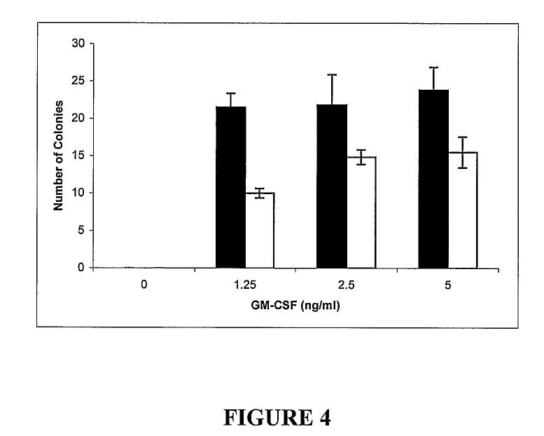

The ED50 of IL-3 of the present invention was approximately 0.12-0.18 ng/ml, compared with the ED50 of the R & D Systems human IL-3 (0.20-0.30 ng/ml) and WHO human IL-3 (0.20-0.30 ng/ml), both expressed in E. coli (FIG. 5). Thus, the IL-3 of the present invention displayed an approximate 1.1-2.5 fold greater proliferative activity than a human IL-3 expressed in E. coli.

### (e) Comparing the Bioactivities of IL-4 of the Present Invention and IL-4 Expressed Using Non-Human Systems

(i) IL-4 of the present invention was tested against IL-4 expressed using non-human Systems (CHO and E. coli). TF-1 cells (100000 cells/ml) were grown in the presence of 0-2 ng/ml IL-4 of the present invention for 7 days at 37° C. in a 96-well plate. Cell viability was then measured using the CellTiter 96 Aqueous One Solution Cell Proliferation Assay (Promega). In this assay a tetrazolium compound MTS ((3-(4,5-dimethylthiazol-2-yl)-5-(3-carboxymethoxyphenyl)-2-(4-sulfophenyl)-2H-tetrazolium) in the presence of an electron coupling reagent (phenazine methosulfate) is bioreduced by cells into a formazan product. The concentration of the formazan is determined by reading the absorbance of the resultant solution at 490 nm by a spectrophotometer (E max precision microplate reader, Molecular Devices).

The above assay was repeated using two different human IL-4 molecules expressed in non-human cells (R & D Systems Cat # 204-IL, expressed in E. coli; WHO #88/656, expressed in Chinese Hamster Ovary (CHO) cells).

ED₅₀ was calculated after curve fitting the absorbance and the IL-4 concentration values using a 4-parameter equation.

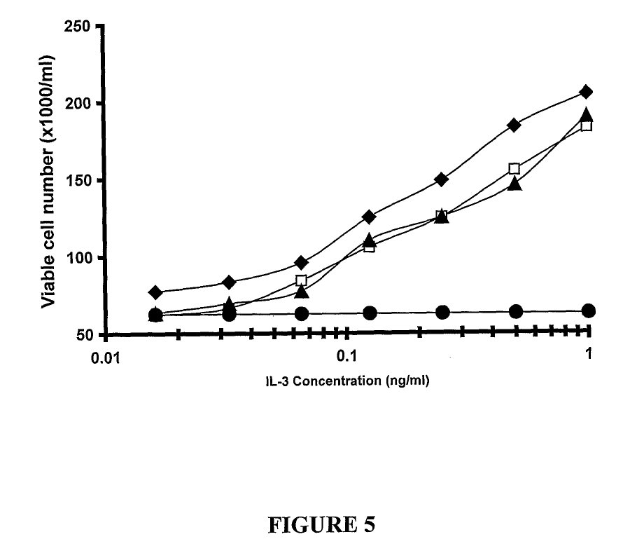

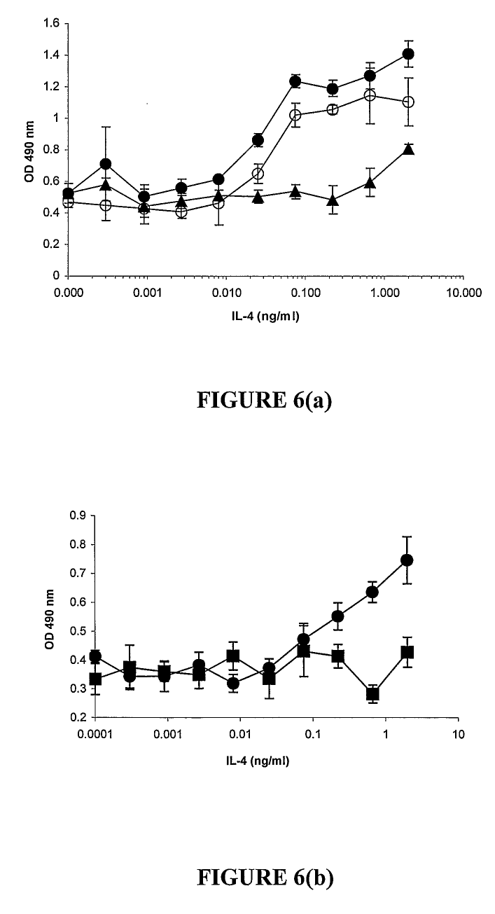

The ED₅₀ of IL-4 of the present invention was approximately 0.024-0.036 ng/ml, compared with the ED₅₀ of the R & D Systems IL-4 (0.9-1.3 ng/ml; expressed in E. coli) and WHO IL-4 (ED₅₀ 0.028-0.042 ng/ml; expressed in CHO cells) (FIG. 6(a)). Thus, the IL-4 of the present invention displayed an approximate 25-54 fold greater proliferative activity than human IL-4 expressed in E. coli, and up to 1.75 fold greater proliferative activity than human IL-4 expressed in CHO cells.

(ii) To further test stability under standard cell culture conditions, IL-4 of the present invention was pre-incubated at 37° C. in cell culture medium (RPMI 1640 medium/10% foetal bovine serum) for 4 days prior to addition to TF-1 cells. The pre-incubated medium containing IL-4 of the present invention was then added to TF-1 cells (200,000 cells/ml) in a 96-well plate at 0-2 ng/ml. Cells were grown for 3 days then cell viability was measured using the CellTiter 96 Aqueous One Solution Cell Proliferation Assay (Promega). In this assay a tetrazolium compound MTS ((3-(4,5-dimethylthiazol-2-yl)-5-(3-carboxymethoxyphenyl)-2-(4-sulfophenyl)-2H-tetrazolium) in the presence of an electron coupling reagent (phenazine methosulfate) is bioreduced by cells into a formazan product. The concentration of the formazan was determined by reading the absorbance of the resultant solution at 490 nm by a spectrophotometer (E max precision microplate reader, Molecular Devices).

The above assay was repeated using a human IL-4 molecule expressed in E. coli (R & D Systems Cat # 204-IL).

The ED₅₀ was calculated after curve fitting the absorbance and the IL-4 concentration values using a 4-parameter equation. The ED₅₀ of IL-4 of the present invention under cell culture conditions was 0.24-0.36 ng/ml whereas the ED₅₀ of the E. coli expressed IL-4 was 4.7-7.1 ng/ml (FIG. 6(b)). Thus the IL-4 of the present invention displayed a 13-30 fold greater ability to induce TF-1 cell proliferation than a human IL-4 molecule expressed in E. coli, demonstrating its increased stability in cell culture.

### (f) Comparing the Bioactivities of IL-5 of the Present Invention and IL-5 Expressed Using Non-Human Systems

The human erythroleukaemia cell line TF-1 responds to a range of cytokines. IL-5 has been reported to induce proliferation in TF-1 cells. In a 96-well plate, 10000 TF-1 cells/well are treated with 0-100 ng/ml IL-5 for 68 hours at 37° C.

Cell numbers are then measured using the CellTiter 96 Aqueous One Solution Cell Proliferation Assay (Promega). In this assay a tetrazolium compound MTS ((3-(4,5-dimethylthiazol-2-yl)-5-(3-carboxymethoxyphenyl)-2-(4-sulfophenyl)-2H-tetrazolium) in the presence of an electron coupling reagent (phenazine methosulfate) is bioreduced by cells into a formazan product. The concentration of the formazan is determined by reading the absorbance of the resultant solution at 490 nm by a spectrophotometer (E max precision microplate reader, Molecular Devices).

ED50 is calculated after curve fitting the absorbance and the IL-5 concentration values using a 4 parameter equation.

The above assay is repeated using IL-5 expressed in non-human cell systems, e.g. E. coli, yeast or CHO cells and the ED50s are found to be significantly different.

### Example 13

### (a) In Vitro Comparison of Immunoreactivity Profiles Between GM-CSF of the Present Invention and Human GM-CSF Expressed Using Non-Human Systems

Protein estimation of GM-CSF of the present invention is determined using a standard protein assay technique, for example, the Bradford protein assay (Bradford M Anal Biochem 72:248-254, 1976).

GM-CSF of the present invention, standardised using the standard protein assay results, is diluted and tested in a commercially available ELISA kit, for example, a R&D Systems human GM-CSF DuoSet® ELISA kit (Cat # DY215) in accordance with the manufacturer's instructions. The above-mentioned ELISA kit employs a human GM-CSF expressed in E. coli as a standard.

It is found that the R&D Systems human GM-CSF DuoSet® ELISA kit incorrectly estimates the concentration of GM-CSF of the present invention at an OD450 nm when compared with the corresponding GM-CSF of the present invention concentration as determined by the standard protein assay.

At a structural level, such a result will indicate different immunoreactivity profiles of GM-CSF of the present invention and a non-human cell expressed human GM-CSF molecule.

### (b) In Vitro Comparison of Immunoreactivity Profiles Between IL-3 of the Present Invention and Human Il-3 Expressed Using Non-Human Systems

Protein estimation of IL-3 of the present invention was determined using the Bradford protein assay (Bradford 1976 supra).

IL-3 of the present invention, standardised using the Bradford assay results, and human IL-3 expressed in insect cells (BD Pharmingen; Cat. # 554604) were diluted and tested in a R&D Systems human IL-3 DuoSet® ELISA kit (Cat.# DY203) in accordance with the manufacturer's instructions. The above-mentioned ELISA kit employs a human IL-3 expressed in E. coli cells as a standard.

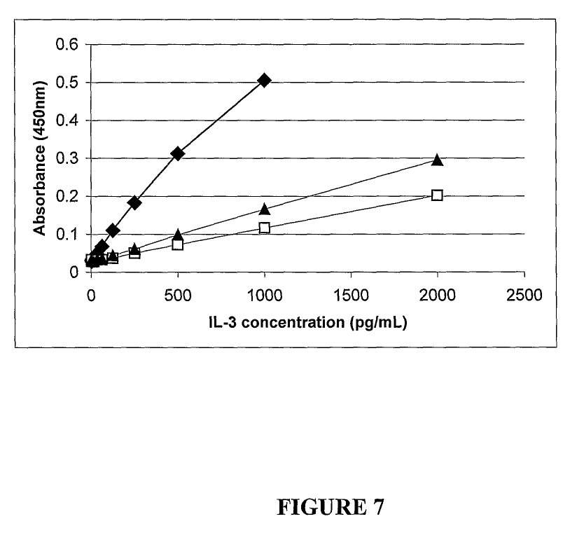

The R&D Systems DuoSet® IL-3 ELISA kit results gave a concentration estimate of IL-3 of the present invention of approximately 466 pg/ml at an OD450 nm of 0.3 (FIG. 7) when estimated from the IL-3 expressed in E. coli standard curve. Whereas, the actual interpolated concentration of IL-3 of the present invention was approximately 2000 pg/ml at a similar OD450 nm value (FIG. 7). Similarly, the R&D Systems DuoSet® IL-3 ELISA kit results gave a concentration estimate of human IL-3 expressed in insect cells of approximately 280 pg/ml at an OD450 nm of 0.2 (FIG. 7) when estimated from the IL-3 expressed in E. coli standard curve. Whereas, the actual interpolated concentration of human IL-3 expressed in insect cells was approximately 2000 pg/ml at a similar OD450 nm value (FIG. 7).

These results represent a greater than 4-fold underestimate of the IL-3 of the present invention concentration by the R&D Systems human IL-3 DuoSet® ELISA kit, a commercial kit employing a E. coli-expressed human IL-3 standard and antibodies against E. coli-expressed human IL-3, that is used to evaluate levels of native human expressed IL-3 in laboratory samples and human patient samples.

These results indicates different immunoreactivity profiles of IL-3 of the present invention and non-human cell expressed human IL-3 molecules.

### (c) In Vitro Comparison of Immunoreactivity Profiles Between IL-4 of the Present Invention and Human IL-4 Expressed Using Non-Human Systems

Protein estimation of IL-4 of the present invention was determined using the Bradford protein assay (Bradford 1976 supra).

IL-4 of the present invention, standardised using the Bradford assay results, was diluted and tested in a R&D Systems human IL-4 DuoSet® ELISA kit (Cat.# DY204) in accordance with the manufacturer's instructions. The above-mentioned ELISA kit employs a human IL-4 expressed in E. coli as a standard.

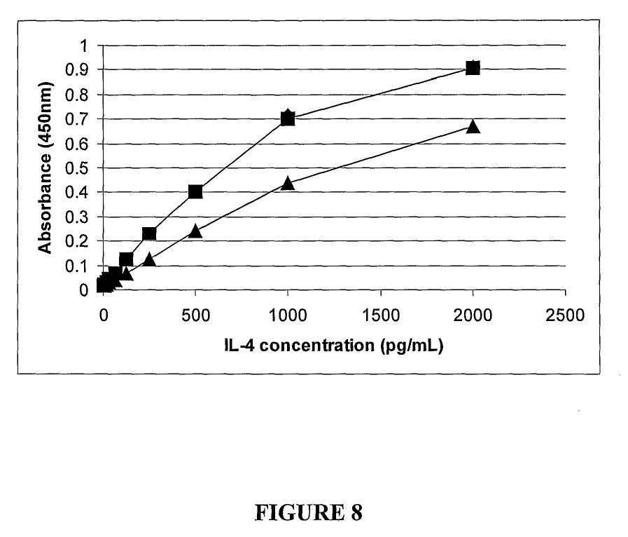

The R&D Systems DuoSet® IL-4 ELISA kit results gave a concentration estimate of IL-4 of the present invention of approximately 780 pg/ml at an OD450 nm of 0.55 (FIG. 8) when estimated from the IL-4 expressed in E. coli standard curve. Whereas, the actual interpolated concentration of IL-4 of the present invention was approximately 1500 pg/ml at a similar OD450 nm value (FIG. 8).

These results represent an approximate 2-fold underestimate of the IL-4 of the present invention concentration by the R&D Systems human IL-4 DuoSet® ELISA kit, a commercial kit employing a E. coli-expressed human IL-4 standard and antibodies against E. coli-expressed human IL-4, that is used to evaluate levels of native human expressed IL-4 in laboratory samples and human patient samples.

This result indicates different immunoreactivity profiles of IL-4 of the present invention and a non-human cell expressed human IL-4 molecule.

### (d) In Vitro Comparison of Immunoreactivity Profiles Between IL-5 of the Present Invention and Human IL-5 Expressed Using Non-Human Systems

Protein estimation of IL-5 of the present invention is determined using a standard protein assay technique, for example, the Bradford protein assay (Bradford 1976 supra).

IL-5 of the present invention, standardised using the standard protein assay results, is diluted and tested in a commercially available ELISA kit, for example, a R&D Systems human IL-5 DuoSet® ELISA kit (Cat # DY218) in accordance with the manufacturer's instructions. The above-mentioned ELISA kit employs a human IL-5 expressed in E. coli as a standard.

It is found that the R&D Systems human IL-5 DuoSet® ELISA kit incorrectly estimates the concentration of IL-5 of the present invention at an OD450 nm when compared with the corresponding IL-5 of the present invention concentration as determined by the standard protein assay.

The protein concentrations of IL-5 of the present invention determined by the commercially available ELISA kit will differ from that determined by a standard protein assay method as the capture and/or detection antibodies employed in the commercially available ELISA kit are raised against a non-human cell expressed human IL-5 protein.

At a structural level, such a result will indicate different immunoreactivity profiles of IL-5 of the present invention and a non-human cell expressed human IL-5 molecule.

### Example 14

Further Purification of Target Molecule of the Present Invention and Peptide Mass Fingerprinting by ESI-MS/MS

In addition to the purification protocol as described in Example 2, purification of the target molecule of the present invention is further performed by RP-HPLC, using a commercially available column. Eluting proteins are monitored by the absorbance at 215 or 280 nm and collected with correction being made for the delay due to tubing volume between the flow cell and the collection port.

A gel piece containing the protein sample from a 1D or 2D gel is digested in trypsin solution as described in Example 3. Alternatively, a solution containing the protein sample is digested with trypsin in an ammonium bicarbonate buffer (10-25 mM, pH 7.5-9). The solution is incubated at 37° C. overnight. The reaction is then stopped by adding acetic acid until the pH is in the range 4-5. The peptide samples are concentrated and desalted using C18 Zip-Tips (Millipore, Bedford, Mass.) or pre-fabricated micro-columns containing Poros R2 chromatography resin (Perspetive Biosystems, Framingham, Mass.) as described in Example 3.

The protein sample (2-5 μl) is injected onto a micro C18 precolumn and washed with 0.1% formic acid at 30 μl/min to concentrate and desalt. After a 3 min wash the pre-column is switched into line with the analytical column containing C18 RP silica (Atlantis, 75 μm×100 mm, Waters Corporation). Peptides are eluted from the column using a linear solvent gradient, with steps, from H₂O:CH₃CN (95:5; +0.1% formic acid) to H₂O:CH₃CN (20:80, +0.1% formic acid) at 200 nl/min over a 40 min period. The LC eluent is subject to positive ion nanoflow electrospray analysis on a Micromass QTOF Ultima mass spectrometer (Micromass, Manchester, UK).

Tandem MS is performed using a Q-T of hybrid quadrupole/orthogonal-acceleration TOF mass spectrometer (Micromass). The QTOF is operated in a data dependent acquisition mode (DDA). A TOFMS survey scan was acquired (m/z 400-2000, 1.0s), with the three largest multiply charged ions (counts >15) in the survey scan sequentially subjected to MS/MS analysis. MS/MS spectra were accumulated for 8 s (m/z 50-2000).

The LC/MS/MS data are searched using Mascot (Matrix Science, London, UK) and Protein Lynx Global Server (“PLGS”) (Micromass). The protein sample is anticipated to be the target molecule.

### Example 15

### (a) Immunogenicity in Non-Human Animals

(i) Animal Immunization with Target Protein

Separate groups of non-human animals, for example, mice are immunized either sub-cutaneously, intramuscularly or intraperitoneally (IP) with 1-100 ug of protein of the present invention and the protein expressed in non-human cells, respectively. Animals receive a secondary immunization one month following immunization. Prior to immunization, protein is emulsified in an adjuvant, for example, complete Freud's adjuvant for the primary immunization and incomplete Freud's adjuvant for the secondary immunization.

### (ii) Detection of Antibodies Directed to Target Protein

For the detection of antibody response, animals from each group are bled from the tail and sera pooled. Protein-specific antibodies are detected by a solid phase ELISA using 50 ng/well of protein of the present invention. Different immunoglobulin isotypes are detected by using labelled detection antibodies raised against IgG1, IgG2, IgG2b, IgG3, IgM, IgA, IgD. Alternatively, antibody response is measured against protein of the present invention blotted onto a membrane either as a dot blot or Western blot. Detection of different immunoglobulin isotypes are detected as described above. It is anticipated that the protein of the present invention will elicit an antibody response that is distinct to that of protein expressed in non-human cells.

(iii) T Cell Proliferation Assay

Immunised animals are euthanised and spleen cells prepared. A suitable number of spleen cells, for example, 5×10⁵ cells, from animals immunized with protein of the present invention are cultured with various concentrations of protein of the present invention while and equivalent number of spleen cells from animals immunized with protein expressed in non-human cells are cultured with various concentrations of protein expressed in non-human cells. For T cell proliferation assays, spleen cells are cultured for 96 hours and treated with 1 μCi [³H] thymidine (6-7 μCi/umol) during the final 16 hours. The cells are harvested onto filter strips and [³H] thymidine incorporation determined using standard methods. It is anticipated that the protein of the present invention will elicit a different proliferation response compared to the protein expressed in non-human cells.

### (iv) IFN Gamma Assay

For the IFN gamma assay, culture supernatant from spleen cells incubated with either the protein of the present invention or protein expressed in non-human cells are harvested at 96 hours and IFN gamma production is detected by a sandwich ELISA, for example, a R&D Systems anti-IFN gamma Quantikine® ELISA kit (Cat # DIF50) in accordance with the manufacturer's instructions. It is anticipated that IFN gamma production will be different in culture supernatant derived from cells incubated with protein of the present invention compared with culture supernatant derived from cells incubated with protein expressed in non-human cells.

### (b) In Vitro Human Immunogenicity Assays

### (i) Human T-Cell Response Assay

Human dendritic cells and CD4⁺ T cells are prepared from human blood as described in Stickler et al. Toxicological Sciences 77:280-289, 2004. Co-cultures of dendritic cells and CD4⁺ T cells are plated out in 96 well plates containing 2×10⁴ dendritic cells and 2×10⁵ CD4⁺ T cells. The protein of the present invention and protein expressed in non-human cells undergo enzymatic digestion into peptide fragments using a suitable enzyme determined by cleavage site prediction software, for example, Peptide Cutter (http://au.expasv.org/tools/peptidecutter). The resulting peptide fragments are purified by a suitable technique, for example, liquid chromatography and added to the co-cultures to a final concentration of 5 ug/ml. Cultures are incubated for 5 days and 0.5uCi ³H thymidine is then added to each culture. The cells are harvested onto filter strips and cell proliferation is determined by [³H] thymidine incorporation.

It is anticipated that the peptides derived from protein of the present invention will elicit a weaker proliferation response compared to peptides derived from the protein expressed in non-human cells.

### (ii) Human Antibody Response Assay

Human donors undergoing treatment with protein expressed in non-human cells are bled and sera prepared. Protein-specific antibodies are detected by a solid phase ELISA against both 50 ng/well of protein of the present invention and protein expressed in non-human cells. Different immunoglobulin isotypes are detected by using labelled detection antibodies raised against human IgG1, IgG2, IgG3, IgG4, IgM, IgA, IgD.

Alternatively, antibody response is measured against protein of the present invention and protein expressed in non-human cells blotted onto a membrane either as a dot blot or Western blot. Detection of different immunoglobulin isotypes are detected as described above.

It is anticipated that the immunoglobulin present in the sera of people treated with protein expressed in non-human cells will bind to protein expressed in non-human cells while either binding weakly or not binding with protein of the present invention.

### Example 16

### Preparation of Protein of the Present Invention from Recombinant Genomic Constructs

The genomic sequences encoding the GM-CSF, IL-3, IL-4 and IL-5 of the present invention (SEQ ID NOs: 69, 70, 71, 72) are amplified by PCR and cloned into appropriate expression vectors, for instance pIRESbleo3, pCMV-SPORT6, pUMCV3, pORF, pORF9, pcDNA3.1/GS, pCEP4, pIRESpuro3, pIRESpuro4, pcDNA3.1/Hygro(+), pcDNA3.1/Hygro(−), pEF6/V5-His. These recombinant constructs are then prepared for human cell transformation as described above in Example 1(c). Production and purification of GM-CSF, IL-3, IL-4 and IL-5 of the present invention from the recombinant DNA construct are carried out as described above in Example 2.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Ackland et al. Chromatogr 540:187-198, 1991
Aloj et al. J Biol Chem 247:1146-1151, 1971
Altschul et al. Nucl Acids Res 25:389, 1997
Antibodies: A Laboratory Manual, Harlow and Lane (eds.), Cold Spring Harbor Laboratory Press, Cold Spring Harbor, N.Y., (1988).
Aronsson et al. FEBS Lett 411:359-364, 1997
Atherton and Shephard Synthetic Vaccines 9: Blackwell Scientific Publications
Ausubel et al. In: Current Protocols in Molecular Biology John Wiley & Sons Inc. 1994-1998)
Baneyx Current Opinion in Biotechnology, 10:411-421, 1999
Bernstein Methods Mol Biol 237:195-204, 2004
Bird Science 242:423, 1988
Blenis and Resh Curr Opin Cell Biol 5(6):984-9, 1993
Bonner and Laskey Eur J Biochem 46:83, 1974
Bradford M Anal Biochem 72:248-254, 1976
Caprioli et al. Biochem Biophys Res Commun 146:291-299, 1987
Carr et al. Anal Biochem 175: 492-499, 1988
Carr et al. Anal Chem 63:2802-2824, 1991
Carr et al. J Biol Chem 264(35):21286-21295, 1989
Clackson et al. Nature 352:624-628, 1991
Clarke Curr Opin Cell Biol 5:977 983, 1993
Datta et al. J Biol Chem 270:1497-1500, 1995
Dedov et al. Apoptosis 8:399-406, 2003
Edman Mol Biol Biochem Biophys 8:211-55, 1970
Erickson et al. Science 249:527-533, 1990
Farruggia et al. Int J Biol Macromol 20:43-51, 1997
Figeys and Aebersold, Electrophoresis 19:885-892, 1998
Franks et al. Characterization of proteins, Humana Press, Clifton, N.J., 1988
Fritz et al. PNAS 95:12283-12288, 1998
Fukuhara et al. J Biol Chem 260:10487-10494, 1985
Gelb et al. Curr Opin Chem Biol 2(1):40-8, 1998
Gramer et al. Biotechnology 13 (7):692-8, 1995
Guedez et al. Am J Pathol 162:1431-1439, 2003
Harrison and Packer Methods Mol Biol 125:211-216, 2000
Hearn et al. Methods in Enzymol 104:190-212, 1984
Herscovics et al. FASEB J 7:540-550, 1993
Herzberg et al. Infrared and Raman Spectra of Polyatomic Molecules, Van Nostrand Reinhold, New York, N.Y., 1945
Hodgson Bio/Technology 9:19-21, 1991
Holzwarth et al. J Am Chem. Soc 178:350, 1965
Honroe et al. Biochem J 258:99-204, 1989
Huston et al. Proc Natl Acad Sci USA 85:5879, 1988
J Biochem 336:647-658, 1998
J Biochem 363:619-631, 2002
James and Bottomley Arch Biochem Biophy 356:296-300, 1998
Jones et al. Nature 321:522-525, 1986
Kennet et al. Monoclonal Antibodies, Hybridomas: A New Dimension in Biological Analyses, Plenum Press, New York, 1980
Kivirikko et al. FASEB Journal 3:1609-1617, 1989
Kohler et al. Nature 256:495, 1975
Kortt et al. Protein Engineering 10:423, 1997
Krimm and Bandekar Adv Protein Chem 38:181-364, 1986
Kronman Gene 121:295-304, 1992
Kurochkin et al. J Mol Biol 248:414-430, 1995
Kwon and Yu Biophim Biophys Acta 1335:265-272, 1997
Larrick et al. Bio/Technology 7:934, 1989
Larsen et al. J Biol Chem 265:7055-7061, 1990
Li et al. Biochemistry 34:5762-5772, 1995
Liu et al. J Immunol 158:604-613, 1994
Lucka et al. Glycobiology 15(1):87-100, 2005
Marks et al. J Mol Biol 222:581-597, 1991
Marmur and Doty J Mol Biol 5:109, 1962
Martin et al. Nature Medicine 11 (2):228-232, 2005
Mas et al. Glycobiology 8(6):605-13, 1998
McGettrick et al. Methods Mol Biol 244:151-7 2004
Mire-Sluis et al. J Immunol Methods 289(1-2):1-16, 2004
Moore J Biol Chem 278(27):24243-24246, 2003
Morrison et al. Proc Natl Acad Sci USA 81:6851-6855, 1984
Nguyen et al. J Chromatogr A. 705:21-45, 1995
Packer et al. Glycoconj J5(8):737-47, 1998
Phillies Anal Chem 62:1049A-1057A, 1990
Pikal et al. Pharm Res 8:427-436, 1991
Presta, Curr Op Struct Biol 2:593-596, 1992
Rando Biochim Biophys Acta 1300(1):5-16, 1996
Reichmann et al. Nature 332:323-329, 1988
Sambrook et al. Molecular Cloning—A Laboratory Manual, Cold Spring Harbour, New York, USA, 1990
Schmid et al. Protein structure, a practical approach, Creighton Ed., IRI Press, Oxford, England, 1989
Shepherd et al. Arterioscler Thromb Vasc Biol 24:898-904, 2004
Stickler et al. Toxicological Sciences 77:280-289, 2004
Triguero et al. J of Neurochemistry 54:1882-1888 1990
Ward et al. Nature 334:544, 1989
Wells Methods Enzymol 202:2699-2705, 1991
Wilkinson Annu Rev Nutr 15:161-89, 1995
Winter and Harris TIPS14:139, 1993
Yoshioka et al. Pharm Res 10:103-108, 1993

1. An isolated protein comprising a profile of measurable physiochemical parameters, wherein said profile is indicative of, associated with or forms the basis of one or more distinctive pharmacological traits, wherein said isolated protein comprises a physiochemical profile comprising a number of measurable physiochemical parameters, {[Pₓ]₁, [Pₓ]₂, . . . [Pₓ]ₙ,}, wherein Pₓ represents a measurable physiochemical parameter and “n” is an integer ≧1, wherein each of [Pₓ]₁ to [Pₓ]ₙ is a different measurable physiochemical parameter, wherein the value of any one of the measurable physiochemical characteristics or an array of values of more than one measurable physiochemical characteristics is indicative of, associated with, or forms the basis of, a distinctive pharmacological trait, T_{y}, or an array of distinctive physiochemical traits {[T_{y}]₁, [T_{y}]₂, . . . [T_{y}]ₘ} wherein T_{y} represents a distinctive pharmacological trait and m is an integer ≧1 and each of [T_{y}]₁ to [T_{y}]ₘ is a different pharmacological trait, wherein the isolated protein is selected from the group comprising GM-CSF, IL-3, IL-4 and IL-5.

2. The isolated protein of claim 1, wherein said protein comprises one or more of the measurable physiochemical parameters set forth in Table 2.

3. The isolated protein of claim 1 wherein said protein comprises one or more of the distinctive pharmacological traits set forth in Table 3.

4. A chimeric molecule comprising the isolated protein of claim 1, or fragment thereof, fused to one or more peptide, polypeptide or protein moieties.

5. The chimeric molecule of claim 4 wherein the peptide, polypeptide or protein moiety comprises the constant (Fc) or framework region of a human immunoglobulin.

6. The chimeric molecule of claim 4 wherein the chimeric molecule is selected from the group comprising GM-CSF-Fc, IL-3-Fc, IL-4-Fc and IL-5-Fc.

7. A pharmaceutical composition comprising the isolated protein or chimeric molecule of any one of claims 1 to 6.

8. A method of treating or preventing a condition in a mammalian subject, wherein said condition can be ameliorated by increasing the amount or activity of a protein, said method comprising administering to said mammalian subject an effective amount of an isolated protein according to any one of claims 1 to 3, a chimeric molecule according to any one of claims 4 to 6 or the pharmaceutical composition of claim 7.

9. A nucleotide sequence selected from the list consisting of SEQ ID NOs: 25, 27, 29, 31, 35, 37, 39, 41, 43, 45, 47, 51, 53, 55, 57, 61, 63, 65 or 67, or a nucleotide sequence having at least about 90% identity to any one of the above-listed sequences or a nucleotide sequence capable of hybridizing to any one of the above sequences or their complementary forms under high stringency conditions.

10. An isolated protein or chimeric molecule encoded by a nucleotide sequence selected from the list consisting of SEQ ID NOs: 25, 27, 29, 31, 35, 37, 39, 41, 43, 45, 47, 51, 53, 55, 57, 61, 63, 65 or 67, or a nucleotide sequence having at least about 90% identity to any one of the above-listed sequence or a nucleotide sequence capable of hybridizing to any one of the above sequences or their complementary forms under high stringency conditions.

11. An isolated nucleic acid molecule encoding a protein or chimeric molecule or a functional part thereof comprising a sequence of nucleotides having at least 90% similarity SEQ ID NOs: 25, 27, 29, 31, 35, 37, 39, 41, 43, 45, 47, 51, 53, 55, 57, 61, 63, 65 or 67 or after optimal alignment and/or being capable of hybridizing to one or more of SEQ ID NOs: 25, 27, 29, 31, 35, 37, 39, 41, 43, 45, 47, 51, 53, 55, 57, 61, 63, 65 or 67 or their complementary forms under high stringency conditions.

12. An isolated nucleic acid molecule comprising a sequence of nucleotides encoding a protein or chimeric molecule having an amino acid sequence substantially as set forth in one or more of SEQ ID NOs: 26, 28, 30, 32, 36, 38, 40, 42, 44, 46, 48, 52, 54, 56, 58, 62, 64, 66 or 68 or an amino acid sequence having at least about 90% similarity to one or more of SEQ ID NOs: 26, 28, 30, 32, 36, 38, 40, 42, 44, 46, 48, 52, 54, 56, 58, 62, 64, 66 or 68 after optimal alignment.

13. A kit for determining the level of human cell expressed human protein or chimeric molecule present in a biological preparation comprising (a) a solid phase support matrix; (b) one or more antibodies directed against a human protein according to any one of claims 1 to 3 or chimeric molecule according to any one of claims 4 to 6; (c) a blocking solution; (d) one or more stock solutions of substrate; (e) a solution of substrate buffer; (f) a standard human protein or chimeric molecule sample; and (g) instructions for use.

14. The kit of claim 13, wherein the standard human protein or chimeric molecule sample is a preparation of the isolated protein of any one of claim 2 or 3 or the chimeric molecule of claim 4.

15. The kit of claim 13 or 14, wherein the or each antibody is derived from an immunization of a mammal with a preparation comprising the isolated protein of any one of claims 2 or 3 or the chimeric molecule of claim 4.

16. The kit of any of claims 13 to 15, wherein the human cell expressed human protein is naturally occurring human GM-CSF, IL-3, IL-4 and IL-5.


SEQUENCE LISTING (72 sequences)

SEQ ID NO: 1 - DNA, 717 bp, Homo sapiens
aaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 238 aa, Homo sapiens
KVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 717 bp, Homo sapiens
aaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcagggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 238 aa, Homo sapiens
KVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 705 bp, Homo sapiens
aaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 234 aa, Homo sapiens
KVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - DNA, 768 bp, Homo sapiens
aaggtggacaagagagttgagctcaaaaccccacttggtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgctacacgcagaagagcctctccctgtctccgggtaaatga

SEQ ID NO: 8 - protein, 255 aa, Homo sapiens
KVDKRVELKTPLGDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAKGQPREPQVYTLPPSREEMTKNQVSLTCLVKGFYPSDIAVEWESNGQPENNYNTTPPMLDSDGSFFLYSKLTVDKSRWQQGNIFSCSVMHEALHNRYTQKSLSLSPGK

SEQ ID NO: 9 - DNA, 708 bp, Homo sapiens
aaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 10 - protein, 235 aa, Homo sapiens
KVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - DNA, 822 bp, Homo sapiens
aagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 12 - protein, 273 aa, Homo sapiens
KSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 13 - DNA, 783 bp, Homo sapiens
aagtccgtgacatgccacgtgaagcactacacgaatcccagccaggatgtgactgtgccctgcccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 14 - protein, 260 aa, Homo sapiens
KSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 15 - DNA, 1548 bp, Homo sapiens
aagagtcgagtcaccatatcagtagacacgtccaagaagcagctctccctgaagttgagctctgtgaacgccgcggacacggctgtgtattactgtgcgagagttattactagggcgagtcctggcacagacgggaggtacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctactga

SEQ ID NO: 16 - protein, 515 aa, Homo sapiens
KSRVTISVDTSKKQLSLKLSSVNAADTAVYYCARVITRASPGTDGRYGMDVWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 17 - DNA, 1029 bp, Homo sapiens
gtggcacacactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatga

SEQ ID NO: 18 - protein, 342 aa, Homo sapiens
VAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 19 - DNA, 1047 bp, Homo sapiens
aaatgcgtggtccagcacaccgccagcaagagtaagaaggagatcttccgctggccagagtctccaaaggcacaggcctcctccgtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccgagccacacccagcctcttggcgtctacctgctaacccctgcagtgcaggacctgtggctccgggacaaagccaccttcacctgcttcgtggtgggcagtgacctgaaggatgctcacctgacctgggaggtggctgggaaggtccccacagggggcgtggaggaagggctgctggagcggcacagcaacggctcccagagccagcacagccgtctgaccctgcccaggtccttgtggaacgcggggacctccgtcacctgcacactgaaccatcccagcctcccaccccagaggttgatggcgctgagagaacccgctgcgcaggcacccgtcaagctttctctgaacctgctggcctcgtctgaccctcccgaggcggcctcgtggctcctgtgtgaggtgtctggcttctcgccccccaacatcctcctgatgtggctggaggaccagcgtgaggtgaacacttctgggtttgcccccgcacgcccccctccacagcccaggagcaccacgttctgggcctggagtgtgctgcgtgtcccagccccgcccagccctcagccagccacctacacgtgtgtggtcagccacgaggactcccggactctgctcaacgccagccggagcctagaagtcagctacctggccatgacccccctgatccctcagagcaaggatgagaacagcgatgactacacgacctttgatgatgtgggcagcctgtggaccaccctgtccacgtttgtggccctcttcatcctcaccctcctctacagcggcattgtcactttcatcaaggtgaagtag

SEQ ID NO: 20 - protein, 348 aa, Homo sapiens
KCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYLAMTPLIPQSKDENSDDYTTFDDVGSLWTTLSTFVALFILTLLYSGIVTFIKVK

SEQ ID NO: 21 - DNA, 24 bp, Artificial Sequence
  Primer
aaaggatccagcaacaccaaggtg

SEQ ID NO: 22 - DNA, 41 bp, Artificial Sequence
  Primer
aaattaattccagcacactggtcatttacccggagacaggg

SEQ ID NO: 23 - DNA, 39 bp, Artificial Sequence
  Primer
aaaagatatcgctggaggatgtggctgcagagcctgctg

SEQ ID NO: 24 - DNA, 38 bp, Artificial Sequence
  Primer
aaaaggatcctcactcctggactggctcccagcagtca

SEQ ID NO: 25 - DNA, 51 bp, Homo sapiens
atgtggctgcagagcctgctgctcttgggcactgtggcctgcagcatctct

SEQ ID NO: 26 - protein, 17 aa, Homo sapiens
MWLQSLLLLGTVACSIS

SEQ ID NO: 27 - DNA, 381 bp, Homo sapiens
gcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 28 - protein, 127 aa, Homo sapiens [sequence omitted]

SEQ ID NO: 29 - DNA, 432 bp, Artificial Sequence
  GM-CSF nucleotide sequence
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 30 - protein, 144 aa, Artificial Sequence
  GM-CSF amino acid sequence
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 31 - DNA, 1161 bp, Artificial Sequence
  GM-CSF-Fc nucleotide sequence for whole construct
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggatccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - protein, 387 aa, Artificial Sequence [sequence omitted]

SEQ ID NO: 33 - DNA, 35 bp, Artificial Sequence
  Primer
aaaaggatccaaacatgagccgcctgcccgtcctg

SEQ ID NO: 34 - DNA, 35 bp, Artificial Sequence
  Primer
gtggtgagaaggatccgagaacgagctggacgttg

SEQ ID NO: 35 - DNA, 57 bp, Homo sapiens
atgagccgcctgcccgtcctgctcctgctccaactcctggtccgccccggactccaa

SEQ ID NO: 36 - protein, 16 aa, Homo sapiens
MSRLPVLLLLQLLVRP

SEQ ID NO: 37 - DNA, 399 bp, Homo sapiens
gctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttt

SEQ ID NO: 38 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 39 - DNA, 399 bp, Homo sapiens
gctcccatgacccagacaacgtccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttt

SEQ ID NO: 40 - protein, 133 aa, Homo sapiens
APMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 41 - DNA, 456 bp, Artificial Sequence
  IL-3 nucleotide sequence
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttt

SEQ ID NO: 42 - protein, 152 aa, Artificial Sequence
  IL-3 amino acid sequence
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 43 - DNA, 456 bp, Artificial Sequence
  IL-3-Fc nucleotide sequence
atgagccgcctgcccgtcctgctcctgctccaactcctggtccgccccggactccaagctcccatgacccagacaacgtccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttt

SEQ ID NO: 44 - protein, 152 aa, Artificial Sequence
  IL-3-Fc amino acid sequence
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 45 - DNA, 1185 bp, Artificial Sequence
  IL-3-Fc nucleotide sequence for whole construct
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatctttggatccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - protein, 395 aa, Artificial Sequence
  IL-3-Fc amino acid sequence for whole construct
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIFGSSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1185 bp, Artificial Sequence
  IL-3-Fc nucleotide sequence for whole construct (variant)
atgagccgcctgcccgtcctgctcctgctccaactcctggtccgccccggactccaagctcccatgacccagacaacgtccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatctttggatccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 395 aa, Artificial Sequence [sequence omitted]

SEQ ID NO: 49 - DNA, 29 bp, Artificial Sequence
  Primer
gtaccgtatgcggccgcgaccatgggtct

SEQ ID NO: 50 - DNA, 28 bp, Artificial Sequence
  Primer
gagggaaaaagatctggatcctcagctc

SEQ ID NO: 51 - DNA, 72 bp, Homo sapiens
atgggtctcacctcccaactgcttccccctctgttcttcctgctagcatgtgccggcaactttgtccacgga

SEQ ID NO: 52 - protein, 24 aa, Homo sapiens
MGLTSQLLPPLFFLLACAGNFVHG

SEQ ID NO: 53 - DNA, 387 bp, Homo sapiens
cacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 54 - protein, 129 aa, Homo sapiens [sequence omitted]

SEQ ID NO: 55 - DNA, 459 bp, Artificial Sequence
  IL-4 nucleotide sequence
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 56 - protein, 153 aa, Artificial Sequence [sequence omitted]

SEQ ID NO: 57 - DNA, 1188 bp, Artificial Sequence
  IL-4-Fc nucleotide sequence for whole construct
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagcggatccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 58 - protein, 396 aa, Artificial Sequence [sequence omitted]

SEQ ID NO: 59 - DNA, 28 bp, Artificial Sequence
  Primer
gccgaattcgaacgcagaacgtttcaga

SEQ ID NO: 60 - DNA, 28 bp, Artificial Sequence
  Primer
tctcggatcccaacaaaccagtttagtc

SEQ ID NO: 61 - DNA, 57 bp, Homo sapiens
atgaggatgcttctgcatttgagtttgctagctcttggagctgcctacgtgtatgcc

SEQ ID NO: 62 - protein, 19 aa, Homo sapiens
MRMLLHLSLLALGAAYVYA

SEQ ID NO: 63 - DNA, 345 bp, Homo sapiens
atccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagt

SEQ ID NO: 64 - protein, 115 aa, Homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 65 - DNA, 402 bp, Artificial Sequence
  IL-5 nucleotide sequence
atgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagt

SEQ ID NO: 66 - protein, 134 aa, Artificial Sequence
  IL-5 amino acid sequence
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 67 - DNA, 1131 bp, Artificial Sequence
  IL-5-Fc nucleotide sequence for whole construct
atgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtggatccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - protein, 377 aa, Artificial Sequence [sequence omitted]

SEQ ID NO: 69 - DNA, 2024 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatggtaagtgagagaatgtgggcctgtgctaggcaccagtggccctgactggccacgcctgtcagcttgataacatgacattttccttttctacagaatgaaacagtagaagtcatctcagaaatgtttgacctccaggtaagatgcttctctctgacatagctttccagaagcccctgccctggggtggaggtggggactccattttagatggcaccacacagggttgtccactttctctccagtcagctggctgcaggaggagggggtagcaactgggtgctcaagaggctgctggccgtgcccctatggcagtcacatgagctcctttatcagctgagcggccatgggcagacctagcattcaatggccaggagtcaccaggggacaggtggtaaagtgggggtcacttcatgagacaggagctgtgggtttggggcgctcactgtgccccgagaccaagtcctgttgagacagtgctgactacagagaggcacagaggggtttcaggaacaacccttgcccacccagcaggtccaggtgaggccccacccccctctccctgaatgatggggtgagagtcacctccttccctaaggctgggctcctctccaggtgccgctgagggtggcctgggcggggcagtgagaagggcaggttcgtgcctgccatggacagggcagggtctatgactggacccagcctgtgcccctcccaagccctactcctgggggctgggggcagcagcaaaaaggagtggtggagagttcttgtaccactgtgggcacttggccactgctcaccgacgaacgacattttccacaggagccgacctgcctacagacccgcctggagctgtacaagcagggcctgcggggcagcctcaccaagctcaagggccccttgaccatgatggccagccactacaagcagcactgccctccaaccccggtgagtgcctacggcagggcctccagcaggaatgtcttaatctagggggtggggtcgacatggggagagatctatggctgtggctgttcaggaccccagggggtttctgtgccaacagttatgtaatgattagccctccagagaggaggcagacagcccatttcatcccaaggagtcagagccacagagcgctgaagcccacagtgctccccagcaggagctgctcctatcctggtcattattgtcattacggttaatgaggtcagaggtgagggcaaacccaaggaaacttggggcctgcccaaggcccagaggaagtgcccaggcccaagtgccaccttctggcaggactttcctctggccccacatggggtgcttgaattgcagaggatcaaggaagggaggctacttggaatggacaaggacctcaggcactccttcctgcgggaagggagcaaagtttgtggccttgactccactccttctgggtgcccagagacgacctcagcccagctgccctgctctgccctgggaccaaaaaggcaggcgtttgactgcccagaaggccaacctcaggctggcacttaagtcaggcccttgactctggctgccactggcagagctatgcactccttggggaacacgtgggtggcagcagcgtcacctgacccaggtcagtgggtgtgtcctggagtgggcctcctggcctctgagttctaagaggcagtagagaaacatgctggtgcttccttcccccacgttacccacttgcctggactcaagtgttttttatttttctttttttaaaggaaacttcctgtgcaacccagattatcacctttgaaagtttcaaagagaacctgaaggactttctgcttgtcatcccctttgactgctgggagccagtccaggagtga

SEQ ID NO: 70 - DNA, 2087 bp, Homo sapiens
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggtgagtagcttggataagactggcctgcagcagtgaggggtggtggctgcctaaggccaaaaggcctcatgggcctttctctcccttcacccccacaggacttcaacaacctcaatggggaagaccaagacattctgatggtaagagctcagcccgtggatcccgatccacttcctgcctgggtgacttcagccatgtcattccatcttacctagccttgctttcttcatctgtaaaatagggttaatagcacctatctcagtgggattgttatgacaatcaaatggcacaatgtgcatgttctggcccagcatctggcacttaagagttcaatacatggccacagccatggctataataatgaaaatgacttttaaattagaaaatgaaaaggcagttctaggtgaggaataccaagggccttaaggtcagtgtttggtagggcagactctggactgacagacagaaatttccccctcagatgtgccagcttctttttcccttaaaaaattgagtttgtgttttccaccctcattggctgtggcattatcatttacagttatttttcatggttccagttggttgcagttgttctgtctcacctttgcatgacatttcttgggtcaaacaagaagtggaagcctttgctgagttcctgggccatctgtgtttggggcactcacagctcacatgctgcacccatttccatgctggggtctctgtgattttcttcttgatttctaggagactttaattcagtcagggcatgaacactgttatcagcctctgagctacaaatacttcccttggaaaaccccctgtcttttgtagagcttctttttggagatattttttccccccaagtgcagaaagatccacctaggtacccccttccccacccaattttttttttttttttgagacaagagtctcactctgttgcccaggctggagtgcagtggcacgatctcagctcactgctacctctgcctcctgagttcaagcaattctcctgtctcagcctcccaagtagctaggattacaggcatgcaccaccactcccatttttgtatttttagtagagacggggtttcaccatattggtcaggctggtcttgaactcgacctcaggttatctgcctgcctcggcctcccaaagtgctgggattacaggcttgagccactgcgcccagcccacctaggccctttatgtagctcaaatggagccagagactgggggcttgaggaaaccaggtcctgcctgccactcacttctaggcctgtgcccttgggcagggacccacctgaggcaagaacgggactaggagggaacccgaggatgtccccaacagtgggcttgggaaactgtgggggtgacttccacctgcttgtgggagggatactctgtaacctttcccccttaagtgtattctctgccccgttaggaaaataaccttcgaaggccaaacctggaggcattcaacagggctgtcaagagtttacagaacgcatcagcaattgagagcattcttaaagtatgtgaagctgttgagggtttgggatccctgtgttggccctgccctgcctctggggaggagagcagggcccactccctttccaagggaatctctgaccatctgctttggtctctttccacagaatctcctgccatgtctgcccctggccacggccgcacccacggtaagctgtcccccaagatgcccgtcatggcttgctcctcagctggtcatcaccattacagcctggactcacctaatgccaccttcttggtttctttatagcgacatccaatccatatcaaggacggtgactggaatgaattccggaggaaactgacgttctatctgaaaacccttgagaatgcgcaggctcaacagacgactttgagcctcgcgatcttttga

SEQ ID NO: 71 - DNA, 8571 bp, Homo sapiens
atgggtctcacctcccaactgcttccccctctgttcttcctgctagcatgtgccggcaactttgtccacggacacaagtgcgatatcaccttacaggagatcatcaaaactttgaacagcctcacagagcagaaggtgagtacctatctggcaccatctctccagatgttctggtgatgctctcagtatttctaggcatgaaaacgttaacagctgctagagaagttggaactggtggttggtggcagtccagggcacacagcgaggcttctcccctgccactcttttttctgagggtttgtaggaagtttcctcagttggagggagtgagagctgctcatcaaggacttctctgtccggttggaggttaactctgtctcttgctctctcatttctgcctggaccaagactctgtgcaccgagttgaccgtaacagacatctttgctgcctccaaggtaagaagccgtcccacggtctgttttagcaaatggggagatccatccccaaatgtctgaacaagaaacttgtctaatggaaaacgagcgggcccaaattaactctaaggtgttagatgttttcaaagaacgagaagtctgatctttactcttaagcatgttttggtctttctggtttcacttgatttagaagacatgtaatagaaagcttacatgctgtagtcctgactcagatcctggtcaaagaaaagccctcttgggttttacttagctttggcatagtgcctggaacgtaggaggcactcaataaatgcctgttgaatgagagaatttttctggcccatacatttctgaaaaaccaaatactctcacagaaacagatattgagatgacaggttgagggagctttcattttgtctaagagacttcctatggcaacagaaaaggtatcgccagagcccctcctcttccacagcctggccacctaacagccctctgggttccggggctggccgtccagagctcctcagcttgctctggccggccgaactcccctccagctcggtctggaaccatcctgctgggcagcgtccagcacatccctgcttcgggctgcctgggcacctcgcctctctgcctcctgtgctgcctcacccccacccctctatctgtagtgggaggagatagatttgacagctgatagtgcattttctctgacaaacacatgactacagccgtatcaatagttttgtgcatttcagttcctgttttcatggaaacacacggctgagaatgaaagccccaaagcctcaatttcacagtggtctcctaactacctgctttccatgcaaactagggagatgatatggccaggagtgaagccctgtgtgttgggcagggtcacactccagcacccagaccatagaacagggcccatcctgcttcatgagggaaactgctcttcgggcctttagctggactatctcatttcattagttatcccgggagtccgatacaggatgagattctgaagggcaaatacacacttttttttttttttgagatagggtcttgttctgtcacccaggctggagtgcagtggtgcgatttcagctcatagcagcctccacctcccaggctcaagctatcttcctacctcagcctcccaagtagccgggacgacaggtgtgcaccaccacgcctggctaatttttgtatttttttgtagagatggagtcttgccattttgcccaggcttgtctcgaacttctgggctcaagcaatccgtccacctcggcctctcaaagtgctgggattagccactgcacctgggcaacagtttatgtgtgtgtgtgtgtgtgtgtgtgtgtgtgtgtatatatgtgtgtgtgtatatatatgtgtatgtatatatatgtgtatatatatatgtgtgtgtgtgtgtgtgtgtgtgtgtgtgtgtgtgtgtgtgtgtgtgtgtgtgtataaaatctccaagtccatccaaccgagatggctcctactagaagccaagagtccaccgggttgagcactgggtctctggaggcctgtggcactgctgagaaggctctaacaaagccaagggaagggccacctcactagaagccaggcctggaggaagggtgagggctgagggcctggaggtaagactgcctgtggttttagacccagctctgccactgactagctgtgtggctggccttcagcacatcttcacacctctctgcacctcagtttccacatgtgaagatatgaaagtgattctgaaggtgattgcaaggttgattggaatccagctcttgagttagtgcaaagtgttattgtgagatgatataaccacgattaaaagcaagaacaggtgcagagaagcgatgattctaagaaggaggggaccgggttggaaaggatcaaaccatccaggatgccgagtctggggcaatccatctgggctgtttctggaagacccccgggtgcaggccaggacactgctgccctcccgtccttaactcccctcttcactcagtcctcactcacctccctctcacacacacaaacatctcctagaataatccccactgcctgccttcactcttacccgtctcatttgcctcccctgaacttcatcctcctggagttcacgatctcactcttcactcttttcttcccctcgaagattcagcactgcttacttacatgttaagatatttcagaacagtgaaatgttgctattttcaaaaacctacaaaggtggtatgcagaggaaaaggtacttctttgtgttcccaaagaaaacatctttccaaaatccagcctattgattttatttcttcgggggaacaagaattttagtatctctaagttgggtagcattctactcttggcagttgctggaaagaaggcactggtctaggtcctgggcttcacaggtaacacctgtcagggtgtctatgaagtcaaggctgtctgaggaacagcaaagtgggaagaagcaagctggctggctgatgaagggtttcttgggtggacaagtagttggagctatttcctatttaccaaagagagctaaagttcataattctacagagagttccataatgaacctcaaatacctctgttttttgaaggagtttctcatatacagcactagctgactatcctgggcaggatgggagataatgaatgcagtgccaatcgggctggatttatatggtcctcagtgaggctggtcaagaaccgagttagaactctcacagagtcactgccacagaagaaatctcccaagtggctgtttcctgacattcccgggagggacaggcctccttctgagtcactccctaagcagttctgaactgtgaggtcagccaggctgtccaagtgcactccctgagccactggcagacacactcagcagccagagctagacaggcaggtggtaggagtccagggccacggcagggatggagtgtcgccccctcgctgcgataccagagcaactaaaacgttaaggccttgcactaaagctgcccttaggatgcattcttttaaagtttttccatttaatgcagactcttttcaattcttattttatccttgtttcctttagaaagtcctttgaaaaatatctttagagggttttttcctatactatgtggccatatacgggtcaaaattaagtttaatttccaggctccaagccagcgtttcagaaaaatctcaccaaggtttgtggtaaaagaagcaaagggctgactttttggttttcttgaatctcactgttccctctgcagcagcatgcatgtctgcccacctccagacacacaggcaccatctgccgccccccatcagcccgtgtcccttccacctcgactcgcctacaaagcccagagaggtctgtttcttggcccccagagcccaaagatactgacacactcttacatttccaactagaatcaggaacgaggagtgactctcagtcagttcattaagtaaatgtctttctaaccgctctgcccatgggacatcacgccccacaggggaaaggggaagcttctgtagcctgggattctggtgcctcagtctgggtctagactttcctgaaaaaacgttaaaatatgaactgcattcctagaatttagcctacataaataagagatgaacacaaagatttctatagtttactcactgccgcttatttacagaagcaaaaatctgccacgataggggcctgacaaatgacagtaccactgtgcaatgcgtttctacgcagctctcaatcccatgttctctaataccaccgaagggcttaggaaatgcttatggtatatgtaaagagtaaagaagttacaaaacagtatcaacagttgacccctattttaaaaagtatttttaaaagtgtgacgatatttaccaaaatattaacagcaatagttacctctggctggtgggatgagtgaatgtatttttgttgaatatatgttacctttatagtaaatatatgttatcttgatcatcagaaaaaaaaatatgtaagaacttgaaagctgcttggacagcgctgctgatagaaacccctgagcatcttgtcactgttcttctgattcagagggtctgggtggggcaggggtggtctgagattctgtatttctaagaagctcccagtgatgtccatgctgctggtccatggaccacactttgagtatcaagggaccagagcatgtcgggggagaggctggggatagctttctttatctgaactggataaaggaactgggctcaagctaagaaccctctccaggttctgcatctttgttcttcagtgaaaaatgagaggacacaccaggccaggttcagactgagacacaatccctctcctgggttcccaatgacttgtctcttgtccattcccttctctaaggctaagggcccccaggaagagccatgtggccagaccctcacagttgctggcattccaaggagattctcactccgcatcatttggggccaaaaggccccttacagaagctctgcccaaggctcagatcaatggcacctgctcccagagcctcctctgatctcccaggacacctttccctgatctgtgcacttatctcttgctgcctggcaaaatgtcttagctcctcacttgggccatgtgctgctctcctctcccatggggagagccacacggagagtgctggccaaagcagcagagttcaggccaaaggatgtgcactcatttattcaacaggcatgcaggatttccagggaaagctggattttaaaacctctgggaacaagagcagaacctgactgagagctcatgtgggcacttttcatagcagaatagctcatgaggtatagagacacggacgcagaacgtgggctgtagcgacagatggtcctgcattctagtccccactgtgccttttcctcatgggatgactttattcaggtaccctttcggcaaaatcctccaagagaaaggaaactgggaggttctggggagaaggctgctgcgtttgcaattgggagaggttgttgacagaggtttatgtctgtggcaagcagccttccttcagtggaatacttgaagacaggtctgtagttgagcaaactcacctccatttgtcctcctggaaagaagaaatcaagaggaaaaatctctctcccatcctccaaatggagctggcacattgctatctgtggcatttgtctttccagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttggtaagctgcactgtattcctggcaagccggccgcgtggctcctggtggacagcagcctcacttctaaacactccttaggagctgcagcacccttggtcaacccattcattcattcactcattcaataagtatttgctgaagttccacaagtgctgggtgtggttctaggtgctgaggacgtgtcactaaagacacgcaggccgagtccctgttctcatggaatgttctaatgggagagttagaaaaacaaacatgtaaaatgatggccagcagtgatacgtgctacaaagaaaaacatagaaataaagaacataagagtcatgggggagggggctgacttaggagctggtgacattatctgagcagatatttgaattgagggagcaggccacatgactaactagggagaccattccaggcagaaggaggaggtatgcaaaggccttaggatggaaatgaactaacttcctgtatttaaagaccagtaggaaggccagtgtggctggatcagagtgagtgaggggtagtttccaggacagcagatcacacaaggcctttagattccaccacgagtatggcagggaacacctgcagagctttgggcaggacaaagactgtacaatctgatttacgtgatttaaaagggtcagtctggctactgtgtggtaaataggctgaaagggggaaagcatagaagcaagatggcctgttgggaggctaccacagtaaaccaggctagagatgatggtggcgtggacagaatgaagcaagatggcctgttgggaggctaccacagtaaaccaggctagagatgatggtggcgtggacagaatgaagcaagatggcctgttgggaggctaccacagtaaaccaggctagagatgatggtggcgtggacagaatgggagcagttgaggtgaacagatttgggatatgactaaaaataaaaccagaaggatttgctgacagatcggttgtagggggtaagatacaggggaggaaaagatgacctctttgttcctgcccaaacccctctggcgatggtcagtactgtttacagagagatgaaagactggcggcaaggcagggctggaggttcagcagaagatcaagagttcaattttgtacatcgtacatgtaaggtggctcttggatagccaagtgaaggtgttgagaagatggttagaaaagtctggaacttaggggagaggtcagaacttgcaatacaaaaaggagagtccttagatagatactgctgaaaatctgaatgacagaaagggagagatcaaaggactgagcctgagatcaacacatggaggtcaggagaggaggatccagccaaggggcctgaggaggagtgaccagtgaggcaggagaacactggagagtgggcggtaccccaggaagccggtgaggacactcaaggagggagggttgactgtgtcaaatgtactgaaaggacaggtcaggtgaggaccaagaaaggcccctgggtttggctgatggaggccatgggtgaggctgatgtaaatggagaggcaggaaggaaagcccagctggagtgggctcaccgaggatagggtggcgagaggagacaaagaaggaacagtgagggcagaacactctttgaagatgtttagctataaggctgcagagaaactgacccacagctgcagggtggttatggagtgagggaagctcttttaaggttgggggtatacccagcatgttaatgcacctgggggaatggtccagtggagcaggaagaactgaagagagcagaaagaggaagaatcattagggggcagaagtccttgtagcccagagtggatgttatctaatatcgagtggaggaattaattggctttagaggagaacaaggacatgtatcccctctctgggcctatcaccttgtagacaatgggataggtcatgggataggaacttggcacaacacatgttctctcttttaattctctccattatcttatgaagcaggcaagtaggcaaacaattgtcccaactttacaaaagaaactgaagcttttataaattaagtagtacatcctaagcaatacaattaataaatggtagagctgagattcaaactgaagcagtggcctgggggtagcatctggaatccttcccacctttagggctgctgtgctgcggtgctgctgtttaatggcacaggagggccacatgactgaatctctctcagcagtccaggcagtcatgcagaaggcccagtagagcaccgggcaggtctgagccagcatcttcaagttccaccctctgagcaagcacctagctgtgacacacctctccagagactgcactcccccccgcgccacccaccccaaaagcagataggtaatggtatacagtaaccatttctagaagtgtaagtagtatgcacccaaaataggcaaaacctgctggcctagtgatagagacaactcccagtcaggctagactggaggccttggttttataagtgttcaggtgacaagtgccacagtaggcttgatcaagtagacaggcaggcaagacaaatgcttaccaatgcaagctaatgaaatgtttcttttgcagaattcctgtcctgtgaaggaagccaaccagagtacgttggaaaacttcttggaaaggctaaagacgatcatgagagagaaatattcaaagtgttcgagctga

SEQ ID NO: 72 - DNA, 1668 bp, Homo sapiens
atgaggatgcttctgcatttgagtttgctagctcttggagctgcctacgtgtatgccatccccacagaaattcccacaagtgcattggtgaaagagaccttggcactgctttctactcatcgaactctgctgatagccaatgaggtaattttctttatgattcctacagtctgtaaagtgcataggtaatcatttgtgatggttcctttactatatatagagatctgttataaataataagattctgagcacattagtacatgggtgataactacatcaccagcaaacattctgttaaaagttatgaatgctggtgtgctgtaaaaatgattgtatttcctttcctctccagactctgaggattcctgttcctgtacataaaaatgtaagttaaattatgattcagtaaaatgatggcatgaataagtaaatttcctgttttaagctgtaaatcattagttatcattggaactatttaattttctatattttgttttcatatgggtggctgtgaatgtctgtacttataaatatgaggaatgactttttatcaagtagaatcctttaaacaagtggattaggctctttggtgatgttgttagtttgcctcccaaagagcatcgtgtcagggattctttccagaaggattccacactgagtgagaggtgcgtgctagtctccgtgcagttctgactctttctcactctaacgtgtttctgaaagtattagcaactcagaattatatttttagaaccatgatcagtagacattaaaatatataacaaatgccctatattaataatttctgcatacttaaataattatgactatatgatggtgttgtatgcatttgaatatgtcctggtcatattaaaatgtaaaatatatagttttattagtctaaatagaataaaactaccagctagaactgtagaaacacattgatatgagtttaatgtataatgcattacacttccaaaacatttttttccagttacataattaagttatatcctttataaaactcctcagtaatcatataagcttcatctactttttgaaaattttatcttaatatgtggtggtttgttgcctagaaaacaaacaaaaaactctttggagaagggaactcatgtaaataccacaaaacaaagcctaactttgtggaccaaaattgttttaataattattttttaattgatgaattaaaaagtatatatatttattgtgtacaatatgatgttttgaagtatgtatacattgcagaatggacaatggaccaaatttttataccttgtcttgattatttgcattttaaaaattttcctcatttagcaccaactgtgcactgaagaaatctttcagggaataggcacactggagagtcaaactgtgcaagggggtactgtggaaagactattcaaaaacttgtccttaataaagaaatacattgacggccaaaaagtaagttacacacattcaatggaagctatatttgtctggctgtgcctatttctatggaattgacagtttcctgtaatacctattgtcatttttcttttttcacagaaaaagtgtggagaagaaagacggagagtaaaccaattcctagactacctgcaagagtttcttggtgtaatgaacaccgagtggataatagaaagttga